SEQUENCE LISTING (193 sequences)

SEQ ID NO: 1 - DNA, 1000 bp, Saccharomyces cerevisiae
  source 1..1000: mol_type = genomic DNA
ttccttacagttgaaaacaaaatcaaatcacggtatggcatttctttacatattctcaatacctgactgacatagtatgcgctacatagttttgtcctattttttttacaccgcgatttgaagtctaaagccagagaatgaggaaaaacgaaacaaactgagatcatttgtataaggggagtcactgtacagctgccaataccaattttccttctgccaaatcaaaaaagtatccgcatacacatacacccctccctctacaatcttaattatttactgattcctttcgtatttgtccagtatgagcaatactaagcataccacttctcatcacatggaactcaaaaggatcattatccttacacttttattcatactcataatgttgatatttcgaaactcagtgtccttcaaaatgacatttcaagaactactgccacgattttacaaaaagaattcaaactcagttagtaataataacaggccctcatctattttctcggaaaacttggtggattttgatgatgttaacatggtcgataagaccagactgtttatttttttatttttcagtttcatcattactataccgtttatggtataaattagaaaagttaaagcagcatgttttcatttgaaacaaatactaatgcaataataggtacaccatctactacataattaattgatagtttccttttccgttaagtgcatgcataggacgcccataattttttaagttaaaaaagcatgtatttacctattcgggaaatttacatgacatggatgccataaggaacgaaaatgaaacatgcatgtcagaggaaaaagaacaaagcgagaggaaaaagctgttgcattaccacgtaattttgtatataaatatctgataaataaccattttatttccatccacttcttctgtcgttcatccgttcattgacatcactagagacaccagcgagctatcatcttcatacaacaataactaccaacctta

SEQ ID NO: 2 - DNA, 1000 bp, Saccharomyces cerevisiae
  source 1..1000: mol_type = genomic DNA
cgggatcgatgtggctaagaccgaggagaaactacgcacctacgaggaattaaacaagcaactgattatgaataacttggagagaagcagaacagagatagaaagtttcgaacagagacaaaaattcgaaaaggagatgaaattgaagaagcgactgttggaaagacaaatcgaagaggaggaaagaatgaacaaagagtggacaaagaaagaaatcgtcaaccggttgagtacgaccacccaggatatcaatgaaacaatcgaaggcgttaaaaatacggttaaattgaaaaaatcgtccgctagaaggaaattggaagaactaaatagagttctgaaaaacaatccatatttcaattcaaatgtgaacgtacagaattcgagactcaaagatgctgtaccgttcacgccgtttaacggtgatagagaagcacacccaaggtttacgttgaaaggttcagtatacaatgacccattcatcaaagatcttgagcacaggaaagaatttattgcgtctgggttcaacactaattatgcgtacgaaagggtgttgacagaggcatttatgggcttaggatgtgttatatccgaggagctttaaaacatcaggatagtgtgcaacgtggcataagctatgtaatcaactactttttattttctatgtacgcatatacatgcattcacgatctgtttcagtgttcagaaaaaaggcacctactgctacggttggcccatacctttattctttgttcttgttacaaacgagtgtgtaattacccaaaaaggaaatttacatgttaaatgaaacccagtaatcagaaaaaacagttaagaaacctaaaatggtagagataaagatacagattcagtggttgctgaaaatcaagtaaaaaaatgaaatagagtcttcatatataaaccgccagaaatgaattaatgagagggatctgtaactgtttctcggataaaaccaaaataagtacaaagccatcgaatagaa

SEQ ID NO: 3 - DNA, 1000 bp, Saccharomyces cerevisiae
  source 1..1000: mol_type = genomic DNA
caaatctgcctctttgttcaagcaaagaaagaacccaagaagaatcgcttggactgtcttattcagaaagcatcacaagaagggtatcaccgaagaagttgctaagaagagatctagaaaaaccgttaaggcccaaagaccaattaccggtgcttctttggacttgatcaaggaaagaagatctttgaagccagaagttagaaaggctaacagagaagaaaaattgaaggccaacaaagaaaagaagaaggctgaaaaggctgctagaaaggctgaaaaggctaagtctgctggtactcaaagttctaagttctccaagcaacaagctaagggtgctttccaaaaggttgctgctacttctcgttaagatttatgctcgaacttattatgtacaatgaatatttttcttttaaatcatttttaaatatttcaatttagtattattttctgtataattcattcgcgaggtataatctaccatccacttctctatcattatactcccatatttttttattcacaggctagacaatggtaatggtgctttagatgatttcagtggattacattcgcacaatagaataaatgactaattttcaagaatttgtttcaacctgccggcattcagttattataagttattgtttgatggtaaaacttcgccatcaccggttgctgcgtttattgcaaaattactgaaaccatgtaatttccgaatacggtaattacacgcagtttgaaatttgacatgcaagtttaacaaaaaaacttctgtccgcatctaaagcttaattaagggactgtttcataccttcagatccaatatactgaatatcaggcaattgatttcccttgtaaatatacaatataaatagttgtaagaagaggtgatatgttaagtttccattttgacgtattcctcattctagaatgattgtaagctctcaacagtcacttgtgtgccaaaatattatacctacttcatacattttcaattaag

SEQ ID NO: 4 - DNA, 1000 bp, Saccharomyces cerevisiae
  source 1..1000: mol_type = genomic DNA
aaccctgattttacctgtctcccaacttactctccattaccctacctctccactcgttaccctgtctcattcaaccgtaccactcccaaccaccatccatctctctacttactaccaccaacccaccgtccaccataaccgttaccctccaactacccgtatccaactccactaccgcttaccctaccatcgaccatgtcctactcactgtactgttgttcacccaccatattgaaacgtctacaaatgatcgtaaataatacacatatacttatcctaccactctaatcccactaccacatgccatactcaccttcacttgtattctgatcggtcatacgcacacggatgctacagtatataccatctcaaacttaccctactttcatattccactccatcacccatctctcaccatcagtaccaaatgcactcgcatcattatgcacggcacttgcctcagcggtctataccctgtgccatttacgcataacgcccatcattatccacattttaatatctatatctcattcggcgacaccaaatattgtataactgcccttaatacatacgttataccacttttacaccatatactaaccactcaatttatacacacttatgtcaatataaccacaaaatcaccactaaaatcacctaaacataaaaatattctactcttcaacaataatacataaacacactcaattgcgtatctataccaccatgacgtcattaacgtaaaagttccttaatattgcaaattgcttgaacggatgccatttcatgttgaatgtggtaacccaatagcatgatatgagtaatgctttagtattgtttcagagttgtttcagtaatgttttagacaaggagaacatatagtagcaaacctctaatccggtagtacttaagaaactacagtttctatgtacgaaagcagtaactatgtaattattacatttacatgacatataggaaggtccaataaacttactacatt

SEQ ID NO: 5 - DNA, 1000 bp, Saccharomyces cerevisiae
  source 1..1000: mol_type = genomic DNA
ttgcatgatgaagaccaccagaccaatttatcgctatgtccgattgattgttgattagtttcctggtggcgtcaaggctcgcaccagtgtatagtgttgtataatcatataaattttgaaaaataggacagtcgtctccaatgttaaaattttcaagtgtgcctctgggcagtttgtttgcattttctggtgaaaccttcgagagaaaattcacgtagtcttccgagtgaaattgtagtaattcgtctctggtagcgcttcttgtttcataaaggtccataatcttgtgcaatccataagaggaaactaggtgatccgttagcattaaccgaaacggcttcattggatgcttaacaccataatggtaatgtgaaactttggagttaaaatgatacgataccctgggagaataagcagaattaaactcaaataatggttcgttctcctttgttttcacatcataactaaatgttccagacatgttgcgtgttgttattcacttgatacctgattttaacgtacttgagtaatctggcgtcttttctataactgtacattggcgacacacctaaaactgattagaatcgtttcaagatgctttcagttcaacatttagtaacatagtaggaatatcacgtttaacgatgccccaaagaacatcgagaatcctctatatagagtataaacagggcattctacattggtatagaggtgccttaggtatatagtggtaccggtcacataaattgacgggaatatgcgtctttaggcggcttttggcgtacagtattgttctttctttcgtgcttgtgaaacttgttataaacaaagaaagagaaagatctccccggcattctggaagatcggtaaccgctggagctataggaaagggagatggataacagtaaagaaaacggaccatataagttagcattgggggttttcgaaaaaaataagaattggcattcagacttgaattcataaaagttgtaagcaggct

SEQ ID NO: 6 - DNA, 1000 bp, Saccharomyces cerevisiae
  source 1..1000: mol_type = genomic DNA
atgtgaccgtgccttttttgcacttttagagaaacagtgttaagaactgtaataactgttcaattctaagcactcaaaccgtgcttgtacatatgcttcaatggcctgctcgatagaccgatcacgtttaatttttctttaattttgccaagggaacgttcttttcgtaggcaccgcaatttatttctaatggataagttctgaattttgctatgaattagttagtagggcctactaaaatggcgtggtatttttaaagggcctttaccagccactcctcaacagaaaaccgtgctcgcgttgcagaaatacgaggtagacacgcgccttaggaacataaaaataaatatctcgcgctcacggaaggcaaccactaactccttttcatcgaaaagtgaggtgtagcttactaaagcattcctctttatccaagtcgtatatttttctttatactgctagttcattctgagcggagattcctggctaactctccaattggaaataaggtaataatgacttttcatgacgaattgggtgaaaatgcttggtgctaggccctgtgttgtggcttttttcggtaatagtttaattttccaaaaaggttcctgtgactatcttatcaaagtagcttagaccacgtcctgcacggttttgcagactgcttagggggtctcttgttgttattagcagcggaatatctttgtttactcattaaaatgcccccccgttcgttgcaaggaacgcgcgcgcacagaacttgcggatgcttccgcagaagtgcccgtaggagatttaagctagcccagcggttaatctacgcagttgcgtattcggagtccgctcacacttgcaaaaagcatataaaaagcttcatattcatctcattagtatcggttctgccatcccattatcttaggtttatgccttttctggttctcaagtcagtaacaacaaatacccatcgttgattctttttggtaagatctcgcctgtcaaagag

SEQ ID NO: 7 - DNA, 1000 bp, Saccharomyces cerevisiae
  source 1..1000: mol_type = genomic DNA
cgctagagaatttttttcggaaagatttggaatatcggcgcacggaattatgttccttgctttctctcgtggataggtgcaaataacgttaaataatatgataattccaagatccatgaatcatgtttgcaaattgggtggcatttcaaattattttaggaaaaaaatggcaaaagtttctttagttaggtgcttaattttttttgccaagggacgagtcttgggaggagtcctaaactttgtttgattatgaaagcaacccggaaggtgcggtattttacagggagtggcttttgaagggacttactagccactcgctaacgcaaaaattatcgcgttcgcgtcacagtgaagtcaggcagccgtcccctcagcaatgttgaagattcacttcgcgaccaagagcaaccaccatttttttttgctccaaaggcgggagatgcctatttctatcttttaatcgtatatgatcctgtagtgagtccaaacggctcattttgagtacggattactgtaattgctcgtgttggattgagcgaaagtagcgtaagtctcaaaaaaaaattgggttactactcataatactcctggttctttctcatgaaccattttggtcatagcctattggtttgtgccgttctgcacgttttcgtataatattaaagggatcgtgttgttattcacggcaaccattgcgtttgcataaaattactacccaatttcctacttaccaaacgcgcgcgcatagacctcgcaggctttgaagagtcctgtggaattgccgtacaaaaaatcgatctagctcaacagtcctttcagtaattacatatcggaagcccacaaataactgcaacagcgcctacaatgatataaaaggctttgtcttgattcattgctatggggggttttggacagtatgtgttgtttttatttcgagtaaatttagaaacagcaagcctcatcattgcaaatttggtaagatctcgcctaattgaaagag

SEQ ID NO: 8 - DNA, 1000 bp, Saccharomyces cerevisiae
  source 1..1000: mol_type = genomic DNA
taataatcagaactccaacaatagtcaacagggctataaccaatcctatcaaaacggtaaccaaaatagtcaaggttacaataatcaacagtaccaaggtggcaacggtggttaccaacaacaacagggacaatctggtggtgctttttcctcattggcctccatggctcaatcttacttaggtggtggacaaactcaatccaaccaacagcaatacaatcaacaaggccaaaacaaccagcagcaataccagcaacaaggccaaaactatcagcatcaacaacagggtcagcagcagcaacaaggccactccagttcattctcagctttggcttccatggcaagttcctacctgggcaataactccaattcaaattcgagttatgggggccagcaacaggctaatgagtatggtagaccgcaacagaatggtcaacagcaatccaatgagtacggaagaccgcaatacggcggaaaccagaactccaatggacagcacgaatccttcaatttttctggcaacttttctcaacagaacaataacggcaaccagaaccgctactgaacgatgattcagttcgccttctatcctttgtttacgtatttgtttatatatataactttatttttttttattaattgggctgcaagacaattttgttgtcagtgatgcctcaatccttcttttgcttccatatttaccatgtggaccctttcaaaacagagttgtatctctgcaggatgccctttttgacgtattgaatggcataattgcactgtcacttttcgcgctgtctcattttggtgcgatgatgaaacaaacatgaaacgtctgtaatttgaaacaaataacgtaattctcgggattggttttatttaaatgacaatgtaagagtggctttgtaaggtatgtgttgctcttaaaatatttggatacgacatcctttatcttttttcctttaagagcaggatataagccatcaagtttctgaaaatcaaa

SEQ ID NO: 9 - DNA, 1000 bp, Saccharomyces cerevisiae
  source 1..1000: mol_type = genomic DNA
ttgaatgatcaaccaaacgccgatatagtcactattggtgataaaattaaaatgcagctattgagaacccatcctaacaacattaaattgtctattaatggaattggtaaagatgccccaactttccaagaatctgctttgattgccgataagttattgagtgtcatgaaggccggcacttacccaaagatttccattttctacaatgacccagtgtcttccctatcttttgaaccatctgaaaaaccgatctttaacgccaagaccattgaacaatccccatcattcggcaaatttgagatcgacacggacgcaaacgttccaagagatttgtttgaatatactttggctaaccaaatgttgacagcaatggctcaaggttatgctgctgaaatttccgccagaagaaacgctatggataacgcttccaagaatgccggtgatatgatcaatcgttactctatcttgtacaacagaacaagacaagctgtcattactaatgaactggttgatattattactggtgcttcctctttgggatgataaaaaaatcaccctgcattgcctctttatttgacgttgtttttgtagaacatgaaacgaattttgacttgatgagacgaagtatatatccaaagaataccttaaatagaaaaggaaagataataaatactaaacactactatatattcaggtaaaatacaaaaattataacatttttaaaactttttttttttgaaagtccttctcgctttaggattttttcccattaagattatgatggtttcatgtatgtgtcagttaaaaaaaatatggctaagtagcaatgaaacgaaccaagaaaatgaaaacaatatatagtgctgttgaaataacaaagacattggtatatatttgtaaaatgtctgttaaatgttttttatctcaggttcttgcttgtctttggtagaagaaattatagtaaacaaacaaacaaacaaacaaaaaaaaaaaaaaaaaa

SEQ ID NO: 10 - DNA, 1000 bp, Saccharomyces cerevisiae
  source 1..1000: mol_type = genomic DNA
ctattttcgaggaccttgtcaccttgagcccaagagagccaagatttaaattttcctatgacttgatgcaaattcccaaagctaataacatgcaagacacgtacggtcaagaagacatatttgacctcttaacaggttcagacgcgactgcctcatcagtaagacccgttgaaaagaacttacctgaaaaaaacgaatatatactagcgttgaatgttagcgtcaacaacaagaagtttaatgacgcggaggccaaggcaaaaagattccttgattacgtaagggagttagaatcattttgaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaa

SEQ ID NO: 11 - DNA, 1000 bp, Saccharomyces cerevisiae
  source 1..1000: mol_type = genomic DNA
agacgcgaatttttcgaagaagtaccttcaaagaatggggtcttatcttgttttgcaagtaccactgagcaggataataatagaaatgataatatactatagtagagataacgtcgatgacttcccatactgtaattgcttttagttgtgtatttttagtgtgcaagtttctgtaaatcgattaatttttttttctttcctctttttattaaccttaatttttattttagattcctgacttcaactcaagacgcacagatattataacatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaaca

SEQ ID NO: 12 - DNA, 1000 bp, Saccharomyces cerevisiae
  source 1..1000: mol_type = genomic DNA
tccattcactggcaattcatataaacttttaaccgcgataggatcaagaattctctcgatcacgggtgcgcgaataaatgtagcaggaaaattatcacagccaggtatgaaattggaaaaatcacacttttgtgtaaaagattgagcttgtcttccaaatgcgtttcttctcacgtcgaccttcaacacacctaaagtctttactagggcactttcgttttctaattgcgcgcttaaaaagatgagaccagcacaagtaccccaaactaccttttccggattatgaacaaattcgtataaacaaggatataagcctgttctttgagcgatgagggacatcgacgtagattctcctccgggaataattaaggcgtcgcactgggctagatcctcaggagtttttacagttttgatttctatctttattccgtagtcgttttcagccaaacacctttttaaatggttggtatgctccaaaaaggcaccttgcaacgccaaaaccccaattactttcatcgactttccggacattgtactgtgggttttgtgcatactttaagatatggtttttctttctatttaacgaaatgaaataataagaaaaaaaaacctggtggcatcaacgtgatttttggaagaaagaataaaacattcaaagagttcgtgaccagttattgctgataaatgccgtttactgactcagttggaatttatggcactagttggaatagctcatcatgacataatgactcagccgacaagggtaagggttgaaagccgggctttttcactacttttcaggtagcgccgccatttcttcatgactctgacacactctgccgcaccgttagttacgtctgtaattctggctgcgaagtaccattttcgatataaataggaggacgctgagagctatggacgaagtctatggtattgttggagaaacctttaggaacgactagcaaatatacacagtactaatattcagttaattatcacg

SEQ ID NO: 13 - DNA, 1000 bp, Saccharomyces cerevisiae
  source 1..1000: mol_type = genomic DNA
cgttagtcaacgactgttggctgtcatctgttcttgttgggtgcttgcagctgcatcgacaaacatggatggatgcctctacatatatatatatatttatgtaatggacctctgaatattttactatgtaactgactataggagataccactggtgctgtagtacaatttagcacttgcaagcgttgacacagttgttgcgaaggcctctttcttgtagcacttaattggagtatgactgccatcatgtagggaaattatgaaagcataggatcaatcttgacgttgcaaagaagaagagacttgctgttccctattgatcacttaccgttattggagttcatatggtacttttttcataattttacgagtaatccctagtggtagttaatattcagcaaataaaataccagtttgagaccaaaggtaagataagagtttactgataaagacaaagaattgggatgacctagtccaagggattatagtaccttcgcgagtattgcatataagcttttagcgatacacaaacaaagcgacaccgggatttgcatatcgcttgttcctacgacgttgtcagttccataacctctgatttcccatacggattttgaaaatcagctacgaatagatagtctttttctattttcagtaaatgtgtcattgaaaaaagtttctttagaacgacgctttttgcaggctactagaaacacccacttctagtagctttacgtgacgaattgaaccgcgtgatcttgtgcactgttatcctaaatgctcaatgctcagaacatcttcttgttatctcagcctcgccacccttatcttatcatagcggccttttattttcccctttgggcaaaatacgatacagccttccaaggtcgggcaagaaagtttatgacatataaaagaaacgggtaccccaaatacgtctcttcccattgtaaaactaatcctgcatcatcacattaaggaatcattcatctaacaattacatt

SEQ ID NO: 14 - DNA, 1000 bp, Saccharomyces cerevisiae
  source 1..1000: mol_type = genomic DNA
aaccagggcaaagcaaaataaaagaaacttaatacgttatgccgtaatgaagggctaccaaaaacgataatctcaactgtaaacaggtacaatgcggacccttttgccacaaaacatacatcattcattgccggaaaaagaaagaagtgaagacagcagtgcagccagccatgttgcgccaatctaattatagatgctggtgccctgaggatgtatctggagccagccatggcatcatgcgctaccgccggatgtaaaatccgacacgcaaaagaaaaccttcgaggttgcgcacttcgcccacccatgaaccacacggttagtccaaaaggggcagttcagattccagatgcgggaattagcttgctgccaccctcacctcactaacgctgcggtgtgcggatacttcatgctatttatagacgcgcgtgtcggaatcagcacgcgcaagaaccaaatgggaaaatcggaatgggtccagaactgctttgagtgctggctattggcgtctgatttccgttttgggaatcctttgccgcgcgcccctctcaaaactccgcacaagtcccagaaagcgggaaagaaataaaacgccaccaaaaaaaaaaaaataaaagccaatcctcgaagcgtgggtggtaggccctggattatcccgtacaagtatttctcaggagtaaaaaaaccgtttgttttggaattccccatttcgcggccacctacgccgctatctttgcaacaactatctgcgataactcagcaaattttgcatattcgtgttgcagtattgcgataatgggagtcttacttccaacataacggcagaaagaaatgtgagaaaattttgcatcctttgcctccgttcaagtatataaagtcggcatgcttgataatctttctttccatcctacattgttctaattattcttattctcctttattctttcctaacataccaagaaattaatcttctgtcattcgcttaaacactatatcaata

SEQ ID NO: 15 - DNA, 421 bp, Synthetic construct
  source 1..421: mol_type = other DNA
agtgcgatccctgaattcgcatctagactgaactggccgataattgcagacgaacgggtagtccatcgttgtaggatactgtatatacacccagtagagtaggtgactactgtatgagcatacagtagtgacaaccacttactgtatataaatacagtagtggtcatcgatactgtatataaaaccagtaggcaatcgtttgtactgtatgtacatacagtatacctcgcaactactgtatataaacacagtaattaacttgtaatattctaatcaattgacaatattatttaaggacctattgttttttccaataggtggttagcaatcgtcttactttctaacttttcttaccttttacatttcagcaatatatatatatatatttcaaggatataccattctaagatctacctgcact

SEQ ID NO: 16 - DNA, 552 bp, Saccharomyces cerevisiae
  source 1..552: mol_type = genomic DNA
gttgtggaaatgtaaagagccccattatcttagcctaaaaaaaccttctctttggaactttcagtaatacgcttaactgctcattgctatattgaagtacggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaactata

SEQ ID NO: 17 - DNA, 1000 bp, Saccharomyces cerevisiae
  source 1..1000: mol_type = genomic DNA
cttcatcggtatcttcgctatattctttttagtcgaatttgcggggagaagatggatctatgctaaactaaataggcatttgaaaaacgacgacgagttacacgacatatcgccatctttaaatgagcaaccacactgggacctcatagaggacgggtctcgctggagtaaatttttcaacgggataattaagacgacaagaaggttcacgaaatctttaatgaggtctttagtcagaggcaggaacagccgtcaagggggcataagactacggtcatccccatctgcctcttcgtccagccttgccaacagggagttcttcagagacatggaggctcaaaacgaaattattgacagcctagacatcaatagtcatacaacagaaagcgaccacccaactttggctgataatagcgtataaacaatgcatactttgtacgttcaaaatacaatgcagtagatatatttatgcatattacatataatacatatcacataggaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaa

SEQ ID NO: 18 - DNA, 694 bp, Saccharomyces cerevisiae
  source 1..694: mol_type = genomic DNA
ccttgtgaaattgcaaatatggtgatttgaaacgtttcctagtgcagcaggatcacagataacgtgtaaagggcttagcagttgataatcctctctagttaagacctaaacaaaatgctgtcactaaccgtagtattaaatgacacactttggtgactttcgttaatggggatgtggtagtggccattgccaataaacaaaaagaacagggaaagaagtagaaagtgatataagtttgcttgccacttttcgtttttcacgaaaaaaacaggcgaaaaaaaatgctagacaagtacccggctgaatcacacctcgttaacagtgactttcggtgacagatacccgattgggcacccggctggtaagttatgatagaaagccaacgctgtactattggcttagctatggcaatattttgattatcagctagttttattaacgttataattagtgtaaccagtttttcatctatttcatttatttcatttatttactttaattgcagatccccctaacgcgtttaaagcttttattcactagcttatgtattttttataggaaacgcgacgcgtaacatcgcgcaaatgaaggttttgatgtattataatgaggtattcttccttatatacatcgatgaaaagcgttgacagcatacattggaaagaaataggaaacggacaccggaagaaaaaat

SEQ ID NO: 19 - DNA, 251 bp, Saccharomyces cerevisiae
  source 1..251: mol_type = genomic DNA
gcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 20 - DNA, 190 bp, Saccharomyces cerevisiae
  source 1..190: mol_type = genomic DNA
atccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaag

SEQ ID NO: 21 - DNA, 195 bp, Saccharomyces cerevisiae
  source 1..195: mol_type = genomic DNA
gtagatacgttgttgacacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctc

SEQ ID NO: 22 - DNA, 225 bp, Saccharomyces cerevisiae
  source 1..225: mol_type = genomic DNA
agcttttgattaagccttctagtccaaaaaacacgtttttttgtcatttatttcattttcttagaatagtttagtttattcattttatagtcacgaatgttttatgattctatatagggttgcaaacaagcatttttcattttatgttaaaacaatttcaggtttaccttttattctgcttgtggtgacgcgtgtatccgcccgctcttttggtcacccatgtat

SEQ ID NO: 23 - DNA, 307 bp, Saccharomyces cerevisiae
  source 1..307: mol_type = genomic DNA
atctgataacaacagtgtagatgtaacaaaatcgactttgttcccactgtacttttagctcgtacaaaatacaatatacttttcatttctccgtaaacaacatgttttcccatgtaatatccttttctatttttcgttccgttaccaactttacacatactttatatagctattcacttctatacactaaaaaactaagacaattttaattttgctgcctgccatatttcaatttgttataaattcctataatttatcctattagtagctaaaaaaagatgaatgtgaatcgaatcctaagagaatt

SEQ ID NO: 24 - protein, 431 aa, Saccharomyces cerevisiae
MSDAAPSLSNLFYDPTYNPGQSTINYTSIYGNGSTITFDELQGLVNSTVTQAIMFGVRCGAAALTLIVMWMTSRSRKTPIFIINQVSLFLIILHSALYFKYLLSNYSSVTYALTGFPQFISRGDVHVYGATNIIQVLLVASIETSLVFQIKVIFTGDNFKRIGLMLTSISFTLGIATVTMYFVSAVKGMIVTYNDVSATQDKYFNASTILLASSINFMSFVLVVKLILAIRSRRFLGLKQFDSFHILLIMSCQSLLVPSIIFILAYSLKPNQGTDVLTTVATLLAVLSLPLSSMWATAANNASKTNTITSDFTTSTDRFYPGTLSSFQTDSINNDAKSSLRSRLYDLYPRRKETTSDKHSERTFVSETADDIEKNQFYQLPTPTSSKNTRIGPFADASYKEGEVEPVDMYTPDTAADEEARKFWTEDNNNL

SEQ ID NO: 25 - protein, 350 aa, Schizosaccharomyces pombe
MRQPWWKDFTIPDASAIIHQNITIVSIVGEIEVPVSTIDAYERDRLLTGMTLSAQLALGVLTILMVCLLSSSEKRKHPVFVFNSASIVAMCLRAILNIVTICSNSYSILVNYGFILNMVHMYVHVFNILILLLAPVIIFTAEMSMMIQVRIICAHDRKTQRIMTVISACLTVLVLAFWITNMCQQIQYLLWLTPLSSKTIVGYSWPYFIAKILFAFSIIFHSGVFSYKLFRAILIRKKIGQFPFGPMQCILVISCQCLIVPATFTIIDSFIHTYDGFSSMTQCLLIISLPLSSLWASSTALKLQSMKTSSAQGETTEVSIRVDRTFDIKHTPSDDYSISDESETKKWTGS

SEQ ID NO: 26 - protein, 352 aa, Homo sapiens
MQGNGSALPNASQPVLRGDGARPSWLASALACVLIFTIVVDILGNLLVILSVYRNKKLRNAGNIFVVSLAVADLVVAIYPYPLVLMSIFNNGWNLGYLHCQVSGFLMGLSVIGSIFNITGIAINRYCYICHSLKYDKLYSSKNSLCYVLLIWLLTLAAVLPNLRAGTLQYDPRIYSCTFAQSVSSAYTIAVVVFHFLVPMIIVIFCYLRIWILVLQVRQRVKPDRKPKLKPQDFRNFVTMFVVFVLFAICWAPLNFIGLAVASDPASMVPRIPEWLFVASYYMAYFNSCLNAIIYGLLNQNFRKEYRRIIVSLCTARVFFVDSSNDVADRVKWKPSPLMTNNNVVKVDSVGS

SEQ ID NO: 27 - protein, 334 aa, Homo sapiens
MLLETQDALYVALELVIAALSVAGNVLVCAAVGTANTLQTPTNYFLVSLAAADVAVGLFAIPFAITISLGFCTDFYGCLFLACFVLVLTQSSIFSLLAVAVDRYLAICVPLRYKSLVTGTRARGVIAVLWVLAFGIGLTPFLGWNSKDSATNNCTEPWDGTTNESCCLVKCLFENVVPMSYMVYFNFFGCVLPPLLIMLVIYIKIFLVACRQLQRTELMDHSRTTLQREIHAAKSLAMIVGIFALCWLPVHAVNCVTLFQPAQGKNKPKWAMNMAILLSHANSVVNPIVYAYRNRDFRYTFHKIISRYLLCQADVKSGNGQAGVQPALGVGLGS

SEQ ID NO: 28 - protein, 465 aa, Homo sapiens
MFRQEQPLAEGSFAPMGSLQPDAGNASWNGTEAPGGGARATPYSLQVTLTLVCLAGLLMLLTVFGNVLVIIAVFTSRALKAPQNLFLVSLASADILVATLVIPFSLANEVMGYWYFGKAWCEIYLALDVLFCTSSIVHLCAISLDRYWSITQAIEYNLKRTPRRIKAIIITVWVISAVISFPPLISIEKKGGGGGPQPAEPRCEINDQKWYVISSCIGSFFAPCLIMILVYVRIYQIAKRRTRVPPSRRGPDAVAAPPGGTERRPNGLGPERSAGPGGAEAEPLPTQLNGAPGEPAPAGPRDTDALDLEESSSSDHAERPPGPRRPERGPRGKGKARASQVKPGDSLPRRGPGATGIGTPAAGPGEERVGAAKASRWRGRQNREKRFTFVLAVVIGVFVVCWFPFFFTYTLTAVGCSVPRTLFKFFFWFGYCNSSLNPVIYTIFNHDFRRAFKKILCRGDRKRIV

SEQ ID NO: 29 - protein, 388 aa, Homo sapiens
MDKLDANVSSEEGFGSVEKVVLLTFLSTVILMAILGNLLVMVAVCWDRQLRKIKTNYFIVSLAFADLLVSVLVMPFGAIELVQDIWIYGEVFCLVRTSLDVLLTTASIFHLCCISLDRYYAICCQPLVYRNKMTPLRIALMLGGCWVIPTFISFLPIMQGWNNIGIIDLIEKRKFNQNSNSTYCVFMVNKPYAITCSVVAFYIPFLLMVLAYYRIYVTAKEHAHQIQMLQRAGASSESRPQSADQHSTHRMRTETKAAKTLCIIMGCFCLCWAPFFVTNIVDPFIDYTVPGQVWTAFLWLGYINSGLNPFLYAFLNKSFRRAFLIILCCDDERYRRPSILGQTVPCSTTTINGSTHVLRDAVECGGQWESQCHPPATSPLVAAQPSDT

SEQ ID NO: 30 - protein, 356 aa, Homo sapiens
MSIPLPLLQIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 31 - protein, 472 aa, Saccharomyces cerevisiae
MGCTVSTQTIGDESDPFLQNKRANDVIEQSLQLEKQRDKNEIKLLLLGAGESGKSTVLKQLKLLHQGGFSHQERLQYAQVIRADAIQSMKILIIQARKLGIQLDCDDPINNKDLFACKRILLKAKALDYINASVAGGSDFLNDYVLKYSERYETRRRVQSTGRAKAAFDEDGNISNVKSDTDRDAETVTQNEDVDRNNSSRINLQDICKDLNQEGDDQMFVRKTSREIQGQNRRNLIHEDIAKAIKQLWNNDKGIKQCFARSNEFQLEGSAAYYFDNIEKFASPNYVCTDEDILKGRIKTTGITETEFNIGSSKFKVLDAGGQRSERKKWIHCFEGITAVLFVLAMSEYDQMLFEDERVNRMHESIMLFDTLLNSKWFKDTPFILFLNKIDLFEEKVKSMPIRKYFPDYQGRVGDAEAGLKYFEKIFLSLNKTNKPIYVKRTCATDTQTMKFVLSAVTDLIIQQNLKKSGII

SEQ ID NO: 32 - protein, 472 aa, Synthetic construct
MGCTVSTQTIGDESDPFLQNKRANDVIEQSLQLEKQRDKNEIKLLLLGAGESGKSTVLKQLKLLHQGGFSHQERLQYAQVIRADAIQSMKILIIQARKLGIQLDCDDPINNKDLFACKRILLKAKALDYINASVAGGSDFLNDYVLKYSERYETRRRVQSTGRAKAAFDEDGNISNVKSDTDRDAETVTQNEDVDRNNSSRINLQDICKDLNQEGDDQMFVRKTSREIQGQNRRNLIHEDIAKAIKQLWNNDKGIKQCFARSNEFQLEGSAAYYFDNIEKFASPNYVCTDEDILKGRIKTTGITETEFNIGSSKFKVLDAGGQRSERKKWIHCFEGITAVLFVLAMSEYDQMLFEDERVNRMHESIMLFDTLLNSKWFKDTPFILFLNKIDLFEEKVKSMPIRKYFPDYQGRVGDAEAGLKYFEKIFLSLNKTNKPIYVKRTCATDTQTMKFVLSAVTDLIIQQNLKYIGLC

SEQ ID NO: 33 - protein, 472 aa, Synthetic construct
MGCTVSTQTIGDESDPFLQNKRANDVIEQSLQLEKQRDKNEIKLLLLGAGESGKSTVLKQLKLLHQGGFSHQERLQYAQVIRADAIQSMKILIIQARKLGIQLDCDDPINNKDLFACKRILLKAKALDYINASVAGGSDFLNDYVLKYSERYETRRRVQSTGRAKAAFDEDGNISNVKSDTDRDAETVTQNEDVDRNNSSRINLQDICKDLNQEGDDQMFVRKTSREIQGQNRRNLIHEDIAKAIKQLWNNDKGIKQCFARSNEFQLEGSAAYYFDNIEKFASPNYVCTDEDILKGRIKTTGITETEFNIGSSKFKVLDAGGQRSERKKWIHCFEGITAVLFVLAMSEYDQMLFEDERVNRMHESIMLFDTLLNSKWFKDTPFILFLNKIDLFEEKVKSMPIRKYFPDYQGRVGDAEAGLKYFEKIFLSLNKTNKPIYVKRTCATDTQTMKFVLSAVTDLIIQQNLKDCGLF

SEQ ID NO: 34 - protein, 725 aa, Saccharomyces cerevisiae
MTLSFAHFTYLFTILLGLTNIALASDPETILVTITKTNDANGVVTTTVSPALVSTSTIVQAGTTTLYTTWCPLTVSTSSAAEISPSISYATTLSRFSTLTLSTEVCSHEACPSSSTLPTTTLSVTSKFTSYICPTCHTTAISSLSEVGTTTVVSSSAIEPSSASIISPVTSTLSSTTSSNPTTTSLSSTSTSPSSTSTSPSSTSTSSSSTSTSSSSTSTSSSSTSTSPSSTSTSSSLTSTSSSSTSTSQSSTSTSSSSTSTSPSSTSTSSSSTSTSPSSKSTSASSTSTSSYSTSTSPSLTSSSPTLASTSPSSTSISSTFTDSTSSLGSSIASSSTSVSLYSPSTPVYSVPSTSSNVATPSMTSSTVETTVSSQSSSEYITKSSISTTIPSFSMSTYFTTVSGVTTMYTTWCPYSSESETSTLTSMHETVTTDATVCTHESCMPSQTTSLITSSIKMSTKNVATSVSTSTVESSYACSTCAETSHSYSSVQTASSSSVTQQTTSTKSWVSSMTTSDEDFNKHATGKYHVTSSGTSTISTSVSEATSTSSIDSESQEQSSHLLSTSVLSSSSLSATLSSDSTILLFSSVSSLSVEQSPVTTLQISSTSEILQPTSSTAIATISASTSSLSATSISTPSTSVESTIESSSLTPTVSSIFLSSSSAPSSLQTSVTTTEVSTTSISIQYQTSSMVTISQYMGSGSQTRLPLGKLVFAIMAVACNVIFS

SEQ ID NO: 35 - protein, 688 aa, Saccharomyces cerevisiae
MKVQITNSRTEEILKVQANNENDEVSKATPGEVEESLRLIGDLKFFLATAPVNWQENQIIRRYYLNSGQGFVSCVFWNNLYYITGTDIVKCCLYRMQKFGREVVQKKKFEEGIFSDLRNLKCGIDATLEQPKSEFLSFLFRNMCLKTQKKQKVFFWFSVAHDKLFADALERDLKRESLNQPSTTKPVNEPALSFSYDSSSDKPLYDQLLQHLDSRRPSSTTKSDNSPPKLESENFKDNELVTVTNQPLLGVGLMDDDAPESPSQINDFIPQKLIIEPNTLELNGLTEETPHDLPKNTAKGRDEEDFPLDYFPVSVEYPTEENAFDPFPPQAFTPAAPSMPISYDNVNERDSMPVNSLLNRYPYQLSVAPTFPVPPSSSRQHFMTNRDFYSSNNNKEKLVSPSDPTSYMKYDEPVMDFDESRPNENCTNAKSHNSGQQTKQHQLYSNNFQQSYPNGMVPGYYPKMPYNPMGGDPLLDQAFYGADDFFFPPEGCDNNMLYPQTATSWNVLPPQAMQPAPTYVGRPYTPNYRSTPGSAMFPYMQSSNSMQWNTAVSPYSSRAPSTTAKNYPPSTFYSQNINQYPRRRTVGMKSSQGNVPTGNKQSVGKSAKISKPLHIKTSAYQKQYKINLETKARPSAGDEDSAHPDKNKEISMPTPDSNTLVVQSEEGGAHSLEVDTNRRSDKNLPDAT

SEQ ID NO: 36 - protein, 473 aa, Saccharomyces cerevisiae
RPSSTTKSDNSPPKLESENFKDNELVTVTNQPLLGVGLMDDDAPESPSQINDFIPQKLIIEPNTLELNGLTEETPHDLPKNTAKGRDEEDFPLDYFPVSVEYPTEENAFDPFPPQAFTPAAPSMPISYDNVNERDSMPVNSLLNRYPYQLSVAPTFPVPPSSSRQHFMTNRDFYSSNNNKEKLVSPSDPTSYMKYDEPVMDFDESRPNENCTNAKSHNSGQQTKQHQLYSNNFQQSYPNGMVPGYYPKMPYNPMGGDPLLDQAFYGADDFFFPPEGCDNNMLYPQTATSWNVLPPQAMQPAPTYVGRPYTPNYRSTPGSAMFPYMQSSNSMQWNTAVSPYSSRAPSTTAKNYPPSTFYSQNINQYPRRRTVGMKSSQGNVPTGNKQSVGKSAKISKPLHIKTSAYQKQYKINLETKARPSAGDEDSAHPDKNKEISMPTPDSNTLVVQSEEGGAHSLEVDTNRRSDKNLPDAT

SEQ ID NO: 37 - protein, 693 aa, Synthetic construct
MKALTARQQEVFDLIRDHISQTGMPPTRAEIAQRLGFRSPNAAEEHLKALARKGVIEIVSGASRGIRLLQEEEEGLPLVGRVAAGEPLLAQQHIEGHYQVDPSLFKPNADFLLRVSGMSMKDIGIMDGDLLAVHKTQDVRNGQVVVARIDDEVTVKRLKKQGNKVELLPENSEFKPIVVDLRQQSFTIEGLAVGVIRNGDWLGSGSPPKKKRKVGSGSRPSSTTKSDNSPPKLESENFKDNELVTVTNQPLLGVGLMDDDAPESPSQINDFIPQKLIIEPNTLELNGLTEETPHDLPKNTAKGRDEEDFPLDYFPVSVEYPTEENAFDPFPPQAFTPAAPSMPISYDNVNERDSMPVNSLLNRYPYQLSVAPTFPVPPSSSRQHFMTNRDFYSSNNNKEKLVSPSDPTSYMKYDEPVMDFDESRPNENCTNAKSHNSGQQTKQHQLYSNNFQQSYPNGMVPGYYPKMPYNPMGGDPLLDQAFYGADDFFFPPEGCDNNMLYPQTATSWNVLPPQAMQPAPTYVGRPYTPNYRSTPGSAMFPYMQSSNSMQWNTAVSPYSSRAPSTTAKNYPPSTFYSQNINQYPRRRTVGMKSSQGNVPTGNKQSVGKSAKISKPLHIKTSAYQKQYKINLETKARPSAGDEDSAHPDKNKEISMPTPDSNTLVVQSEEGGAHSLEVDTNRRSDKNLPDATGS

SEQ ID NO: 38 - protein, 238 aa, Synthetic construct
MSKGEELFTGVVPILVELDGDVNGHKFSVSGEGEGDATYGKLTLKFICTTGKLPVPWPTLVTTFGYGVQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 39 - protein, 232 aa, Synthetic construct
MVSELIKENMHMKLYMEGTVNNHHFKCTSEGEGKPYEGTQTMRIKAVEGGPLPFAFDILATSFMYGSKTFINHTQGIPDFFKQSFPEGFTWERVTTYEDGGVLTATQDTSLQDGCLIYNVKIRGVNFPSNGPVMQKKTLGWEASTETLYPADGGLEGRADMALKLVGGGHLICNLKTTYRSKKPAKNLKMPGVYYVDRRLERIKEADKETYVEQHEVAVARYCDLPSKLGHR

SEQ ID NO: 40 - protein, 87 aa, Saccharomyces cerevisiae
MQLLRCFSIFSVIASVLAQELTTICEQIPSPTLESTPYSLSTTTILANGKAMQGVFEYYKSVTFVSNCGSHPSTTSKGSPINTQYVF

SEQ ID NO: 41 - protein, 38 aa, Synthetic construct
MKVLIVLLAIFAALPLALAQPVISTTVGSAAEGSLDKR

SEQ ID NO: 42 - protein, 89 aa, Saccharomyces cerevisiae
MRFPSIFTAVLFAASSALAAPVNTTTEDETAQIPAEAVIGYSDLEGDFDVAVLPFSNSTNNGLLFINTTIASIAAKEEGVSLDKREAEA

SEQ ID NO: 43 - protein, 92 aa, Synthetic construct
MRQVWFSWIVGLFLCFFNVSSAAPVNTTTEDETAQIPAEAVIGYSDLEGDFDVAVLPFSNSTNNGLLFINTTIASIAAKEEGVSLDKREAEA

SEQ ID NO: 44 - protein, 22 aa, Synthetic construct
KDNSSTIEGRYPYDVPDYALQA

SEQ ID NO: 45 - protein, 9 aa, Human influenza A
YPYDVPDYA

SEQ ID NO: 46 - protein, 41 aa, Synthetic construct
SASPAAPAPASPAAPAPSAPAASPAAPAPASPAAPAPSAPA

SEQ ID NO: 47 - protein, 15 aa, Synthetic construct
GGGGSGGGGSGGGGS

SEQ ID NO: 48 - protein, 15 aa, Synthetic construct
GGGGSGGGGSGGGGS

SEQ ID NO: 49 - protein, 15 aa, Synthetic construct
GGGGSGGGGSGGGGS

SEQ ID NO: 50 - protein, 10 aa, Homo sapiens
EQKLISEEDL

SEQ ID NO: 51 - protein, 14 aa, Simian Virus 5
GKPIPNPLLGLDST

SEQ ID NO: 52 - protein, 8 aa, Synthetic construct
DYKDDDDK

SEQ ID NO: 53 - protein, 272 aa, Synthetic construct
PEEPLVVKVEEGDEAWLPCLKGTSDGPTQQLTWSRESPLKPFLKVSFGVPGLGVHVRPNAVSLVISNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDMTVAPGSTLWLSCGVPPDSVSRGPLSWTHVHPKGPKSLLSLELKDDRPARDMWVTGTRLFLPRATAQDAGKYYCHRGNLTMSFHLEVKARPVSAHTKLRTGGWK

SEQ ID NO: 54 - protein, 133 aa, Homo sapiens
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 55 - protein, 1255 aa, Homo sapiens [sequence omitted]

SEQ ID NO: 56 - protein, 630 aa, Homo sapiens
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLT

SEQ ID NO: 57 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 58 - protein, 220 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 59 - protein, 241 aa, Synthetic construct
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 60 - protein, 235 aa, Synthetic construct
VQLQQSGPELVKPGASVKMSCKASGYTFTSYVIQWVKQKPGQGLEWIGSINPYNDYTKYNEKFKGKATLTSDKSSITAYMEFSSLTSEDSALYYCARWGDGYWGRGTTLTVSSGGGGSGGGGSGGGGSDIEMTQSPAIMSASLGERVTMTCTASSSVSSSYFHWYQQKPGSSPKLCIYSTSNLASGVPPRFSGSGSTSYSLTISSMEAEDAATYFCHQYHRSPTFGGGTKLETKR

SEQ ID NO: 61 - protein, 64 aa, Synthetic construct
MATVKFKYKGEEKQVDISKIRRVRRKGKCIRFYYDLGGGKYGRGIVSEKDAPKELLQMLEKQKK

SEQ ID NO: 62 - protein, 140 aa, Synthetic construct
MQLLRCFSIFSVIASVLAQELTTICEQIPSPTLESTPYSLSTTTILANGKAMQGVFEYYKSVTFVSNCGSHPSTTSKGSPINTQYVFKDNSSTIEGRYPYDVPDYALQASGGGGSGGGGSGGGGSASMGSEQKLISEEDL

SEQ ID NO: 63 - protein, 180 aa, Synthetic construct
MQLLRCFSIFSVIASVLAQELTTICEQIPSPTLESTPYSLSTTTILANGKAMQGVFEYYKSVTFVSNCGSHPSTTSKGSPINTQYVFKDNSSTIEGRYPYDVPDYALQASASPAAPAPASPAAPAPSAPAASPAAPAPASPAAPAPSAPAGGGGSGGGGSGGGGSASMGSEQKLISEEDL

SEQ ID NO: 64 - protein, 411 aa, Synthetic construct
MQLLRCFSIFSVIASVLAQELTTICEQIPSPTLESTPYSLSTTTILANGKAMQGVFEYYKSVTFVSNCGSHPSTTSKGSPINTQYVFKDNSSTIEGRYPYDVPDYALQASGGGGSGGGGSGGGGSASPEEPLVVKVEEGDEAWLPCLKGTSDGPTQQLTWSRESPLKPFLKVSFGVPGLGVHVRPNAVSLVISNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDMTVAPGSTLWLSCGVPPDSVSRGPLSWTHVHPKGPKSLLSLELKDDRPARDMWVTGTRLFLPRATAQDAGKYYCHRGNLTMSFHLEVKARPVSAHTKLRTGGWKGSEQKLISEEDL

SEQ ID NO: 65 - protein, 451 aa, Synthetic construct
MQLLRCFSIFSVIASVLAQELTTICEQIPSPTLESTPYSLSTTTILANGKAMQGVFEYYKSVTFVSNCGSHPSTTSKGSPINTQYVFKDNSSTIEGRYPYDVPDYALQASASPAAPAPASPAAPAPSAPAASPAAPAPASPAAPAPSAPAGGGGSGGGGSGGGGSASPEEPLVVKVEEGDEAWLPCLKGTSDGPTQQLTWSRESPLKPFLKVSFGVPGLGVHVRPNAVSLVISNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDMTVAPGSTLWLSCGVPPDSVSRGPLSWTHVHPKGPKSLLSLELKDDRPARDMWVTGTRLFLPRATAQDAGKYYCHRGNLTMSFHLEVKARPVSAHTKLRTGGWKGSEQKLISEEDL

SEQ ID NO: 66 - protein, 377 aa, Synthetic construct
MQLLRCFSIFSVIASVLAQELTTICEQIPSPTLESTPYSLSTTTILANGKAMQGVFEYYKSVTFVSNCGSHPSTTSKGSPINTQYVFKDNSSTIEGRYPYDVPDYALQASGGGGSGGGGSGGGGSASMSKGEELFTGVVPILVELDGDVNGHKFSVSGEGEGDATYGKLTLKFICTTGKLPVPWPTLVTTFGYGVQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKGSEQKLISEEDL

SEQ ID NO: 67 - protein, 417 aa, Synthetic construct
MQLLRCFSIFSVIASVLAQELTTICEQIPSPTLESTPYSLSTTTILANGKAMQGVFEYYKSVTFVSNCGSHPSTTSKGSPINTQYVFKDNSSTIEGRYPYDVPDYALQASASPAAPAPASPAAPAPSAPAASPAAPAPASPAAPAPSAPAGGGGSGGGGSGGGGSASMSKGEELFTGVVPILVELDGDVNGHKFSVSGEGEGDATYGKLTLKFICTTGKLPVPWPTLVTTFGYGVQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKGSEQKLISEEDL

SEQ ID NO: 68 - protein, 175 aa, Synthetic construct
MQLLRCFSIFSVIASVLAQELTTICEQIPSPTLESTPYSLSTTTILANGKAMQGVFEYYKSVTFVSNCGSHPSTTSKGSPINTQYVFDYKDDDDKGGGGSGGGGSGGGGSPRATVKFKYKGEEKQVDISKIRRVRRKGKCIRFYYDLGGGKYGRGIVSEKDAPKELLQMLEKQKK

SEQ ID NO: 69 - protein, 211 aa, Synthetic construct
MKVLIVLLAIFAALPLALAQPVISTTVGSAAEGSLDKRQELTTICEQIPSPTLESTPYSLSTTTILANGKAMQGVFEYYKSVTFVSNCGSHPSTTSKGSPINTQYVFDYKDDDDKGGGGSGGGGSGGGGSPRATVKFKYKGEEKQVDISKIRRVRRKGKCIRFYYDLGGGKYGRGIVSEKDAPKELLQMLEKQKKPWGKPIPNPLLGLDST

SEQ ID NO: 70 - protein, 368 aa, Synthetic construct
MQLLRCFSIFSVIASVLAQELTTICEQIPSPTLESTPYSLSTTTILANGKAMQGVFEYYKSVTFVSNCGSHPSTTSKGSPINTQYVFKDNSSTIEGRYPYDVPDYALQASGGGGSGGGGSGGGGSASQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 71 - protein, 356 aa, Synthetic construct
MQLLRCFSIFSVIASVLAASQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSYPYDVPDYAGSQELTTICEQIPSPTLESTPYSLSTTTILANGKAMQGVFEYYKSVTFVSNCGSHPSTTSKGSPINTQYVF

SEQ ID NO: 72 - protein, 362 aa, Synthetic construct
MQLLRCFSIFSVIASVLAQELTTICEQIPSPTLESTPYSLSTTTILANGKAMQGVFEYYKSVTFVSNCGSHPSTTSKGSPINTQYVFKDNSSTIEGRYPYDVPDYALQASGGGGSGGGGSGGGGSASVQLQQSGPELVKPGASVKMSCKASGYTFTSYVIQWVKQKPGQGLEWIGSINPYNDYTKYNEKFKGKATLTSDKSSITAYMEFSSLTSEDSALYYCARWGDGYWGRGTTLTVSSGGGGSGGGGSGGGGSDIEMTQSPAIMSASLGERVTMTCTASSSVSSSYFHWYQQKPGSSPKLCIYSTSNLASGVPPRFSGSGSTSYSLTISSMEAEDAATYFCHQYHRSPTFGGGTKLETKR

SEQ ID NO: 73 - protein, 350 aa, Synthetic construct
MQLLRCFSIFSVIASVLAASVQLQQSGPELVKPGASVKMSCKASGYTFTSYVIQWVKQKPGQGLEWIGSINPYNDYTKYNEKFKGKATLTSDKSSITAYMEFSSLTSEDSALYYCARWGDGYWGRGTTLTVSSGGGGSGGGGSGGGGSDIEMTQSPAIMSASLGERVTMTCTASSSVSSSYFHWYQQKPGSSPKLCIYSTSNLASGVPPRFSGSGSTSYSLTISSMEAEDAATYFCHQYHRSPTFGGGTKLETKRGGGGSGGGGSGGGGSYPYDVPDYAGSQELTTICEQIPSPTLESTPYSLSTTTILANGKAMQGVFEYYKSVTFVSNCGSHPSTTSKGSPINTQYVF

SEQ ID NO: 74 - protein, 260 aa, Synthetic construct
MQLLRCFSIFSVIASVLAQELTTICEQIPSPTLESTPYSLSTTTILANGKAMQGVFEYYKSVTFVSNCGSHPSTTSKGSPINTQYVFKDNSSTIEGRYPYDVPDYALQASGGGGSGGGGSGGGGSASAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 75 - protein, 347 aa, Synthetic construct
MQLLRCFSIFSVIASVLAQELTTICEQIPSPTLESTPYSLSTTTILANGKAMQGVFEYYKSVTFVSNCGSHPSTTSKGSPINTQYVFKDNSSTIEGRYPYDVPDYALQASGGGGSGGGGSGGGGSASFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 76 - protein, 387 aa, Synthetic construct
MQLLRCFSIFSVIASVLAQELTTICEQIPSPTLESTPYSLSTTTILANGKAMQGVFEYYKSVTFVSNCGSHPSTTSKGSPINTQYVFKDNSSTIEGRYPYDVPDYALQASASPAAPAPASPAAPAPSAPAASPAAPAPASPAAPAPSAPAGGGGSGGGGSGGGGSASFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 77 - protein, 757 aa, Synthetic construct
MQLLRCFSIFSVIASVLAQELTTICEQIPSPTLESTPYSLSTTTILANGKAMQGVFEYYKSVTFVSNCGSHPSTTSKGSPINTQYVFKDNSSTIEGRYPYDVPDYALQASGGGGSGGGGSGGGGSASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLT

SEQ ID NO: 78 - protein, 797 aa, Synthetic construct
MQLLRCFSIFSVIASVLAQELTTICEQIPSPTLESTPYSLSTTTILANGKAMQGVFEYYKSVTFVSNCGSHPSTTSKGSPINTQYVFKDNSSTIEGRYPYDVPDYALQASASPAAPAPASPAAPAPSAPAASPAAPAPASPAAPAPSAPAGGGGSGGGGSGGGGSASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLT

SEQ ID NO: 79 - protein, 222 aa, Synthetic construct
MRFPSIFTAVLFAASSALAAPVNTTTEDETAQIPAEAVIGYSDLEGDFDVAVLPFSNSTNNGLLFINTTIASIAAKEEGVSLDKREAEAAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 80 - protein, 225 aa, Synthetic construct
MRQVWFSWIVGLFLCFFNVSSAAPVNTTTEDETAQIPAEAVIGYSDLEGDFDVAVLPFSNSTNNGLLFINTTIASIAAKEEGVSLDKREAEAAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 81 - DNA, 1296 bp, Saccharomyces cerevisiae
  source 1..1296: mol_type = genomic DNA
atgtctgatgcggctccttcattgagcaatctattttatgatccaacgtataatcctggtcaaagcaccattaactacacttccatatatgggaatggatctaccatcactttcgatgagttgcaaggtttagttaacagtactgttactcaggccattatgtttggtgtcagatgtggtgcagctgctttgactttgattgtcatgtggatgacatcgagaagcagaaaaacgccgattttcattatcaaccaagtttcattgtttttaatcattttgcattctgcactctattttaaatatttactgtctaattactcttcagtgacttacgctctcaccggatttcctcagttcatcagtagaggtgacgttcatgtttatggtgctacaaatataattcaagtccttcttgtggcttctattgagacttcactggtgtttcagataaaagttattttcacaggcgacaacttcaaaaggataggtttgatgctgacgtcgatatctttcactttagggattgctacagttaccatgtattttgtaagcgctgttaaaggtatgattgtgacttataatgatgttagtgccacccaagataaatacttcaatgcatccacaattttacttgcatcctcaataaactttatgtcatttgtcctggtagttaaattgattttagctattagatcaagaagattccttggtctcaagcagttcgatagtttccatattttactcataatgtcatgtcaatctttgttggttccatcgataatattcatcctcgcatacagtttgaaaccaaaccagggaacagatgtcttgactactgttgcaacattacttgctgtattgtctttaccattatcatcaatgtgggccacggctgctaataatgcatccaaaacaaacacaattacttcagactttacaacatccacagataggttttatccaggcacgctgtctagctttcaaactgatagtatcaacaacgatgctaaaagcagtctcagaagtagattatatgacctatatcctagaaggaaggaaacaacatcggataaacattcggaaagaacttttgtttctgagactgcagatgatatagagaaaaatcagttttatcagttgcccacacctacgagttcaaaaaatactaggataggaccgtttgctgatgcaagttacaaagagggagaagttgaacccgtcgacatgtacactcccgatacggcagctgatgaggaagccagaaagttctggactgaagataataataatttatga

SEQ ID NO: 82 - DNA, 1053 bp, Schizosaccharomyces pombe
  source 1..1053: mol_type = genomic DNA
atgaggcaaccttggtggaaagactttactattcccgatgcaagcgcaattattcaccaaaatattaccattgtgtctattgtcggagagattgaagtgcctgtttcaacaattgatgcatatgaaagggataggttattaactggaatgactttatctgcccaattagctttaggagtgttaaccattttaatggtttgtctgttatcatcaagcgaaaaacgtaaacaccctgtttttgtttttaattcggcaagtattgttgcaatgtgtttacgcgccattttaaatatcgtgaccatctgctcgaattcgtacagtatcctcgttaattacgggtttatcttaaacatggttcatatgtatgtgcatgtgtttaatattttaattttattattagcaccggtgatcatttttactgctgagatgtcgatgatgattcaagttaggatcatttgtgcacatgataggaaaacacaacgtatcatgactgttattagtgcctgcttaactgttttagttctcgcattttggattactaacatgtgtcaacagattcagtatctcttatggttaactcctttatcgtcgaaaaccattgttggatactcttggccctactttattgctaaaatcttatttgctttttcgattatttttcacagtggtgttttttcatacaaactctttagggccatcttaatccgcaaaaaaattgggcaatttccttttggtccgatgcagtgtattttagttatttcgtgccaatgtttaattgttcctgctacctttactatcatcgatagttttatccatacgtatgatggcttttcgtctatgactcaatgtctcctgatcatttctttaccattatcgagtttatgggcgtctagtacagctctcaaattacaatcgatgaaaacttcatctgcgcaaggagaaaccaccgaggtttcgattagggttgataggacgtttgatatcaaacatactcccagtgacgattattcgatttctgatgaatctgaaactaaaaagtggacgggatcctga

SEQ ID NO: 83 - DNA, 1059 bp, Homo sapiens
  source 1..1059: mol_type = genomic DNA
atgcaaggtaatggttctgctttgccaaatgcttctcaaccagttttgagaggtgatggtgctagaccttcttggttggcttctgctttagcttgtgttttgattttcaccatcgttgtcgatatcttgggtaacttgttggttatcttgtccgtctaccgtaacaagaaattgagaaacgctggtaacatcttcgttgtttctttggctgttgctgatttggttgttgctatctatccatatccactggtcttgatgtccatttttaacaacggttggaacttgggttacttgcattgtcaagtttctggtttcttgatgggtttgtccgttattggttccattttcaacattaccggtatcgccattaacaggtactgttacatttgccactcactgaagtacgacaagttgtactcttctaagaactccttgtgctacgttttgttgatctggttgttaactttggctgctgttttgcctaatttgagagctggtacattgcaatacgatccaagaatctactcttgtaccttcgctcaatctgtttcttctgcttacactattgccgttgtcgttttccattttttggtcccaatgattatcgtcatcttctgctacttgagaatctggattttggtcttgcaagtcagacaaagagttaagccagatagaaagccaaaattgaagccacaagacttcagaaacttcgttaccatgtttgtggttttcgttttgttcgctatttgttgggctccattgaactttattggtttggcagttgcttctgatccagcttctatggttccaagaattccagaatggttgttcgttgcttcttactacatggcttacttcaactcttgtttgaacgccattatctacggcttgttgaatcagaactttaggaaagagtacaggcgtatcatcgtttctttgtgtactgctagagttttcttcgtcgattcctctaatgatgttgccgatagagttaagtggaaaccatctccattgatgaccaacaacaatgttgtcaaggttgactccgttggatcctga

SEQ ID NO: 84 - DNA, 1005 bp, Homo sapiens
  source 1..1005: mol_type = genomic DNA
atgttgttggaaacacaagatgcgttgtatgttgcgttggaattggtaattgctgcgctatcggttgcgggaaatgttttggtttgtgctgcggttggaacggcgaataccttgcaaacgcctactaattattttttggtttcattggcagcggctgatgttgctgttggactatttgctattcctttcgctattactatttctttgggattttgtaccgatttttatggatgtctatttctagcttgttttgttttggttctaacgcaatcttcaattttttctctattggctgttgccgtagataggtatttggctatttgcgtaccgctaaggtacaagtcccttgtaacgggaactcgtgccaggggagtaatagcagtactatgggtactagctttcggaattggacttaccccttttttgggatggaattccaaggattccgctactaataattgtacagagccttgggacggaactacgaacgagtcttgttgtctagttaaatgcctattcgaaaacgttgtacctatgtcttatatggtgtactttaactttttcggatgcgtgttgcctcctttgctaatcatgttggttatttatataaaaatttttttggttgcttgtaggcaactacaacgtaccgaattgatggatcattcgaggactactctacaaagggaaattcacgccgctaaatccttggctatgatagttggaatattcgctttgtgttggctccctgttcacgcagtgaattgcgtaaccctatttcaacctgcacaaggcaagaacaaacctaaatgggccatgaacatggctatactattgtcccacgctaactccgtggtaaaccctatagtatacgcatataggaatcgtgattttcgttataccttccataagataatttcaaggtacctactatgtcaggccgacgtaaaatccggaaacggccaagcaggagtgcaacctgcactaggcgttggactcggatcctga

SEQ ID NO: 85 - DNA, 1398 bp, Homo sapiens
  source 1..1398: mol_type = genomic DNA
atgttccggcaggagcagccactcgccgagggttcctttgcccccatgggaagtcttcagcctgacgccggcaacgctagctggaatggcaccgaagcgcccggtggaggggctcgggctaccccatattctctccaggtcacactgaccctggtttgtctggccggtctcctgatgctgctgacggtctttggcaatgttttggtgataattgcggtgttcacctccagagcactcaaggctccccagaacctgttcttggtcagcctggcgtctgccgatattctggtcgccaccctcgtaattccgttttctcttgccaatgaggttatgggttactggtattttggtaaggcatggtgcgagatataccttgctctcgacgttctcttctgcaccagcagtattgtgcatctgtgcgcaattagtctggacagatattggtccatcacgcaggcaatagaatataacttgaaaaggactcctcggcgcattaaggctatcatcatcactgtctgggtcattagcgctgtgataagcttccctccacttatttccattgaaaaaaaaggaggaggcggagggccgcagcctgcggagccccgctgtgagatcaacgatcagaaatggtatgtcatctcctcctgcattgggtccttcttcgccccatgtctgatcatgatcctcgtatatgttaggatttatcagatagctaagcgaaggacccgggtccccccatccaggagaggtccagacgcagtggccgctccccccggaggcacagaaaggagacctaacggtctgggaccagagcgctccgcaggacctggaggtgcagaagccgagcccttgccgacacaactgaacggagctcccggcgaaccagccccagcaggtcctcgcgatactgacgcactggacctcgaagagtcctctagcagcgatcatgcagagcgccctccagggcctaggagacctgaaagaggacccagaggtaaaggcaaggcccgggcaagtcaagtgaaaccgggagactctctgcctcggaggggtccaggtgctactggtatagggactcctgccgccggtcccggagaagagcgggtcggcgccgctaaggcaagtcgctggcggggcagacaaaaccgggaaaaaaggtttacattcgtgctcgcggtagtgattggcgtcttcgtcgtgtgctggtttccattcttttttacctataccctgacagccgtcggctgttctgttccacggacactgttcaagttctttttctggtttggctactgtaactcaagcctgaacccggtaatctatacaatatttaaccacgattttcggagggcttttaaaaagatcctgtgcagaggcgataggaaacgaatcgtttaa

SEQ ID NO: 86 - DNA, 1167 bp, Homo sapiens
  source 1..1167: mol_type = genomic DNA
atggataaattggatgctaatgtttcttctgaagaaggttttggttctgttgaaaaagttgttttgttgacttttttgtctactgttattttgatggctattttgggtaatttgttggttatggttgctgtttgttgggatagacaattgagaaaaattaaaactaattattttattgtttctttggcttttgctgatttgttggtttctgttttggttatgccatttggtgctattgaattggttcaagatatttggatttatggtgaagttttttgtttggttagaacttctttggatgttttgttgactactgcttctatttttcatttgtgttgtatttctttggatagatattatgctatttgttgtcaaccattggtttatagaaataaaatgactccattgagaattgctttgatgttgggtggttgttgggttattccaacttttatttcttttttgccaattatgcaaggttggaataatattggtattattgatttgattgaaaaaagaaaatttaatcaaaattctaattctacttattgtgtttttatggttaataaaccatatgctattacttgttctgttgttgctttttatattccatttttgttgatggttttggcttattatagaatttatgttactgctaaagaacatgctcatcaaattcaaatgttgcaaagagctggtgcttcttctgaatctagaccacaatctgctgatcaacattctactcatagaatgagaactgaaactaaagctgctaaaactttgtgtattattatgggttgtttttgtttgtgttgggctccattttttgttactaatattgttgatccatttattgattatactgttccaggtcaagtttggactgcttttttgtggttgggttatattaattctggtttgaatccatttttgtatgcttttttgaataaatcttttagaagagcttttttgattattttgtgttgtgatgatgaaagatatagaagaccatctattttgggtcaaactgttccatgttctactactactattaatggttctactcatgttttgagagatgctgttgaatgtggtggtcaatgggaatctcaatgtcatccaccagctacttctccattggttgctgctcaaccatctgatacttga

SEQ ID NO: 87 - DNA, 1071 bp, Homo sapiens
  source 1..1071: mol_type = genomic DNA
atgtccataccgttgccgcttcttcagatatacacgtctgataattacacggaggagatgggctctggcgattatgactctatgaaggaaccctgctttagagaagagaacgcaaacttcaacaaaatttttctgcctacgatatattccataatattccttacggggatcgtcggcaatgggttagttatactggtcatgggttaccaaaagaaattgcgtagtatgaccgacaaatatcgtcttcatttgagcgttgccgacttgttatttgtcatcacactgcccttttgggctgtagacgccgtagcgaactggtactttgggaactttttgtgtaaagctgttcatgttatctacaccgttaatctatattcctccgtattgattttagcttttatatccttggatagatacctggcaatagtacatgcaacgaattcccaacgtcctagaaagttactggccgagaaagtagtgtacgtgggggtatggattccggccctgttgttgacgataccggattttatattcgctaatgtcagcgaggcagacgatagatatatatgcgacagattctaccctaacgacctgtgggtcgttgtattccaattccaacacattatggtgggactgatcttgcccggcatagtaatcttgagctgctattgtataatcataagtaagctgtcccatagtaagggacaccaaaaaaggaaagccttgaaaaccaccgtgatactaattcttgctttctttgcgtgttggctaccttactacattgggataagcattgactctttcatcctgttagagattataaagcagggatgtgagttcgagaacaccgtgcacaagtggatctccatcactgaggcgcttgcattcttccattgttgccttaaccccattctatatgccttcttaggcgctaaattcaaaacatctgctcaacatgcccttacttcagttagtagaggtagttctctgaaaattttgtctaagggtaaaaggggcggacattcctccgttagtactgaaagtgaatcatcctcttttcacagctcctga

SEQ ID NO: 88 - DNA, 1419 bp, Saccharomyces cerevisiae
  source 1..1419: mol_type = genomic DNA
atggggtgtacagtgagcacgcaaacaataggagacgaaagtgatccttttctacagaacaaaagagccaatgatgtcatcgagcaatcgttgcagctggagaaacaacgtgacaagaatgaaataaaactgttactattaggtgccggtgagtcaggtaaatcaacggttttaaaacaattaaaattattacatcaaggcggtttctcccatcaagaaaggttacagtatgctcaagtgatacgggcagatgccatacaatcaatgaaaattttgattattcaggccagaaaactaggtattcaacttgactgtgatgatccgatcaacaataaagatttgtttgcatgtaagagaatactgctaaaggctaaagctttagattatatcaacgccagtgttgccggtggttctgattttctaaatgattatgtactgaagtactcagaaaggtatgaaactaggaggcgtgttcagagtaccggacgagcaaaagctgctttcgatgaagacggaaatatttctaatgtcaaaagtgacactgacagagatgctgaaacggtgacgcaaaatgaggatgttgatagaaacaacagtagtagaattaacctacaggatatttgcaaggacttgaaccaagaaggcgatgaccagatgtttgttagaaaaacatcaagggaaattcaaggacaaaatagacgaaatcttattcacgaagacattgctaaggcaataaagcaactttggaataacgacaaaggtataaagcagtgctttgcacgttctaatgagtttcaattggagggctcagctgcatactactttgataacattgagaaatttgctagtccgaattatgtctgtacggatgaagacattttgaagggccgtataaagactacaggcattacagaaaccgaatttaacatcggctcgtccaaattcaaggttctcgacgctggtgggcagcgttctgaacgtaagaagtggattcattgtttcgaaggaattacagcagttttatttgttttagcaatgagtgaatacgaccagatgttgtttgaggatgaaagagtgaacagaatgcatgaatcaataatgctatttgacacgttattgaactctaagtggttcaaagatacaccgtttattttgtttttaaataaaattgatttgttcgaggaaaaggtaaaaagcatgcccataagaaagtactttcctgattaccagggacgtgtcggcgatgcagaagcgggtctaaaatattttgagaagatatttttgagcttgaataagacaaacaaaccaatctacgtgaaacgaacctgcgctaccgatacccaaactatgaagttcgtattgagtgcagtcaccgatctaatcatccagcaaaaccttaaaaaaagtggtattatatga

SEQ ID NO: 89 - DNA, 1419 bp, Synthetic construct
  source 1..1419: mol_type = other DNA
atggggtgtacagtgagcacgcaaacaataggagacgaaagtgatccttttctacagaacaaaagagccaatgatgtcatcgagcaatcgttgcagctggagaaacaacgtgacaagaatgaaataaaactgttactattaggtgccggtgagtcaggtaaatcaacggttttaaaacaattaaaattattacatcaaggcggtttctcccatcaagaaaggttacagtatgctcaagtgatacgggcagatgccatacaatcaatgaaaattttgattattcaggccagaaaactaggtattcaacttgactgtgatgatccgatcaacaataaagatttgtttgcatgtaagagaatactgctaaaggctaaagctttagattatatcaacgccagtgttgccggtggttctgattttctaaatgattatgtactgaagtactcagaaaggtatgaaactaggaggcgtgttcagagtaccggacgagcaaaagctgctttcgatgaagacggaaatatttctaatgtcaaaagtgacactgacagagatgctgaaacggtgacgcaaaatgaggatgttgatagaaacaacagtagtagaattaacctacaggatatttgcaaggacttgaaccaagaaggcgatgaccagatgtttgttagaaaaacatcaagggaaattcaaggacaaaatagacgaaatcttattcacgaagacattgctaaggcaataaagcaactttggaataacgacaaaggtataaagcagtgctttgcacgttctaatgagtttcaattggagggctcagctgcatactactttgataacattgagaaatttgctagtccgaattatgtctgtacggatgaagacattttgaagggccgtataaagactacaggcattacagaaaccgaatttaacatcggctcgtccaaattcaaggttctcgacgctggtgggcagcgttctgaacgtaagaagtggattcattgtttcgaaggaattacagcagttttatttgttttagcaatgagtgaatacgaccagatgttgtttgaggatgaaagagtgaacagaatgcatgaatcaataatgctatttgacacgttattgaactctaagtggttcaaagatacaccgtttattttgtttttaaataaaattgatttgttcgaggaaaaggtaaaaagcatgcccataagaaagtactttcctgattaccagggacgtgtcggcgatgcagaagcgggtctaaaatattttgagaagatatttttgagcttgaataagacaaacaaaccaatctacgtgaaacgaacctgcgctaccgatacccaaactatgaagttcgtattgagtgcagtcactgacctaatcatccagcaaaaccttaaatatattggtttgtgttga

SEQ ID NO: 90 - DNA, 1419 bp, Synthetic construct
  source 1..1419: mol_type = other DNA
atggggtgtacagtgagcacgcaaacaataggagacgaaagtgatccttttctacagaacaaaagagccaatgatgtcatcgagcaatcgttgcagctggagaaacaacgtgacaagaatgaaataaaactgttactattaggtgccggtgagtcaggtaaatcaacggttttaaaacaattaaaattattacatcaaggcggtttctcccatcaagaaaggttacagtatgctcaagtgatacgggcagatgccatacaatcaatgaaaattttgattattcaggccagaaaactaggtattcaacttgactgtgatgatccgatcaacaataaagatttgtttgcatgtaagagaatactgctaaaggctaaagctttagattatatcaacgccagtgttgccggtggttctgattttctaaatgattatgtactgaagtactcagaaaggtatgaaactaggaggcgtgttcagagtaccggacgagcaaaagctgctttcgatgaagacggaaatatttctaatgtcaaaagtgacactgacagagatgctgaaacggtgacgcaaaatgaggatgttgatagaaacaacagtagtagaattaacctacaggatatttgcaaggacttgaaccaagaaggcgatgaccagatgtttgttagaaaaacatcaagggaaattcaaggacaaaatagacgaaatcttattcacgaagacattgctaaggcaataaagcaactttggaataacgacaaaggtataaagcagtgctttgcacgttctaatgagtttcaattggagggctcagctgcatactactttgataacattgagaaatttgctagtccgaattatgtctgtacggatgaagacattttgaagggccgtataaagactacaggcattacagaaaccgaatttaacatcggctcgtccaaattcaaggttctcgacgctggtgggcagcgttctgaacgtaagaagtggattcattgtttcgaaggaattacagcagttttatttgttttagcaatgagtgaatacgaccagatgttgtttgaggatgaaagagtgaacagaatgcatgaatcaataatgctatttgacacgttattgaactctaagtggttcaaagatacaccgtttattttgtttttaaataaaattgatttgttcgaggaaaaggtaaaaagcatgcccataagaaagtactttcctgattaccagggacgtgtcggcgatgcagaagcgggtctaaaatattttgagaagatatttttgagcttgaataagacaaacaaaccaatctacgtgaaacgaacctgcgctaccgatacccaaactatgaagttcgtattgagtgcagtcactgacctaatcatccagcaaaaccttaaagattgtggtttgttttga

SEQ ID NO: 91 - DNA, 2178 bp, Saccharomyces cerevisiae
  source 1..2178: mol_type = genomic DNA
atgacattatctttcgctcattttacctacctgttcacaatattgttgggattaactaatattgccttggcatctgatccagaaacgattctagtgacgataaccaagacaaacgatgcaaatggggttgttacaactacagtttcacccgcgctagtctccacatccactatcgttcaagctggcactacgacattgtatacgacttggtgtccattgacggtatccacttcatctgctgccgaaataagtccttcaatatcgtacgctactaccctatccagatttagtactttgacattatctacagaagtctgctcccatgaggcatgtccttcgtcatcgacgttgccaaccaccaccttatctgtgacttccaagttcacttcatatatttgccctacttgtcacacaaccgctatcagctcattatccgaagtaggaactacaaccgtggtatcatccagcgccattgaaccatcaagtgcctctataatctcacctgtcacctctacactttcgagtacaacatcgtccaatccaactactacctccctaagttcgacatctacatctccaagctctacatctacatctccaagctctacatctacctcatcaagttcgacatctacctcatcaagttcgacatctacctcatcaagttcgacatctacatctccaagttcgacatccacatcttcaagtttgacatccacatcttcaagttctacatctacatcccaaagttctacatctacctcatcaagttcgacatctacatctccaagctctacatctacctcatcaagttcaacatctacatctccaagttctaaatctacttctgcaagctccacttccacttcttcatattcaacatctacatccccaagtttgacttcttcatctccaactttggcttccacttctccaagttcaacatctattagctctacttttactgattcaacttcatcccttggctcctctatagcatcttcatcaacgtctgtgtcattatacagcccatccacacctgtttactccgtcccttcgacttcgtcaaatgttgcaactccttctatgacttcttcaactgttgaaacaactgttagttcacaaagttcgtctgaatatatcaccaaatcctcaatttctactactatcccatcattttccatgtctacatatttcaccactgttagtggagtcactacaatgtatacgacatggtgtccttatagctctgaatctgagactagcacattaaccagtatgcatgaaacggttacaacagacgctacagtctgcactcacgagtcttgcatgccctcgcagacaacaagtttgattacatcttctataaaaatgtccactaaaaacgtcgcaacttctgtaagcacctcaacggttgaatcctcatatgcatgctccacatgtgctgaaacgtcacactcgtattcttccgtgcaaacagcttcatcaagttctgtaacacagcagaccacatccacaaagagttgggtaagttcaatgacaacttcggatgaagatttcaataagcacgctaccggtaagtatcatgtaacatcttcaggtacctcaaccatttcgactagtgtaagtgaagccacgagtacatcaagcattgactcagaatctcaagaacaatcatcacacttattatcgacatcggtcctttcatcctcctccttgtctgctacattatcctctgacagtactattttgctattcagttctgtatcatcactaagtgtcgaacagtcaccagttaccacacttcaaatttcttcaacatcagagattttacaacccacttcttccacagctattgctacaatatctgcctctacatcatcactttccgcaacatctatctctacaccatctacctctgtggaatcgactattgaatcttcatcattgactccgacggtatcttctattttcctctcatcatcatctgctccctcttctctacaaacatctgttaccactacagaagtttccactacttcaatctccatacaataccaaacttcatcaatggtaacaattagccaatatatgggcagtggatcgcaaacgcgtttgccattaggaaagttggtcttcgccatcatggcagttgcttgcaatgtaattttcagttaa

SEQ ID NO: 92 - DNA, 2067 bp, Saccharomyces cerevisiae
  source 1..2067: mol_type = genomic DNA
atgaaagtccaaataaccaatagtagaacagaggaaatcttaaaagttcaagctaataatgaaaacgatgaagtcagtaaagctactccgggcgaagttgaagaatcgctaaggttaatcggcgatctaaaattctttttagccacagcgccggtaaattggcaagaaaaccaaattataaggcgatactatctgaatagtggacaaggctttgtctcttgtgtattttggaacaatctatactatattacaggtactgatattgtcaaatgttgtctttacagaatgcaaaagtttgggagagaagtagttcaaaagaaaaaatttgaagagggtattttttcagatttaagaaatctcaaatgtggtatagatgcaactttagaacaaccaaagtccgaatttttgtcgtttctattcagaaatatgtgtctgaaaacccaaaaaaagcagaaagtatttttttggttcagtgtagcacatgataagttgtttgcggatgcgttggaaagagatttaaaaagagaaagtttgaatcagccttcaacgactaagcccgttaatgagcccgccttatctttttcatatgattcctcatctgataagcctctctacgatcagttacttcaacatttagattctagaagaccatctagtacaacaaaatcagataattcgcctccaaaattagaaagcgagaattttaaggataatgagttggtaacagtaactaatcagccgcttttaggcgttggcctcatggatgacgatgcgccagaatccccctctcaaattaatgattttattcctcagaaattgattatagaacccaatactctcgaattgaatggtctcacagaagaaacgcctcatgacttacccaagaataccgctaagggcagagacgaagaagattttcctctcgactattttcctgtatctgttgaataccctacggaggaaaatgcgtttgatccgttccctccacaggcttttacgccagctgccccttccatgcctatttcctatgataacgtgaatgaaagggattctatgcccgttaattctcttcttaatagatacccctatcagttatcagtggcacccactttcccagtgccaccatcatcatcgaggcaacattttatgacaaatcgggatttttattcatctaacaataacaaggaaaaattggtatctcctagcgaccctaccagctacatgaagtatgacgaaccagttatggattttgatgaatctcggccaaatgaaaactgtacaaatgcaaaatctcacaactctggccagcaaactaaacaacaccaattatattctaacaacttccagcaatcttacccaaacggaatggttccaggatactacccaaaaatgccgtataatcccatggggggggatcctctactcgatcaagccttttatggcgcggacgattttttctttccaccagaaggatgtgataacaatatgctgtatccacaaactgcaacttcatggaatgttttgccccctcaagctatgcaaccagctccaacctatgttgggagaccatacacaccgaattatagatcgacaccaggttccgcgatgttcccatacatgcaaagttcaaattccatgcagtggaacactgctgtttcaccttatagttcgagagcaccatctacaactgctaaaaactatcctcctagcacattttattctcaaaatataaatcaatacccacggcgaagaactgtgggaatgaagtcttcacaaggaaatgttccaacaggtaataaacaatctgtgggcaagtctgcaaaaatttcaaagcctctacatattaagacaagtgcttatcagaagcaatacaaaatcaacttggaaacgaaagccaggccaagtgctggtgacgaagattctgctcatcctgataagaacaaagaaatttcgatgcctactccggattccaatactttggtggtccagtcagaagaaggtggagctcattcacttgaggtagataccaatcgaaggtccgataaaaaccttccagatgcaacctga

SEQ ID NO: 93 - DNA, 1419 bp, Saccharomyces cerevisiae
  source 1..1419: mol_type = genomic DNA
agaccatctagtacaacaaaatcagataattcgcctccaaaattagaaagcgagaattttaaggataatgagttggtaacagtaactaatcagccgcttttaggcgttggcctcatggatgacgatgcgccagaatccccctctcaaattaatgattttattcctcagaaattgattatagaacccaatactctcgaattgaatggtctcacagaagaaacgcctcatgacttacccaagaataccgctaagggcagagacgaagaagattttcctctcgactattttcctgtatctgttgaataccctacggaggaaaatgcgtttgatccgttccctccacaggcttttacgccagctgccccttccatgcctatttcctatgataacgtgaatgaaagggattctatgcccgttaattctcttcttaatagatacccctatcagttatcagtggcacccactttcccagtgccaccatcatcatcgaggcaacattttatgacaaatcgggatttttattcatctaacaataacaaggaaaaattggtatctcctagcgaccctaccagctacatgaagtatgacgaaccagttatggattttgatgaatctcggccaaatgaaaactgtacaaatgcaaaatctcacaactctggccagcaaactaaacaacaccaattatattctaacaacttccagcaatcttacccaaacggaatggttccaggatactacccaaaaatgccgtataatcccatggggggggatcctctactcgatcaagccttttatggcgcggacgattttttctttccaccagaaggatgtgataacaatatgctgtatccacaaactgcaacttcatggaatgttttgccccctcaagctatgcaaccagctccaacctatgttgggagaccatacacaccgaattatagatcgacaccaggttccgcgatgttcccatacatgcaaagttcaaattccatgcagtggaacactgctgtttcaccttatagttcgagagcaccatctacaactgctaaaaactatcctcctagcacattttattctcaaaatataaatcaatacccacggcgaagaactgtgggaatgaagtcttcacaaggaaatgttccaacaggtaataaacaatctgtgggcaagtctgcaaaaatttcaaagcctctacatattaagacaagtgcttatcagaagcaatacaaaatcaacttggaaacgaaagccaggccaagtgctggtgacgaagattctgctcatcctgataagaacaaagaaatttcgatgcctactccggattccaatactttggtggtccagtcagaagaaggtggagctcattcacttgaggtagataccaatcgaaggtccgataaaaaccttccagatgcaacc

SEQ ID NO: 94 - DNA, 2082 bp, Synthetic construct
  source 1..2082: mol_type = other DNA
atgaaggctttaactgcaagacaacaggaagtttttgatttgataagagatcatatatctcaaactggaatgccaccaacaagagctgaaatagctcaaagattaggatttagatctccaaacgctgcagaagaacatttgaaagctttggcaaggaaaggtgtcatcgaaattgtctctggtgcttcaagaggtattagactattacaagaagaggaagaaggattgccattggttggtagagttgctgctggtgaaccattattagcacagcaacatattgagggtcattatcaagtagatccatctttattcaaaccaaatgctgattttttgttgagagtaagtggtatgtctatgaaagatattggtatcatggatggtgacttattggctgttcataaaactcaagatgttagaaacggtcaagttgtcgttgccagaattgatgatgaagtcactgttaaaagattgaagaaacaaggtaataaagtggaattgttaccagaaaattctgaatttaagccaattgttgttgatttaaggcaacaatcttttactattgaaggtttagctgtcggtgttatcagaaatggtgattggttaggttcaggttctcctccaaaaaaaaagagaaaagttggatctggaagtagaccatctagtacaacaaaatcagataattcgcctccaaaattagaaagcgagaattttaaggataatgagttggtaacagtaactaatcagccgcttttaggcgttggcctcatggatgacgatgcgccagaatccccctctcaaattaatgattttattcctcagaaattgattatagaacccaatactctcgaattgaatggtctaacagaagaaacgcctcatgacttacccaagaataccgctaagggcagagatgaagaagattttcctctcgactattttcctgtatctgttgaataccctacggaggaaaatgcgtttgatccgttccctccacaggcttttacgccagctgccccttccatgcctatttcctatgataacgtgaatgaaagggattctatgcccgttaattctcttcttaatagatacccctatcagttatcagtggcacccactttcccagtgccaccatcatcatcgaggcaacattttatgacaaatcgggatttttattcatctaacaataacaaggaaaaattggtatctcctagcgaccctaccagctacatgaagtatgacgaaccagttatggattttgatgaatctcggccaaatgaaaactgtacaaatgcaaaatctcacaactctggccagcaaactaaacaacaccaattatattctaacaacttccagcaatcttacccaaacggaatggttccaggatactacccaaaaatgccgtataatcccatggggggggatcctctactcgatcaagccttttatggcgcggacgattttttctttccaccagaaggatgtgataacaatatgctgtatccacaaactgcaacttcatggaatgttttgccccctcaagctatgcaaccagctccaacctatgttgggaggccatacacaccgaattatagatcgacaccaggttccgcgatgttcccatacatgcaaagttcaaattccatgcagtggaacactgctgtttcaccttatagttcgagagcaccatctacaactgctaaaaactatcctcctagcacattttattctcaaaatataaatcaatacccacggcgaagaactgtgggaatgaagtcttcacaaggaaatgttccaacaggtaataaacaatctgtgggcaagtctgcaaaaatttcaaagcctctacatattaagacaagtgcttatcagaagcaatacaaaatcaacttggaaacgaaagccaggccaagtgctggtgacgaagattctgctcatcctgataagaacaaagaaatttcgatgcctactccggattccaatactttggtggtccagtcagaagaaggtggagctcattcacttgaggtagataccaatcgaaggtccgataaaaaccttccagatgcaaccggatcctaa

SEQ ID NO: 95 - DNA, 717 bp, Synthetic construct
  source 1..717: mol_type = other DNA
atgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataa

SEQ ID NO: 96 - DNA, 699 bp, Synthetic construct
  source 1..699: mol_type = other DNA
atggtttctgaactcatcaaggaaaacatgcacatgaaactttacatggaaggtactgtgaacaatcatcattttaagtgtacatccgagggtgaaggcaaaccttacgaaggaactcaaactatgagaattaaagctgtagaaggtggaccattaccttttgcatttgatatcttggcaacatcattcatgtatgggagcaagacattcataaaccatactcaaggtataccagactttttcaaacagagttttccagagggttttacatgggaaagagtaacaacgtacgaggatggaggtgtattgacagccactcaagacacatcacttcaagatgggtgtttaatctacaatgtcaagattagaggcgtcaatttcccttctaatggtccagttatgcagaaaaagacattaggctgggaagcgtcaaccgaaaccctgtaccctgctgatggtggcctagaaggcagagctgacatggcccttaaactggttggtggagggcatctaatctgcaatttgaaaaccacttatcgttctaaaaagccagccaaaaacctaaagatgccaggtgtttactacgtcgaccgaagattagaaaggattaaagaggctgataaagagacttatgttgaacaacacgaagtggcagtggctagatactgtgatttgccatctaagttgggacacagataa

SEQ ID NO: 97 - DNA, 264 bp, Saccharomyces cerevisiae
  source 1..264: mol_type = genomic DNA
atgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgttttttga

SEQ ID NO: 98 - DNA, 114 bp, Synthetic construct
  source 1..114: mol_type = other DNA
atgaaggtgctgattgttctattggctatatttgctgccttgcccctggccctggcacagccagtcataagtacaacagtgggcagtgctgcggaaggttcactagataaaaga

SEQ ID NO: 99 - DNA, 267 bp, Saccharomyces cerevisiae
  source 1..267: mol_type = genomic DNA
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgataaaagagaggctgaagct

SEQ ID NO: 100 - DNA, 276 bp, Synthetic construct
  source 1..276: mol_type = other DNA
atgaggcaggtttggttctcttggattgtgggattgttcctatgttttttcaacgtgtcttctgctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgataaaagagaggctgaagct

SEQ ID NO: 101 - DNA, 66 bp, Synthetic construct
  source 1..66: mol_type = other DNA
aaggacaatagctcgacgattgaaggtagatacccatacgacgttccagactacgctctgcaggct

SEQ ID NO: 102 - DNA, 27 bp, Human influenza A
  source 1..27: mol_type = genomic DNA
tacccatacgacgttccagactacgct

SEQ ID NO: 103 - DNA, 123 bp, Synthetic construct
  source 1..123: mol_type = other DNA
tcagcctctccagcggcacctgctcccgcaagccctgctgcaccagctccgtcagctcctgctgcctctccagctgcacctgctccagcttctccagcagctcctgcacctagtgctcctgct

SEQ ID NO: 104 - DNA, 45 bp, Synthetic construct
  source 1..45: mol_type = other DNA
gggggtggaggctctggcggaggtgggtctggtgggggcggatct

SEQ ID NO: 105 - DNA, 45 bp, Synthetic construct
  source 1..45: mol_type = other DNA
ggcgggggaggatctggtggaggaggatcagggggaggcgggtcc

SEQ ID NO: 106 - DNA, 45 bp, Synthetic construct
  source 1..45: mol_type = other DNA
ggtggtggcggctccggcggcggtggctcaggagggggcggatca

SEQ ID NO: 107 - DNA, 30 bp, Homo sapiens
  source 1..30: mol_type = genomic DNA
gaacaaaagcttatttctgaagaggacttg

SEQ ID NO: 108 - DNA, 42 bp, Simian Virus 5
  source 1..42: mol_type = genomic DNA
ggtaaacccatacccaatcccctgctgggtcttgatagtacg

SEQ ID NO: 109 - DNA, 24 bp, Synthetic construct
  source 1..24: mol_type = other DNA
gactataaggatgacgatgacaag

SEQ ID NO: 110 - DNA, 816 bp, Synthetic construct
  source 1..816: mol_type = other DNA
cccgaggaacctctagtggtgaaggtggaagagggagatgaggcttggctcccctgcctcaaggggacctccgatggccccactcagcagctgacctggtctcgggagtccccgcttaagcccttcctaaaagtgagtttcggggtgccaggactgggcgtccacgtgaggcccaacgccgtctctcttgtcatctctaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagcagggacatgactgtggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaactggtactcggctgtttctgcctcgggccacagctcaagacgctggaaagtattattgccaccgtggcaacctgaccatgtcattccacctggaggtcaaagctcggccagtctctgctcacactaaactaagaactggtggctggaag

SEQ ID NO: 111 - DNA, 405 bp, Homo sapiens
  source 1..405: mol_type = genomic DNA
gcgccgacttctagtagtacaaaaaagacccaattgcagttggaacacttgctgctggatcttcagatgatccttaatgggataaataactataagaaccccaaactaacgagaatgctgacgttcaaattctacatgccgaaaaaagcaacagaattaaaacacttgcaatgtctagaggaggagctaaagccccttgaagaagtactaaacttggcgcagtcaaagaatttccatttgagacccagggacctaatctccaacatcaatgtcatcgtactagaactgaaaggatctgaaacaaccttcatgtgcgaatatgcagacgaaaccgcaactatagttgaatttctgaatcgttggataacattttctcagtccattatctcaacattgacgtaatag

SEQ ID NO: 112 - DNA, 3765 bp, Homo sapiens
  source 1..3765: mol_type = genomic DNA
atggaattggctgctttgtgtagatggggtttgttgttggctttgttgccaccaggtgctgcttctactcaagtttgtactggtactgatatgaaattgagattgccagcttctccagaaactcatttggatatgttgagacatttgtatcaaggttgtcaagttgttcaaggtaatttggaattgacttatttgccaactaatgcttctttgtcttttttgcaagatattcaagaagttcaaggttatgttttgattgctcataatcaagttagacaagttccattgcaaagattgagaattgttagaggtactcaattgtttgaagataattatgctttggctgttttggataatggtgatccattgaataatactactccagttactggtgcttctccaggtggtttgagagaattgcaattgagatctttgactgaaattttgaaaggtggtgttttgattcaaagaaatccacaattgtgttatcaagatactattttgtggaaagatatttttcataaaaataatcaattggctttgactttgattgatactaatagatctagagcttgtcatccatgttctccaatgtgtaaaggttctagatgttggggtgaatcttctgaagattgtcaatctttgactagaactgtttgtgctggtggttgtgctagatgtaaaggtccattgccaactgattgttgtcatgaacaatgtgctgctggttgtactggtccaaaacattctgattgtttggcttgtttgcattttaatcattctggtatttgtgaattgcattgtccagctttggttacttataatactgatacttttgaatctatgccaaatccagaaggtagatatacttttggtgcttcttgtgttactgcttgtccatataattatttgtctactgatgttggttcttgtactttggtttgtccattgcataatcaagaagttactgctgaagatggtactcaaagatgtgaaaaatgttctaaaccatgtgctagagtttgttatggtttgggtatggaacatttgagagaagttagagctgttacttctgctaatattcaagaatttgctggttgtaaaaaaatttttggttctttggcttttttgccagaatcttttgatggtgatccagcttctaatactgctccattgcaaccagaacaattgcaagtttttgaaactttggaagaaattactggttatttgtatatttctgcttggccagattctttgccagatttgtctgtttttcaaaatttgcaagttattagaggtagaattttgcataatggtgcttattctttgactttgcaaggtttgggtatttcttggttgggtttgagatctttgagagaattgggttctggtttggctttgattcatcataatactcatttgtgttttgttcatactgttccatgggatcaattgtttagaaatccacatcaagctttgttgcatactgctaatagaccagaagatgaatgtgttggtgaaggtttggcttgtcatcaattgtgtgctagaggtcattgttggggtccaggtccaactcaatgtgttaattgttctcaatttttgagaggtcaagaatgtgttgaagaatgtagagttttgcaaggtttgccaagagaatatgttaatgctagacattgtttgccatgtcatccagaatgtcaaccacaaaatggttctgttacttgttttggtccagaagctgatcaatgtgttgcttgtgctcattataaagatccaccattttgtgttgctagatgtccatctggtgttaaaccagatttgtcttatatgccaatttggaaatttccagatgaagaaggtgcttgtcaaccatgtccaattaattgtactcattcttgtgttgatttggatgataaaggttgtccagctgaacaaagagcttctccattgacttctattatttctgctgttgttggtattttgttggttgttgttttgggtgttgtttttggtattttgattaaaagaagacaacaaaaaattagaaaatatactatgagaagattgttgcaagaaactgaattggttgaaccattgactccatctggtgctatgccaaatcaagctcaaatgagaattttgaaagaaactgaattgagaaaagttaaagttttgggttctggtgcttttggtactgtttataaaggtatttggattccagatggtgaaaatgttaaaattccagttgctattaaagttttgagagaaaatacttctccaaaagctaataaagaaattttggatgaagcttatgttatggctggtgttggttctccatatgtttctagattgttgggtatttgtttgacttctactgttcaattggttactcaattgatgccatatggttgtttgttggatcatgttagagaaaatagaggtagattgggttctcaagatttgttgaattggtgtatgcaaattgctaaaggtatgtcttatttggaagatgttagattggttcatagagatttggctgctagaaatgttttggttaaatctccaaatcatgttaaaattactgattttggtttggctagattgttggatattgatgaaactgaatatcatgctgatggtggtaaagttccaattaaatggatggctttggaatctattttgagaagaagatttactcatcaatctgatgtttggtcttatggtgttactgtttgggaattgatgacttttggtgctaaaccatatgatggtattccagctagagaaattccagatttgttggaaaaaggtgaaagattgccacaaccaccaatttgtactattgatgtttatatgattatggttaaatgttggatgattgattctgaatgtagaccaagatttagagaattggtttctgaattttctagaatggctagagatccacaaagatttgttgttattcaaaatgaagatttgggtccagcttctccattggattctactttttatagatctttgttggaagatgatgatatgggtgatttggttgatgctgaagaatatttggttccacaacaaggttttttttgtccagatccagctccaggtgctggtggtatggttcatcatagacatagatcttcttctactagatctggtggtggtgatttgactttgggtttggaaccatctgaagaagaagctccaagatctccattggctccatctgaaggtgctggttctgatgtttttgatggtgatttgggtatgggtgctgctaaaggtttgcaatctttgccaactcatgatccatctccattgcaaagatattctgaagatccaactgttccattgccatctgaaactgatggttatgttgctccattgacttgttctccacaaccagaatatgttaatcaaccagatgttagaccacaaccaccatctccaagagaaggtccattgccagctgctagaccagctggtgctactttggaaagaccaaaaactttgtctccaggtaaaaatggtgttgttaaagatgtttttgcttttggtggtgctgttgaaaatccagaatatttgactccacaaggtggtgctgctccacaaccacatccaccaccagctttttctccagcttttgataatttgtattattgggatcaagatccaccagaaagaggtgctccaccatctacttttaaaggtactccaactgctgaaaatccagaatatttgggtttggatgttccagtt

SEQ ID NO: 113 - DNA, 1890 bp, Homo sapiens
  source 1..1890: mol_type = genomic DNA
actcaagtttgtactggtactgatatgaaattgagattgccagcttctccagaaactcatttggatatgttgagacatttgtatcaaggttgtcaagttgttcaaggtaatttggaattgacttatttgccaactaatgcttctttgtcttttttgcaagatattcaagaagttcaaggttatgttttgattgctcataatcaagttagacaagttccattgcaaagattgagaattgttagaggtactcaattgtttgaagataattatgctttggctgttttggataatggtgatccattgaataatactactccagttactggtgcttctccaggtggtttgagagaattgcaattgagatctttgactgaaattttgaaaggtggtgttttgattcaaagaaatccacaattgtgttatcaagatactattttgtggaaagatatttttcataaaaataatcaattggctttgactttgattgatactaatagatctagagcttgtcatccatgttctccaatgtgtaaaggttctagatgttggggtgaatcttctgaagattgtcaatctttgactagaactgtttgtgctggtggttgtgctagatgtaaaggtccattgccaactgattgttgtcatgaacaatgtgctgctggttgtactggtccaaaacattctgattgtttggcttgtttgcattttaatcattctggtatttgtgaattgcattgtccagctttggttacttataatactgatacttttgaatctatgccaaatccagaaggtagatatacttttggtgcttcttgtgttactgcttgtccatataattatttgtctactgatgttggttcttgtactttggtttgtccattgcataatcaagaagttactgctgaagatggtactcaaagatgtgaaaaatgttctaaaccatgtgctagagtttgttatggtttgggtatggaacatttgagagaagttagagctgttacttctgctaatattcaagaatttgctggttgtaaaaaaatttttggttctttggcttttttgccagaatcttttgatggtgatccagcttctaatactgctccattgcaaccagaacaattgcaagtttttgaaactttggaagaaattactggttatttgtatatttctgcttggccagattctttgccagatttgtctgtttttcaaaatttgcaagttattagaggtagaattttgcataatggtgcttattctttgactttgcaaggtttgggtatttcttggttgggtttgagatctttgagagaattgggttctggtttggctttgattcatcataatactcatttgtgttttgttcatactgttccatgggatcaattgtttagaaatccacatcaagctttgttgcatactgctaatagaccagaagatgaatgtgttggtgaaggtttggcttgtcatcaattgtgtgctagaggtcattgttggggtccaggtccaactcaatgtgttaattgttctcaatttttgagaggtcaagaatgtgttgaagaatgtagagttttgcaaggtttgccaagagaatatgttaatgctagacattgtttgccatgtcatccagaatgtcaaccacaaaatggttctgttacttgttttggtccagaagctgatcaatgtgttgcttgtgctcattataaagatccaccattttgtgttgctagatgtccatctggtgttaaaccagatttgtcttatatgccaatttggaaatttccagatgaagaaggtgcttgtcaaccatgtccaattaattgtactcattcttgtgttgatttggatgataaaggttgtccagctgaacaaagagcttctccattgact

SEQ ID NO: 114 - DNA, 870 bp, Homo sapiens
  source 1..870: mol_type = genomic DNA
atgagaatttttgctgtttttatttttatgacttattggcatttgttgaatgcttttactgttactgttccaaaagatttgtatgttgttgaatatggttctaatatgactattgaatgtaaatttccagttgaaaaacaattggatttggctgctttgattgtttattgggaaatggaagataaaaatattattcaatttgttcatggtgaagaagatttgaaagttcaacattcttcttatagacaaagagctagattgttgaaagatcaattgtctttgggtaatgctgctttgcaaattactgatgttaaattgcaagatgctggtgtttatagatgtatgatttcttatggtggtgctgattataaaagaattactgttaaagttaatgctccatataataaaattaatcaaagaattttggttgttgatccagttacttctgaacatgaattgacttgtcaagctgaaggttatccaaaagctgaagttatttggacttcttctgatcatcaagttttgtctggtaaaactactactactaattctaaaagagaagaaaaattgtttaatgttacttctactttgagaattaatactactactaatgaaattttttattgtacttttagaagattggatccagaagaaaatcatactgctgaattggttattccagaattgccattggctcatccaccaaatgaaagaactcatttggttattttgggtgctattttgttgtgtttgggtgttgctttgacttttatttttagattgagaaaaggtagaatgatggatgttaaaaaatgtggtattcaagatactaattctaaaaaacaatctgatactcatttggaagaaact

SEQ ID NO: 115 - DNA, 660 bp, Homo sapiens
  source 1..660: mol_type = genomic DNA
tttactgttactgttccaaaagatttgtatgttgttgaatatggttctaatatgactattgaatgtaaatttccagttgaaaaacaattggatttggctgctttgattgtttattgggaaatggaagataaaaatattattcaatttgttcatggtgaagaagatttgaaagttcaacattcttcttatagacaaagagctagattgttgaaagatcaattgtctttgggtaatgctgctttgcaaattactgatgttaaattgcaagatgctggtgtttatagatgtatgatttcttatggtggtgctgattataaaagaattactgttaaagttaatgctccatataataaaattaatcaaagaattttggttgttgatccagttacttctgaacatgaattgacttgtcaagctgaaggttatccaaaagctgaagttatttggacttcttctgatcatcaagttttgtctggtaaaactactactactaattctaaaagagaagaaaaattgtttaatgttacttctactttgagaattaatactactactaatgaaattttttattgtacttttagaagattggatccagaagaaaatcatactgctgaattggttattccagaattgccattggctcatccaccaaatgaaaga

SEQ ID NO: 116 - DNA, 729 bp, Synthetic construct
  source 1..729: mol_type = other DNA
cagatcgtcctaacccagtcacctgctataatgtccgcctctccgggtgagaaagttacgatgacatgctcagcatcaagtagcgtaagttacatgaattggtatcaacagaagtctggaacctcccccaagcgttggatctatgacacttcaaagctggcatctggagtgccagctcatttcagggggtcaggcagtggtacgagctacagtctgacgatctcaggaatggaagcagaagatgcagctacatattactgtcagcagtggtcatccaacccgtttactttcggttctggtaccaagctagagataaatagaggtggtggcggctccggcggcggtggctcaggagggggcggatcacaagtccaattgcaacaatcaggtgcggagttagctcgtcctggggctagcgttaagatgagctgcaaggcgagcggttacacgttcactaggtatacgatgcactgggtaaaacaaagacccgggcagggtttggaatggataggctatataaacccgtcaagaggatataccaattacaatcagaaattcaaagacaaagcaacgcttaccaccgacaagtcatcatcaacggcctacatgcaattgagtagtctaacctccgaagatagcgcggtgtattactgtgcaagatactatgacgaccactactgtctggattattggggtcaaggaacgacgcttacggtaagtagctaatag

SEQ ID NO: 117 - DNA, 711 bp, Synthetic construct
  source 1..711: mol_type = other DNA
gtacaattacaacagtctggacccgaacttgtaaaaccgggcgccagcgtaaagatgagctgtaaggcttccggttatacttttacctcctatgtcatccaatgggtgaaacaaaaacctggacagggacttgagtggataggcagtatcaacccctataacgactacacaaagtataatgagaaatttaaaggtaaagcaacccttacgagtgataaatcttcaattacagcatacatggagttcagtagcctgacgagcgaagatagcgccttatattactgcgcacgttggggcgatggttattgggggcgtggcactactttaacggtaagctctggtggtggcggctccggcggcggtggctcaggagggggcggatcagacattgaaatgacgcagtcccccgccatcatgtcagccagtcttggagaaagggtcaccatgacttgcacggcttctagtagtgtaagctcatcctacttccactggtaccagcaaaaaccaggtagttccccaaagctttgcatatacagtacgtcaaatctagcctctggagtgcccccgcgtttcagcggctccggcagcacttcatacagtcttacgataagctcaatggaggcagaggacgctgcgacttacttttgtcatcagtatcatcgttcacctacttttgggggtggcactaagcttgaaactaaaagataatag

SEQ ID NO: 118 - DNA, 198 bp, Synthetic construct
  source 1..198: mol_type = other DNA
atggcgaccgtgaaatttaaatataaaggcgaagaaaaacaggtggatattagcaaaattagacgggtgcgtcgcaagggcaaatgcattaggttttactatgatctgggcggcggcaaatatggcaggggcatagtgagcgaaaaagatgcgccgaaagaactgctgcagatgctggaaaaacagaaaaaataatag

SEQ ID NO: 119 - DNA, 426 bp, Synthetic construct
  source 1..426: mol_type = other DNA
atgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggctagtggtggtggtggttctggtggtggtggttctggtggtggtggttcagctagcatgggatccgaacaaaagcttatttctgaagaggacttgtaatag

SEQ ID NO: 120 - DNA, 546 bp, Synthetic construct
  source 1..546: mol_type = other DNA
atgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcagcctctccagcggcacctgctcccgcaagccctgctgcaccagctccgtcagctcctgctgcctctccagctgcacctgctccagcttctccagcagctcctgcacctagtgctcctgctgggggtggaggctctggcggaggtgggtctggtgggggcggatcagctagcatgggatccgaacaaaagcttatttctgaagaggacttgtaatag

SEQ ID NO: 121 - DNA, 1239 bp, Synthetic construct
  source 1..1239: mol_type = other DNA
atgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcagggggtggaggctctggcggaggtgggtctggtgggggcggatctgctagccccgaggaacctctagtggtgaaggtggaagagggagatgaggcttggctcccctgcctcaaggggacctccgatggccccactcagcagctgacctggtctcgggagtccccgcttaagcccttcctaaaagtgagtttcggggtgccaggactgggcgtccacgtgaggcccaacgccgtctctcttgtcatctctaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagcagggacatgactgtggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaactggtactcggctgtttctgcctcgggccacagctcaagacgctggaaagtattattgccaccgtggcaacctgaccatgtcattccacctggaggtcaaagctcggccagtctctgctcacactaaactaagaactggtggctggaagggatccgaacaaaagcttatttctgaagaggacttgtaatag

SEQ ID NO: 122 - DNA, 1359 bp, Synthetic construct
  source 1..1359: mol_type = other DNA
atgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcagcctctccagcggcacctgctcccgcaagccctgctgcaccagctccgtcagctcctgctgcctctccagctgcacctgctccagcttctccagcagctcctgcacctagtgctcctgctgggggtggaggctctggcggaggtgggtctggtgggggcggatctgctagccccgaggaacctctagtggtgaaggtggaagagggagatgaggcttggctcccctgcctcaaggggacctccgatggccccactcagcagctgacctggtctcgggagtccccgcttaagcccttcctaaaagtgagtttcggggtgccaggactgggcgtccacgtgaggcccaacgccgtctctcttgtcatctctaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagcagggacatgactgtggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaactggtactcggctgtttctgcctcgggccacagctcaagacgctggaaagtattattgccaccgtggcaacctgaccatgtcattccacctggaggtcaaagctcggccagtctctgctcacactaaactaagaactggtggctggaagggatccgaacaaaagcttatttctgaagaggacttgtaatag

SEQ ID NO: 123 - DNA, 1137 bp, Synthetic construct
  source 1..1137: mol_type = other DNA
atgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcagggggtggaggctctggcggaggtgggtctggtgggggcggatctgctagcatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaaggatccgaacaaaagcttatttctgaagaggacttgtaatag

SEQ ID NO: 124 - DNA, 1257 bp, Synthetic construct
  source 1..1257: mol_type = other DNA
atgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcagcctctccagcggcacctgctcccgcaagccctgctgcaccagctccgtcagctcctgctgcctctccagctgcacctgctccagcttctccagcagctcctgcacctagtgctcctgctgggggtggaggctctggcggaggtgggtctggtgggggcggatcagctagcatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaaggatccgaacaaaagcttatttctgaagaggacttgtaatag

SEQ ID NO: 125 - DNA, 531 bp, Synthetic construct
  source 1..531: mol_type = other DNA
atgcagttacttcgctgtttttcaatattttctgttattgcttcagttttagcacaggaacttactacaatttgtgaacaaatacctagtcccacgttggagtcaacaccgtactccctgtctactacaacaatacttgcgaacggtaaggccatgcaaggagtctttgaatactacaagtcagttaccttcgtatctaactgcgggagtcacccgagcaccacatccaaggggagtcctataaacactcagtatgtattcgactataaggatgacgatgacaagggcggcggaggctccggtggaggcggaagcggcggtggaggtagtcctagggcgaccgtgaaatttaaatataaaggcgaagaaaaacaggtggatattagcaaaattagacgggtgcgtcgcaagggcaaatgcattaggttttactatgatctgggcggcggcaaatatggcaggggcatagtgagcgaaaaagatgcgccgaaagaactgctgcagatgctggaaaaacagaaaaaataatag

SEQ ID NO: 126 - DNA, 639 bp, Synthetic construct
  source 1..639: mol_type = other DNA
atgaaggtgctgattgttctattggctatatttgctgccttgcccctggccctggcacagccagtcataagtacaacagtgggcagtgctgcggaaggttcactagataaaagacaggaacttactacaatttgtgaacaaatacctagtcccacgttggagtcaacaccgtactccctgtctactacaacaatacttgcgaacggtaaggccatgcaaggagtctttgaatactacaagtcagttaccttcgtatctaactgcgggagtcacccgagcaccacatccaaggggagtcctataaacactcagtatgtattcgactataaggatgacgatgacaagggcggcggaggctccggtggaggcggaagcggcggtggaggtagtcctagggcgaccgtgaaatttaaatataaaggcgaagaaaaacaggtggatattagcaaaattagacgggtgcgtcgcaagggcaaatgcattaggttttactatgatctgggcggcggcaaatatggcaggggcatagtgagcgaaaaagatgcgccgaaagaactgctgcagatgctggaaaaacagaaaaaaccatggggtaaacccatacccaatcccctgctgggtcttgatagtacgtaatag

SEQ ID NO: 127 - DNA, 1110 bp, Synthetic construct
  source 1..1110: mol_type = other DNA
atgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcaggcgggggaggatctggtggaggaggatcagggggaggcgggtccgctagccagatcgtcctaacccagtcacctgctataatgtccgcctctccgggtgagaaagttacgatgacatgctcagcatcaagtagcgtaagttacatgaattggtatcaacagaagtctggaacctcccccaagcgttggatctatgacacttcaaagctggcatctggagtgccagctcatttcagggggtcaggcagtggtacgagctacagtctgacgatctcaggaatggaagcagaagatgcagctacatattactgtcagcagtggtcatccaacccgtttactttcggttctggtaccaagctagagataaatagaggtggtggcggctccggcggcggtggctcaggagggggcggatcacaagtccaattgcaacaatcaggtgcggagttagctcgtcctggggctagcgttaagatgagctgcaaggcgagcggttacacgttcactaggtatacgatgcactgggtaaaacaaagacccgggcagggtttggaatggataggctatataaacccgtcaagaggatataccaattacaatcagaaattcaaagacaaagcaacgcttaccaccgacaagtcatcatcaacggcctacatgcaattgagtagtctaacctccgaagatagcgcggtgtattactgtgcaagatactatgacgaccactactgtctggattattggggtcaaggaacgacgcttacggtaagtagctaatag

SEQ ID NO: 128 - DNA, 1074 bp, Synthetic construct
  source 1..1074: mol_type = other DNA
atgcagttacttcgctgtttttcaatattttctgttattgcttcagttttagcagctagccagatcgtcctaacccagtcacctgctataatgtccgcctctccgggtgagaaagttacgatgacatgctcagcatcaagtagcgtaagttacatgaattggtatcaacagaagtctggaacctcccccaagcgttggatctatgacacttcaaagctggcatctggagtgccagctcatttcagggggtcaggcagtggtacgagctacagtctgacgatctcaggaatggaagcagaagatgcagctacatattactgtcagcagtggtcatccaacccgtttactttcggttctggtaccaagctagagataaatagaggtggtggcggctccggcggcggtggctcaggagggggcggatcacaagtccaattgcaacaatcaggtgcggagttagctcgtcctggggctagcgttaagatgagctgcaaggcgagcggttacacgttcactaggtatacgatgcactgggtaaaacaaagacccgggcagggtttggaatggataggctatataaacccgtcaagaggatataccaattacaatcagaaattcaaagacaaagcaacgcttaccaccgacaagtcatcatcaacggcctacatgcaattgagtagtctaacctccgaagatagcgcggtgtattactgtgcaagatactatgacgaccactactgtctggattattggggtcaaggaacgacgcttacggtaagtagcggcgggggaggatctggtggaggaggatcagggggaggcgggtcctacccatacgacgttccagactacgctggatcccaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgttttttaatag

SEQ ID NO: 129 - DNA, 1092 bp, Synthetic construct
  source 1..1092: mol_type = other DNA
atgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcaggcgggggaggatctggtggaggaggatcagggggaggcgggtccgctagcgtacaattacaacagtctggacccgaacttgtaaaaccgggcgccagcgtaaagatgagctgtaaggcttccggttatacttttacctcctatgtcatccaatgggtgaaacaaaaacctggacagggacttgagtggataggcagtatcaacccctataacgactacacaaagtataatgagaaatttaaaggtaaagcaacccttacgagtgataaatcttcaattacagcatacatggagttcagtagcctgacgagcgaagatagcgccttatattactgcgcacgttggggcgatggttattgggggcgtggcactactttaacggtaagctctggtggtggcggctccggcggcggtggctcaggagggggcggatcagacattgaaatgacgcagtcccccgccatcatgtcagccagtcttggagaaagggtcaccatgacttgcacggcttctagtagtgtaagctcatcctacttccactggtaccagcaaaaaccaggtagttccccaaagctttgcatatacagtacgtcaaatctagcctctggagtgcccccgcgtttcagcggctccggcagcacttcatacagtcttacgataagctcaatggaggcagaggacgctgcgacttacttttgtcatcagtatcatcgttcacctacttttgggggtggcactaagcttgaaactaaaagataatag

SEQ ID NO: 130 - DNA, 1056 bp, Synthetic construct
  source 1..1056: mol_type = other DNA
atgcagttacttcgctgtttttcaatattttctgttattgcttcagttttagcagctagcgtacaattacaacagtctggacccgaacttgtaaaaccgggcgccagcgtaaagatgagctgtaaggcttccggttatacttttacctcctatgtcatccaatgggtgaaacaaaaacctggacagggacttgagtggataggcagtatcaacccctataacgactacacaaagtataatgagaaatttaaaggtaaagcaacccttacgagtgataaatcttcaattacagcatacatggagttcagtagcctgacgagcgaagatagcgccttatattactgcgcacgttggggcgatggttattgggggcgtggcactactttaacggtaagctctggtggtggcggctccggcggcggtggctcaggagggggcggatcagacattgaaatgacgcagtcccccgccatcatgtcagccagtcttggagaaagggtcaccatgacttgcacggcttctagtagtgtaagctcatcctacttccactggtaccagcaaaaaccaggtagttccccaaagctttgcatatacagtacgtcaaatctagcctctggagtgcccccgcgtttcagcggctccggcagcacttcatacagtcttacgataagctcaatggaggcagaggacgctgcgacttacttttgtcatcagtatcatcgttcacctacttttgggggtggcactaagcttgaaactaaaagaggcgggggaggatctggtggaggaggatcagggggaggcgggtcctacccatacgacgttccagactacgctggatcccaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgttttttaatag

SEQ ID NO: 131 - DNA, 786 bp, Synthetic construct
  source 1..786: mol_type = other DNA
atgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcaggcgggggaggatctggtggaggaggatcagggggaggcgggtccgctagcgcgccgacttctagtagtacaaaaaagacccaattgcagttggaacacttgctgctggatcttcagatgatccttaatgggataaataactataagaaccccaaactaacgagaatgctgacgttcaaattctacatgccgaaaaaagcaacagaattaaaacacttgcaatgtctagaggaggagctaaagccccttgaagaagtactaaacttggcgcagtcaaagaatttccatttgagacccagggacctaatctccaacatcaatgtcatcgtactagaactgaaaggatctgaaacaaccttcatgtgcgaatatgcagacgaaaccgcaactatagttgaatttctgaatcgttggataacattttctcagtccattatctcaacattgacgtaatag

SEQ ID NO: 132 - DNA, 1047 bp, Synthetic construct
  source 1..1047: mol_type = other DNA
atgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcaggcgggggaggatctggtggaggaggatcagggggaggcgggtccgctagctttactgttactgttccaaaagatttgtatgttgttgaatatggttctaatatgactattgaatgtaaatttccagttgaaaaacaattggatttggctgctttgattgtttattgggaaatggaagataaaaatattattcaatttgttcatggtgaagaagatttgaaagttcaacattcttcttatagacaaagagctagattgttgaaagatcaattgtctttgggtaatgctgctttgcaaattactgatgttaaattgcaagatgctggtgtttatagatgtatgatttcttatggtggtgctgattataaaagaattactgttaaagttaatgctccatataataaaattaatcaaagaattttggttgttgatccagttacttctgaacatgaattgacttgtcaagctgaaggttatccaaaagctgaagttatttggacttcttctgatcatcaagttttgtctggtaaaactactactactaattctaaaagagaagaaaaattgtttaatgttacttctactttgagaattaatactactactaatgaaattttttattgtacttttagaagattggatccagaagaaaatcatactgctgaattggttattccagaattgccattggctcatccaccaaatgaaagataatag

SEQ ID NO: 133 - DNA, 1167 bp, Synthetic construct
  source 1..1167: mol_type = other DNA
atgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcagcctctccagcggcacctgctcccgcaagccctgctgcaccagctccgtcagctcctgctgcctctccagctgcacctgctccagcttctccagcagctcctgcacctagtgctcctgctgggggtggaggctctggcggaggtgggtctggtgggggcggatctgctagctttactgttactgttccaaaagatttgtatgttgttgaatatggttctaatatgactattgaatgtaaatttccagttgaaaaacaattggatttggctgctttgattgtttattgggaaatggaagataaaaatattattcaatttgttcatggtgaagaagatttgaaagttcaacattcttcttatagacaaagagctagattgttgaaagatcaattgtctttgggtaatgctgctttgcaaattactgatgttaaattgcaagatgctggtgtttatagatgtatgatttcttatggtggtgctgattataaaagaattactgttaaagttaatgctccatataataaaattaatcaaagaattttggttgttgatccagttacttctgaacatgaattgacttgtcaagctgaaggttatccaaaagctgaagttatttggacttcttctgatcatcaagttttgtctggtaaaactactactactaattctaaaagagaagaaaaattgtttaatgttacttctactttgagaattaatactactactaatgaaattttttattgtacttttagaagattggatccagaagaaaatcatactgctgaattggttattccagaattgccattggctcatccaccaaatgaaagataatag

SEQ ID NO: 134 - DNA, 2277 bp, Synthetic construct
  source 1..2277: mol_type = other DNA
atgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcaggcgggggaggatctggtggaggaggatcagggggaggcgggtccgctagcactcaagtttgtactggtactgatatgaaattgagattgccagcttctccagaaactcatttggatatgttgagacatttgtatcaaggttgtcaagttgttcaaggtaatttggaattgacttatttgccaactaatgcttctttgtcttttttgcaagatattcaagaagttcaaggttatgttttgattgctcataatcaagttagacaagttccattgcaaagattgagaattgttagaggtactcaattgtttgaagataattatgctttggctgttttggataatggtgatccattgaataatactactccagttactggtgcttctccaggtggtttgagagaattgcaattgagatctttgactgaaattttgaaaggtggtgttttgattcaaagaaatccacaattgtgttatcaagatactattttgtggaaagatatttttcataaaaataatcaattggctttgactttgattgatactaatagatctagagcttgtcatccatgttctccaatgtgtaaaggttctagatgttggggtgaatcttctgaagattgtcaatctttgactagaactgtttgtgctggtggttgtgctagatgtaaaggtccattgccaactgattgttgtcatgaacaatgtgctgctggttgtactggtccaaaacattctgattgtttggcttgtttgcattttaatcattctggtatttgtgaattgcattgtccagctttggttacttataatactgatacttttgaatctatgccaaatccagaaggtagatatacttttggtgcttcttgtgttactgcttgtccatataattatttgtctactgatgttggttcttgtactttggtttgtccattgcataatcaagaagttactgctgaagatggtactcaaagatgtgaaaaatgttctaaaccatgtgctagagtttgttatggtttgggtatggaacatttgagagaagttagagctgttacttctgctaatattcaagaatttgctggttgtaaaaaaatttttggttctttggcttttttgccagaatcttttgatggtgatccagcttctaatactgctccattgcaaccagaacaattgcaagtttttgaaactttggaagaaattactggttatttgtatatttctgcttggccagattctttgccagatttgtctgtttttcaaaatttgcaagttattagaggtagaattttgcataatggtgcttattctttgactttgcaaggtttgggtatttcttggttgggtttgagatctttgagagaattgggttctggtttggctttgattcatcataatactcatttgtgttttgttcatactgttccatgggatcaattgtttagaaatccacatcaagctttgttgcatactgctaatagaccagaagatgaatgtgttggtgaaggtttggcttgtcatcaattgtgtgctagaggtcattgttggggtccaggtccaactcaatgtgttaattgttctcaatttttgagaggtcaagaatgtgttgaagaatgtagagttttgcaaggtttgccaagagaatatgttaatgctagacattgtttgccatgtcatccagaatgtcaaccacaaaatggttctgttacttgttttggtccagaagctgatcaatgtgttgcttgtgctcattataaagatccaccattttgtgttgctagatgtccatctggtgttaaaccagatttgtcttatatgccaatttggaaatttccagatgaagaaggtgcttgtcaaccatgtccaattaattgtactcattcttgtgttgatttggatgataaaggttgtccagctgaacaaagagcttctccattgacttaatag

SEQ ID NO: 135 - DNA, 2397 bp, Synthetic construct
  source 1..2397: mol_type = other DNA
atgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcagcctctccagcggcacctgctcccgcaagccctgctgcaccagctccgtcagctcctgctgcctctccagctgcacctgctccagcttctccagcagctcctgcacctagtgctcctgctgggggtggaggctctggcggaggtgggtctggtgggggcggatctgctagcactcaagtttgtactggtactgatatgaaattgagattgccagcttctccagaaactcatttggatatgttgagacatttgtatcaaggttgtcaagttgttcaaggtaatttggaattgacttatttgccaactaatgcttctttgtcttttttgcaagatattcaagaagttcaaggttatgttttgattgctcataatcaagttagacaagttccattgcaaagattgagaattgttagaggtactcaattgtttgaagataattatgctttggctgttttggataatggtgatccattgaataatactactccagttactggtgcttctccaggtggtttgagagaattgcaattgagatctttgactgaaattttgaaaggtggtgttttgattcaaagaaatccacaattgtgttatcaagatactattttgtggaaagatatttttcataaaaataatcaattggctttgactttgattgatactaatagatctagagcttgtcatccatgttctccaatgtgtaaaggttctagatgttggggtgaatcttctgaagattgtcaatctttgactagaactgtttgtgctggtggttgtgctagatgtaaaggtccattgccaactgattgttgtcatgaacaatgtgctgctggttgtactggtccaaaacattctgattgtttggcttgtttgcattttaatcattctggtatttgtgaattgcattgtccagctttggttacttataatactgatacttttgaatctatgccaaatccagaaggtagatatacttttggtgcttcttgtgttactgcttgtccatataattatttgtctactgatgttggttcttgtactttggtttgtccattgcataatcaagaagttactgctgaagatggtactcaaagatgtgaaaaatgttctaaaccatgtgctagagtttgttatggtttgggtatggaacatttgagagaagttagagctgttacttctgctaatattcaagaatttgctggttgtaaaaaaatttttggttctttggcttttttgccagaatcttttgatggtgatccagcttctaatactgctccattgcaaccagaacaattgcaagtttttgaaactttggaagaaattactggttatttgtatatttctgcttggccagattctttgccagatttgtctgtttttcaaaatttgcaagttattagaggtagaattttgcataatggtgcttattctttgactttgcaaggtttgggtatttcttggttgggtttgagatctttgagagaattgggttctggtttggctttgattcatcataatactcatttgtgttttgttcatactgttccatgggatcaattgtttagaaatccacatcaagctttgttgcatactgctaatagaccagaagatgaatgtgttggtgaaggtttggcttgtcatcaattgtgtgctagaggtcattgttggggtccaggtccaactcaatgtgttaattgttctcaatttttgagaggtcaagaatgtgttgaagaatgtagagttttgcaaggtttgccaagagaatatgttaatgctagacattgtttgccatgtcatccagaatgtcaaccacaaaatggttctgttacttgttttggtccagaagctgatcaatgtgttgcttgtgctcattataaagatccaccattttgtgttgctagatgtccatctggtgttaaaccagatttgtcttatatgccaatttggaaatttccagatgaagaaggtgcttgtcaaccatgtccaattaattgtactcattcttgtgttgatttggatgataaaggttgtccagctgaacaaagagcttctccattgacttaatag

SEQ ID NO: 136 - DNA, 672 bp, Synthetic construct
  source 1..672: mol_type = other DNA
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgataaaagagaggctgaagctgcgccgacttctagtagtacaaaaaagacccaattgcagttggaacacttgctgctggatcttcagatgatccttaatgggataaataactataagaaccccaaactaacgagaatgctgacgttcaaattctacatgccgaaaaaagcaacagaattaaaacacttgcaatgtctagaggaggagctaaagccccttgaagaagtactaaacttggcgcagtcaaagaatttccatttgagacccagggacctaatctccaacatcaatgtcatcgtactagaactgaaaggatctgaaacaaccttcatgtgcgaatatgcagacgaaaccgcaactatagttgaatttctgaatcgttggataacattttctcagtccattatctcaacattgacgtaatag

SEQ ID NO: 137 - DNA, 681 bp, Synthetic construct
  source 1..681: mol_type = other DNA
atgaggcaggtttggttctcttggattgtgggattgttcctatgttttttcaacgtgtcttctgctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgataaaagagaggctgaagctgcgccgacttctagtagtacaaaaaagacccaattgcagttggaacacttgctgctggatcttcagatgatccttaatgggataaataactataagaaccccaaactaacgagaatgctgacgttcaaattctacatgccgaaaaaagcaacagaattaaaacacttgcaatgtctagaggaggagctaaagccccttgaagaagtactaaacttggcgcagtcaaagaatttccatttgagacccagggacctaatctccaacatcaatgtcatcgtactagaactgaaaggatctgaaacaaccttcatgtgcgaatatgcagacgaaaccgcaactatagttgaatttctgaatcgttggataacattttctcagtccattatctcaacattgacgtaatag

SEQ ID NO: 138 - DNA, 1701 bp, Synthetic construct
  source 1..1701: mol_type = other DNA
agacgcgaatttttcgaagaagtaccttcaaagaatggggtcttatcttgttttgcaagtaccactgagcaggataataatagaaatgataatatactatagtagagataacgtcgatgacttcccatactgtaattgcttttagttgtgtatttttagtgtgcaagtttctgtaaatcgattaatttttttttctttcctctttttattaaccttaatttttattttagattcctgacttcaactcaagacgcacagatattataacatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaacctgcactaaaacaatgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggctagtggtggtggtggttctggtggtggtggttctggtggtggtggttcagctagcatgggatccgaacaaaagcttatttctgaagaggacttgtaatagaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 139 - DNA, 1821 bp, Synthetic construct
  source 1..1821: mol_type = other DNA
agacgcgaatttttcgaagaagtaccttcaaagaatggggtcttatcttgttttgcaagtaccactgagcaggataataatagaaatgataatatactatagtagagataacgtcgatgacttcccatactgtaattgcttttagttgtgtatttttagtgtgcaagtttctgtaaatcgattaatttttttttctttcctctttttattaaccttaatttttattttagattcctgacttcaactcaagacgcacagatattataacatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaacctgcactaaaacaatgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcagcctctccagcggcacctgctcccgcaagccctgctgcaccagctccgtcagctcctgctgcctctccagctgcacctgctccagcttctccagcagctcctgcacctagtgctcctgctgggggtggaggctctggcggaggtgggtctggtgggggcggatcagctagcatgggatccgaacaaaagcttatttctgaagaggacttgtaatagaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 140 - DNA, 2634 bp, Synthetic construct
  source 1..2634: mol_type = other DNA
ttccttacagttgaaaacaaaatcaaatcacggtatggcatttctttacatattctcaatacctgactgacatagtatgcgctacatagttttgtcctattttttttacaccgcgatttgaagtctaaagccagagaatgaggaaaaacgaaacaaactgagatcatttgtataaggggagtcactgtacagctgccaataccaattttccttctgccaaatcaaaaaagtatccgcatacacatacacccctccctctacaatcttaattatttactgattcctttcgtatttgtccagtatgagcaatactaagcataccacttctcatcacatggaactcaaaaggatcattatccttacacttttattcatactcataatgttgatatttcgaaactcagtgtccttcaaaatgacatttcaagaactactgccacgattttacaaaaagaattcaaactcagttagtaataataacaggccctcatctattttctcggaaaacttggtggattttgatgatgttaacatggtcgataagaccagactgtttatttttttatttttcagtttcatcattactataccgtttatggtataaattagaaaagttaaagcagcatgttttcatttgaaacaaatactaatgcaataataggtacaccatctactacataattaattgatagtttccttttccgttaagtgcatgcataggacgcccataattttttaagttaaaaaagcatgtatttacctattcgggaaatttacatgacatggatgccataaggaacgaaaatgaaacatgcatgtcagaggaaaaagaacaaagcgagaggaaaaagctgttgcattaccacgtaattttgtatataaatatctgataaataaccattttatttccatccacttcttctgtcgttcatccgttcattgacatcactagagacaccagcgagctatcatcttcatacaacaataactaccaaccttaacctgcactaaaacaatgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcagcctctccagcggcacctgctcccgcaagccctgctgcaccagctccgtcagctcctgctgcctctccagctgcacctgctccagcttctccagcagctcctgcacctagtgctcctgctgggggtggaggctctggcggaggtgggtctggtgggggcggatctgctagccccgaggaacctctagtggtgaaggtggaagagggagatgaggcttggctcccctgcctcaaggggacctccgatggccccactcagcagctgacctggtctcgggagtccccgcttaagcccttcctaaaagtgagtttcggggtgccaggactgggcgtccacgtgaggcccaacgccgtctctcttgtcatctctaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagcagggacatgactgtggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaactggtactcggctgtttctgcctcgggccacagctcaagacgctggaaagtattattgccaccgtggcaacctgaccatgtcattccacctggaggtcaaagctcggccagtctctgctcacactaaactaagaactggtggctggaagggatccgaacaaaagcttatttctgaagaggacttgtaatagaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 141 - DNA, 2634 bp, Synthetic construct
  source 1..2634: mol_type = other DNA
taataatcagaactccaacaatagtcaacagggctataaccaatcctatcaaaacggtaaccaaaatagtcaaggttacaataatcaacagtaccaaggtggcaacggtggttaccaacaacaacagggacaatctggtggtgctttttcctcattggcctccatggctcaatcttacttaggtggtggacaaactcaatccaaccaacagcaatacaatcaacaaggccaaaacaaccagcagcaataccagcaacaaggccaaaactatcagcatcaacaacagggtcagcagcagcaacaaggccactccagttcattctcagctttggcttccatggcaagttcctacctgggcaataactccaattcaaattcgagttatgggggccagcaacaggctaatgagtatggtagaccgcaacagaatggtcaacagcaatccaatgagtacggaagaccgcaatacggcggaaaccagaactccaatggacagcacgaatccttcaatttttctggcaacttttctcaacagaacaataacggcaaccagaaccgctactgaacgatgattcagttcgccttctatcctttgtttacgtatttgtttatatatataactttatttttttttattaattgggctgcaagacaattttgttgtcagtgatgcctcaatccttcttttgcttccatatttaccatgtggaccctttcaaaacagagttgtatctctgcaggatgccctttttgacgtattgaatggcataattgcactgtcacttttcgcgctgtctcattttggtgcgatgatgaaacaaacatgaaacgtctgtaatttgaaacaaataacgtaattctcgggattggttttatttaaatgacaatgtaagagtggctttgtaaggtatgtgttgctcttaaaatatttggatacgacatcctttatcttttttcctttaagagcaggatataagccatcaagtttctgaaaatcaaaacctgcactaaaacaatgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcagcctctccagcggcacctgctcccgcaagccctgctgcaccagctccgtcagctcctgctgcctctccagctgcacctgctccagcttctccagcagctcctgcacctagtgctcctgctgggggtggaggctctggcggaggtgggtctggtgggggcggatctgctagccccgaggaacctctagtggtgaaggtggaagagggagatgaggcttggctcccctgcctcaaggggacctccgatggccccactcagcagctgacctggtctcgggagtccccgcttaagcccttcctaaaagtgagtttcggggtgccaggactgggcgtccacgtgaggcccaacgccgtctctcttgtcatctctaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagcagggacatgactgtggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaactggtactcggctgtttctgcctcgggccacagctcaagacgctggaaagtattattgccaccgtggcaacctgaccatgtcattccacctggaggtcaaagctcggccagtctctgctcacactaaactaagaactggtggctggaagggatccgaacaaaagcttatttctgaagaggacttgtaatagaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 142 - DNA, 2634 bp, Synthetic construct
  source 1..2634: mol_type = other DNA
ttgaatgatcaaccaaacgccgatatagtcactattggtgataaaattaaaatgcagctattgagaacccatcctaacaacattaaattgtctattaatggaattggtaaagatgccccaactttccaagaatctgctttgattgccgataagttattgagtgtcatgaaggccggcacttacccaaagatttccattttctacaatgacccagtgtcttccctatcttttgaaccatctgaaaaaccgatctttaacgccaagaccattgaacaatccccatcattcggcaaatttgagatcgacacggacgcaaacgttccaagagatttgtttgaatatactttggctaaccaaatgttgacagcaatggctcaaggttatgctgctgaaatttccgccagaagaaacgctatggataacgcttccaagaatgccggtgatatgatcaatcgttactctatcttgtacaacagaacaagacaagctgtcattactaatgaactggttgatattattactggtgcttcctctttgggatgataaaaaaatcaccctgcattgcctctttatttgacgttgtttttgtagaacatgaaacgaattttgacttgatgagacgaagtatatatccaaagaataccttaaatagaaaaggaaagataataaatactaaacactactatatattcaggtaaaatacaaaaattataacatttttaaaactttttttttttgaaagtccttctcgctttaggattttttcccattaagattatgatggtttcatgtatgtgtcagttaaaaaaaatatggctaagtagcaatgaaacgaaccaagaaaatgaaaacaatatatagtgctgttgaaataacaaagacattggtatatatttgtaaaatgtctgttaaatgttttttatctcaggttcttgcttgtctttggtagaagaaattatagtaaacaaacaaacaaacaaacaaaaaaaaaaaaaaaaaaacctgcactaaaacaatgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcagcctctccagcggcacctgctcccgcaagccctgctgcaccagctccgtcagctcctgctgcctctccagctgcacctgctccagcttctccagcagctcctgcacctagtgctcctgctgggggtggaggctctggcggaggtgggtctggtgggggcggatctgctagccccgaggaacctctagtggtgaaggtggaagagggagatgaggcttggctcccctgcctcaaggggacctccgatggccccactcagcagctgacctggtctcgggagtccccgcttaagcccttcctaaaagtgagtttcggggtgccaggactgggcgtccacgtgaggcccaacgccgtctctcttgtcatctctaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagcagggacatgactgtggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaactggtactcggctgtttctgcctcgggccacagctcaagacgctggaaagtattattgccaccgtggcaacctgaccatgtcattccacctggaggtcaaagctcggccagtctctgctcacactaaactaagaactggtggctggaagggatccgaacaaaagcttatttctgaagaggacttgtaatagaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 143 - DNA, 2634 bp, Synthetic construct
  source 1..2634: mol_type = other DNA
cgggatcgatgtggctaagaccgaggagaaactacgcacctacgaggaattaaacaagcaactgattatgaataacttggagagaagcagaacagagatagaaagtttcgaacagagacaaaaattcgaaaaggagatgaaattgaagaagcgactgttggaaagacaaatcgaagaggaggaaagaatgaacaaagagtggacaaagaaagaaatcgtcaaccggttgagtacgaccacccaggatatcaatgaaacaatcgaaggcgttaaaaatacggttaaattgaaaaaatcgtccgctagaaggaaattggaagaactaaatagagttctgaaaaacaatccatatttcaattcaaatgtgaacgtacagaattcgagactcaaagatgctgtaccgttcacgccgtttaacggtgatagagaagcacacccaaggtttacgttgaaaggttcagtatacaatgacccattcatcaaagatcttgagcacaggaaagaatttattgcgtctgggttcaacactaattatgcgtacgaaagggtgttgacagaggcatttatgggcttaggatgtgttatatccgaggagctttaaaacatcaggatagtgtgcaacgtggcataagctatgtaatcaactactttttattttctatgtacgcatatacatgcattcacgatctgtttcagtgttcagaaaaaaggcacctactgctacggttggcccatacctttattctttgttcttgttacaaacgagtgtgtaattacccaaaaaggaaatttacatgttaaatgaaacccagtaatcagaaaaaacagttaagaaacctaaaatggtagagataaagatacagattcagtggttgctgaaaatcaagtaaaaaaatgaaatagagtcttcatatataaaccgccagaaatgaattaatgagagggatctgtaactgtttctcggataaaaccaaaataagtacaaagccatcgaatagaaacctgcactaaaacaatgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcagcctctccagcggcacctgctcccgcaagccctgctgcaccagctccgtcagctcctgctgcctctccagctgcacctgctccagcttctccagcagctcctgcacctagtgctcctgctgggggtggaggctctggcggaggtgggtctggtgggggcggatctgctagccccgaggaacctctagtggtgaaggtggaagagggagatgaggcttggctcccctgcctcaaggggacctccgatggccccactcagcagctgacctggtctcgggagtccccgcttaagcccttcctaaaagtgagtttcggggtgccaggactgggcgtccacgtgaggcccaacgccgtctctcttgtcatctctaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagcagggacatgactgtggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaactggtactcggctgtttctgcctcgggccacagctcaagacgctggaaagtattattgccaccgtggcaacctgaccatgtcattccacctggaggtcaaagctcggccagtctctgctcacactaaactaagaactggtggctggaagggatccgaacaaaagcttatttctgaagaggacttgtaatagaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 144 - DNA, 2634 bp, Synthetic construct
  source 1..2634: mol_type = other DNA
atgtgaccgtgccttttttgcacttttagagaaacagtgttaagaactgtaataactgttcaattctaagcactcaaaccgtgcttgtacatatgcttcaatggcctgctcgatagaccgatcacgtttaatttttctttaattttgccaagggaacgttcttttcgtaggcaccgcaatttatttctaatggataagttctgaattttgctatgaattagttagtagggcctactaaaatggcgtggtatttttaaagggcctttaccagccactcctcaacagaaaaccgtgctcgcgttgcagaaatacgaggtagacacgcgccttaggaacataaaaataaatatctcgcgctcacggaaggcaaccactaactccttttcatcgaaaagtgaggtgtagcttactaaagcattcctctttatccaagtcgtatatttttctttatactgctagttcattctgagcggagattcctggctaactctccaattggaaataaggtaataatgacttttcatgacgaattgggtgaaaatgcttggtgctaggccctgtgttgtggcttttttcggtaatagtttaattttccaaaaaggttcctgtgactatcttatcaaagtagcttagaccacgtcctgcacggttttgcagactgcttagggggtctcttgttgttattagcagcggaatatctttgtttactcattaaaatgcccccccgttcgttgcaaggaacgcgcgcgcacagaacttgcggatgcttccgcagaagtgcccgtaggagatttaagctagcccagcggttaatctacgcagttgcgtattcggagtccgctcacacttgcaaaaagcatataaaaagcttcatattcatctcattagtatcggttctgccatcccattatcttaggtttatgccttttctggttctcaagtcagtaacaacaaatacccatcgttgattctttttggtaagatctcgcctgtcaaagagacctgcactaaaacaatgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcagcctctccagcggcacctgctcccgcaagccctgctgcaccagctccgtcagctcctgctgcctctccagctgcacctgctccagcttctccagcagctcctgcacctagtgctcctgctgggggtggaggctctggcggaggtgggtctggtgggggcggatctgctagccccgaggaacctctagtggtgaaggtggaagagggagatgaggcttggctcccctgcctcaaggggacctccgatggccccactcagcagctgacctggtctcgggagtccccgcttaagcccttcctaaaagtgagtttcggggtgccaggactgggcgtccacgtgaggcccaacgccgtctctcttgtcatctctaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagcagggacatgactgtggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaactggtactcggctgtttctgcctcgggccacagctcaagacgctggaaagtattattgccaccgtggcaacctgaccatgtcattccacctggaggtcaaagctcggccagtctctgctcacactaaactaagaactggtggctggaagggatccgaacaaaagcttatttctgaagaggacttgtaatagaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 145 - DNA, 2634 bp, Synthetic construct
  source 1..2634: mol_type = other DNA
ctattttcgaggaccttgtcaccttgagcccaagagagccaagatttaaattttcctatgacttgatgcaaattcccaaagctaataacatgcaagacacgtacggtcaagaagacatatttgacctcttaacaggttcagacgcgactgcctcatcagtaagacccgttgaaaagaacttacctgaaaaaaacgaatatatactagcgttgaatgttagcgtcaacaacaagaagtttaatgacgcggaggccaaggcaaaaagattccttgattacgtaagggagttagaatcattttgaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaacctgcactaaaacaatgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcagcctctccagcggcacctgctcccgcaagccctgctgcaccagctccgtcagctcctgctgcctctccagctgcacctgctccagcttctccagcagctcctgcacctagtgctcctgctgggggtggaggctctggcggaggtgggtctggtgggggcggatctgctagccccgaggaacctctagtggtgaaggtggaagagggagatgaggcttggctcccctgcctcaaggggacctccgatggccccactcagcagctgacctggtctcgggagtccccgcttaagcccttcctaaaagtgagtttcggggtgccaggactgggcgtccacgtgaggcccaacgccgtctctcttgtcatctctaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagcagggacatgactgtggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaactggtactcggctgtttctgcctcgggccacagctcaagacgctggaaagtattattgccaccgtggcaacctgaccatgtcattccacctggaggtcaaagctcggccagtctctgctcacactaaactaagaactggtggctggaagggatccgaacaaaagcttatttctgaagaggacttgtaatagaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 146 - DNA, 2634 bp, Synthetic construct
  source 1..2634: mol_type = other DNA
agacgcgaatttttcgaagaagtaccttcaaagaatggggtcttatcttgttttgcaagtaccactgagcaggataataatagaaatgataatatactatagtagagataacgtcgatgacttcccatactgtaattgcttttagttgtgtatttttagtgtgcaagtttctgtaaatcgattaatttttttttctttcctctttttattaaccttaatttttattttagattcctgacttcaactcaagacgcacagatattataacatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaacctgcactaaaacaatgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcagcctctccagcggcacctgctcccgcaagccctgctgcaccagctccgtcagctcctgctgcctctccagctgcacctgctccagcttctccagcagctcctgcacctagtgctcctgctgggggtggaggctctggcggaggtgggtctggtgggggcggatctgctagccccgaggaacctctagtggtgaaggtggaagagggagatgaggcttggctcccctgcctcaaggggacctccgatggccccactcagcagctgacctggtctcgggagtccccgcttaagcccttcctaaaagtgagtttcggggtgccaggactgggcgtccacgtgaggcccaacgccgtctctcttgtcatctctaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagcagggacatgactgtggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaactggtactcggctgtttctgcctcgggccacagctcaagacgctggaaagtattattgccaccgtggcaacctgaccatgtcattccacctggaggtcaaagctcggccagtctctgctcacactaaactaagaactggtggctggaagggatccgaacaaaagcttatttctgaagaggacttgtaatagaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 147 - DNA, 2186 bp, Synthetic construct
  source 1..2186: mol_type = other DNA
gttgtggaaatgtaaagagccccattatcttagcctaaaaaaaccttctctttggaactttcagtaatacgcttaactgctcattgctatattgaagtacggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaactataacctgcactaaaacaatgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcagcctctccagcggcacctgctcccgcaagccctgctgcaccagctccgtcagctcctgctgcctctccagctgcacctgctccagcttctccagcagctcctgcacctagtgctcctgctgggggtggaggctctggcggaggtgggtctggtgggggcggatctgctagccccgaggaacctctagtggtgaaggtggaagagggagatgaggcttggctcccctgcctcaaggggacctccgatggccccactcagcagctgacctggtctcgggagtccccgcttaagcccttcctaaaagtgagtttcggggtgccaggactgggcgtccacgtgaggcccaacgccgtctctcttgtcatctctaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagcagggacatgactgtggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaactggtactcggctgtttctgcctcgggccacagctcaagacgctggaaagtattattgccaccgtggcaacctgaccatgtcattccacctggaggtcaaagctcggccagtctctgctcacactaaactaagaactggtggctggaagggatccgaacaaaagcttatttctgaagaggacttgtaatagaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 148 - DNA, 2046 bp, Synthetic construct
  source 1..2046: mol_type = other DNA
agtgcgatccctgaattcgcatctagactgaactggccgataattgcagacgaacgggtagtccatcgttgtaggatactgtatatacacccagtagagtaggtgactactgtatgagcatacagtagtgacaaccacttactgtatataaatacagtagtggtcatcgatactgtatataaaaccagtaggcaatcgtttgtactgtatgtacatacagtatacctcgcaactactgtatataaacacagtaattaacttgtaatattctaatcaattgacaatattatttaaggacctattgttttttccaataggtggttagcaatcgtcttactttctaacttttcttaccttttacatttcagcaatatatatatatatatttcaaggatataccattctaagatctacctgcactaaaacaatgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcagcctctccagcggcacctgctcccgcaagccctgctgcaccagctccgtcagctcctgctgcctctccagctgcacctgctccagcttctccagcagctcctgcacctagtgctcctgctgggggtggaggctctggcggaggtgggtctggtgggggcggatctgctagccccgaggaacctctagtggtgaaggtggaagagggagatgaggcttggctcccctgcctcaaggggacctccgatggccccactcagcagctgacctggtctcgggagtccccgcttaagcccttcctaaaagtgagtttcggggtgccaggactgggcgtccacgtgaggcccaacgccgtctctcttgtcatctctaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagcagggacatgactgtggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaactggtactcggctgtttctgcctcgggccacagctcaagacgctggaaagtattattgccaccgtggcaacctgaccatgtcattccacctggaggtcaaagctcggccagtctctgctcacactaaactaagaactggtggctggaagggatccgaacaaaagcttatttctgaagaggacttgtaatagaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 149 - DNA, 2532 bp, Synthetic construct
  source 1..2532: mol_type = other DNA
ttgaatgatcaaccaaacgccgatatagtcactattggtgataaaattaaaatgcagctattgagaacccatcctaacaacattaaattgtctattaatggaattggtaaagatgccccaactttccaagaatctgctttgattgccgataagttattgagtgtcatgaaggccggcacttacccaaagatttccattttctacaatgacccagtgtcttccctatcttttgaaccatctgaaaaaccgatctttaacgccaagaccattgaacaatccccatcattcggcaaatttgagatcgacacggacgcaaacgttccaagagatttgtttgaatatactttggctaaccaaatgttgacagcaatggctcaaggttatgctgctgaaatttccgccagaagaaacgctatggataacgcttccaagaatgccggtgatatgatcaatcgttactctatcttgtacaacagaacaagacaagctgtcattactaatgaactggttgatattattactggtgcttcctctttgggatgataaaaaaatcaccctgcattgcctctttatttgacgttgtttttgtagaacatgaaacgaattttgacttgatgagacgaagtatatatccaaagaataccttaaatagaaaaggaaagataataaatactaaacactactatatattcaggtaaaatacaaaaattataacatttttaaaactttttttttttgaaagtccttctcgctttaggattttttcccattaagattatgatggtttcatgtatgtgtcagttaaaaaaaatatggctaagtagcaatgaaacgaaccaagaaaatgaaaacaatatatagtgctgttgaaataacaaagacattggtatatatttgtaaaatgtctgttaaatgttttttatctcaggttcttgcttgtctttggtagaagaaattatagtaaacaaacaaacaaacaaacaaaaaaaaaaaaaaaaaaacctgcactaaaacaatgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcagcctctccagcggcacctgctcccgcaagccctgctgcaccagctccgtcagctcctgctgcctctccagctgcacctgctccagcttctccagcagctcctgcacctagtgctcctgctgggggtggaggctctggcggaggtgggtctggtgggggcggatcagctagcatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaaggatccgaacaaaagcttatttctgaagaggacttgtaatagaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 150 - DNA, 2400 bp, Synthetic construct
  source 1..2400: mol_type = other DNA
gaattttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcgaattccctacttcatacattttcacctgcactaaaacaatgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcagcctctccagcggcacctgctcccgcaagccctgctgcaccagctccgtcagctcctgctgcctctccagctgcacctgctccagcttctccagcagctcctgcacctagtgctcctgctgggggtggaggctctggcggaggtgggtctggtgggggcggatctgctagccccgaggaacctctagtggtgaaggtggaagagggagatgaggcttggctcccctgcctcaaggggacctccgatggccccactcagcagctgacctggtctcgggagtccccgcttaagcccttcctaaaagtgagtttcggggtgccaggactgggcgtccacgtgaggcccaacgccgtctctcttgtcatctctaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagcagggacatgactgtggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaactggtactcggctgtttctgcctcgggccacagctcaagacgctggaaagtattattgccaccgtggcaacctgaccatgtcattccacctggaggtcaaagctcggccagtctctgctcacactaaactaagaactggtggctggaagggatccgaacaaaagcttatttctgaagaggacttgtaatagaggacagattagctcgagatctgataacaacagtgtagatgtaacaaaatcgactttgttcccactgtacttttagctcgtacaaaatacaatatacttttcatttctccgtaaacaacatgttttcccatgtaatatccttttctatttttcgttccgttaccaactttacacatactttatatagctattcacttctatacactaaaaaactaagacaattttaattttgctgcctgccatatttcaatttgttataaattcctataatttatcctattagtagctaaaaaaagatgaatgtgaatcgaatcctaagagaatt

SEQ ID NO: 151 - DNA, 2298 bp, Synthetic construct
  source 1..2298: mol_type = other DNA
gaattttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcgaattccctacttcatacattttcacctgcactaaaacaatgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcagcctctccagcggcacctgctcccgcaagccctgctgcaccagctccgtcagctcctgctgcctctccagctgcacctgctccagcttctccagcagctcctgcacctagtgctcctgctgggggtggaggctctggcggaggtgggtctggtgggggcggatcagctagcatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaaggatccgaacaaaagcttatttctgaagaggacttgtaatagaggacagattagctcgagatctgataacaacagtgtagatgtaacaaaatcgactttgttcccactgtacttttagctcgtacaaaatacaatatacttttcatttctccgtaaacaacatgttttcccatgtaatatccttttctatttttcgttccgttaccaactttacacatactttatatagctattcacttctatacactaaaaaactaagacaattttaattttgctgcctgccatatttcaatttgttataaattcctataatttatcctattagtagctaaaaaaagatgaatgtgaatcgaatcctaagagaatt

SEQ ID NO: 152 - DNA, 2385 bp, Synthetic construct
  source 1..2385: mol_type = other DNA
ctattttcgaggaccttgtcaccttgagcccaagagagccaagatttaaattttcctatgacttgatgcaaattcccaaagctaataacatgcaagacacgtacggtcaagaagacatatttgacctcttaacaggttcagacgcgactgcctcatcagtaagacccgttgaaaagaacttacctgaaaaaaacgaatatatactagcgttgaatgttagcgtcaacaacaagaagtttaatgacgcggaggccaaggcaaaaagattccttgattacgtaagggagttagaatcattttgaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaacctgcactaaaacaatgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcaggcgggggaggatctggtggaggaggatcagggggaggcgggtccgctagccagatcgtcctaacccagtcacctgctataatgtccgcctctccgggtgagaaagttacgatgacatgctcagcatcaagtagcgtaagttacatgaattggtatcaacagaagtctggaacctcccccaagcgttggatctatgacacttcaaagctggcatctggagtgccagctcatttcagggggtcaggcagtggtacgagctacagtctgacgatctcaggaatggaagcagaagatgcagctacatattactgtcagcagtggtcatccaacccgtttactttcggttctggtaccaagctagagataaatagaggtggtggcggctccggcggcggtggctcaggagggggcggatcacaagtccaattgcaacaatcaggtgcggagttagctcgtcctggggctagcgttaagatgagctgcaaggcgagcggttacacgttcactaggtatacgatgcactgggtaaaacaaagacccgggcagggtttggaatggataggctatataaacccgtcaagaggatataccaattacaatcagaaattcaaagacaaagcaacgcttaccaccgacaagtcatcatcaacggcctacatgcaattgagtagtctaacctccgaagatagcgcggtgtattactgtgcaagatactatgacgaccactactgtctggattattggggtcaaggaacgacgcttacggtaagtagctaatagaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 153 - DNA, 2349 bp, Synthetic construct
  source 1..2349: mol_type = other DNA
ctattttcgaggaccttgtcaccttgagcccaagagagccaagatttaaattttcctatgacttgatgcaaattcccaaagctaataacatgcaagacacgtacggtcaagaagacatatttgacctcttaacaggttcagacgcgactgcctcatcagtaagacccgttgaaaagaacttacctgaaaaaaacgaatatatactagcgttgaatgttagcgtcaacaacaagaagtttaatgacgcggaggccaaggcaaaaagattccttgattacgtaagggagttagaatcattttgaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaacctgcactaaaacaatgcagttacttcgctgtttttcaatattttctgttattgcttcagttttagcagctagccagatcgtcctaacccagtcacctgctataatgtccgcctctccgggtgagaaagttacgatgacatgctcagcatcaagtagcgtaagttacatgaattggtatcaacagaagtctggaacctcccccaagcgttggatctatgacacttcaaagctggcatctggagtgccagctcatttcagggggtcaggcagtggtacgagctacagtctgacgatctcaggaatggaagcagaagatgcagctacatattactgtcagcagtggtcatccaacccgtttactttcggttctggtaccaagctagagataaatagaggtggtggcggctccggcggcggtggctcaggagggggcggatcacaagtccaattgcaacaatcaggtgcggagttagctcgtcctggggctagcgttaagatgagctgcaaggcgagcggttacacgttcactaggtatacgatgcactgggtaaaacaaagacccgggcagggtttggaatggataggctatataaacccgtcaagaggatataccaattacaatcagaaattcaaagacaaagcaacgcttaccaccgacaagtcatcatcaacggcctacatgcaattgagtagtctaacctccgaagatagcgcggtgtattactgtgcaagatactatgacgaccactactgtctggattattggggtcaaggaacgacgcttacggtaagtagcggcgggggaggatctggtggaggaggatcagggggaggcgggtcctacccatacgacgttccagactacgctggatcccaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgttttttaatagaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 154 - DNA, 2367 bp, Synthetic construct
  source 1..2367: mol_type = other DNA
ctattttcgaggaccttgtcaccttgagcccaagagagccaagatttaaattttcctatgacttgatgcaaattcccaaagctaataacatgcaagacacgtacggtcaagaagacatatttgacctcttaacaggttcagacgcgactgcctcatcagtaagacccgttgaaaagaacttacctgaaaaaaacgaatatatactagcgttgaatgttagcgtcaacaacaagaagtttaatgacgcggaggccaaggcaaaaagattccttgattacgtaagggagttagaatcattttgaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaacctgcactaaaacaatgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcaggcgggggaggatctggtggaggaggatcagggggaggcgggtccgctagcgtacaattacaacagtctggacccgaacttgtaaaaccgggcgccagcgtaaagatgagctgtaaggcttccggttatacttttacctcctatgtcatccaatgggtgaaacaaaaacctggacagggacttgagtggataggcagtatcaacccctataacgactacacaaagtataatgagaaatttaaaggtaaagcaacccttacgagtgataaatcttcaattacagcatacatggagttcagtagcctgacgagcgaagatagcgccttatattactgcgcacgttggggcgatggttattgggggcgtggcactactttaacggtaagctctggtggtggcggctccggcggcggtggctcaggagggggcggatcagacattgaaatgacgcagtcccccgccatcatgtcagccagtcttggagaaagggtcaccatgacttgcacggcttctagtagtgtaagctcatcctacttccactggtaccagcaaaaaccaggtagttccccaaagctttgcatatacagtacgtcaaatctagcctctggagtgcccccgcgtttcagcggctccggcagcacttcatacagtcttacgataagctcaatggaggcagaggacgctgcgacttacttttgtcatcagtatcatcgttcacctacttttgggggtggcactaagcttgaaactaaaagataatagaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 155 - DNA, 2331 bp, Synthetic construct
  source 1..2331: mol_type = other DNA
ctattttcgaggaccttgtcaccttgagcccaagagagccaagatttaaattttcctatgacttgatgcaaattcccaaagctaataacatgcaagacacgtacggtcaagaagacatatttgacctcttaacaggttcagacgcgactgcctcatcagtaagacccgttgaaaagaacttacctgaaaaaaacgaatatatactagcgttgaatgttagcgtcaacaacaagaagtttaatgacgcggaggccaaggcaaaaagattccttgattacgtaagggagttagaatcattttgaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaacctgcactaaaacaatgcagttacttcgctgtttttcaatattttctgttattgcttcagttttagcagctagcgtacaattacaacagtctggacccgaacttgtaaaaccgggcgccagcgtaaagatgagctgtaaggcttccggttatacttttacctcctatgtcatccaatgggtgaaacaaaaacctggacagggacttgagtggataggcagtatcaacccctataacgactacacaaagtataatgagaaatttaaaggtaaagcaacccttacgagtgataaatcttcaattacagcatacatggagttcagtagcctgacgagcgaagatagcgccttatattactgcgcacgttggggcgatggttattgggggcgtggcactactttaacggtaagctctggtggtggcggctccggcggcggtggctcaggagggggcggatcagacattgaaatgacgcagtcccccgccatcatgtcagccagtcttggagaaagggtcaccatgacttgcacggcttctagtagtgtaagctcatcctacttccactggtaccagcaaaaaccaggtagttccccaaagctttgcatatacagtacgtcaaatctagcctctggagtgcccccgcgtttcagcggctccggcagcacttcatacagtcttacgataagctcaatggaggcagaggacgctgcgacttacttttgtcatcagtatcatcgttcacctacttttgggggtggcactaagcttgaaactaaaagaggcgggggaggatctggtggaggaggatcagggggaggcgggtcctacccatacgacgttccagactacgctggatcccaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgttttttaatagaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 156 - DNA, 2061 bp, Synthetic construct
  source 1..2061: mol_type = other DNA
ctattttcgaggaccttgtcaccttgagcccaagagagccaagatttaaattttcctatgacttgatgcaaattcccaaagctaataacatgcaagacacgtacggtcaagaagacatatttgacctcttaacaggttcagacgcgactgcctcatcagtaagacccgttgaaaagaacttacctgaaaaaaacgaatatatactagcgttgaatgttagcgtcaacaacaagaagtttaatgacgcggaggccaaggcaaaaagattccttgattacgtaagggagttagaatcattttgaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaacctgcactaaaacaatgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcaggcgggggaggatctggtggaggaggatcagggggaggcgggtccgctagcgcgccgacttctagtagtacaaaaaagacccaattgcagttggaacacttgctgctggatcttcagatgatccttaatgggataaataactataagaaccccaaactaacgagaatgctgacgttcaaattctacatgccgaaaaaagcaacagaattaaaacacttgcaatgtctagaggaggagctaaagccccttgaagaagtactaaacttggcgcagtcaaagaatttccatttgagacccagggacctaatctccaacatcaatgtcatcgtactagaactgaaaggatctgaaacaaccttcatgtgcgaatatgcagacgaaaccgcaactatagttgaatttctgaatcgttggataacattttctcagtccattatctcaacattgacgtaatagaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 157 - DNA, 1745 bp, Synthetic construct
  source 1..1745: mol_type = other DNA
ctattttcgaggaccttgtcaccttgagcccaagagagccaagatttaaattttcctatgacttgatgcaaattcccaaagctaataacatgcaagacacgtacggtcaagaagacatatttgacctcttaacaggttcagacgcgactgcctcatcagtaagacccgttgaaaagaacttacctgaaaaaaacgaatatatactagcgttgaatgttagcgtcaacaacaagaagtttaatgacgcggaggccaaggcaaaaagattccttgattacgtaagggagttagaatcattttgaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaacctgcactaaaacaatgcagttacttcgctgtttttcaatattttctgttattgcttcagttttagcacaggaacttactacaatttgtgaacaaatacctagtcccacgttggagtcaacaccgtactccctgtctactacaacaatacttgcgaacggtaaggccatgcaaggagtctttgaatactacaagtcagttaccttcgtatctaactgcgggagtcacccgagcaccacatccaaggggagtcctataaacactcagtatgtattcgactataaggatgacgatgacaagggcggcggaggctccggtggaggcggaagcggcggtggaggtagtcctagggcgaccgtgaaatttaaatataaaggcgaagaaaaacaggtggatattagcaaaattagacgggtgcgtcgcaagggcaaatgcattaggttttactatgatctgggcggcggcaaatatggcaggggcatagtgagcgaaaaagatgcgccgaaagaactgctgcagatgctggaaaaacagaaaaaataatagaggacagatatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaag

SEQ ID NO: 158 - DNA, 1745 bp, Synthetic construct
  source 1..1745: mol_type = other DNA
taataatcagaactccaacaatagtcaacagggctataaccaatcctatcaaaacggtaaccaaaatagtcaaggttacaataatcaacagtaccaaggtggcaacggtggttaccaacaacaacagggacaatctggtggtgctttttcctcattggcctccatggctcaatcttacttaggtggtggacaaactcaatccaaccaacagcaatacaatcaacaaggccaaaacaaccagcagcaataccagcaacaaggccaaaactatcagcatcaacaacagggtcagcagcagcaacaaggccactccagttcattctcagctttggcttccatggcaagttcctacctgggcaataactccaattcaaattcgagttatgggggccagcaacaggctaatgagtatggtagaccgcaacagaatggtcaacagcaatccaatgagtacggaagaccgcaatacggcggaaaccagaactccaatggacagcacgaatccttcaatttttctggcaacttttctcaacagaacaataacggcaaccagaaccgctactgaacgatgattcagttcgccttctatcctttgtttacgtatttgtttatatatataactttatttttttttattaattgggctgcaagacaattttgttgtcagtgatgcctcaatccttcttttgcttccatatttaccatgtggaccctttcaaaacagagttgtatctctgcaggatgccctttttgacgtattgaatggcataattgcactgtcacttttcgcgctgtctcattttggtgcgatgatgaaacaaacatgaaacgtctgtaatttgaaacaaataacgtaattctcgggattggttttatttaaatgacaatgtaagagtggctttgtaaggtatgtgttgctcttaaaatatttggatacgacatcctttatcttttttcctttaagagcaggatataagccatcaagtttctgaaaatcaaaacctgcactaaaacaatgcagttacttcgctgtttttcaatattttctgttattgcttcagttttagcacaggaacttactacaatttgtgaacaaatacctagtcccacgttggagtcaacaccgtactccctgtctactacaacaatacttgcgaacggtaaggccatgcaaggagtctttgaatactacaagtcagttaccttcgtatctaactgcgggagtcacccgagcaccacatccaaggggagtcctataaacactcagtatgtattcgactataaggatgacgatgacaagggcggcggaggctccggtggaggcggaagcggcggtggaggtagtcctagggcgaccgtgaaatttaaatataaaggcgaagaaaaacaggtggatattagcaaaattagacgggtgcgtcgcaagggcaaatgcattaggttttactatgatctgggcggcggcaaatatggcaggggcatagtgagcgaaaaagatgcgccgaaagaactgctgcagatgctggaaaaacagaaaaaataatagaggacagatatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaag

SEQ ID NO: 159 - DNA, 2322 bp, Synthetic construct
  source 1..2322: mol_type = other DNA
ctattttcgaggaccttgtcaccttgagcccaagagagccaagatttaaattttcctatgacttgatgcaaattcccaaagctaataacatgcaagacacgtacggtcaagaagacatatttgacctcttaacaggttcagacgcgactgcctcatcagtaagacccgttgaaaagaacttacctgaaaaaaacgaatatatactagcgttgaatgttagcgtcaacaacaagaagtttaatgacgcggaggccaaggcaaaaagattccttgattacgtaagggagttagaatcattttgaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaacctgcactaaaacaatgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcaggcgggggaggatctggtggaggaggatcagggggaggcgggtccgctagctttactgttactgttccaaaagatttgtatgttgttgaatatggttctaatatgactattgaatgtaaatttccagttgaaaaacaattggatttggctgctttgattgtttattgggaaatggaagataaaaatattattcaatttgttcatggtgaagaagatttgaaagttcaacattcttcttatagacaaagagctagattgttgaaagatcaattgtctttgggtaatgctgctttgcaaattactgatgttaaattgcaagatgctggtgtttatagatgtatgatttcttatggtggtgctgattataaaagaattactgttaaagttaatgctccatataataaaattaatcaaagaattttggttgttgatccagttacttctgaacatgaattgacttgtcaagctgaaggttatccaaaagctgaagttatttggacttcttctgatcatcaagttttgtctggtaaaactactactactaattctaaaagagaagaaaaattgtttaatgttacttctactttgagaattaatactactactaatgaaattttttattgtacttttagaagattggatccagaagaaaatcatactgctgaattggttattccagaattgccattggctcatccaccaaatgaaagataatagaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 160 - DNA, 2442 bp, Synthetic construct
  source 1..2442: mol_type = other DNA
ctattttcgaggaccttgtcaccttgagcccaagagagccaagatttaaattttcctatgacttgatgcaaattcccaaagctaataacatgcaagacacgtacggtcaagaagacatatttgacctcttaacaggttcagacgcgactgcctcatcagtaagacccgttgaaaagaacttacctgaaaaaaacgaatatatactagcgttgaatgttagcgtcaacaacaagaagtttaatgacgcggaggccaaggcaaaaagattccttgattacgtaagggagttagaatcattttgaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaacctgcactaaaacaatgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcagcctctccagcggcacctgctcccgcaagccctgctgcaccagctccgtcagctcctgctgcctctccagctgcacctgctccagcttctccagcagctcctgcacctagtgctcctgctgggggtggaggctctggcggaggtgggtctggtgggggcggatctgctagctttactgttactgttccaaaagatttgtatgttgttgaatatggttctaatatgactattgaatgtaaatttccagttgaaaaacaattggatttggctgctttgattgtttattgggaaatggaagataaaaatattattcaatttgttcatggtgaagaagatttgaaagttcaacattcttcttatagacaaagagctagattgttgaaagatcaattgtctttgggtaatgctgctttgcaaattactgatgttaaattgcaagatgctggtgtttatagatgtatgatttcttatggtggtgctgattataaaagaattactgttaaagttaatgctccatataataaaattaatcaaagaattttggttgttgatccagttacttctgaacatgaattgacttgtcaagctgaaggttatccaaaagctgaagttatttggacttcttctgatcatcaagttttgtctggtaaaactactactactaattctaaaagagaagaaaaattgtttaatgttacttctactttgagaattaatactactactaatgaaattttttattgtacttttagaagattggatccagaagaaaatcatactgctgaattggttattccagaattgccattggctcatccaccaaatgaaagataatagaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 161 - DNA, 3552 bp, Synthetic construct
  source 1..3552: mol_type = other DNA
ctattttcgaggaccttgtcaccttgagcccaagagagccaagatttaaattttcctatgacttgatgcaaattcccaaagctaataacatgcaagacacgtacggtcaagaagacatatttgacctcttaacaggttcagacgcgactgcctcatcagtaagacccgttgaaaagaacttacctgaaaaaaacgaatatatactagcgttgaatgttagcgtcaacaacaagaagtttaatgacgcggaggccaaggcaaaaagattccttgattacgtaagggagttagaatcattttgaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaacctgcactaaaacaatgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcaggcgggggaggatctggtggaggaggatcagggggaggcgggtccgctagcactcaagtttgtactggtactgatatgaaattgagattgccagcttctccagaaactcatttggatatgttgagacatttgtatcaaggttgtcaagttgttcaaggtaatttggaattgacttatttgccaactaatgcttctttgtcttttttgcaagatattcaagaagttcaaggttatgttttgattgctcataatcaagttagacaagttccattgcaaagattgagaattgttagaggtactcaattgtttgaagataattatgctttggctgttttggataatggtgatccattgaataatactactccagttactggtgcttctccaggtggtttgagagaattgcaattgagatctttgactgaaattttgaaaggtggtgttttgattcaaagaaatccacaattgtgttatcaagatactattttgtggaaagatatttttcataaaaataatcaattggctttgactttgattgatactaatagatctagagcttgtcatccatgttctccaatgtgtaaaggttctagatgttggggtgaatcttctgaagattgtcaatctttgactagaactgtttgtgctggtggttgtgctagatgtaaaggtccattgccaactgattgttgtcatgaacaatgtgctgctggttgtactggtccaaaacattctgattgtttggcttgtttgcattttaatcattctggtatttgtgaattgcattgtccagctttggttacttataatactgatacttttgaatctatgccaaatccagaaggtagatatacttttggtgcttcttgtgttactgcttgtccatataattatttgtctactgatgttggttcttgtactttggtttgtccattgcataatcaagaagttactgctgaagatggtactcaaagatgtgaaaaatgttctaaaccatgtgctagagtttgttatggtttgggtatggaacatttgagagaagttagagctgttacttctgctaatattcaagaatttgctggttgtaaaaaaatttttggttctttggcttttttgccagaatcttttgatggtgatccagcttctaatactgctccattgcaaccagaacaattgcaagtttttgaaactttggaagaaattactggttatttgtatatttctgcttggccagattctttgccagatttgtctgtttttcaaaatttgcaagttattagaggtagaattttgcataatggtgcttattctttgactttgcaaggtttgggtatttcttggttgggtttgagatctttgagagaattgggttctggtttggctttgattcatcataatactcatttgtgttttgttcatactgttccatgggatcaattgtttagaaatccacatcaagctttgttgcatactgctaatagaccagaagatgaatgtgttggtgaaggtttggcttgtcatcaattgtgtgctagaggtcattgttggggtccaggtccaactcaatgtgttaattgttctcaatttttgagaggtcaagaatgtgttgaagaatgtagagttttgcaaggtttgccaagagaatatgttaatgctagacattgtttgccatgtcatccagaatgtcaaccacaaaatggttctgttacttgttttggtccagaagctgatcaatgtgttgcttgtgctcattataaagatccaccattttgtgttgctagatgtccatctggtgttaaaccagatttgtcttatatgccaatttggaaatttccagatgaagaaggtgcttgtcaaccatgtccaattaattgtactcattcttgtgttgatttggatgataaaggttgtccagctgaacaaagagcttctccattgacttaatagaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 162 - DNA, 3672 bp, Synthetic construct
  source 1..3672: mol_type = other DNA
ctattttcgaggaccttgtcaccttgagcccaagagagccaagatttaaattttcctatgacttgatgcaaattcccaaagctaataacatgcaagacacgtacggtcaagaagacatatttgacctcttaacaggttcagacgcgactgcctcatcagtaagacccgttgaaaagaacttacctgaaaaaaacgaatatatactagcgttgaatgttagcgtcaacaacaagaagtttaatgacgcggaggccaaggcaaaaagattccttgattacgtaagggagttagaatcattttgaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaacctgcactaaaacaatgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggcttcagcctctccagcggcacctgctcccgcaagccctgctgcaccagctccgtcagctcctgctgcctctccagctgcacctgctccagcttctccagcagctcctgcacctagtgctcctgctgggggtggaggctctggcggaggtgggtctggtgggggcggatctgctagcactcaagtttgtactggtactgatatgaaattgagattgccagcttctccagaaactcatttggatatgttgagacatttgtatcaaggttgtcaagttgttcaaggtaatttggaattgacttatttgccaactaatgcttctttgtcttttttgcaagatattcaagaagttcaaggttatgttttgattgctcataatcaagttagacaagttccattgcaaagattgagaattgttagaggtactcaattgtttgaagataattatgctttggctgttttggataatggtgatccattgaataatactactccagttactggtgcttctccaggtggtttgagagaattgcaattgagatctttgactgaaattttgaaaggtggtgttttgattcaaagaaatccacaattgtgttatcaagatactattttgtggaaagatatttttcataaaaataatcaattggctttgactttgattgatactaatagatctagagcttgtcatccatgttctccaatgtgtaaaggttctagatgttggggtgaatcttctgaagattgtcaatctttgactagaactgtttgtgctggtggttgtgctagatgtaaaggtccattgccaactgattgttgtcatgaacaatgtgctgctggttgtactggtccaaaacattctgattgtttggcttgtttgcattttaatcattctggtatttgtgaattgcattgtccagctttggttacttataatactgatacttttgaatctatgccaaatccagaaggtagatatacttttggtgcttcttgtgttactgcttgtccatataattatttgtctactgatgttggttcttgtactttggtttgtccattgcataatcaagaagttactgctgaagatggtactcaaagatgtgaaaaatgttctaaaccatgtgctagagtttgttatggtttgggtatggaacatttgagagaagttagagctgttacttctgctaatattcaagaatttgctggttgtaaaaaaatttttggttctttggcttttttgccagaatcttttgatggtgatccagcttctaatactgctccattgcaaccagaacaattgcaagtttttgaaactttggaagaaattactggttatttgtatatttctgcttggccagattctttgccagatttgtctgtttttcaaaatttgcaagttattagaggtagaattttgcataatggtgcttattctttgactttgcaaggtttgggtatttcttggttgggtttgagatctttgagagaattgggttctggtttggctttgattcatcataatactcatttgtgttttgttcatactgttccatgggatcaattgtttagaaatccacatcaagctttgttgcatactgctaatagaccagaagatgaatgtgttggtgaaggtttggcttgtcatcaattgtgtgctagaggtcattgttggggtccaggtccaactcaatgtgttaattgttctcaatttttgagaggtcaagaatgtgttgaagaatgtagagttttgcaaggtttgccaagagaatatgttaatgctagacattgtttgccatgtcatccagaatgtcaaccacaaaatggttctgttacttgttttggtccagaagctgatcaatgtgttgcttgtgctcattataaagatccaccattttgtgttgctagatgtccatctggtgttaaaccagatttgtcttatatgccaatttggaaatttccagatgaagaaggtgcttgtcaaccatgtccaattaattgtactcattcttgtgttgatttggatgataaaggttgtccagctgaacaaagagcttctccattgacttaatagaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 163 - DNA, 1992 bp, Synthetic construct
  source 1..1992: mol_type = other DNA
ttccttacagttgaaaacaaaatcaaatcacggtatggcatttctttacatattctcaatacctgactgacatagtatgcgctacatagttttgtcctattttttttacaccgcgatttgaagtctaaagccagagaatgaggaaaaacgaaacaaactgagatcatttgtataaggggagtcactgtacagctgccaataccaattttccttctgccaaatcaaaaaagtatccgcatacacatacacccctccctctacaatcttaattatttactgattcctttcgtatttgtccagtatgagcaatactaagcataccacttctcatcacatggaactcaaaaggatcattatccttacacttttattcatactcataatgttgatatttcgaaactcagtgtccttcaaaatgacatttcaagaactactgccacgattttacaaaaagaattcaaactcagttagtaataataacaggccctcatctattttctcggaaaacttggtggattttgatgatgttaacatggtcgataagaccagactgtttatttttttatttttcagtttcatcattactataccgtttatggtataaattagaaaagttaaagcagcatgttttcatttgaaacaaatactaatgcaataataggtacaccatctactacataattaattgatagtttccttttccgttaagtgcatgcataggacgcccataattttttaagttaaaaaagcatgtatttacctattcgggaaatttacatgacatggatgccataaggaacgaaaatgaaacatgcatgtcagaggaaaaagaacaaagcgagaggaaaaagctgttgcattaccacgtaattttgtatataaatatctgataaataaccattttatttccatccacttcttctgtcgttcatccgttcattgacatcactagagacaccagcgagctatcatcttcatacaacaataactaccaaccttaacctgcactaaaacaatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataaaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 164 - DNA, 1992 bp, Synthetic construct
  source 1..1992: mol_type = other DNA
taataatcagaactccaacaatagtcaacagggctataaccaatcctatcaaaacggtaaccaaaatagtcaaggttacaataatcaacagtaccaaggtggcaacggtggttaccaacaacaacagggacaatctggtggtgctttttcctcattggcctccatggctcaatcttacttaggtggtggacaaactcaatccaaccaacagcaatacaatcaacaaggccaaaacaaccagcagcaataccagcaacaaggccaaaactatcagcatcaacaacagggtcagcagcagcaacaaggccactccagttcattctcagctttggcttccatggcaagttcctacctgggcaataactccaattcaaattcgagttatgggggccagcaacaggctaatgagtatggtagaccgcaacagaatggtcaacagcaatccaatgagtacggaagaccgcaatacggcggaaaccagaactccaatggacagcacgaatccttcaatttttctggcaacttttctcaacagaacaataacggcaaccagaaccgctactgaacgatgattcagttcgccttctatcctttgtttacgtatttgtttatatatataactttatttttttttattaattgggctgcaagacaattttgttgtcagtgatgcctcaatccttcttttgcttccatatttaccatgtggaccctttcaaaacagagttgtatctctgcaggatgccctttttgacgtattgaatggcataattgcactgtcacttttcgcgctgtctcattttggtgcgatgatgaaacaaacatgaaacgtctgtaatttgaaacaaataacgtaattctcgggattggttttatttaaatgacaatgtaagagtggctttgtaaggtatgtgttgctcttaaaatatttggatacgacatcctttatcttttttcctttaagagcaggatataagccatcaagtttctgaaaatcaaaacctgcactaaaacaatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataaaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 165 - DNA, 1992 bp, Synthetic construct
  source 1..1992: mol_type = other DNA
caaatctgcctctttgttcaagcaaagaaagaacccaagaagaatcgcttggactgtcttattcagaaagcatcacaagaagggtatcaccgaagaagttgctaagaagagatctagaaaaaccgttaaggcccaaagaccaattaccggtgcttctttggacttgatcaaggaaagaagatctttgaagccagaagttagaaaggctaacagagaagaaaaattgaaggccaacaaagaaaagaagaaggctgaaaaggctgctagaaaggctgaaaaggctaagtctgctggtactcaaagttctaagttctccaagcaacaagctaagggtgctttccaaaaggttgctgctacttctcgttaagatttatgctcgaacttattatgtacaatgaatatttttcttttaaatcatttttaaatatttcaatttagtattattttctgtataattcattcgcgaggtataatctaccatccacttctctatcattatactcccatatttttttattcacaggctagacaatggtaatggtgctttagatgatttcagtggattacattcgcacaatagaataaatgactaattttcaagaatttgtttcaacctgccggcattcagttattataagttattgtttgatggtaaaacttcgccatcaccggttgctgcgtttattgcaaaattactgaaaccatgtaatttccgaatacggtaattacacgcagtttgaaatttgacatgcaagtttaacaaaaaaacttctgtccgcatctaaagcttaattaagggactgtttcataccttcagatccaatatactgaatatcaggcaattgatttcccttgtaaatatacaatataaatagttgtaagaagaggtgatatgttaagtttccattttgacgtattcctcattctagaatgattgtaagctctcaacagtcacttgtgtgccaaaatattatacctacttcatacattttcaattaagacctgcactaaaacaatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataaaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 166 - DNA, 1992 bp, Synthetic construct
  source 1..1992: mol_type = other DNA
ttgaatgatcaaccaaacgccgatatagtcactattggtgataaaattaaaatgcagctattgagaacccatcctaacaacattaaattgtctattaatggaattggtaaagatgccccaactttccaagaatctgctttgattgccgataagttattgagtgtcatgaaggccggcacttacccaaagatttccattttctacaatgacccagtgtcttccctatcttttgaaccatctgaaaaaccgatctttaacgccaagaccattgaacaatccccatcattcggcaaatttgagatcgacacggacgcaaacgttccaagagatttgtttgaatatactttggctaaccaaatgttgacagcaatggctcaaggttatgctgctgaaatttccgccagaagaaacgctatggataacgcttccaagaatgccggtgatatgatcaatcgttactctatcttgtacaacagaacaagacaagctgtcattactaatgaactggttgatattattactggtgcttcctctttgggatgataaaaaaatcaccctgcattgcctctttatttgacgttgtttttgtagaacatgaaacgaattttgacttgatgagacgaagtatatatccaaagaataccttaaatagaaaaggaaagataataaatactaaacactactatatattcaggtaaaatacaaaaattataacatttttaaaactttttttttttgaaagtccttctcgctttaggattttttcccattaagattatgatggtttcatgtatgtgtcagttaaaaaaaatatggctaagtagcaatgaaacgaaccaagaaaatgaaaacaatatatagtgctgttgaaataacaaagacattggtatatatttgtaaaatgtctgttaaatgttttttatctcaggttcttgcttgtctttggtagaagaaattatagtaaacaaacaaacaaacaaacaaaaaaaaaaaaaaaaaaacctgcactaaaacaatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataaaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 167 - DNA, 1992 bp, Synthetic construct
  source 1..1992: mol_type = other DNA
cgggatcgatgtggctaagaccgaggagaaactacgcacctacgaggaattaaacaagcaactgattatgaataacttggagagaagcagaacagagatagaaagtttcgaacagagacaaaaattcgaaaaggagatgaaattgaagaagcgactgttggaaagacaaatcgaagaggaggaaagaatgaacaaagagtggacaaagaaagaaatcgtcaaccggttgagtacgaccacccaggatatcaatgaaacaatcgaaggcgttaaaaatacggttaaattgaaaaaatcgtccgctagaaggaaattggaagaactaaatagagttctgaaaaacaatccatatttcaattcaaatgtgaacgtacagaattcgagactcaaagatgctgtaccgttcacgccgtttaacggtgatagagaagcacacccaaggtttacgttgaaaggttcagtatacaatgacccattcatcaaagatcttgagcacaggaaagaatttattgcgtctgggttcaacactaattatgcgtacgaaagggtgttgacagaggcatttatgggcttaggatgtgttatatccgaggagctttaaaacatcaggatagtgtgcaacgtggcataagctatgtaatcaactactttttattttctatgtacgcatatacatgcattcacgatctgtttcagtgttcagaaaaaaggcacctactgctacggttggcccatacctttattctttgttcttgttacaaacgagtgtgtaattacccaaaaaggaaatttacatgttaaatgaaacccagtaatcagaaaaaacagttaagaaacctaaaatggtagagataaagatacagattcagtggttgctgaaaatcaagtaaaaaaatgaaatagagtcttcatatataaaccgccagaaatgaattaatgagagggatctgtaactgtttctcggataaaaccaaaataagtacaaagccatcgaatagaaacctgcactaaaacaatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataaaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 168 - DNA, 1992 bp, Synthetic construct
  source 1..1992: mol_type = other DNA
aaccctgattttacctgtctcccaacttactctccattaccctacctctccactcgttaccctgtctcattcaaccgtaccactcccaaccaccatccatctctctacttactaccaccaacccaccgtccaccataaccgttaccctccaactacccgtatccaactccactaccgcttaccctaccatcgaccatgtcctactcactgtactgttgttcacccaccatattgaaacgtctacaaatgatcgtaaataatacacatatacttatcctaccactctaatcccactaccacatgccatactcaccttcacttgtattctgatcggtcatacgcacacggatgctacagtatataccatctcaaacttaccctactttcatattccactccatcacccatctctcaccatcagtaccaaatgcactcgcatcattatgcacggcacttgcctcagcggtctataccctgtgccatttacgcataacgcccatcattatccacattttaatatctatatctcattcggcgacaccaaatattgtataactgcccttaatacatacgttataccacttttacaccatatactaaccactcaatttatacacacttatgtcaatataaccacaaaatcaccactaaaatcacctaaacataaaaatattctactcttcaacaataatacataaacacactcaattgcgtatctataccaccatgacgtcattaacgtaaaagttccttaatattgcaaattgcttgaacggatgccatttcatgttgaatgtggtaacccaatagcatgatatgagtaatgctttagtattgtttcagagttgtttcagtaatgttttagacaaggagaacatatagtagcaaacctctaatccggtagtacttaagaaactacagtttctatgtacgaaagcagtaactatgtaattattacatttacatgacatataggaaggtccaataaacttactacattacctgcactaaaacaatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataaaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 169 - DNA, 1992 bp, Synthetic construct
  source 1..1992: mol_type = other DNA
atgtgaccgtgccttttttgcacttttagagaaacagtgttaagaactgtaataactgttcaattctaagcactcaaaccgtgcttgtacatatgcttcaatggcctgctcgatagaccgatcacgtttaatttttctttaattttgccaagggaacgttcttttcgtaggcaccgcaatttatttctaatggataagttctgaattttgctatgaattagttagtagggcctactaaaatggcgtggtatttttaaagggcctttaccagccactcctcaacagaaaaccgtgctcgcgttgcagaaatacgaggtagacacgcgccttaggaacataaaaataaatatctcgcgctcacggaaggcaaccactaactccttttcatcgaaaagtgaggtgtagcttactaaagcattcctctttatccaagtcgtatatttttctttatactgctagttcattctgagcggagattcctggctaactctccaattggaaataaggtaataatgacttttcatgacgaattgggtgaaaatgcttggtgctaggccctgtgttgtggcttttttcggtaatagtttaattttccaaaaaggttcctgtgactatcttatcaaagtagcttagaccacgtcctgcacggttttgcagactgcttagggggtctcttgttgttattagcagcggaatatctttgtttactcattaaaatgcccccccgttcgttgcaaggaacgcgcgcgcacagaacttgcggatgcttccgcagaagtgcccgtaggagatttaagctagcccagcggttaatctacgcagttgcgtattcggagtccgctcacacttgcaaaaagcatataaaaagcttcatattcatctcattagtatcggttctgccatcccattatcttaggtttatgccttttctggttctcaagtcagtaacaacaaatacccatcgttgattctttttggtaagatctcgcctgtcaaagagacctgcactaaaacaatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataaaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 170 - DNA, 1992 bp, Synthetic construct
  source 1..1992: mol_type = other DNA
ttgcatgatgaagaccaccagaccaatttatcgctatgtccgattgattgttgattagtttcctggtggcgtcaaggctcgcaccagtgtatagtgttgtataatcatataaattttgaaaaataggacagtcgtctccaatgttaaaattttcaagtgtgcctctgggcagtttgtttgcattttctggtgaaaccttcgagagaaaattcacgtagtcttccgagtgaaattgtagtaattcgtctctggtagcgcttcttgtttcataaaggtccataatcttgtgcaatccataagaggaaactaggtgatccgttagcattaaccgaaacggcttcattggatgcttaacaccataatggtaatgtgaaactttggagttaaaatgatacgataccctgggagaataagcagaattaaactcaaataatggttcgttctcctttgttttcacatcataactaaatgttccagacatgttgcgtgttgttattcacttgatacctgattttaacgtacttgagtaatctggcgtcttttctataactgtacattggcgacacacctaaaactgattagaatcgtttcaagatgctttcagttcaacatttagtaacatagtaggaatatcacgtttaacgatgccccaaagaacatcgagaatcctctatatagagtataaacagggcattctacattggtatagaggtgccttaggtatatagtggtaccggtcacataaattgacgggaatatgcgtctttaggcggcttttggcgtacagtattgttctttctttcgtgcttgtgaaacttgttataaacaaagaaagagaaagatctccccggcattctggaagatcggtaaccgctggagctataggaaagggagatggataacagtaaagaaaacggaccatataagttagcattgggggttttcgaaaaaaataagaattggcattcagacttgaattcataaaagttgtaagcaggctacctgcactaaaacaatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataaaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 171 - DNA, 1992 bp, Synthetic construct
  source 1..1992: mol_type = other DNA
cgttagtcaacgactgttggctgtcatctgttcttgttgggtgcttgcagctgcatcgacaaacatggatggatgcctctacatatatatatatatttatgtaatggacctctgaatattttactatgtaactgactataggagataccactggtgctgtagtacaatttagcacttgcaagcgttgacacagttgttgcgaaggcctctttcttgtagcacttaattggagtatgactgccatcatgtagggaaattatgaaagcataggatcaatcttgacgttgcaaagaagaagagacttgctgttccctattgatcacttaccgttattggagttcatatggtacttttttcataattttacgagtaatccctagtggtagttaatattcagcaaataaaataccagtttgagaccaaaggtaagataagagtttactgataaagacaaagaattgggatgacctagtccaagggattatagtaccttcgcgagtattgcatataagcttttagcgatacacaaacaaagcgacaccgggatttgcatatcgcttgttcctacgacgttgtcagttccataacctctgatttcccatacggattttgaaaatcagctacgaatagatagtctttttctattttcagtaaatgtgtcattgaaaaaagtttctttagaacgacgctttttgcaggctactagaaacacccacttctagtagctttacgtgacgaattgaaccgcgtgatcttgtgcactgttatcctaaatgctcaatgctcagaacatcttcttgttatctcagcctcgccacccttatcttatcatagcggccttttattttcccctttgggcaaaatacgatacagccttccaaggtcgggcaagaaagtttatgacatataaaagaaacgggtaccccaaatacgtctcttcccattgtaaaactaatcctgcatcatcacattaaggaatcattcatctaacaattacattacctgcactaaaacaatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataaaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 172 - DNA, 1992 bp, Synthetic construct
  source 1..1992: mol_type = other DNA
cgctagagaatttttttcggaaagatttggaatatcggcgcacggaattatgttccttgctttctctcgtggataggtgcaaataacgttaaataatatgataattccaagatccatgaatcatgtttgcaaattgggtggcatttcaaattattttaggaaaaaaatggcaaaagtttctttagttaggtgcttaattttttttgccaagggacgagtcttgggaggagtcctaaactttgtttgattatgaaagcaacccggaaggtgcggtattttacagggagtggcttttgaagggacttactagccactcgctaacgcaaaaattatcgcgttcgcgtcacagtgaagtcaggcagccgtcccctcagcaatgttgaagattcacttcgcgaccaagagcaaccaccatttttttttgctccaaaggcgggagatgcctatttctatcttttaatcgtatatgatcctgtagtgagtccaaacggctcattttgagtacggattactgtaattgctcgtgttggattgagcgaaagtagcgtaagtctcaaaaaaaaattgggttactactcataatactcctggttctttctcatgaaccattttggtcatagcctattggtttgtgccgttctgcacgttttcgtataatattaaagggatcgtgttgttattcacggcaaccattgcgtttgcataaaattactacccaatttcctacttaccaaacgcgcgcgcatagacctcgcaggctttgaagagtcctgtggaattgccgtacaaaaaatcgatctagctcaacagtcctttcagtaattacatatcggaagcccacaaataactgcaacagcgcctacaatgatataaaaggctttgtcttgattcattgctatggggggttttggacagtatgtgttgtttttatttcgagtaaatttagaaacagcaagcctcatcattgcaaatttggtaagatctcgcctaattgaaagagacctgcactaaaacaatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataaaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 173 - DNA, 1544 bp, Synthetic construct
  source 1..1544: mol_type = other DNA
gttgtggaaatgtaaagagccccattatcttagcctaaaaaaaccttctctttggaactttcagtaatacgcttaactgctcattgctatattgaagtacggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaactataacctgcactaaaacaatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataaaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 174 - DNA, 1992 bp, Synthetic construct
  source 1..1992: mol_type = other DNA
agacgcgaatttttcgaagaagtaccttcaaagaatggggtcttatcttgttttgcaagtaccactgagcaggataataatagaaatgataatatactatagtagagataacgtcgatgacttcccatactgtaattgcttttagttgtgtatttttagtgtgcaagtttctgtaaatcgattaatttttttttctttcctctttttattaaccttaatttttattttagattcctgacttcaactcaagacgcacagatattataacatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaacctgcactaaaacaatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataaaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 175 - DNA, 1992 bp, Synthetic construct
  source 1..1992: mol_type = other DNA
ctattttcgaggaccttgtcaccttgagcccaagagagccaagatttaaattttcctatgacttgatgcaaattcccaaagctaataacatgcaagacacgtacggtcaagaagacatatttgacctcttaacaggttcagacgcgactgcctcatcagtaagacccgttgaaaagaacttacctgaaaaaaacgaatatatactagcgttgaatgttagcgtcaacaacaagaagtttaatgacgcggaggccaaggcaaaaagattccttgattacgtaagggagttagaatcattttgaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaacctgcactaaaacaatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataaaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 176 - DNA, 1992 bp, Synthetic construct
  source 1..1992: mol_type = other DNA
tccattcactggcaattcatataaacttttaaccgcgataggatcaagaattctctcgatcacgggtgcgcgaataaatgtagcaggaaaattatcacagccaggtatgaaattggaaaaatcacacttttgtgtaaaagattgagcttgtcttccaaatgcgtttcttctcacgtcgaccttcaacacacctaaagtctttactagggcactttcgttttctaattgcgcgcttaaaaagatgagaccagcacaagtaccccaaactaccttttccggattatgaacaaattcgtataaacaaggatataagcctgttctttgagcgatgagggacatcgacgtagattctcctccgggaataattaaggcgtcgcactgggctagatcctcaggagtttttacagttttgatttctatctttattccgtagtcgttttcagccaaacacctttttaaatggttggtatgctccaaaaaggcaccttgcaacgccaaaaccccaattactttcatcgactttccggacattgtactgtgggttttgtgcatactttaagatatggtttttctttctatttaacgaaatgaaataataagaaaaaaaaacctggtggcatcaacgtgatttttggaagaaagaataaaacattcaaagagttcgtgaccagttattgctgataaatgccgtttactgactcagttggaatttatggcactagttggaatagctcatcatgacataatgactcagccgacaagggtaagggttgaaagccgggctttttcactacttttcaggtagcgccgccatttcttcatgactctgacacactctgccgcaccgttagttacgtctgtaattctggctgcgaagtaccattttcgatataaataggaggacgctgagagctatggacgaagtctatggtattgttggagaaacctttaggaacgactagcaaatatacacagtactaatattcagttaattatcacgacctgcactaaaacaatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataaaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 177 - DNA, 1404 bp, Synthetic construct
  source 1..1404: mol_type = other DNA
agtgcgatccctgaattcgcatctagactgaactggccgataattgcagacgaacgggtagtccatcgttgtaggatactgtatatacacccagtagagtaggtgactactgtatgagcatacagtagtgacaaccacttactgtatataaatacagtagtggtcatcgatactgtatataaaaccagtaggcaatcgtttgtactgtatgtacatacagtatacctcgcaactactgtatataaacacagtaattaacttgtaatattctaatcaattgacaatattatttaaggacctattgttttttccaataggtggttagcaatcgtcttactttctaacttttcttaccttttacatttcagcaatatatatatatatatttcaaggatataccattctaagatctacctgcactaaaacaatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgcgagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataaaggacagatgcgcaatgattgaatagtcaaagattttttttttttaattttttttttttaattttttttttttttcatagaactttttatttaaataaatcacgtctatatatgtatcagtataacgtaaaaaaaaaaacaccgtcagttaaacaaaacataaataaaaaaaaaaagaagtgtcaaatcaagtgtcaaatcaaaatttaaatgatgatgataatatgaccatacaatgacttaaatcaaatcaaaaccac

SEQ ID NO: 178 - DNA, 3397 bp, Synthetic construct
  source 1..3397: mol_type = other DNA
ctattttcgaggaccttgtcaccttgagcccaagagagccaagatttaaattttcctatgacttgatgcaaattcccaaagctaataacatgcaagacacgtacggtcaagaagacatatttgacctcttaacaggttcagacgcgactgcctcatcagtaagacccgttgaaaagaacttacctgaaaaaaacgaatatatactagcgttgaatgttagcgtcaacaacaagaagtttaatgacgcggaggccaaggcaaaaagattccttgattacgtaagggagttagaatcattttgaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaacctgcactaaaacaatgacattatctttcgctcattttacctacctgttcacaatattgttgggattaactaatattgccttggcatctgatccagaaacgattctagtgacgataaccaagacaaacgatgcaaatggggttgttacaactacagtttcacccgcgctagtctccacatccactatcgttcaagctggcactacgacattgtatacgacttggtgtccattgacggtatccacttcatctgctgccgaaataagtccttcaatatcgtacgctactaccctatccagatttagtactttgacattatctacagaagtctgctcccatgaggcatgtccttcgtcatcgacgttgccaaccaccaccttatctgtgacttccaagttcacttcatatatttgccctacttgtcacacaaccgctatcagctcattatccgaagtaggaactacaaccgtggtatcatccagcgccattgaaccatcaagtgcctctataatctcacctgtcacctctacactttcgagtacaacatcgtccaatccaactactacctccctaagttcgacatctacatctccaagctctacatctacatctccaagctctacatctacctcatcaagttcgacatctacctcatcaagttcgacatctacctcatcaagttcgacatctacatctccaagttcgacatccacatcttcaagtttgacatccacatcttcaagttctacatctacatcccaaagttctacatctacctcatcaagttcgacatctacatctccaagctctacatctacctcatcaagttcaacatctacatctccaagttctaaatctacttctgcaagctccacttccacttcttcatattcaacatctacatccccaagtttgacttcttcatctccaactttggcttccacttctccaagttcaacatctattagctctacttttactgattcaacttcatcccttggctcctctatagcatcttcatcaacgtctgtgtcattatacagcccatccacacctgtttactccgtcccttcgacttcgtcaaatgttgcaactccttctatgacttcttcaactgttgaaacaactgttagttcacaaagttcgtctgaatatatcaccaaatcctcaatttctactactatcccatcattttccatgtctacatatttcaccactgttagtggagtcactacaatgtatacgacatggtgtccttatagctctgaatctgagactagcacattaaccagtatgcatgaaacggttacaacagacgctacagtctgcactcacgagtcttgcatgccctcgcagacaacaagtttgattacatcttctataaaaatgtccactaaaaacgtcgcaacttctgtaagcacctcaacggttgaatcctcatatgcatgctccacatgtgctgaaacgtcacactcgtattcttccgtgcaaacagcttcatcaagttctgtaacacagcagaccacatccacaaagagttgggtaagttcaatgacaacttcggatgaagatttcaataagcacgctaccggtaagtatcatgtaacatcttcaggtacctcaaccatttcgactagtgtaagtgaagccacgagtacatcaagcattgactcagaatctcaagaacaatcatcacacttattatcgacatcggtcctttcatcctcctccttgtctgctacattatcctctgacagtactattttgctattcagttctgtatcatcactaagtgtcgaacagtcaccagttaccacacttcaaatttcttcaacatcagagattttacaacccacttcttccacagctattgctacaatatctgcctctacatcatcactttccgcaacatctatctctacaccatctacctctgtggaatcgactattgaatcttcatcattgactccgacggtatcttctattttcctctcatcatcatctgctccctcttctctacaaacatctgttaccactacagaagtttccactacttcaatctccatacaataccaaacttcatcaatggtaacaattagccaatatatgggcagtggatcgcaaacgcgtttgccattaggaaagttggtcttcgccatcatggcagttgcttgcaatgtaattttcagttaaaggacagatgtagatacgttgttgacacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctc

SEQ ID NO: 179 - DNA, 3397 bp, Synthetic construct
  source 1..3397: mol_type = other DNA
ttccttacagttgaaaacaaaatcaaatcacggtatggcatttctttacatattctcaatacctgactgacatagtatgcgctacatagttttgtcctattttttttacaccgcgatttgaagtctaaagccagagaatgaggaaaaacgaaacaaactgagatcatttgtataaggggagtcactgtacagctgccaataccaattttccttctgccaaatcaaaaaagtatccgcatacacatacacccctccctctacaatcttaattatttactgattcctttcgtatttgtccagtatgagcaatactaagcataccacttctcatcacatggaactcaaaaggatcattatccttacacttttattcatactcataatgttgatatttcgaaactcagtgtccttcaaaatgacatttcaagaactactgccacgattttacaaaaagaattcaaactcagttagtaataataacaggccctcatctattttctcggaaaacttggtggattttgatgatgttaacatggtcgataagaccagactgtttatttttttatttttcagtttcatcattactataccgtttatggtataaattagaaaagttaaagcagcatgttttcatttgaaacaaatactaatgcaataataggtacaccatctactacataattaattgatagtttccttttccgttaagtgcatgcataggacgcccataattttttaagttaaaaaagcatgtatttacctattcgggaaatttacatgacatggatgccataaggaacgaaaatgaaacatgcatgtcagaggaaaaagaacaaagcgagaggaaaaagctgttgcattaccacgtaattttgtatataaatatctgataaataaccattttatttccatccacttcttctgtcgttcatccgttcattgacatcactagagacaccagcgagctatcatcttcatacaacaataactaccaaccttaacctgcactaaaacaatgacattatctttcgctcattttacctacctgttcacaatattgttgggattaactaatattgccttggcatctgatccagaaacgattctagtgacgataaccaagacaaacgatgcaaatggggttgttacaactacagtttcacccgcgctagtctccacatccactatcgttcaagctggcactacgacattgtatacgacttggtgtccattgacggtatccacttcatctgctgccgaaataagtccttcaatatcgtacgctactaccctatccagatttagtactttgacattatctacagaagtctgctcccatgaggcatgtccttcgtcatcgacgttgccaaccaccaccttatctgtgacttccaagttcacttcatatatttgccctacttgtcacacaaccgctatcagctcattatccgaagtaggaactacaaccgtggtatcatccagcgccattgaaccatcaagtgcctctataatctcacctgtcacctctacactttcgagtacaacatcgtccaatccaactactacctccctaagttcgacatctacatctccaagctctacatctacatctccaagctctacatctacctcatcaagttcgacatctacctcatcaagttcgacatctacctcatcaagttcgacatctacatctccaagttcgacatccacatcttcaagtttgacatccacatcttcaagttctacatctacatcccaaagttctacatctacctcatcaagttcgacatctacatctccaagctctacatctacctcatcaagttcaacatctacatctccaagttctaaatctacttctgcaagctccacttccacttcttcatattcaacatctacatccccaagtttgacttcttcatctccaactttggcttccacttctccaagttcaacatctattagctctacttttactgattcaacttcatcccttggctcctctatagcatcttcatcaacgtctgtgtcattatacagcccatccacacctgtttactccgtcccttcgacttcgtcaaatgttgcaactccttctatgacttcttcaactgttgaaacaactgttagttcacaaagttcgtctgaatatatcaccaaatcctcaatttctactactatcccatcattttccatgtctacatatttcaccactgttagtggagtcactacaatgtatacgacatggtgtccttatagctctgaatctgagactagcacattaaccagtatgcatgaaacggttacaacagacgctacagtctgcactcacgagtcttgcatgccctcgcagacaacaagtttgattacatcttctataaaaatgtccactaaaaacgtcgcaacttctgtaagcacctcaacggttgaatcctcatatgcatgctccacatgtgctgaaacgtcacactcgtattcttccgtgcaaacagcttcatcaagttctgtaacacagcagaccacatccacaaagagttgggtaagttcaatgacaacttcggatgaagatttcaataagcacgctaccggtaagtatcatgtaacatcttcaggtacctcaaccatttcgactagtgtaagtgaagccacgagtacatcaagcattgactcagaatctcaagaacaatcatcacacttattatcgacatcggtcctttcatcctcctccttgtctgctacattatcctctgacagtactattttgctattcagttctgtatcatcactaagtgtcgaacagtcaccagttaccacacttcaaatttcttcaacatcagagattttacaacccacttcttccacagctattgctacaatatctgcctctacatcatcactttccgcaacatctatctctacaccatctacctctgtggaatcgactattgaatcttcatcattgactccgacggtatcttctattttcctctcatcatcatctgctccctcttctctacaaacatctgttaccactacagaagtttccactacttcaatctccatacaataccaaacttcatcaatggtaacaattagccaatatatgggcagtggatcgcaaacgcgtttgccattaggaaagttggtcttcgccatcatggcagttgcttgcaatgtaattttcagttaaaggacagatgtagatacgttgttgacacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctc

SEQ ID NO: 180 - DNA, 3397 bp, Synthetic construct
  source 1..3397: mol_type = other DNA
agacgcgaatttttcgaagaagtaccttcaaagaatggggtcttatcttgttttgcaagtaccactgagcaggataataatagaaatgataatatactatagtagagataacgtcgatgacttcccatactgtaattgcttttagttgtgtatttttagtgtgcaagtttctgtaaatcgattaatttttttttctttcctctttttattaaccttaatttttattttagattcctgacttcaactcaagacgcacagatattataacatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaacctgcactaaaacaatgacattatctttcgctcattttacctacctgttcacaatattgttgggattaactaatattgccttggcatctgatccagaaacgattctagtgacgataaccaagacaaacgatgcaaatggggttgttacaactacagtttcacccgcgctagtctccacatccactatcgttcaagctggcactacgacattgtatacgacttggtgtccattgacggtatccacttcatctgctgccgaaataagtccttcaatatcgtacgctactaccctatccagatttagtactttgacattatctacagaagtctgctcccatgaggcatgtccttcgtcatcgacgttgccaaccaccaccttatctgtgacttccaagttcacttcatatatttgccctacttgtcacacaaccgctatcagctcattatccgaagtaggaactacaaccgtggtatcatccagcgccattgaaccatcaagtgcctctataatctcacctgtcacctctacactttcgagtacaacatcgtccaatccaactactacctccctaagttcgacatctacatctccaagctctacatctacatctccaagctctacatctacctcatcaagttcgacatctacctcatcaagttcgacatctacctcatcaagttcgacatctacatctccaagttcgacatccacatcttcaagtttgacatccacatcttcaagttctacatctacatcccaaagttctacatctacctcatcaagttcgacatctacatctccaagctctacatctacctcatcaagttcaacatctacatctccaagttctaaatctacttctgcaagctccacttccacttcttcatattcaacatctacatccccaagtttgacttcttcatctccaactttggcttccacttctccaagttcaacatctattagctctacttttactgattcaacttcatcccttggctcctctatagcatcttcatcaacgtctgtgtcattatacagcccatccacacctgtttactccgtcccttcgacttcgtcaaatgttgcaactccttctatgacttcttcaactgttgaaacaactgttagttcacaaagttcgtctgaatatatcaccaaatcctcaatttctactactatcccatcattttccatgtctacatatttcaccactgttagtggagtcactacaatgtatacgacatggtgtccttatagctctgaatctgagactagcacattaaccagtatgcatgaaacggttacaacagacgctacagtctgcactcacgagtcttgcatgccctcgcagacaacaagtttgattacatcttctataaaaatgtccactaaaaacgtcgcaacttctgtaagcacctcaacggttgaatcctcatatgcatgctccacatgtgctgaaacgtcacactcgtattcttccgtgcaaacagcttcatcaagttctgtaacacagcagaccacatccacaaagagttgggtaagttcaatgacaacttcggatgaagatttcaataagcacgctaccggtaagtatcatgtaacatcttcaggtacctcaaccatttcgactagtgtaagtgaagccacgagtacatcaagcattgactcagaatctcaagaacaatcatcacacttattatcgacatcggtcctttcatcctcctccttgtctgctacattatcctctgacagtactattttgctattcagttctgtatcatcactaagtgtcgaacagtcaccagttaccacacttcaaatttcttcaacatcagagattttacaacccacttcttccacagctattgctacaatatctgcctctacatcatcactttccgcaacatctatctctacaccatctacctctgtggaatcgactattgaatcttcatcattgactccgacggtatcttctattttcctctcatcatcatctgctccctcttctctacaaacatctgttaccactacagaagtttccactacttcaatctccatacaataccaaacttcatcaatggtaacaattagccaatatatgggcagtggatcgcaaacgcgtttgccattaggaaagttggtcttcgccatcatggcagttgcttgcaatgtaattttcagttaaaggacagatgtagatacgttgttgacacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctc

SEQ ID NO: 181 - DNA, 3013 bp, Synthetic construct
  source 1..3013: mol_type = other DNA
ccttgtgaaattgcaaatatggtgatttgaaacgtttcctagtgcagcaggatcacagataacgtgtaaagggcttagcagttgataatcctctctagttaagacctaaacaaaatgctgtcactaaccgtagtattaaatgacacactttggtgactttcgttaatggggatgtggtagtggccattgccaataaacaaaaagaacagggaaagaagtagaaagtgatataagtttgcttgccacttttcgtttttcacgaaaaaaacaggcgaaaaaaaatgctagacaagtacccggctgaatcacacctcgttaacagtgactttcggtgacagatacccgattgggcacccggctggtaagttatgatagaaagccaacgctgtactattggcttagctatggcaatattttgattatcagctagttttattaacgttataattagtgtaaccagtttttcatctatttcatttatttcatttatttactttaattgcagatccccctaacgcgtttaaagcttttattcactagcttatgtattttttataggaaacgcgacgcgtaacatcgcgcaaatgaaggttttgatgtattataatgaggtattcttccttatatacatcgatgaaaagcgttgacagcatacattggaaagaaataggaaacggacaccggaagaaaaaatagatctatgaaggctttaactgcaagacaacaggaagtttttgatttgataagagatcatatatctcaaactggaatgccaccaacaagagctgaaatagctcaaagattaggatttagatctccaaacgctgcagaagaacatttgaaagctttggcaaggaaaggtgtcatcgaaattgtctctggtgcttcaagaggtattagactattacaagaagaggaagaaggattgccattggttggtagagttgctgctggtgaaccattattagcacagcaacatattgagggtcattatcaagtagatccatctttattcaaaccaaatgctgattttttgttgagagtaagtggtatgtctatgaaagatattggtatcatggatggtgacttattggctgttcataaaactcaagatgttagaaacggtcaagttgtcgttgccagaattgatgatgaagtcactgttaaaagattgaagaaacaaggtaataaagtggaattgttaccagaaaattctgaatttaagccaattgttgttgatttaaggcaacaatcttttactattgaaggtttagctgtcggtgttatcagaaatggtgattggttaggttcaggttctcctccaaaaaaaaagagaaaagttggatctggaagtagaccatctagtacaacaaaatcagataattcgcctccaaaattagaaagcgagaattttaaggataatgagttggtaacagtaactaatcagccgcttttaggcgttggcctcatggatgacgatgcgccagaatccccctctcaaattaatgattttattcctcagaaattgattatagaacccaatactctcgaattgaatggtctaacagaagaaacgcctcatgacttacccaagaataccgctaagggcagagatgaagaagattttcctctcgactattttcctgtatctgttgaataccctacggaggaaaatgcgtttgatccgttccctccacaggcttttacgccagctgccccttccatgcctatttcctatgataacgtgaatgaaagggattctatgcccgttaattctcttcttaatagatacccctatcagttatcagtggcacccactttcccagtgccaccatcatcatcgaggcaacattttatgacaaatcgggatttttattcatctaacaataacaaggaaaaattggtatctcctagcgaccctaccagctacatgaagtatgacgaaccagttatggattttgatgaatctcggccaaatgaaaactgtacaaatgcaaaatctcacaactctggccagcaaactaaacaacaccaattatattctaacaacttccagcaatcttacccaaacggaatggttccaggatactacccaaaaatgccgtataatcccatggggggggatcctctactcgatcaagccttttatggcgcggacgattttttctttccaccagaaggatgtgataacaatatgctgtatccacaaactgcaacttcatggaatgttttgccccctcaagctatgcaaccagctccaacctatgttgggaggccatacacaccgaattatagatcgacaccaggttccgcgatgttcccatacatgcaaagttcaaattccatgcagtggaacactgctgtttcaccttatagttcgagagcaccatctacaactgctaaaaactatcctcctagcacattttattctcaaaatataaatcaatacccacggcgaagaactgtgggaatgaagtcttcacaaggaaatgttccaacaggtaataaacaatctgtgggcaagtctgcaaaaatttcaaagcctctacatattaagacaagtgcttatcagaagcaatacaaaatcaacttggaaacgaaagccaggccaagtgctggtgacgaagattctgctcatcctgataagaacaaagaaatttcgatgcctactccggattccaatactttggtggtccagtcagaagaaggtggagctcattcacttgaggtagataccaatcgaaggtccgataaaaaccttccagatgcaaccggatcctaactcgagagcttttgattaagccttctagtccaaaaaacacgtttttttgtcatttatttcattttcttagaatagtttagtttattcattttatagtcacgaatgttttatgattctatatagggttgcaaacaagcatttttcattttatgttaaaacaatttcaggtttaccttttattctgcttgtggtgacgcgtgtatccgcccgctcttttggtcacccatgtat

SEQ ID NO: 182 - DNA, 2604 bp, Synthetic construct
  source 1..2604: mol_type = other DNA
cgaagaagtaccttcaaagaatggggtcttatcttgttttgcaagtaccactgagcaggataataatagaaatgataatatactatagtagagataacgtcgatgacttcccatactgtaattgcttttagttgtgtatttttagtgtgcaagtttctgtaaatcgattaatttttttttctttcctctttttattaaccttaatttttattttagattcctgacttcaactcaagacgcacagatattataacatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaacctgcactaaaacaatggggtgtacagtgagcacgcaaacaataggagacgaaagtgatccttttctacagaacaaaagagccaatgatgtcatcgagcaatcgttgcagctggagaaacaacgtgacaagaatgaaataaaactgttactattaggtgccggtgagtcaggtaaatcaacggttttaaaacaattaaaattattacatcaaggcggtttctcccatcaagaaaggttacagtatgctcaagtgatacgggcagatgccatacaatcaatgaaaattttgattattcaggccagaaaactaggtattcaacttgactgtgatgatccgatcaacaataaagatttgtttgcatgtaagagaatactgctaaaggctaaagctttagattatatcaacgccagtgttgccggtggttctgattttctaaatgattatgtactgaagtactcagaaaggtatgaaactaggaggcgtgttcagagtaccggacgagcaaaagctgctttcgatgaagacggaaatatttctaatgtcaaaagtgacactgacagagatgctgaaacggtgacgcaaaatgaggatgttgatagaaacaacagtagtagaattaacctacaggatatttgcaaggacttgaaccaagaaggcgatgaccagatgtttgttagaaaaacatcaagggaaattcaaggacaaaatagacgaaatcttattcacgaagacattgctaaggcaataaagcaactttggaataacgacaaaggtataaagcagtgctttgcacgttctaatgagtttcaattggagggctcagctgcatactactttgataacattgagaaatttgctagtccgaattatgtctgtacggatgaagacattttgaagggccgtataaagactacaggcattacagaaaccgaatttaacatcggctcgtccaaattcaaggttctcgacgctggtgggcagcgttctgaacgtaagaagtggattcattgtttcgaaggaattacagcagttttatttgttttagcaatgagtgaatacgaccagatgttgtttgaggatgaaagagtgaacagaatgcatgaatcaataatgctatttgacacgttattgaactctaagtggttcaaagatacaccgtttattttgtttttaaataaaattgatttgttcgaggaaaaggtaaaaagcatgcccataagaaagtactttcctgattaccagggacgtgtcggcgatgcagaagcgggtctaaaatattttgagaagatatttttgagcttgaataagacaaacaaaccaatctacgtgaaacgaacctgcgctaccgatacccaaactatgaagttcgtattgagtgcagtcaccgatctaatcatccagcaaaaccttaaaaaaagtggtattatatgaatcgcgtgcattcatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaa

SEQ ID NO: 183 - DNA, 2604 bp, Synthetic construct
  source 1..2604: mol_type = other DNA
cgaagaagtaccttcaaagaatggggtcttatcttgttttgcaagtaccactgagcaggataataatagaaatgataatatactatagtagagataacgtcgatgacttcccatactgtaattgcttttagttgtgtatttttagtgtgcaagtttctgtaaatcgattaatttttttttctttcctctttttattaaccttaatttttattttagattcctgacttcaactcaagacgcacagatattataacatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaacctgcactaaaacaatggggtgtacagtgagcacgcaaacaataggagacgaaagtgatccttttctacagaacaaaagagccaatgatgtcatcgagcaatcgttgcagctggagaaacaacgtgacaagaatgaaataaaactgttactattaggtgccggtgagtcaggtaaatcaacggttttaaaacaattaaaattattacatcaaggcggtttctcccatcaagaaaggttacagtatgctcaagtgatacgggcagatgccatacaatcaatgaaaattttgattattcaggccagaaaactaggtattcaacttgactgtgatgatccgatcaacaataaagatttgtttgcatgtaagagaatactgctaaaggctaaagctttagattatatcaacgccagtgttgccggtggttctgattttctaaatgattatgtactgaagtactcagaaaggtatgaaactaggaggcgtgttcagagtaccggacgagcaaaagctgctttcgatgaagacggaaatatttctaatgtcaaaagtgacactgacagagatgctgaaacggtgacgcaaaatgaggatgttgatagaaacaacagtagtagaattaacctacaggatatttgcaaggacttgaaccaagaaggcgatgaccagatgtttgttagaaaaacatcaagggaaattcaaggacaaaatagacgaaatcttattcacgaagacattgctaaggcaataaagcaactttggaataacgacaaaggtataaagcagtgctttgcacgttctaatgagtttcaattggagggctcagctgcatactactttgataacattgagaaatttgctagtccgaattatgtctgtacggatgaagacattttgaagggccgtataaagactacaggcattacagaaaccgaatttaacatcggctcgtccaaattcaaggttctcgacgctggtgggcagcgttctgaacgtaagaagtggattcattgtttcgaaggaattacagcagttttatttgttttagcaatgagtgaatacgaccagatgttgtttgaggatgaaagagtgaacagaatgcatgaatcaataatgctatttgacacgttattgaactctaagtggttcaaagatacaccgtttattttgtttttaaataaaattgatttgttcgaggaaaaggtaaaaagcatgcccataagaaagtactttcctgattaccagggacgtgtcggcgatgcagaagcgggtctaaaatattttgagaagatatttttgagcttgaataagacaaacaaaccaatctacgtgaaacgaacctgcgctaccgatacccaaactatgaagttcgtattgagtgcagtcactgacctaatcatccagcaaaaccttaaatatattggtttgtgttgaatcgcgtgcattcatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaa

SEQ ID NO: 184 - DNA, 2604 bp, Synthetic construct
  source 1..2604: mol_type = other DNA
cgaagaagtaccttcaaagaatggggtcttatcttgttttgcaagtaccactgagcaggataataatagaaatgataatatactatagtagagataacgtcgatgacttcccatactgtaattgcttttagttgtgtatttttagtgtgcaagtttctgtaaatcgattaatttttttttctttcctctttttattaaccttaatttttattttagattcctgacttcaactcaagacgcacagatattataacatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaacctgcactaaaacaatggggtgtacagtgagcacgcaaacaataggagacgaaagtgatccttttctacagaacaaaagagccaatgatgtcatcgagcaatcgttgcagctggagaaacaacgtgacaagaatgaaataaaactgttactattaggtgccggtgagtcaggtaaatcaacggttttaaaacaattaaaattattacatcaaggcggtttctcccatcaagaaaggttacagtatgctcaagtgatacgggcagatgccatacaatcaatgaaaattttgattattcaggccagaaaactaggtattcaacttgactgtgatgatccgatcaacaataaagatttgtttgcatgtaagagaatactgctaaaggctaaagctttagattatatcaacgccagtgttgccggtggttctgattttctaaatgattatgtactgaagtactcagaaaggtatgaaactaggaggcgtgttcagagtaccggacgagcaaaagctgctttcgatgaagacggaaatatttctaatgtcaaaagtgacactgacagagatgctgaaacggtgacgcaaaatgaggatgttgatagaaacaacagtagtagaattaacctacaggatatttgcaaggacttgaaccaagaaggcgatgaccagatgtttgttagaaaaacatcaagggaaattcaaggacaaaatagacgaaatcttattcacgaagacattgctaaggcaataaagcaactttggaataacgacaaaggtataaagcagtgctttgcacgttctaatgagtttcaattggagggctcagctgcatactactttgataacattgagaaatttgctagtccgaattatgtctgtacggatgaagacattttgaagggccgtataaagactacaggcattacagaaaccgaatttaacatcggctcgtccaaattcaaggttctcgacgctggtgggcagcgttctgaacgtaagaagtggattcattgtttcgaaggaattacagcagttttatttgttttagcaatgagtgaatacgaccagatgttgtttgaggatgaaagagtgaacagaatgcatgaatcaataatgctatttgacacgttattgaactctaagtggttcaaagatacaccgtttattttgtttttaaataaaattgatttgttcgaggaaaaggtaaaaagcatgcccataagaaagtactttcctgattaccagggacgtgtcggcgatgcagaagcgggtctaaaatattttgagaagatatttttgagcttgaataagacaaacaaaccaatctacgtgaaacgaacctgcgctaccgatacccaaactatgaagttcgtattgagtgcagtcactgacctaatcatccagcaaaaccttaaagattgtggtttgttttgaatcgcgtgcattcatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaa

SEQ ID NO: 185 - DNA, 2510 bp, Synthetic construct
  source 1..2510: mol_type = other DNA
aaccagggcaaagcaaaataaaagaaacttaatacgttatgccgtaatgaagggctaccaaaaacgataatctcaactgtaaacaggtacaatgcggacccttttgccacaaaacatacatcattcattgccggaaaaagaaagaagtgaagacagcagtgcagccagccatgttgcgccaatctaattatagatgctggtgccctgaggatgtatctggagccagccatggcatcatgcgctaccgccggatgtaaaatccgacacgcaaaagaaaaccttcgaggttgcgcacttcgcccacccatgaaccacacggttagtccaaaaggggcagttcagattccagatgcgggaattagcttgctgccaccctcacctcactaacgctgcggtgtgcggatacttcatgctatttatagacgcgcgtgtcggaatcagcacgcgcaagaaccaaatgggaaaatcggaatgggtccagaactgctttgagtgctggctattggcgtctgatttccgttttgggaatcctttgccgcgcgcccctctcaaaactccgcacaagtcccagaaagcgggaaagaaataaaacgccaccaaaaaaaaaaaaataaaagccaatcctcgaagcgtgggtggtaggccctggattatcccgtacaagtatttctcaggagtaaaaaaaccgtttgttttggaattccccatttcgcggccacctacgccgctatctttgcaacaactatctgcgataactcagcaaattttgcatattcgtgttgcagtattgcgataatgggagtcttacttccaacataacggcagaaagaaatgtgagaaaattttgcatcctttgcctccgttcaagtatataaagtcggcatgcttgataatctttctttccatcctacattgttctaattattcttattctcctttattctttcctaacataccaagaaattaatcttctgtcattcgcttaaacactatatcaataacctgcactaaaacaatgtctgatgcggctccttcattgagcaatctattttatgatccaacgtataatcctggtcaaagcaccattaactacacttccatatatgggaatggatctaccatcactttcgatgagttgcaaggtttagttaacagtactgttactcaggccattatgtttggtgtcagatgtggtgcagctgctttgactttgattgtcatgtggatgacatcgagaagcagaaaaacgccgattttcattatcaaccaagtttcattgtttttaatcattttgcattctgcactctattttaaatatttactgtctaattactcttcagtgacttacgctctcaccggatttcctcagttcatcagtagaggtgacgttcatgtttatggtgctacaaatataattcaagtccttcttgtggcttctattgagacttcactggtgtttcagataaaagttattttcacaggcgacaacttcaaaaggataggtttgatgctgacgtcgatatctttcactttagggattgctacagttaccatgtattttgtaagcgctgttaaaggtatgattgtgacttataatgatgttagtgccacccaagataaatacttcaatgcatccacaattttacttgcatcctcaataaactttatgtcatttgtcctggtagttaaattgattttagctattagatcaagaagattccttggtctcaagcagttcgatagtttccatattttactcataatgtcatgtcaatctttgttggttccatcgataatattcatcctcgcatacagtttgaaaccaaaccagggaacagatgtcttgactactgttgcaacattacttgctgtattgtctttaccattatcatcaatgtgggccacggctgctaataatgcatccaaaacaaacacaattacttcagactttacaacatccacagataggttttatccaggcacgctgtctagctttcaaactgatagtatcaacaacgatgctaaaagcagtctcagaagtagattatatgacctatatcctagaaggaaggaaacaacatcggataaacattcggaaagaacttttgtttctgagactgcagatgatatagagaaaaatcagttttatcagttgcccacacctacgagttcaaaaaatactaggataggaccgtttgctgatgcaagttacaaagagggagaagttgaacccgtcgacatgtacactcccgatacggcagctgatgaggaagccagaaagttctggactgaagataataataatttatgaatgacagatatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaag

SEQ ID NO: 186 - DNA, 2267 bp, Synthetic construct
  source 1..2267: mol_type = other DNA
aaccagggcaaagcaaaataaaagaaacttaatacgttatgccgtaatgaagggctaccaaaaacgataatctcaactgtaaacaggtacaatgcggacccttttgccacaaaacatacatcattcattgccggaaaaagaaagaagtgaagacagcagtgcagccagccatgttgcgccaatctaattatagatgctggtgccctgaggatgtatctggagccagccatggcatcatgcgctaccgccggatgtaaaatccgacacgcaaaagaaaaccttcgaggttgcgcacttcgcccacccatgaaccacacggttagtccaaaaggggcagttcagattccagatgcgggaattagcttgctgccaccctcacctcactaacgctgcggtgtgcggatacttcatgctatttatagacgcgcgtgtcggaatcagcacgcgcaagaaccaaatgggaaaatcggaatgggtccagaactgctttgagtgctggctattggcgtctgatttccgttttgggaatcctttgccgcgcgcccctctcaaaactccgcacaagtcccagaaagcgggaaagaaataaaacgccaccaaaaaaaaaaaaataaaagccaatcctcgaagcgtgggtggtaggccctggattatcccgtacaagtatttctcaggagtaaaaaaaccgtttgttttggaattccccatttcgcggccacctacgccgctatctttgcaacaactatctgcgataactcagcaaattttgcatattcgtgttgcagtattgcgataatgggagtcttacttccaacataacggcagaaagaaatgtgagaaaattttgcatcctttgcctccgttcaagtatataaagtcggcatgcttgataatctttctttccatcctacattgttctaattattcttattctcctttattctttcctaacataccaagaaattaatcttctgtcattcgcttaaacactatatcaataacctgcactaaaacaatgaggcaaccttggtggaaagactttactattcccgatgcaagcgcaattattcaccaaaatattaccattgtgtctattgtcggagagattgaagtgcctgtttcaacaattgatgcatatgaaagggataggttattaactggaatgactttatctgcccaattagctttaggagtgttaaccattttaatggtttgtctgttatcatcaagcgaaaaacgtaaacaccctgtttttgtttttaattcggcaagtattgttgcaatgtgtttacgcgccattttaaatatcgtgaccatctgctcgaattcgtacagtatcctcgttaattacgggtttatcttaaacatggttcatatgtatgtgcatgtgtttaatattttaattttattattagcaccggtgatcatttttactgctgagatgtcgatgatgattcaagttaggatcatttgtgcacatgataggaaaacacaacgtatcatgactgttattagtgcctgcttaactgttttagttctcgcattttggattactaacatgtgtcaacagattcagtatctcttatggttaactcctttatcgtcgaaaaccattgttggatactcttggccctactttattgctaaaatcttatttgctttttcgattatttttcacagtggtgttttttcatacaaactctttagggccatcttaatccgcaaaaaaattgggcaatttccttttggtccgatgcagtgtattttagttatttcgtgccaatgtttaattgttcctgctacctttactatcatcgatagttttatccatacgtatgatggcttttcgtctatgactcaatgtctcctgatcatttctttaccattatcgagtttatgggcgtctagtacagctctcaaattacaatcgatgaaaacttcatctgcgcaaggagaaaccaccgaggtttcgattagggttgataggacgtttgatatcaaacatactcccagtgacgattattcgatttctgatgaatctgaaactaaaaagtggacgggatcctgaatgacagatatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaag

SEQ ID NO: 187 - DNA, 2273 bp, Synthetic construct
  source 1..2273: mol_type = other DNA
aaccagggcaaagcaaaataaaagaaacttaatacgttatgccgtaatgaagggctaccaaaaacgataatctcaactgtaaacaggtacaatgcggacccttttgccacaaaacatacatcattcattgccggaaaaagaaagaagtgaagacagcagtgcagccagccatgttgcgccaatctaattatagatgctggtgccctgaggatgtatctggagccagccatggcatcatgcgctaccgccggatgtaaaatccgacacgcaaaagaaaaccttcgaggttgcgcacttcgcccacccatgaaccacacggttagtccaaaaggggcagttcagattccagatgcgggaattagcttgctgccaccctcacctcactaacgctgcggtgtgcggatacttcatgctatttatagacgcgcgtgtcggaatcagcacgcgcaagaaccaaatgggaaaatcggaatgggtccagaactgctttgagtgctggctattggcgtctgatttccgttttgggaatcctttgccgcgcgcccctctcaaaactccgcacaagtcccagaaagcgggaaagaaataaaacgccaccaaaaaaaaaaaaataaaagccaatcctcgaagcgtgggtggtaggccctggattatcccgtacaagtatttctcaggagtaaaaaaaccgtttgttttggaattccccatttcgcggccacctacgccgctatctttgcaacaactatctgcgataactcagcaaattttgcatattcgtgttgcagtattgcgataatgggagtcttacttccaacataacggcagaaagaaatgtgagaaaattttgcatcctttgcctccgttcaagtatataaagtcggcatgcttgataatctttctttccatcctacattgttctaattattcttattctcctttattctttcctaacataccaagaaattaatcttctgtcattcgcttaaacactatatcaataacctgcactaaaacaatgcaaggtaatggttctgctttgccaaatgcttctcaaccagttttgagaggtgatggtgctagaccttcttggttggcttctgctttagcttgtgttttgattttcaccatcgttgtcgatatcttgggtaacttgttggttatcttgtccgtctaccgtaacaagaaattgagaaacgctggtaacatcttcgttgtttctttggctgttgctgatttggttgttgctatctatccatatccactggtcttgatgtccatttttaacaacggttggaacttgggttacttgcattgtcaagtttctggtttcttgatgggtttgtccgttattggttccattttcaacattaccggtatcgccattaacaggtactgttacatttgccactcactgaagtacgacaagttgtactcttctaagaactccttgtgctacgttttgttgatctggttgttaactttggctgctgttttgcctaatttgagagctggtacattgcaatacgatccaagaatctactcttgtaccttcgctcaatctgtttcttctgcttacactattgccgttgtcgttttccattttttggtcccaatgattatcgtcatcttctgctacttgagaatctggattttggtcttgcaagtcagacaaagagttaagccagatagaaagccaaaattgaagccacaagacttcagaaacttcgttaccatgtttgtggttttcgttttgttcgctatttgttgggctccattgaactttattggtttggcagttgcttctgatccagcttctatggttccaagaattccagaatggttgttcgttgcttcttactacatggcttacttcaactcttgtttgaacgccattatctacggcttgttgaatcagaactttaggaaagagtacaggcgtatcatcgtttctttgtgtactgctagagttttcttcgtcgattcctctaatgatgttgccgatagagttaagtggaaaccatctccattgatgaccaacaacaatgttgtcaaggttgactccgttggatcctgaatgacagatatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaag

SEQ ID NO: 188 - DNA, 2219 bp, Synthetic construct
  source 1..2219: mol_type = other DNA
aaccagggcaaagcaaaataaaagaaacttaatacgttatgccgtaatgaagggctaccaaaaacgataatctcaactgtaaacaggtacaatgcggacccttttgccacaaaacatacatcattcattgccggaaaaagaaagaagtgaagacagcagtgcagccagccatgttgcgccaatctaattatagatgctggtgccctgaggatgtatctggagccagccatggcatcatgcgctaccgccggatgtaaaatccgacacgcaaaagaaaaccttcgaggttgcgcacttcgcccacccatgaaccacacggttagtccaaaaggggcagttcagattccagatgcgggaattagcttgctgccaccctcacctcactaacgctgcggtgtgcggatacttcatgctatttatagacgcgcgtgtcggaatcagcacgcgcaagaaccaaatgggaaaatcggaatgggtccagaactgctttgagtgctggctattggcgtctgatttccgttttgggaatcctttgccgcgcgcccctctcaaaactccgcacaagtcccagaaagcgggaaagaaataaaacgccaccaaaaaaaaaaaaataaaagccaatcctcgaagcgtgggtggtaggccctggattatcccgtacaagtatttctcaggagtaaaaaaaccgtttgttttggaattccccatttcgcggccacctacgccgctatctttgcaacaactatctgcgataactcagcaaattttgcatattcgtgttgcagtattgcgataatgggagtcttacttccaacataacggcagaaagaaatgtgagaaaattttgcatcctttgcctccgttcaagtatataaagtcggcatgcttgataatctttctttccatcctacattgttctaattattcttattctcctttattctttcctaacataccaagaaattaatcttctgtcattcgcttaaacactatatcaataacctgcactaaaacaatgttgttggaaacacaagatgcgttgtatgttgcgttggaattggtaattgctgcgctatcggttgcgggaaatgttttggtttgtgctgcggttggaacggcgaataccttgcaaacgcctactaattattttttggtttcattggcagcggctgatgttgctgttggactatttgctattcctttcgctattactatttctttgggattttgtaccgatttttatggatgtctatttctagcttgttttgttttggttctaacgcaatcttcaattttttctctattggctgttgccgtagataggtatttggctatttgcgtaccgctaaggtacaagtcccttgtaacgggaactcgtgccaggggagtaatagcagtactatgggtactagctttcggaattggacttaccccttttttgggatggaattccaaggattccgctactaataattgtacagagccttgggacggaactacgaacgagtcttgttgtctagttaaatgcctattcgaaaacgttgtacctatgtcttatatggtgtactttaactttttcggatgcgtgttgcctcctttgctaatcatgttggttatttatataaaaatttttttggttgcttgtaggcaactacaacgtaccgaattgatggatcattcgaggactactctacaaagggaaattcacgccgctaaatccttggctatgatagttggaatattcgctttgtgttggctccctgttcacgcagtgaattgcgtaaccctatttcaacctgcacaaggcaagaacaaacctaaatgggccatgaacatggctatactattgtcccacgctaactccgtggtaaaccctatagtatacgcatataggaatcgtgattttcgttataccttccataagataatttcaaggtacctactatgtcaggccgacgtaaaatccggaaacggccaagcaggagtgcaacctgcactaggcgttggactcggatcctgaatgacagatatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaag

SEQ ID NO: 189 - DNA, 2612 bp, Synthetic construct
  source 1..2612: mol_type = other DNA
aaccagggcaaagcaaaataaaagaaacttaatacgttatgccgtaatgaagggctaccaaaaacgataatctcaactgtaaacaggtacaatgcggacccttttgccacaaaacatacatcattcattgccggaaaaagaaagaagtgaagacagcagtgcagccagccatgttgcgccaatctaattatagatgctggtgccctgaggatgtatctggagccagccatggcatcatgcgctaccgccggatgtaaaatccgacacgcaaaagaaaaccttcgaggttgcgcacttcgcccacccatgaaccacacggttagtccaaaaggggcagttcagattccagatgcgggaattagcttgctgccaccctcacctcactaacgctgcggtgtgcggatacttcatgctatttatagacgcgcgtgtcggaatcagcacgcgcaagaaccaaatgggaaaatcggaatgggtccagaactgctttgagtgctggctattggcgtctgatttccgttttgggaatcctttgccgcgcgcccctctcaaaactccgcacaagtcccagaaagcgggaaagaaataaaacgccaccaaaaaaaaaaaaataaaagccaatcctcgaagcgtgggtggtaggccctggattatcccgtacaagtatttctcaggagtaaaaaaaccgtttgttttggaattccccatttcgcggccacctacgccgctatctttgcaacaactatctgcgataactcagcaaattttgcatattcgtgttgcagtattgcgataatgggagtcttacttccaacataacggcagaaagaaatgtgagaaaattttgcatcctttgcctccgttcaagtatataaagtcggcatgcttgataatctttctttccatcctacattgttctaattattcttattctcctttattctttcctaacataccaagaaattaatcttctgtcattcgcttaaacactatatcaataacctgcactaaaacaatgttccggcaggagcagccactcgccgagggttcctttgcccccatgggaagtcttcagcctgacgccggcaacgctagctggaatggcaccgaagcgcccggtggaggggctcgggctaccccatattctctccaggtcacactgaccctggtttgtctggccggtctcctgatgctgctgacggtctttggcaatgttttggtgataattgcggtgttcacctccagagcactcaaggctccccagaacctgttcttggtcagcctggcgtctgccgatattctggtcgccaccctcgtaattccgttttctcttgccaatgaggttatgggttactggtattttggtaaggcatggtgcgagatataccttgctctcgacgttctcttctgcaccagcagtattgtgcatctgtgcgcaattagtctggacagatattggtccatcacgcaggcaatagaatataacttgaaaaggactcctcggcgcattaaggctatcatcatcactgtctgggtcattagcgctgtgataagcttccctccacttatttccattgaaaaaaaaggaggaggcggagggccgcagcctgcggagccccgctgtgagatcaacgatcagaaatggtatgtcatctcctcctgcattgggtccttcttcgccccatgtctgatcatgatcctcgtatatgttaggatttatcagatagctaagcgaaggacccgggtccccccatccaggagaggtccagacgcagtggccgctccccccggaggcacagaaaggagacctaacggtctgggaccagagcgctccgcaggacctggaggtgcagaagccgagcccttgccgacacaactgaacggagctcccggcgaaccagccccagcaggtcctcgcgatactgacgcactggacctcgaagagtcctctagcagcgatcatgcagagcgccctccagggcctaggagacctgaaagaggacccagaggtaaaggcaaggcccgggcaagtcaagtgaaaccgggagactctctgcctcggaggggtccaggtgctactggtatagggactcctgccgccggtcccggagaagagcgggtcggcgccgctaaggcaagtcgctggcggggcagacaaaaccgggaaaaaaggtttacattcgtgctcgcggtagtgattggcgtcttcgtcgtgtgctggtttccattcttttttacctataccctgacagccgtcggctgttctgttccacggacactgttcaagttctttttctggtttggctactgtaactcaagcctgaacccggtaatctatacaatatttaaccacgattttcggagggcttttaaaaagatcctgtgcagaggcgataggaaacgaatcgtttaaatgacagatatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaag

SEQ ID NO: 190 - DNA, 2424 bp, Synthetic construct
  source 1..2424: mol_type = other DNA
aaccagggcaaagcaaaataaaagaaacttaatacgttatgccgtaatgaagggctaccaaaaacgataatctcaactgtaaacaggtacaatgcggacccttttgccacaaaacatacatcattcattgccggaaaaagaaagaagtgaagacagcagtgcagccagccatgttgcgccaatctaattatagatgctggtgccctgaggatgtatctggagccagccatggcatcatgcgctaccgccggatgtaaaatccgacacgcaaaagaaaaccttcgaggttgcgcacttcgcccacccatgaaccacacggttagtccaaaaggggcagttcagattccagatgcgggaattagcttgctgccaccctcacctcactaacgctgcggtgtgcggatacttcatgctatttatagacgcgcgtgtcggaatcagcacgcgcaagaaccaaatgggaaaatcggaatgggtccagaactgctttgagtgctggctattggcgtctgatttccgttttgggaatcctttgccgcgcgcccctctcaaaactccgcacaagtcccagaaagcgggaaagaaataaaacgccaccaaaaaaaaaaaaataaaagccaatcctcgaagcgtgggtggtaggccctggattatcccgtacaagtatttctcaggagtaaaaaaaccgtttgttttggaattccccatttcgcggccacctacgccgctatctttgcaacaactatctgcgataactcagcaaattttgcatattcgtgttgcagtattgcgataatgggagtcttacttccaacataacggcagaaagaaatgtgagaaaattttgcatcctttgcctccgttcaagtatataaagtcggcatgcttgataatctttctttccatcctacattgttctaattattcttattctcctttattctttcctaacataccaagaaattaatcttctgtcattcgcttaaacactatatcaataacctgcactaaaacaatggataaattggatgctaatgtttcttctgaagaaggttttggttctgttgaaaaagttgttttgttgacttttttgtctactgttattttgatggctattttgggtaatttgttggttatggttgctgtttgttgggatagacaattgagaaaaattaaaactaattattttattgtttctttggcttttgctgatttgttggtttctgttttggttatgccatttggtgctattgaattggttcaagatatttggatttatggtgaagttttttgtttggttagaacttctttggatgttttgttgactactgcttctatttttcatttgtgttgtatttctttggatagatattatgctatttgttgtcaaccattggtttatagaaataaaatgactccattgagaattgctttgatgttgggtggttgttgggttattccaacttttatttcttttttgccaattatgcaaggttggaataatattggtattattgatttgattgaaaaaagaaaatttaatcaaaattctaattctacttattgtgtttttatggttaataaaccatatgctattacttgttctgttgttgctttttatattccatttttgttgatggttttggcttattatagaatttatgttactgctaaagaacatgctcatcaaattcaaatgttgcaaagagctggtgcttcttctgaatctagaccacaatctgctgatcaacattctactcatagaatgagaactgaaactaaagctgctaaaactttgtgtattattatgggttgtttttgtttgtgttgggctccattttttgttactaatattgttgatccatttattgattatactgttccaggtcaagtttggactgcttttttgtggttgggttatattaattctggtttgaatccatttttgtatgcttttttgaataaatcttttagaagagcttttttgattattttgtgttgtgatgatgaaagatatagaagaccatctattttgggtcaaactgttccatgttctactactactattaatggttctactcatgttttgagagatgctgttgaatgtggtggtcaatgggaatctcaatgtcatccaccagctacttctccattggttgctgctcaaccatctgatacttgaaggctaggtggaggctcagtggccagttttccctattgagaggatgacagatatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaag

SEQ ID NO: 191 - DNA, 2285 bp, Synthetic construct
  source 1..2285: mol_type = other DNA
aaccagggcaaagcaaaataaaagaaacttaatacgttatgccgtaatgaagggctaccaaaaacgataatctcaactgtaaacaggtacaatgcggacccttttgccacaaaacatacatcattcattgccggaaaaagaaagaagtgaagacagcagtgcagccagccatgttgcgccaatctaattatagatgctggtgccctgaggatgtatctggagccagccatggcatcatgcgctaccgccggatgtaaaatccgacacgcaaaagaaaaccttcgaggttgcgcacttcgcccacccatgaaccacacggttagtccaaaaggggcagttcagattccagatgcgggaattagcttgctgccaccctcacctcactaacgctgcggtgtgcggatacttcatgctatttatagacgcgcgtgtcggaatcagcacgcgcaagaaccaaatgggaaaatcggaatgggtccagaactgctttgagtgctggctattggcgtctgatttccgttttgggaatcctttgccgcgcgcccctctcaaaactccgcacaagtcccagaaagcgggaaagaaataaaacgccaccaaaaaaaaaaaaataaaagccaatcctcgaagcgtgggtggtaggccctggattatcccgtacaagtatttctcaggagtaaaaaaaccgtttgttttggaattccccatttcgcggccacctacgccgctatctttgcaacaactatctgcgataactcagcaaattttgcatattcgtgttgcagtattgcgataatgggagtcttacttccaacataacggcagaaagaaatgtgagaaaattttgcatcctttgcctccgttcaagtatataaagtcggcatgcttgataatctttctttccatcctacattgttctaattattcttattctcctttattctttcctaacataccaagaaattaatcttctgtcattcgcttaaacactatatcaataacctgcactaaaacaatgtccataccgttgccgcttcttcagatatacacgtctgataattacacggaggagatgggctctggcgattatgactctatgaaggaaccctgctttagagaagagaacgcaaacttcaacaaaatttttctgcctacgatatattccataatattccttacggggatcgtcggcaatgggttagttatactggtcatgggttaccaaaagaaattgcgtagtatgaccgacaaatatcgtcttcatttgagcgttgccgacttgttatttgtcatcacactgcccttttgggctgtagacgccgtagcgaactggtactttgggaactttttgtgtaaagctgttcatgttatctacaccgttaatctatattcctccgtattgattttagcttttatatccttggatagatacctggcaatagtacatgcaacgaattcccaacgtcctagaaagttactggccgagaaagtagtgtacgtgggggtatggattccggccctgttgttgacgataccggattttatattcgctaatgtcagcgaggcagacgatagatatatatgcgacagattctaccctaacgacctgtgggtcgttgtattccaattccaacacattatggtgggactgatcttgcccggcatagtaatcttgagctgctattgtataatcataagtaagctgtcccatagtaagggacaccaaaaaaggaaagccttgaaaaccaccgtgatactaattcttgctttctttgcgtgttggctaccttactacattgggataagcattgactctttcatcctgttagagattataaagcagggatgtgagttcgagaacaccgtgcacaagtggatctccatcactgaggcgcttgcattcttccattgttgccttaaccccattctatatgccttcttaggcgctaaattcaaaacatctgctcaacatgcccttacttcagttagtagaggtagttctctgaaaattttgtctaagggtaaaaggggcggacattcctccgttagtactgaaagtgaatcatcctcttttcacagctcctgaatgacagatatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaag

SEQ ID NO: 192 - DNA, 3087 bp, Saccharomyces cerevisiae
  source 1..3087: mol_type = genomic DNA
gatcagaggttacatggccaagattgaaacttagaggagtatagttacataaaagaaggcaaaacgatgtataaatgaaagaaattgagatggtgcacgatgcacagttgaagtgaacttgcggggtttttcagtatctacgattcattttactctttttttgggtttggtggggtatcttcatcatcgaatagatagttatatacatcatccattgtagtggtattaaacatccctgtagtgattccaaacgcgttatacgcagtttggtccgtccaaccaggtgacagtggttttgaattattaccatcatcaattttactagccgtgatttcattattcatgaagttatcatgaacgttagaggaggcaattggttgtgaaagcgcttgagaatttgtttgagttgttatgaggttcggaccgttgctactgttagtgaaagtgaaggacaatgagctatcagcaatattcccactttgattaaaattggcgccaccaaacaaagcagacggggtcagtggcactaatgattgcagctgttgctgttgccctagaaaaggcgtgactgagcgatgcgaaggtgtgcttcttggtattgtcactggagagttacgagagggtggacggttagataacagcttgactagatcactgaaacttgctcctgatttcaatggcacaggtgaaggccctactgagccaggagaaacatatttaacactgatattgttgacattttcctccggaagagtagggtattgggcgatagttgcagaaccgacaatatttttaatggcgctaccattactattgttataactgatatgcggtaatgggattgcacactgtgataacagaaacggcgcacatacctcttccagtacttgaatgtatttttcacaagtctggattttaaaagtggccagtttttttaatagcatcagaacagtgttaatttgttgtaataattgtgcggtctcgttattctcagcattcgattttgagtttgagagtagagtctttatgggtactaggactgcattgaacaagtaataagaacaattccaggcaaaatatggggtgacattatgattgtccatatagctacttacagacataacagttctttgtgctgcatcgcttaacatgatggagcatcgtttaacttcataactttgatgatcattttgatcctgttctagttgtgactttttctgggtaaaattagtgaaaaaatctcttaatacataaatgataagagacaactgtttccacttcagttcgaatcttgtaaaggatagccaagggtgttccttcaacaaattggttagagcggtggtggaaatatccatttgtaaaaactttggtgcctgtctcgaaacctcctcaatctcattacaaatcatcaagcatttttttgcacatataggacttttttctgcagttactgttttgtctagttcatagatttttgtgaaaacttgtaagagccttgctgtttcaatgatgccatgatatatggtgggacctgttgtggtacgctgcacatcgtcgacagaagaagggaaggagattgtattctgagaaagctggatggatcgaccataaagcagggacaattggatctcccaagagtagacagaccaccaaattcggcgtctttgttccagaatgctgctatcactgaaggacgaggggaggtccctattcaagcccaatgatatggccattcttatggaaaagctgtgaaaattatagctagtatttgttttctgcctccactgtgtatatcgcgacagaagatgtagggctgtcaccaaaattatggaacctgactcgaagaccttgctcgtcaaatgagatttagcattttgatagtaaaaaacatctatatcagtagattccccctctatacaccaggctccaatggctaatatgcagttaaaaaggatttgccattgatccttcgacgcgatttcaatctggttattatacaacatcattagcgtcggtgagtgcacgatagggcagtaggggtgaaaattattgagataactttgaagtaaacgggatgttgtggatctagaagccaacgtgtatctatccgtaatcatggtcgggagcctgttaacgttagagttcgtgtaattttccggtttaaagccaatagatcgaagaatacataagagagaaccgtcgccaaagaacccattattgttggggtccgttttcaggaagggcaagccatccgacatgtcatcctcttcagaccaatcaaatccatgaagagcatccctgggcataaaatccaacggaattgtggagttatcatgatgagctgccgagtcaatcgatacagtcaactgtctttgacctttgttactactctcttccgatgatgatgtcgcacttattctatgctgtctcaatgttagaggcatatcagtctccactgaagccaatctatctgtgacggcatctttattcacattatcttgtacaaataatcctgttaacaatgcttttatatcctgtaaagaatccattttcaaaatcatgtcaaggtcttctcgaggaaaaatcagtagaaatagctgttccagtctttctagccttgattccacttctgtcagatgtgccctagtcagcggagaccttttggttttgggagagtagcgacactcccagttgttcttcagacacttggcgcacttcggtttttctttggagcacttgagctttttaagtcggcaaatatcgcatgcttgttcgatagaagacagtagcttcatctttcaggaggcttgcttctctgtcctctcttaaaatgatggcgtgcattacgtagacacaatctggagatgaagctgaaaatctggggaaggatgacggaaaaaatagctcataaaacagaaaaaggcccgaagtaacaataggaaaaattaattgcactaaacaaagaaaacgatattatggtgattaaactgatacagaattatgtaaatactttgaaattatagaaggtttgtagaataaaaaaaatactgggcgaatgctgtctcaaaggacgcggacgttttgaaa

SEQ ID NO: 193 - protein, 881 aa, Saccharomyces cerevisiae
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSIDSAAHHDNSTIPLDFMPRDALHGFDWSEEDDMSDGLPFLKTDPNNNGFFGDGSLLCILRSIGFKPENYTNSNVNRLPTMITDRYTLASRSTTSRLLQSYLNNFHPYCPIVHSPTLMMLYNNQIEIASKDQWQILFNCILAIGAWCIEGESTDIDVFYYQNAKSHLTSKVFESGSIILVTALHLLSRYTQWRQKTNTSYNFHSFSIRMAISLGLNRDLPSSFSDSSILEQRRRIWWSVYSWEIQLSLLYGRSIQLSQNTISFPSSVDDVQRTTTGPTIYHGIIETARLLQVFTKIYELDKTVTAEKSPICAKKCLMICNEIEEVSRQAPKFLQMDISTTALTNLLKEHPWLSFTRFELKWKQLSLIIYVLRDFFTNFTQKKSQLEQDQNDHQSYEVKRCSIMLSDAAQRTVMSVSSYMDNHNVTPYFAWNCSYYLFNAVLVPIKTLLSNSKSNAENNETAQLLQQINTVLMLLKKLATFKIQTCEKYIQVLEEVCAPFLLSQCAIPLPHISYNNSNGSAIKNIVGSATIAQYPTLPEENVNNISVKYVSPGSVGPSPVPLKSGASFSDLVKLLSNRPPSRNSPVTIPRSTPSHRSVTPFLGQQQQLQSLVPLTPSALFGGANFNQSGNIADSSLSFTFTNSSNGPNLITTQTNSQALSQPIASSNVHDNFMNNEITASKIDDGNNSKPLSPGWTDQTAYNAFGITTGMFNTTTMDDVYNYLFDDEDTPPNPKKE

(19) United States
(12) Patent Application Publication
(10) Pub. No.: US 2026/0242721 A1
(43) Pub. Date: Aug. 20, 2026

(54) A SYNTHETIC CELLULAR ADVANCED SIGNAL ADAPTER (SCASA) SYSTEM

(71) Applicant: Danmarks Tekniske Universitet, Kongens Lyngby (DK)

(72) Inventors: Marcus Deichmann, Kongens Lyngby (DK); Michael K. Jensen, Kongens Lyngby (DK); Emil D. Jensen, Kongens Lyngby (DK); Giovanni Schiesaro, Kongens Lyngby (DK)

(21) Appl. No.: 19/489,540

(22) PCT Filed: Jun. 6, 2024
(86) PCT No.: PCT/EP2024/065573

§ 371 Date: Dec. 2, 2025

(30) Foreign Application Priority Data
Jun. 6, 2023 (EP) 23177666.7

(51) Int. Cl. C12N 1/18; A61K 35/17; A61K 40/11; A61K 40/15; A61K 40/31; A61K 40/42; A61P 35/00; C07K 14/72; C12N 5/0783; C12R 1/85

(52) U.S. Cl. CPC C12N 1/18; A61K 35/17; A61K 40/11; A61K 40/15; A61K 40/31; A61K 40/42; A61P 35/00; C07K 14/723; C12N 5/0636; C12N 5/0646; C12N 2502/70; C12N 2830/34; C12R 2001/85

(57) ABSTRACT

The present invention relates to a Synthetic Cellular Advanced Signal Adapter (SCASA) system and method comprising a genetically modified yeast cell capable of modulating a receiver cell, preferably a human immune cell. The invention provides systems to dynamically modulate the phenotype of the receiver cell in response to dynamic stimulation of the genetically modified yeast cell.

### FIELD

The present invention relates to a Synthetic Cellular Advanced Signal Adapter (SCASA) system and method comprising a genetically modified yeast cell capable of modulating a receiver cell, preferably a human immune cell. The invention provides systems to dynamically modulate the phenotype of the receiver cell in response to dynamic stimulation of the genetically modified yeast cell.

### BACKGROUND

In the last decade cell therapies have emerged as a novel therapeutic for treating diseases. Specifically, the use of e.g., manufactured T cells (e.g., TILs, CAR T and NeoTCR engineered cells) has become an area of increasing interest. While many therapies are able to generate small scale, research grade results showing the effectiveness of such cell therapies for the treatment of cancer, a major limitation of getting these therapies to patients is manufacturing capabilities. In addition, a main challenge of existing immunotherapies is lack of spatiotemporal control and intensity-tuning of responses, which for example has led to issues with life-threatening overstimulation of engineered T cells in cancer therapy.

One critical step in the manufacture of cell therapies is in vitro activation of the cells. Repeated or overstimulation using highly immunogenic antigen-presenting cells (APCs), such as e.g., dendritic cells, unavoidably matures T cells and leads to T cell activation-induced cell death (AICD), especially for T cells that possess high antigen-specific avidity. Because of their highly potent immunogenicity, patient-derived APCs also referred to professional APCs are not the best choice for generating in vitro T cells for use in cell therapy products. As a result, a variety of artificial APCs or artificial APC analogs (aAPCs) have been developed, such as e.g., human derived cell lines. However, culturing and maintaining a population of human cells is costly and time consuming. Accordingly, a variety of non-cellular aAPCs have been developed and are commercially available. For example, microbeads or nanoparticles functionalized with activating antibodies for CD3 (ctCD3) and CD28 (aCD28) are commonly used. However, because these commercial products covalently bind the activating antibodies to a solid-phase support, the activating molecules are static, unlike a natural antigen presenting cells, where stimulatory ligands move within the cell membrane to enable TCR clustering, a key step in T cell activation.

The current technologies are limited by the interaction between the T cells and the activation particle since these are either static and non-native or highly heterogeneous due to difficulties controlling the expression levels in the APCs. In addition, aAPCs such as synthetic beads or other inert particles can lead to chronic activation, which may result in overstimulated cells. These current technologies do not allow easy system adaptation, e.g., by encompassing more or different signal molecules, or for dynamic regulation of the presented molecules that interact with the immune cells. This means that it can be difficult to adapt the types and intensities of delivered signals for diverse patient- and assay specific applications, such as those described herein.

In addition, the performance evaluation of many immunotherapies, and especially cell therapies, remain challenging. The main reason for this lies in the difficulty in establishing controlled simulations where the effects of specific factors on immunotherapy performance can be assessed.

### SUMMARY

In a first aspect the present invention relates to a Synthetic Cellular Advanced Signal Adapter (SCASA) system for use in modulating, manipulating, and/or influencing the phenotype of human immune cells, comprising in vitro co-culturing of:
a) a genetically modified yeast cell which,
   i) expresses at least one receptor whose activity regulates the activity of a signalling pathway and/or
   ii) expresses a transcription factor, whose activity is regulated by said signalling pathway,
   iii) expresses one or more signal molecule(s), whose expression is/are regulated by the activity of said transcription factor, and
   wherein said signal molecule(s) is/are presented on the surface and/or secreted from said cell in response to said activity of said transcription factor, and
b) a human immune cell, wherein said cell is modulated, manipulated and/or obtains an altered phenotype in response to said presentation and/or secretion of said signal molecule(s) from said yeast cell.

One particularly interesting use of the SCASA system lies with in vitro co-culturing methods for modulating, manipulating, and/or influencing the phenotype of human immune cells, comprising a genetically modified yeast cell and a human immune cell.

In a second aspect the present invention relates to a method for modulating, manipulating, and/or influencing the phenotype of human immune cells, comprising in vitro co-culturing of:
a) a genetically modified yeast cell which is specifically engineered to facilitate direct intercellular signalling to a human immune cell, wherein said yeast cell comprises
   i) a modified signalling pathway comprising a receptor and/or transcription factor which facilitates and optionally regulates the intensity and/or type of direct intercellular signalling between said yeast cell and said human immune cell, and
   ii) expression of one or more signal molecule(s), which expression is/are regulated by said receptor and/or transcription factor and which signalling molecule(s) facilitates the direct intercellular signalling between said cells, and
b) a human immune cell, which is modulated, manipulated and/or obtains an altered phenotype in response to said direct intercellular signalling with said yeast cell.

Preferably, the human immune cell is selected from the group of immune cells consisting of T lymphocytes (T cells), engineered T cells (e.g., CAR T cells), Jurkat cells, B lymphocytes (B cells), Natural killer (NK) cells, Dendritic cells, Macrophages, Neutrophils, Eosinophils, Basophils, Mast cells, and innate lymphoid cells.

Preferably the genus of the yeast cell is selected from the group consisting of Saccharomyces, Schizosaccharomyces, Eurospora, Gibberella, Fusarium, Podospora, Cryphonectria, and Magnaporthe, such as e.g., a yeast cell of the Saccharomyces genus.

Additionally, the receptor preferably responds to one or more stimuli selected from the group consisting of light, heat, pH, electrochemical changes, small molecules, peptides, proteins, and combinations thereof.

Preferably, the receptor is a G protein-coupled receptor (GPCR) and additionally, the transcription factor is Gal4 with an amino acid sequence according to SEQ ID NO: 193, and/or Ste12 with an amino acid sequence according to SEQ ID NO: 35, or the transcription factor is a synthetic transcription factor which comprises synthetic transcription factors containing the pheromone-responsive domain (PRD) of Ste12 with an amino acid sequence according to SEQ ID NO: 36.

In addition, the signal molecule is preferably selected from the group consisting of small molecules, peptides, proteins, and antibodies, such as e.g., a signal molecule is selected from the group consisting of CD19, yEGFP, anti-CD3, anti-CD28, IL-2, HER2 and PD-L1.

In addition the expression of said receptor may be under control of a first promoter, the expression of said transcription factor may be under control of said first, or a second promoter, and the expression of said signal molecule may be under control of said first, said second, or a third promoter, such as e.g., a first, second and/or third promoter selected from the group of promoters consisting of P_{MFA2}, P_{MFA1}, P_{AGA2}, P_{YCL076W}, P_{IME4}, P_{HPF1}, P_{CSS1}, P_{FUS1}, P_{FIG.1}, P_{TDH3}, P_{PGK1}, P_{SNZ1}, P_{CPS1}, P_{CCW12}, P_{6xLexO-LEU2}, P_{GAL1}, P_{TEF1} and P_{RAD27} comprising or consisting of nucleic acid sequence according to any one of SEQ ID NOs: 1-18 or functional variants thereof with a nucleic acid sequence that is at least 80% identical to any one of SEQ ID NOs: 1-18.

In additional aspects of the invention the SCASA system and/or the method disclosed herein may be for use in assaying the signals influencing the responses, phenotypes and/or activation of T cells and/or CAR T cells. In addition, the SCASA system may be for use in the design and characterization of new CARs and other human immune cell products, such as, e.g., T cells with engineered T-cell receptors (TCRs). In addition, another aspect of the present invention relates to a human immune cell and/or human immune cell with a phenotype, produced by the system as described above, for use as a medicament.

Another aspect of the invention relates to a method for T cell expansion and/or differentiation, such as e.g., naïve T cell expansion and/or differentiation, engineered CAR T cell expansion and/or differentiation, and/or differentiation of immune cells, said method comprises the use of a SCASA system or method according to the present disclosure.

### BRIEF DESCRIPTION OF FIGURES

FIG. 1 Structure of the Synthetic Cellular Advanced Signal Adapter (SCASA) system.

Example of general structure of the SCASA system showing a SCASA yeast cell interacting with an immune cell. The yeast cell is exposed to a simple input (stimuli), through the sensory module, which then activates the receptor to transmit a signal, through the processing module, which control the expression of advanced signals (signal molecules) through the effector module, which confers communication with the immune cell that in response changes its phenotype. The example shows a GPCR-based SCASA yeast cell.

FIG. 2 Processing module.

The processing module of a GPCR-based SCASA yeast cell confers the translation of the GPCR activation into an expressed output, through the pheromone-response pathway (PRP), which was characterized by the induced expression of yEGFP through stimulation of Ste2 with α-factor at different concentrations (0-100 μM) for different processing module promoters.

FIG. 3 Stimulated SCASA yeast cells with different processing modules.

Heat-maps of quantified responses from GPCR-based (Ste2) SCASA yeast cells with different processing modules during stimulation with α-factor at different concentrations (0-100 μM). A. Absolute yEGFP levels (log₁₀-scale) of cells for individual promoters, including effects of the PRP-induced expression boost. The pheromone-responsive promoters are sorted on maximum absolute yEGFP intensity, from low to high. B. Shmoo intensity of cells. The pheromone-responsive promoters are sorted on maximum absolute yEGFP intensity, from low to high. C. Fold change induction of promoters, excluding PRP-induced expression boost (SSC-normalized). Pheromone-responsive promoters are sorted on fold change, from low to high.

FIG. 4 PRP-induced expression boost for yEGFP.

Quantification of the general PRP-induced expression boost (black) across all strains and designs (gray) for yEGFP expression upon stimulation of the GPCR (Ste2) with a 10-fold concentration range of α-factor (0-100 μM).

FIG. 5 Comparisons of processing units.

Comparison of the PF_{US1} processing unit design (black) to orthogonal GPCR signalling that employs an orthogonal signal pathway relying on a LexA-based synthetic transcription factor (sTF) and LexO-containing promoter (P_{6xLexOLEU2}), and importantly has the PRP transcription factor Ste12 deleted (ste12Δ0) (gray), for absolute yEGFP output, promoter activity (SSC-normalized), PRP-induced expression boost, and fold changes in yEGFP output at different concentrations of α-factor (0-100 μM). Fold changes, excluding PRP-induced expression boost (SSC-normalized), are illustrated with dashed lines.

FIG. 6 Comparison of processing module promoters driving yEGFP expression.

Fold changes of individual promoters, excluding PRP-induced expression boost (SSC-normalized), at different concentrations of α-factor (0-100 μM).

FIG. 7 Yeast surface display (YSD) construct for display of CD19.

A fusion protein comprising a hemagglutinin (HA) tag, a PAS40-linker, a (G₄S)₃-linker, CD19.1 extracellular domain (ECD), and a myc tag, fused to the Aga2 C-terminal, bound to Aga1 in the yeast cell wall. Antibodies for characterization are shown.

FIG. 8 System performance in comparison to conventional YSD and cancer cell expression of CD19.

Conventional YSD strain EBY100 was transformed with a pCT-type plasmid expressing the CD19 construct from P_{GAL1} and stained after 24 hrs of 2% Gal induction (light gray). SCASA system strain design, DIX49, expressed the CD19 construct constitutively via P_{TDH3} from a genome-integrated site (dark gray). The human CD19+ NALM6 B cell leukemia cell line was included as a benchmark control (black). The percentage of alive CD19+ cells is provided in the table.

FIG. 9 Investigation of P_{GAL1}-based control of YSD by plasmid and genome-integrated expression.

Investigation of P_{GAL1}-based control of CD19 expression via YSD from a 2μ multicopy expression plasmid (pMAD58) and from a genome-integrated site. Cells were stained after 24 hrs of galactose induction (0.0002%-2% Gal), using raffinose as the compensating carbon source (light gray to black). A negative control was included at which cells were cultured in glucose (2% Gluc) (top, light gray). Histograms are representatives of three biological replicates.

FIG. 10 Constitutive CD19 display levels of yeast library.

Constitutive CD19 display levels of yeast library using different processing module promoters, without any GPCR stimulation (black). All strains were built from the same background strain (DIX41). Two CD19− controls were included (gray), the background strain (DIX41), and P_{PGK1}-empty (DIX44) that displays the YSD construct without CD19 from P_{PGK1}. Histograms are representatives of three biological replicates.

FIG. 11 Dynamically regulated CD19 display of yeast library.

Dynamically regulated CD19 display of the yeast library upon stimulation of the GPCR (Ste2) with a 10-fold concentration range of α-factor (0-100 μM) (light gray to black). Histograms are representatives of three biological replicates.

FIG. 12 Comparison of quantified CD19 display levels of yeast library.

A. Comparison of quantified absolute CD19 display levels during dynamic regulation of the yeast library upon stimulation of the GPCR (Ste2). B. Comparison of fold changes of dynamic regulation of CD19 display of the yeast library upon stimulation of the GPCR (Ste2).

FIG. 13 PRP-induced expression boost for CD19 display.

Quantification of the PRP-induced expression boost (black) across all strains and designs (gray) for yeast surface display of CD19 upon stimulation of the GPCR (Ste2) with a 10-fold concentration range of α-factor (0-100 μM).

FIG. 14 Representative illustration of co-culture of reporter Jurkat cells and SCASA yeast aAPCs.

The Jurkat cells that express anti-CD19 CARs (CAR+) bind to the surface of SCASA yeast aAPCs that display CD19. The Jurkat cells contain a nuclear factor of activated T cells (NFAT) transcription factor luciferase reporter system (NFAT-Luc), which upon CAR activation express luciferase that in turn results in a bioluminescent signal due to oxidation of luciferin. The SCASA yeast aAPCs has the possibility to be stimulated through their GPCRs, in this case Ste2 with ligand α-factor, to increase the CD19 antigen-density.

FIG. 15—Co-cultures of reporter Jurkat cells with SCASA yeast aAPCs.

A. Relative activation of 100% CAR+ Jurkat NFAT-Luc cells co-cultured with three different SCASA yeast aAPCs; P_{FUS1}-CD19, P_{MFA2}-CD19, and P_{TDH3}-CD19, as well as the negative control, P_{PGK1}-Empty, with and without stimulation of the SCASA yeast aAPC GPCR (Ste2) with a 10-fold concentration range of α-factor (0-1 μM). Relative activation level using CD19+ human cancer cell line NALM6 is included as a positive benchmark control. B. Fold change relative activation of 100% CAR+ Jurkat NFAT-Luc cells co-cultured with SCASA yeast aAPCs from FIG. 15A. C. Co-cultures of Jurkat NFAT-Luc cells with three different SCASA yeast aAPCs; P_{MFA2}-CD19, P_{PGK1}-CD19, and P_{TDH3}-CD19, as well as a negative control lacking the CD19 CDS in the display construct; P_{PGK1}-Empty, and NALM6 as a positive benchmark control. The percentage of Jurkat NFAT-Luc cells in the co-culture expressing CARs was graded from none to all (0-100% CAR+).

FIG. 16—Absence of yeast growth in mammalian media.

Assessment of fitness burden of YSD on yeast cells, and their ability to grow in media for human immune cells. A total of 40K or 200K yeast cells were seeded, and their growth monitored by optical density (OD₆₃₀) over 24 hrs. Strains included are DIX41 (No YSD); DIX44 (Empty YSD); and DIX47 (YSD by P_{PGK1}-CD19). Yeast cells were cultured in RPMI+10% FBS, ImmunoCult™-XF, SC and pH-buffered SC.

FIG. 17—Yeast GPCR sensing in mammalian media and T cell co-cultures.

Yeast cells expressing GPCR Ste2 were co-cultivated with T cells at 37° C. or left as monocultures of yeast cells. Cultivations were performed with or without stimuli of Ste2 (α-factor) in RPMI+10% FBS during both monoculture and in co-cultures with 1.0× T cells, and yEGFP signals recorded until day 5 (96 hrs).

FIG. 18—Viability of T cells derived from peripheral blood mononuclear cells (PBMCs) co-cultivated with yeast.

Co-cultivation of 500K naïve T cells with different amounts of yeast; 250K (0.5×), 500K (1.0×), and 5,000K (10.0×). T cells were stained with a Live/Dead dye to determine viability after 5 days (96 hrs.) of co-cultivation.

FIG. 19—Protocol flowsheet for T cell stimulation using SCASA yeast aAPCs.

An example of a SCASA-based protocol for co-cultivation of SCASA yeast aAPCs, presenting signal molecules at different levels caused by stimulation with different amounts of ligand, with human cells. At day 5 the responses of the human cells are characterized.

FIG. 20—Activation of donor-derived CAR T cells using SCASA yeast aAPCs and NALM6: CD69 histograms.

Donor-derived CAR T cells were generated from a Pan T cell isolate of a healthy donor using CRISPR-MAD7 to insert a Hu19-CD828Z CAR, delivered by electroporation. To validate if SCASA yeast aAPCs could confirm successful CAR engineering and a functional CAR T cell product, the CAR T cells were co-cultivated with different target cells: SCASA yeast aAPCs expressing CD19 (P_{PGK1}-CD19), a negative control SCASA yeast aAPC (P_{PGK1}-Empty), and cancer cell line NALM6. A negative control (CTRL) T cell isolate was also employed, having gone through the same engineering procedure but without a CAR transgene. Co-cultivations were standardized to contain 100,000 alive CAR+ T cells, and different target cell ratios: 20,000 (0.2×), 100,000 (1.0×), and 500,000 (5.0×) target cells. After 20 hrs of co-cultivation, cells were stained and analyzed by flow cytometry. Staining for activation marker CD69 after co-cultivation: CTRL T cell culture (alive CD3+ population) (upper row), and CAR T cell culture (alive CD3+ CAR+ population) (lower row). CTRL and CAR T cells were co-cultivated with NALM6 (left), SCASA yeast aAPC P_{PGK1}-CD19 (middle), and negative control SCASA yeast aAPC P_{PGK1}-Empty (right)—at each target cell ratio. The far-right column shows an overlay of CD69 T cell responses for 5.0× co-cultivations, at which the black line histogram represents unstimulated monocultures of the T cells. Histograms are representatives of three biological replicates.

FIG. 21—Activation of donor-derived CAR T cells using SCASA yeast aAPCs and NALM6: CD69 expression intensity.

CD69 expression intensity measured as mMFI (CD69-PE/Cy7) of CAR+CD3+(circle), CAR-CD3+(square), and CTRL (CD3+) (triangle) alive T cells normalized to CTRL T cell monocultures (alive CD3+), for three biological replicates, with lines connecting the means±SD, for cocultivations with target cells NALM6 (left), SCASA yeast aAPC P_{PGK1}-CD19 (middle), and negative control SCASA yeast aAPC P_{PGK1}-Empty (right) at different target cell ratios: 20,000 (0.2×), 100,000 (1.0×), and 500,000 (5.0×) target cells. Horizontal dotted lines represent baseline CD69 levels of CAR− CD3+, CAR+CD3+, and CTRL (CD3+) T cell monocultures.

FIG. 22—Activation of donor-derived CAR T cells using SCASA yeast aAPCs and NALM6: % CD69+ of T cell populations.

Percentage of T cell populations expressing CD69 (% CD69+) of CAR+CD3+(circle), CAR-CD3+(square), and CTRL (CD3+) (triangle) alive T cells, with bars showing the means±SD, for co-cultivations with target cells NALM6 (left), SCASA yeast aAPC P_{PGK1}-CD19 (middle), and negative control SCASA yeast aAPC P_{PGK1}-Empty (right) at different target cell ratios: 20,000 (0.2×), 100,000 (1.0×), and 500,000 (5.0×) target cells. Horizontal dotted lines represent baseline % CD69+ populations of CAR+CD3+ and CAR− CD3+ T cell monocultures and CTRL T cell monocultures.

FIG. 23—Activation of donor-derived CAR T cells using SCASA yeast aAPCs after cryopreservation: CD69 and CD25 histograms.

Staining for activation markers CD69 (left) and CD25 (right) after co-cultivation of 75,000 alive CAR T cells (alive CAR+/−CD3+), after 5 months of cryopreservation in liquid nitrogen, with SCASA yeast aAPC designs P_{MFA2}-CD19 and P_{TDH3}-CD19, as well as negative control P_{PGK1}-Empty at a ratio of 0.3× (25,000 yeast cells). In addition, a lymphocyte activation cocktail (LAC) was used as a positive control. CAR T cells were cultivated with yeast strains with 0.1 μM α-factor pre-stimulation (dark gray) and without (light gray) stimulation and are presented in comparison to unstimulated monocultures of the CAR T cells (blacklines). Histograms are one of two biological replicates.

FIG. 24—Activation of donor-derived CAR T cells using SCASA yeast aAPCs after cryopreservation: CD69 and CD25 expression intensities.

Expression intensity of CD69 (A) and CD25 (B) measured as mMFI (CD69-PE/Cy7 and CD25-BB700, respectively) normalized to CAR T cell monocultures (alive CD3+) for two biological replicates. Co-cultures at which SCASA yeast aAPCs have been pre-stimulated with 0.1 μM α-factor are marked (+α).

FIG. 25—Activation of donor-derived CAR T cells using SCASA yeast aAPCs: yeast CD19 display intensities.

CD19-levels of SCASA yeast aAPCs during CAR T cell co-cultivations (see FIG. 20-22) for different target cell ratios.

FIG. 26—SCASA yeast aAPC GPCR library for induction of CD19 display.

A. CD19 display densities upon stimuli of seven different SCASA sensory module designs. The small library of diverse GPCRs tested cover sensing of small molecules (melatonin, adenosine, adrenaline, serotonin), peptides (P-factor), and complex proteins (CXCL12/SDF-1), with GPCR genes encoding MTNR1A, ADORA2B, ADRA2A, HTR4B, MAM2 and CXCR4A that were expressed like Ste2 of previously characterized SCASA designs, and were coupled to either native Gpa1, or chimeric GαZ- or Gαi2-subunits in the strain background optimized for biosensing and YSD. A negative control without any GPCR was included. These strains were stimulated using cognate GPCR agonists for 20 hrs in media buffered for the functional pH of the individual GPCR, and subsequently CD19 levels were quantified using an anti-HA (16B12) antibody. Relative CD19 levels (B) and dynamic output ranges (fold changes) (C) from the different designs under various concentrations of ligand stimuli is also presented.

FIG. 27—Exemplifications of SCASA concept and applications disclosed herein.

Exemplifying illustrations of the SCASA co-cultivation concept, which involves co-cultivating a SCASA yeast cell with a chosen target cell to facilitate communication via signal molecules. These interactions can result in a modified target cell phenotype and can be used to simulate specific communication scenarios dependent on the chosen signal molecules. This concept can for example have applications in research assays, e.g. to characterize target cell response intensities, to enable design selection through comparison of different signal molecules variants, or to discover new signalling modalities in an unbiased manner. Another example application is in cell product manufacturing, e.g. cell therapies from patient-derived samples, in which progenitor cells are exposed to SCASA yeast cell signalling to obtain a specific phenotype of clinical interest. These phenotype-modified cells now comprise a cell product, which can then be transplanted into the patient with the purpose of treating or curing the medical condition.

FIG. 28—Extended analysis of PGAL1-based control of YSD.

Extension of the analysis presented in FIG. 9; Investigation of PGAL1-based control of CD19 expression via YSD from a 2μ multicopy expression plasmid (pMAD58) and from a genome-integrated site. A. CD19 YSD after 24 hrs of galactose induction (0.0002%-2% Gal), using raffinose as the compensating carbon source (light gray to black). The percentage of CD19 displaying cells (CD19+) is presented for relevant populations based on the shown CD19+ gate. A negative control for induction was included through cultivation in glucose (2% Gluc). A constitutive genome-integrated strain (PTDH3) and a non-displaying control was also included. B. Quantification of % CD19+ cells of entire yeast populations for given conditions—i.e. the amount of displaying cells. C. Quantification of relative CD19 display intensities for the CD19+ populations. Data represents means of cell counts or median fluorescence intensities (mMFI) for three biological replicates.

FIG. 29—Activation of donor-derived CAR T cells using SCASA yeast aAPCs: Robustness of CD19+ SCASA yeast, NALM6 cancer cells, CAR expression in co-cultivations with donor-derived CAR T cells.

A. CD19 levels on target cells after co-cultivations at 5.0× ratios of NALM6 (top) and SCASA yeast cell PPGK1-CD19 (bottom) in CAR T cell (CAR+) and CTRL T cell (CTRL) cultures. See also FIG. 25. B. Characterization of target cell number variation for NALM6 (dark gray), SCASA yeast PPGK1-CD19 (gray), and SCASA yeast PPGK1-Empty (light gray) by assessing fold change in the number of detected target cells per alive CD3+ T cell between the control (CTRL) and CAR T cell cultures, for each initially seeded target-to-effector cell ratio (T/E-ratio). That is, the change in target-to-effector cell ratio caused by CAR expression in the CAR+CD3+ T cell subpopulation for each co-cultivation setup. The absolute fold change variation in target-to-effector cell ratio between CTRL and CAR T cell cultures was quantified, disregarding whether the variation causes increased or diminished levels of target-to-effector cells. The absolute fold change was calculated by acquiring the reciprocal value of fold changes<1. C. Relative CD19 levels to the measured maximum of each target cell type individually after co-cultivations with CTRL T cells (triangle) and CAR T cells (circle), for NALM6 and SCASA yeast PPGK1-CD19. D. Percentage of CAR+ T cells per CD3+ T cell detected after co-cultivations. E. CAR expression intensity of CAR+ population of engineered cells relative to the CAR-population (background) and normalized to the maximal CAR expression measured. All data represents means of cell counts or median fluorescence intensities (mMFI) for three biological replicates (n=3) and standard deviations hereof.

FIG. 30—Example design of a GPCR-based Synthetic Cellular Advanced Signal Adapter (SCASA) system disclosed herein.

Illustration of exemplary designs of SCASA yeast cells using fungal and human GPCRs for controllable CD19 densities. The yeast surface display (YSD) construct enabling a CD19 effector output is composed of a fusion protein of a hemagglutinin (HA) tag, a myc tag, a PAS40-linker, a (G4S)3-linker, and CD19.1 ECD, fused to the Aga2 C-terminal, employing the native N-terminal Aga2 signal peptide. The display fusion protein associates with anchor protein Aga1 for presentation on the yeast cell wall. Strains can be genetically optimized for GPCR-biosensing by overexpression of GPCRs and Ga-protein, which is a subunit of the heterotrimeric Gaβγ-protein that couples GPCRs to the pheromone response pathway (PRP). Additionally, several native genes can be knocked out: ste2Δ0, ste3Δ0, gpa1Δ0, sst2Δ0, bar1Δ0, and far1Δ0. A PRP-induced boost can post-transcriptionally amplify the expression of genes. The intensity of CD19 display can be regulated differentially by different processing modules, which for example can be adapted by using a promoter library. For example, PRP-regulated promoters can be used for the processing module, like PMFA2, PFUS1, PAGA2, PFIG1, and PHPF1, as well as constitutive promoters, like PTDH3 and PPGK1. Heterologous GPCRs with various ligand types, such as small-molecule compounds, peptides, proteins, can be employed to customize the sensory input controlling CD19 expression. In addition to the genetic optimization of the system described, native Aga2 can be knocked out (aga2Δ0) and Aga1 overexpressed to enhance YSD abilities.

FIG. 31—Responses of NF-κB, NFAT and AP-1 in CAR T cells with CD28- and 4-1BB-based co-stimulation in co-cultivation with SCASA yeast cells.

The signalling responses of CAR T cells were characterized for two different co-stimulatory domains, CD28 and 4-1BB, across a variety of different co-cultivation conditions. Firstly, CD19 antigen densities were varied using GPCR stimulation (0-1 μM α-factor) of a constant target cell number that was equal to the amount of CAR T cells (×1.0 mix). Secondly, the total CD19 antigen load was varied by changing the ratios of target cells to a fixed amount of CAR T cells (×0.25-×8.0). The employed SCASA yeast cell designs were PPGK1-Empty (no CD19), PFUS1-CD19, PMFA2-CD19, PPGK1-CD19, PTDH3-CD19, and P6xLexOLEU2-CD19. Controls involved monocultures and cultivation with dynabeads, NALM6, and 1×LAC.

Responses of main transcription factor genes were quantified by using Triple Parameter Reporter Jurkat 76 T cell lines (TPR Jurkat cells) with NFAT-eGFP (B525/45) (A), AP-1-mCherry (Y615/20) (B), and NF-κB-CFP (V525/45) (C), measurable by flow cytometry. All data represents means of median fluorescence intensities (mMFI) of gated CAR Jurkat cells for three biological replicates (n=3) and standard deviations hereof.

FIG. 32—Comparison of CD28 and 4-1BB co-stimulation effects on NF-κB, NFAT and AP-1 responses.

Comparison of the responses between CD28- and 4-1BB-based co-stimulation for CAR T cells; NFAT-eGFP (square), NF-κB-CFP (circle), and AP-1-mCherry (triangle). The comparison shows the fold difference of CD28 over 4-1 BB for each parameter individually. The data represents the same conditions shown and described in FIG. 31.

### DETAILED DESCRIPTION

### Synthetic Cellular Advanced Signal Adapter (SCASA) System

The system described herein aims to provide a platform for scalable activation of cells such as e.g., T cells, in vitro. Such activation using modified yeast cells as described herein enables the development of therapies with higher efficiency, higher personalization, and lower toxicity, by establishing a system for simulating conditions that immunotherapies may be exposed to in vivo, in order to characterize and improve performance of such therapies.

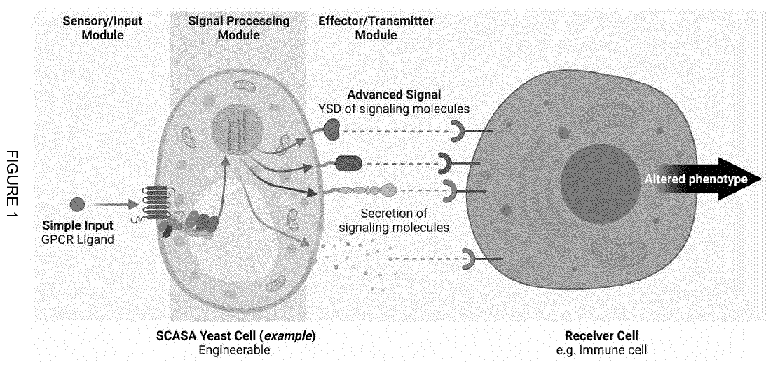

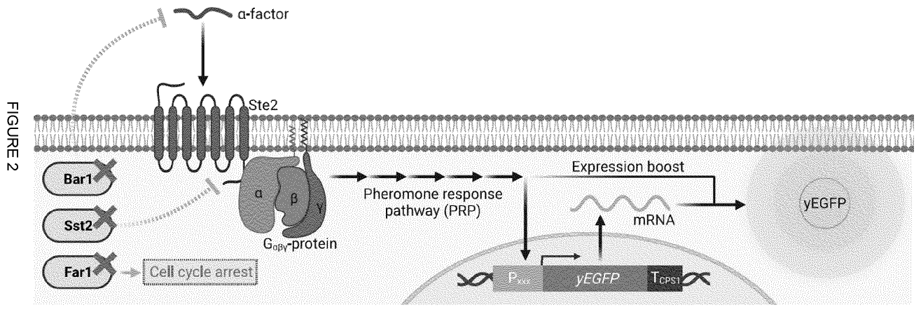

The present inventions relate to a Synthetic Cellular Advanced Signal Adapter (SCASA) system, which is a cellular system comprising a genetically modified yeast cell (SCASA yeast cell), which is used to modulate, manipulate, and/or influences the phenotype of a receiver cell, such as e.g., a human immune cell (see FIG. 1). The phenotype of the receiver cell may be obtained e.g., by co-culturing of the receiver cell with the genetically modified yeast described herein.

Changes in the phenotype of a receiver cell, such as e.g., a human immune cell, may involve changes to the observable and/or detectable characteristics of the receiver cell, such as e.g., changes in its morphology, function, behaviour, localization, motility, adhesion properties, polarization, proliferative state, viability, differentiation state, specialization, activation state, maturation state, apoptotic state, signalling pathways, pigmentation, genes (e.g., mutations or epigenetic modifications), gene expression, protein expression, marker presentation, metabolism, autophagy, secretory activity, endocytosis, exocytosis, sensitivity or resistance to other signals or stimuli, responses, cytotoxicity, exhaustion state, regulatory functions, state of inhibition, effector functions, memory functions, cell-cell communication, cell cycle regulation, and/or stress resistance. Non-limiting examples of phenotype modulation of a receiver includes e.g., inducing pluripotency in somatic cells, altering the expression of genes involved in immune responses, such as e.g., to expand and/or differentiate T cells or to create activated T or CAR T cells, or inducing differentiation of stem cells into specific cell types, such as e.g., neurons or cardiac cells.

An essential component of the SCASA system is the controlled dynamic expression of one or more signal molecule(s) in the genetically modified yeast cell in response to stimulation of the genetically modified yeast cell. Such a response may be obtained in a plethora of ways, such as e.g., by stimulation of a receptor whose activation triggers the expression of a signal molecule through a signalling pathway activating a transcription factor, or by direct activation of a transcription factor, which regulates the expression of said signal molecule or pathway responsible for synthesis of said signal molecule (see FIG. 1).

The SCASA system may in that way be split into three main modules (see FIG. 1):
a sensory module, such as e.g., a module comprising at least one receptor whose activity regulates the activity of a signalling pathway, or may itself be a transcription factor, and
a processing module, which confers the translation of the receptor activation into a signalling pathway that regulates the activity of a transcription factor,
an effector module which confers expression of one or more signal molecule(s) whose expression is/are regulated by the activity of said transcription factor, and which are secreted from and/or presented on the surface of the genetically modified yeast cell.

It will be evident to the person skilled in the art that the SCASA system may be structured in a plethora of ways, such as e.g., the embodiments described herein.

As a non-limiting proof of principle of the SCASA system, the inventors have produced a SCASA system which is suitable for dynamic activation of CAR (chimeric antigen receptor) T cells expressing a CD19 (Cluster of Differentiation 19) specific CAR.

Examples of functional SCASA systems are provided herein, which describes novel sensory modules, processing modules, and signalling modules, which combined allows the SCASA system to be used in the modulation, manipulation, and/or influencing the phenotype of mammalian cells.

The SCASA system can be implemented in many ways, and one particular way of implementing the SCASA system is a method for in vitro modulating, manipulating, and/or influencing the phenotype of a receiver cell (e.g. a human immune cell). As such the terms “system” and “method” may be used interchangeably when referring to SCASA.

The present invention further relates to methods for modulating, manipulating, and/or influencing the phenotype of a receiver cell (e.g. a human immune cell), comprising in vitro co-culturing of a genetically modified yeast and the receiver cell, wherein the genetically modified yeast cell is used to modulate, manipulate, and/or influences the phenotype of the receiver cell.

Additionally, the present invention relates to methods for modulating, manipulating, and/or influencing the phenotype of a human immune cell, comprising in vitro co-culturing of a genetically modified yeast and a human immune cell, wherein the genetically modified yeast cell is used to modulate, manipulate, and/or influences the phenotype of a human immune cell.

In particular, a method for modulating, manipulating and/or influencing the phenotype of human immune cells according to the present invention comprises in vitro co-culturing of:
a) a genetically modified yeast cell which is specifically engineered to facilitate direct intercellular signalling to a human immune cell, wherein said yeast cell comprises
   i) a modified signalling pathway comprising a receptor and/or transcription factor which facilitates and optionally regulates the intensity and/or type of direct intercellular signalling between said yeast cell and said human immune cell, and
   ii) expression of one or more signal molecule(s), which expression is/are regulated by said receptor and/or transcription factor and which signalling molecule(s) facilitates the direct intercellular signalling between said cells, and
b) a human immune cell, which is modulated, manipulated and/or obtains an altered phenotype in response to said direct intercellular signalling with said yeast cell.

It will be evident to the person skilled in the art that the method may be performed in a plethora of ways, such as e.g., the embodiments described herein.

In embodiments, the method may further comprise displaying the signalling molecule(s) on the surface of the genetically modified yeast cell.

In embodiments, the human immune cell of the method is selected from the group of immune cells consisting of T lymphocytes (T cells), engineered T cells, CAR T cells, Jurkat cells, B lymphocytes (B cells), Natural killer (NK) cells, Dendritic cells, Macrophages, Neutrophils, Eosinophils, Basophils, innate lymphoid cells, Mast cells. In further embodiments, the human immune cell of the method is selected from the group consisting of T cells, CAR T cells, and NK cells.

In embodiments, the displaying of the signalling molecule(s) on the surface of the genetically modified yeast cell is dynamically dependent on the stimulation level of said receptor and/or transcription factor. In embodiments, the signal molecule(s) of the method is/are selected from the group consisting of small molecules, peptides, proteins, and antibodies.

In embodiments, the genetically modified yeast cell of the method is a yeast cell as defined in the SCASA system. In embodiments, the genetically modified yeast cell of the method is a yeast aAPC as defined in the SCASA system. In further embodiments, the genetically engineered yeast cell of the method is of a genus selected from the group consisting of Saccharomyces, Eurospora, Gibberella, Fusarium, Podospora, Cryphonectria, and Magnaporthe. In further embodiments, the genetically engineered yeast cell is of the Saccharomyces genus.

In embodiments, the method comprises a receptor, wherein the receptor is exposed to the extracellular environment of the yeast cell and responds to extracellular input and/or changes in said extracellular environment. In further embodiments, the receptor is a G protein-coupled receptor (GPCR). In further embodiments, the receptor responds to stimulation from one or more signalling stimuli selected from the group consisting of light, heat, pH, electrochemical changes, small molecules, peptides, proteins, and/or combinations thereof.

In embodiments, the transcription factor of the method is
a) Gal4 with an amino acid sequence according to SEQ ID NO: 193, or a functional homologue thereof with an amino acid sequence which is at least 80% identical to SEQ ID NO: 193, and/or
b) Ste12 with an amino acid sequence according to SEQ ID NO: 35 or a functional homologue thereof with an amino acid sequence which is at least 80% identical to SEQ ID NO: 35, or
c) the transcription factor is a synthetic transcription factor which comprises synthetic transcription factors containing the pheromone-responsive domain (PRD) of Ste12 with an amino acid sequence according to SEQ ID NO: 36, or a functional homologue thereof with an amino acid sequence which is at least 80% identical to SEQ ID NO: 36.

The process of vaccination relates to a process for introduction of an antigen into a human, resulting in initiation of the adaptive immune response. Initiation of the adaptive immune response comprises antigen processing through the cytosolic pathway and the endogenous pathway, ultimately leading to antigen presentation by human antigen presenting cells.

The present method differs from a vaccination process in that the in vitro use of a genetically modified yeast cell (aAPC) to directly modulate, manipulate and/or influence the phenotype of human immune cells, effectively circumvents the cytosolic and endogenous pathways, and eliminates the need for involvement of human antigen presenting cells in modulating, manipulating or influencing the phenotype of a human immune cell.

Accordingly, in embodiments, the method does not comprise antigen processing by human antigen-presenting cells. Furthermore, in embodiments, the method does not comprise antigen processing of the genetically modified yeast cell or signal molecule(s) by human antigen-presenting cells.

In embodiments, the signal molecule(s) of the method is/are selected from the group consisting of:
a) CD19 with an amino acid sequence according to SEQ ID NO: 53, or a functional homologue thereof with an amino acid sequence which is at least 80% identical to SEQ ID NO: 53,
b) yEGFP, with an amino acid sequence according to SEQ ID NO: 38, or a functional homologue thereof with an amino acid sequence which is at least 80% identical to SEQ ID NO: 38,
c) anti-CD3, with an amino acid sequence according to SEQ ID NO: 59, or a functional homologue thereof with an amino acid sequence which is at least 80% identical to SEQ ID NO: 59,
d) anti-CD28, with an amino acid sequence according to SEQ ID NO: 60, or a functional homologue thereof with an amino acid sequence which is at least 80% identical to SEQ ID NO: 60,
e) IL-2, with an amino acid sequence according to SEQ ID NO: 54, or a functional homologue thereof with an amino acid sequence which is at least 80% identical to SEQ ID NO: 54,
f) HER2 with an amino acid sequence according to SEQ ID NO: 55, or a functional homologue thereof with an amino acid sequence which is at least 80% identical to SEQ ID NO: 55, and
g) PD-L1 with an amino acid sequence according to SEQ ID NO: 57, or a functional homologue thereof with an amino acid sequence which is at least 80% identical to SEQ ID NO: 57.

In embodiments, the method further comprises
a) the expression of said receptor under control of a first promoter,
b) the expression of said transcription factor under control of said first, or a second promoter, and
c) the expression of said signal molecule(s) under control of said first, said second, or a third promoter.

In embodiments, the method further comprises a first, second and/or third promoter selected from the group of promoters consisting of P_{MFA2}, P_{MFA1}, P_{AGA2}, P_{YCL076W}, P_{IME4}, P_{HPF1}, P_{CSS1}, P_{FUS1}, P_{FIG.1}, P_{TDH3}, P_{PGK1}, P_{SNZ1}, P_{CPS1}, P_{CCW12}, P_{6xLexO-LEU2}, P_{GAL1}, P_{TEF1} and P_{RAD27} comprising or consisting of nucleic acid sequence according to any one of SEQ ID NOs: 1-18 or functional variants thereof with a nucleic acid sequence that is at least 80% identical to any one of SEQ ID NOs: 1-18.

In embodiments, the method as disclosed above is used for assaying the signals influencing the phenotype of human immune cells.

In embodiments, the method as disclosed above comprises culturing multiple different yeast cells.

In embodiments, the method as disclosed above comprises culturing multiple different human immune cells.

As a non-limiting proof of principle, the inventors have demonstrated use of the method for in vitro dynamic activation of CAR (chimeric antigen receptor) T cells expressing a CD19 (Cluster of Differentiation 19) specific CAR.

The examples of the SCASA system disclosed herein, also demonstrate ways in which the method can be performed, and describes novel sensory modules, processing modules, and signalling modules, which, when used together in the method, allows for in vitro modulation, manipulation, and/or influencing the phenotype of mammalian cells. In particular the examples disclosed herein, describes novel sensory modules, processing modules, and signalling modules, which, when used together in the method, allows for in vitro modulation, manipulation, and/or influencing the phenotype of human immune cells.

Example 1, as a first step describes the characterization of different processing modules of the SCASA system and methods disclosed herein which may be used to dynamically express a reporter protein, in the example yEGFP, in response to stimulation of the genetically modified yeast cell. The dynamically regulated expression was in Example 1 obtained by coupling of the stimulation of a for the purpose recombinantly expressed, G-protein-coupled receptor (Ste2), which coupled to a recombinantly expressed G protein subunit, to different processing module promoters (see FIG. 2). The genotype of the genetically modified yeast cell is in Example 1 adjusted such that the G protein activates the pheromone response pathway (PRP) in the yeast cell, which activates and boosts the overall expression of genes in the genetically modified yeast cell, and specifically additionally promotes the expression of the reporter gene once the reporter gene is under control of specific promoter elements, which are regulated by said PRP pathway. Accordingly, Example 1 shows a number of exemplified embodiments as to how the second module, i.e., the processing module of the SCASA system, may be obtained.

Example 2 describes how the genetically modified yeast cell can be further modified to express a signal molecule, in the example CD19 fused to the Aga2 surface protein (see FIG. 7 and FIG. 30). The fusion protein was dynamically presented on the surface of the genetically modified yeast cells, in response to stimulation of the sensory module using different processing modules (see FIGS. 10-13 and FIG. 30). In Example 2, the sensory module comprised expression of a GPCR and a G-protein subunit, the processing module comprised a modified PRP and different processing module promoters for expression of the effector module that was CD19.

Example 3 describes the use of the SCASA system and methods disclosed herein, wherein the genetically modified yeast cell described in example 2, was co-cultured with CAR T cells (Jurkat cell lines) to promote the activation of the anti-CD19 CAR expressed in the cells (see FIG. 14). In Example 3 it was shown that the SCASA system could be used to dynamically activate the CAR T cells in response to the specific stimulation of the genetically modified yeast cell, in a dose-response dependent manner (FIG. 15).

Accordingly, the present disclosure shows proof of principle for the customization and applicability of the SCASA system and methods disclosed herein. It is to be understood that the examples presented herein are not meant as limiting in any way but serves as exemplified embodiments of the SCASA system and methods disclosed herein.

Additionally, the examples 1-3 discussed above, along with examples 4 and 6-8 also show proof of principle for the method for modulating, manipulating, and/or influencing the phenotype of human immune cells in vitro as defined herein. It is to be understood that the examples presented herein are not meant as limiting in any way but serves as examples of how to implement the method as defined herein.

### A Genetically Engineered Yeast Cell

In the present context, the terms “a genetically modified cell” and “a genetically engineered cell” are used interchangeably. As used herein “a genetically modified cell” is a cell whose genetic material has been altered by human intervention using a genetic engineering technique, such a technique is for example but not limited to transformation or transfection e.g., with a heterologous polynucleotide sequence, CRISPR/Cas editing and/or random mutagenesis. In one embodiment the genetically engineered cell has been transformed or transfected with a recombinant nucleic acid sequence.

The genetically engineered cell is preferably a Eukaryotic cell, such as e.g., a yeast cell. In embodiments, the genetically engineered cell is a yeast cell.

In further embodiments, the genus of the yeast cell is selected from the group genuses consisting of Saccharomyces, Schizosaccharomyces, Eurospora, Gibberella, Fusarium, Podospora, Cryphonectria, and Magnaporthe. In preferred embodiments, the yeast cell is a cell of the Saccharomyces genus.

In embodiments, a genetically modified yeast cell is a yeast cell that has been genetically modified to comprise
i) a modified signalling pathway comprising a receptor and/or transcription factor which facilitates and optionally regulates the intensity and/or type of direct intercellular signalling between said yeast cell and said human immune cell, and
   ii) expression of one or more signal molecule(s), which expression is/are regulated by said receptor and/or transcription factor pathway and which signalling molecule(s) facilitates the direct intercellular signalling between said cells.

In additional embodiments, the genetically modified yeast cell is a yeast cell that displays the signalling molecule(s) on the surface of said yeast cell.

In additional embodiments, the display of the signalling molecule(s) on the surface of the genetically modified yeast cell is dynamically dependent on the stimulation level of said receptor and/or transcription factor.

In additional embodiments, the genetically modified yeast cell comprises a receptor, where the receptor is exposed to the extracellular environment of the genetically modified yeast cell and responds to extracellular input and/or changes in said extracellular environment.

In embodiments, the genetically modified yeast cell, is a yeast cell as defined in the method for modulating, manipulating, and/or influencing the phenotype of human immune cells in vitro as defined herein.

In embodiments, the genetically modified yeast cell is an aAPC, also referred to herein as SCASA aAPC.

In the present disclosure an “aAPC”, or “artificial antigen-presenting cell”, is to be understood as a laboratory-generated cell designed to mimic the function of natural antigen-presenting cells (APCs). In the present disclosure APC is also to be understood as cell types that participate in communication with receiver cells such as e.g., immune cells. Generally, APCs are a type of immune cell that present foreign antigens to e.g., T cells, which then initiate an immune response. Within the meaning of the present disclosure the meaning of “aAPC” also encompasses cells that present other types of signal molecules.

### Receptors

An embodiment of the present invention is a SCASA system where the genetically engineered yeast cell expresses at least one receptor which regulates the activity of a signalling pathway and expresses a transcription factor, whose function is affected by the signalling pathway. Another embodiment of the invention is a method for modulating, manipulating, and/or influencing the phenotype of human immune cells in vitro, where the genetically engineered yeast cell expresses at least one receptor which regulates the activity of a signalling pathway and expresses a transcription factor, whose function is affected by the signalling pathway.

As such the receptors may be selected from ion channels, G protein-coupled receptors, enzyme-linked receptors, and intracellular receptors. The function of the expressed receptor is to receive a signal or stimuli and relay the incoming signal to the downstream processes in the cell, which modulate the presentation and/or secretion of the signal molecule(s). As such the receptor may respond to stimulus from various sources. In embodiments, the receptor responds to stimulation from one or more signalling stimuli selected from the group consisting of light, heat, pH, electrochemical changes, small molecules, peptides, proteins, and combinations thereof. In that sense the receptor may be a naturally occurring receptor or it may be synthetic. In relation to that a synthetic receptor may be a fully synthetic receptor or a chimeric or recombinant receptor. In addition, the receptor may itself be a transcription factor and may itself directly modulate the presentation and/or secretion of the signal molecule(s).

In embodiments, the receptor is a receptor which responds to light, such as e.g., photoreceptors.

In other embodiments, the receptor is a receptor which responds to pH or electrochemical changes, such as e.g., ion channels. In further embodiments, the receptor is a receptor which responds to mechanical stress, such as e.g., mechanosensitive receptors. In additional embodiments, the receptor is a receptor which responds to small molecules, such as e.g., second messengers, metabolites, amino acids, sugars, lipids and vitamins. In other embodiments, the receptor is a receptor which responds to peptides, such as e.g., hormones, cytokines, neuropeptides, and synthetic peptides. In embodiments, the receptor is a receptor which responds to proteins, such as e.g., receptors activated by protein-protein interactions.

In embodiments, the receptor is selected from the group consisting of Ion channels, G protein-coupled receptors, Enzyme-linked receptors, Extracellular receptors, Transmembrane receptors, Intracellular receptors, Nuclear receptors, Cytokine receptors, Toll-like receptors, Chemokine receptors, Integrin receptors, Adhesion receptors, Receptor tyrosine kinases, Ligand-gated ion channels, Voltage-gated ion channels, Mechanosensitive ion channels and Photoreceptors.

In preferred embodiments, the receptor is a G protein-coupled receptor (GPCR).

### G Protein-Coupled Receptor(s) (GPCR)

G protein-coupled receptors (GPCRs) are a class of transmembrane proteins that play a critical role in a wide range of physiological processes in the body. They are found on the surface of many different cell types and are involved in sensing a diverse array of stimuli, including light, hormones, neurotransmitters, and sensory stimuli. GPCRs transmit stimuli across the cell membrane by activating intracellular signalling pathways through a G protein-mediated mechanism. This can ultimately result in changes in cellular behaviour and function, including regulation of gene expression, cell proliferation, and signal molecule release. Due to their importance in various physiological processes, GPCRs have become an important target for drug development and many pharmaceuticals are designed to target GPCRs.

Example 6 describes a library of diverse GPCRs that cover sensing of small molecules (melatonin, adenosine, adrenaline, serotonin), peptides (P-factor), and complex proteins (CXCL12/SDF-1). In Example 6 it was shown that a diverse set of GPCRs can be used to induce display of CD19 in a stimuli-concentration-dependent manner with the highest fold change acquired for the native Ste2 (Gpa1, pH=5) sensing α-factor at 324-fold. Hence, these results show that the sensory module is customizable by expression of engineered and heterologous GPCRs enabling a myriad of specific signal inputs (stimuli) for the SCASA system.

Accordingly, in embodiments the receptor is a G protein-coupled receptor (GPCR).

The genetically modified cell according to the present invention may comprise a recombinant nucleic acid sequence encoding a GPCR selected from Table 1.

TABLE 1
List of exemplary GPCRs to be expressed

| | | | Exemplified | Amino acid |
|---|---|---|---|---|
| Name | Origin | GenBank ID | RefSeq IDs | SEQ ID NO |
| Ste2 | Saccharomyces | 850518 | NP_116627 | 24 |
| | cerevisiae | | | |
| Mam2 | Schizosaccharomyces | 2541606 | NP_594722 | 25 |
| | pombe | | | |
| MTNR1A/MT1 | Homo sapiens | 4543 | NP_005949 | 26 |
| ADORA2B/ | Homo sapiens | 136 | NP_000667 | 27 |
| A2bR | | | | |
| ADRA2A/α2A | Homo sapiens | 150 | NP_000672 | 28 |
| adrenoceptor | | | | |
| HTR4 variant b/ | Homo sapiens | 3360 | NP_000861 | 29 |
| 5HT4b | | | | |
| CD184/fusin/ | Homo sapiens | 7852 | NP_001008540.1 | 30 |
| CXCR4a | | | | |

In embodiments, the gene encoding the GPCR is selected from the group consisting of genes with GenBank ID 850518, 2541606, 4543, 136, 150, 3360 and 7852. Each of the provided GenBank IDs may give rise to a number of different gene products, i.e., isoforms, sequences of such isoforms annotated to the listed GenBank IDs are incorporated herein by reference. Examples of gene products originating from the GenBank IDs listed in Table 1 may e.g., be gene products with a sequence according to any one or more of the RefSeq IDs provided in table 1, or any one or more of SEQ ID NOs 24-30.

In embodiments, the receptor is a GPCR, which may be selected from the group consisting of Saccharomyces cerevisiae α-factor pheromone receptor (Ste2), Schizosaccharomyces pombe P-factor pheromone receptor (Mam2), Human melatonin receptor 1A (MTNR1A/MT1), Human adenosine A2B receptor (ADORA2B/A2bR), Human alpha-2A adrenergic receptor (ADRA2A/a2A adrenoceptor), Human serotonin/5-hydroxytryptamine receptor isoform b (HTR4 variant b/5HT4b) and Human C—X—C chemokine receptor type 4 isoform a (CD184/fusin/CXCR4a). In preferred embodiments, the G protein-coupled receptor (GPCR) is the Saccharomyces cerevisiae α-factor pheromone receptor (Ste2). Ste2 may be expressed natively, or it may be expressed from a non-native genomic locus, and/or may be under control of promoter(s) and/or terminator(s) not natively associated with the expression of Ste2 gene.

### G Proteins

G proteins are a family of signalling proteins that play a crucial role in transmitting extracellular signals across the cell membrane to initiate intracellular signalling pathways. G proteins are activated by GPCRs, which bind to extracellular ligands and initiate a signalling cascade that leads to the activation of G proteins. G proteins are composed of three subunits, including G-alpha (G_{α}), G-beta, and G-gamma, which function together to regulate various intracellular signalling pathways.

In embodiments, the genetically modified yeast cell further comprises expression of a G-protein. In further embodiments, said G protein is a native G protein, under control of promoter(s) and/or terminator(s) not natively associated with the expression of the native gene. In additional embodiments, said G protein is a heterologous or synthetic G protein, such as e.g., a chimeric G protein.

In embodiments, the genetically modified yeast cell comprises recombinant expression of the G_{α} protein GPA1 of Saccharomyces cerevisiae.

### Transcription Factors

In general, transcription factors are proteins that bind to DNA and regulate the expression of genes. The activity of a transcription factor can be modulated by various mechanisms, including post-translational modifications, protein-protein interactions, light, heat, pH, electrochemical changes and changes in cellular signalling pathways. Accordingly, in the meaning of the present disclosure a transcription factor may act as a receptor.

Accordingly, in embodiments, the genetically modified yeast cell expresses a transcription factor, whose activity is regulated by post-translational modifications, protein-protein interactions, and/or changes in cellular signalling pathways.

In other embodiments, the modified yeast cell of the present disclosure
expresses at least one receptor whose activity regulates the activity of a signalling pathway,
expresses a transcription factor, whose activity is regulated by said signalling pathway.

Additionally, said cell may further express one or more signal molecule(s), which expression is/are regulated by the activity of said transcription factor.

In embodiments, the transcription factor is endogenous to the yeast cell. In other embodiments the transcription factor is of heterologous origin with respect to the yeast cell. Accordingly, said transcription factor may be a naturally occurring transcription factor or it may be of synthetic/artificial origin.

In embodiments, the transcription factor is selected from the group consisting of Gal4, Ste12, and engineered Ste12, with an amino acid sequence according to any one of SEQ ID NOs 193, 35 and 36, or functional variants thereof, which amino acid sequence is at least 80% identical, such as e.g., at least 85%, at least 90%, at least 95%, at least 98%, or such as e.g., at least 99% identical to any one of SEQ ID NOs: 193, 35 and 36.

In particular embodiments, the transcription factor is Ste12 from Saccharomyces cerevisiae, with an amino acid sequence according to SEQ ID NO: 35, or a functional variant thereof which amino acid sequence is at least 80% identical, such as e.g., at least 85%, at least 90%, at least 95%, at least 98%, or such as e.g., at least 99% identical to SEQ ID NO: 35. In particular embodiments, the transcription factor is Gal4 from Saccharomyces cerevisiae, with an amino acid sequence according to SEQ ID NO: 193, or a functional variant thereof which amino acid sequence is at least 80% identical, such as e.g., at least 85%, at least 90%, at least 95%, at least 98%, or such as e.g., at least 99% identical to SEQ ID NO: 193. In embodiments, the transcription factor comprises a fragment of a transcription factor Gal4 or Ste12, or a fragment of Gal4 or Ste12.

In embodiments, the transcription factor is Gal4 or Ste12. In other embodiments, the transcription factor is a synthetic transcription factor which comprises synthetic transcription factors containing pheromone-responsive domain (PRD) of Ste12.

In embodiments, the transcription factor comprises a pheromone-responsive domain from Ste12. In additional embodiments, the pheromone-responsive domain from Ste12 is fused to a DNA-interacting domain. Non-limiting examples of such DNA-interacting domains are any of LexA, TetR, Z3E, Zinc-fingers, VP16, VP64 and Cas9. In embodiments, the transcription factor comprises a domain according to Pfam PF02200, such as e.g., a domain according to SEQ ID NO: 36.

In embodiments, the activity of the transcription factor is regulated by a post-translational modification. In embodiments, the activity of the transcription factor is regulated by protein-protein interactions. In embodiments, the activity of the transcription factor is regulated by light. In embodiments, the activity of the transcription factor is regulated by electrochemical changes in said yeast cell. In embodiments, the activity of the transcription factor is regulated by one or more cellular signalling pathways.

In Example 1-8, for most strains, the signal propagation from the stimulation of the Ste2 GPCR (receptor) is mediated by the Ste12 transcription factor, which is part of the yeast mating pathway. In particular embodiments, the modified yeast cell recombinantly expresses the Ste2 receptor and expresses the Ste12 transcription factor.

In Example 1 and Example 8, for two strains, the signal propagation from the stimulation of the Ste2 GPCR (receptor) is mediated by the LexA-Ste12PRD synthetic transcription factor. In Example 2, for some strains, the signal propagation from stimulation with galactose is mediated by Gal3 (receptor), Gal80 (transcriptional regulator), and the Gal4 transcription factor.

### Yeast Mating Pathway

The yeast mating pathway, e.g., the pheromone response pathway (PRP), is a complex biological process that allows two haploid cells of opposite mating type to mate and form a diploid cell. The process begins with the recognition of a specific pheromone by receptors on the surface of the opposite mating type cell, leading to activation of a signal transduction pathway that ultimately results in the expression of mating-specific genes. The yeast mating pathway may be engineered such that it is responsive to specific stimulation. An example of such a modified pathway is provided in Examples 1-8, wherein the PRP-coupled GPCRs ste2 and ste3, native G_{α}-subunit (gpa1), the negative feedback regulator sst2, the α-factor protease bar1, and the cyclin-dependent kinase inhibitor far1 are deleted to provide a genetically modified yeast cell with a modified yeast mating pathway. To enable specific stimulation with the α-factor, the Ste2 receptor is reintroduced and expressed recombinantly, such that the yeast cell responds to stimulation of the Ste2 receptor. In embodiments, the expression of the Ste2 receptor is controlled by a promoter and/or a terminator sequence, such as e.g., the P_{CCW12} promoter and or the T_{CYC1} terminator. In embodiments, the Ste2 receptor is deleted and one or more of the GPCRs selected from the group consisting of MTNR1A, ADORA2B, ADRA2A, 5HT4B, MAM2 and CXCR4A, is/are expressed. In addition, the native G_{α}-subunit (gpa1) is also reintroduced and recombinantly expressed. In embodiments, the expression of the G_{α}-subunit gpa1 is controlled by a promoter and/or a terminator sequence, such as e.g., the P_{PGK1} promoter and or the T_{CYC1} terminator. In particular embodiments, the G_{α}-subunit (gpa1) is replaced by heterologous chimeric G_{α}-subunits; Gpa1/G_{α}z and Gpa1/G_{αi2}.

### Signal Molecule

In the present invention a signal molecule is a molecule capable of transmitting information or signals between cells, such as e.g., between a yeast cell and a human immune cell. Examples of such molecules can be proteins, peptides, and small molecules, such as e.g., scaffold proteins, cytokines, or growth factors, or small molecules, such as e.g., hormones and neurotransmitters.

Signal molecules can trigger a wide range of cellular responses in a receiver cell, such as e.g., changes in gene expression, cell division, migration, or differentiation, which alone or together can change the phenotype of a cell, such as e.g., a mammalian cell.

In embodiments, the genetically modified yeast cell as described herein expresses one or more signal molecule(s), which expression is/are regulated by the activity of a transcription factor as described herein, wherein said signal molecule(s) is/are presented on the surface and/or secreted from said cell in response to said activity.

In response to presentation and/or secretion of said signal molecule(s), the receiver cell such as e.g., a human immune cell is modulated, manipulated and/or influenced and may obtain an altered phenotype in response to said presentation and/or secretion of said signal molecule(s) from said yeast cell i.e., the presentation and/or secretion of said signal molecule(s) are in some instances influencing the phenotype of the receiver cell, such as e.g., a human immune cell. Examples of such modulation, manipulation and/or influence in the phenotype is exemplified in Example 3, Example 5, Example 7, and Example 8.

Preferably, the signal molecule is presented on the surface and/or secreted from the cell in response to regulation of said receptor and/or transcription factor.

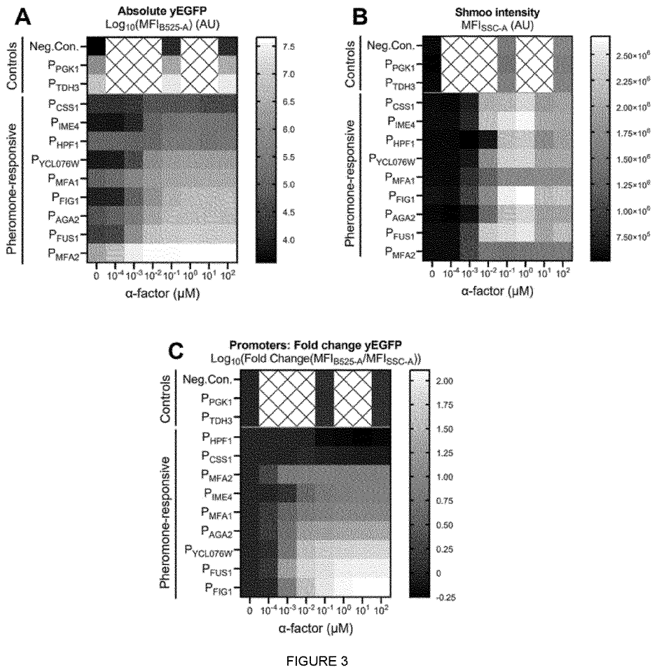

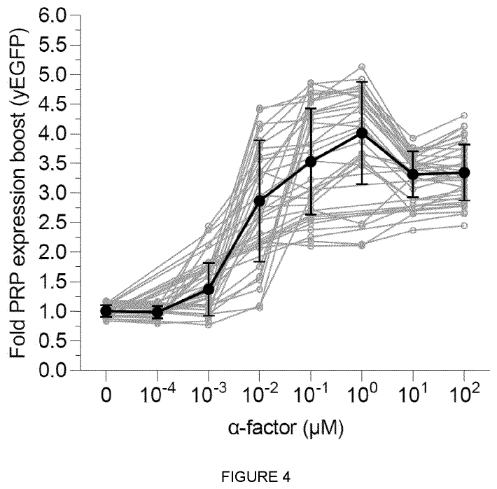

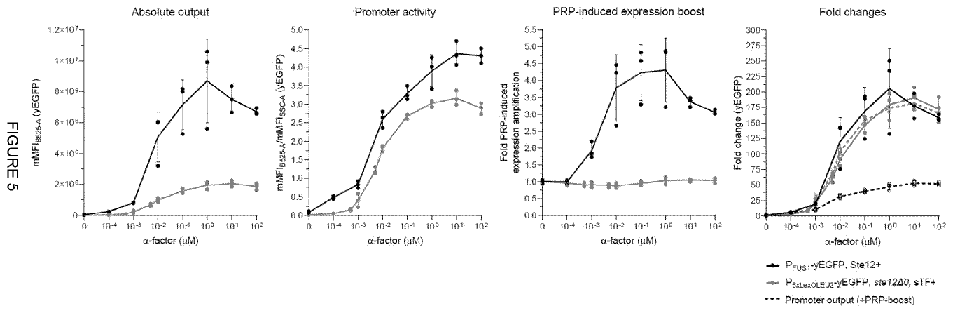

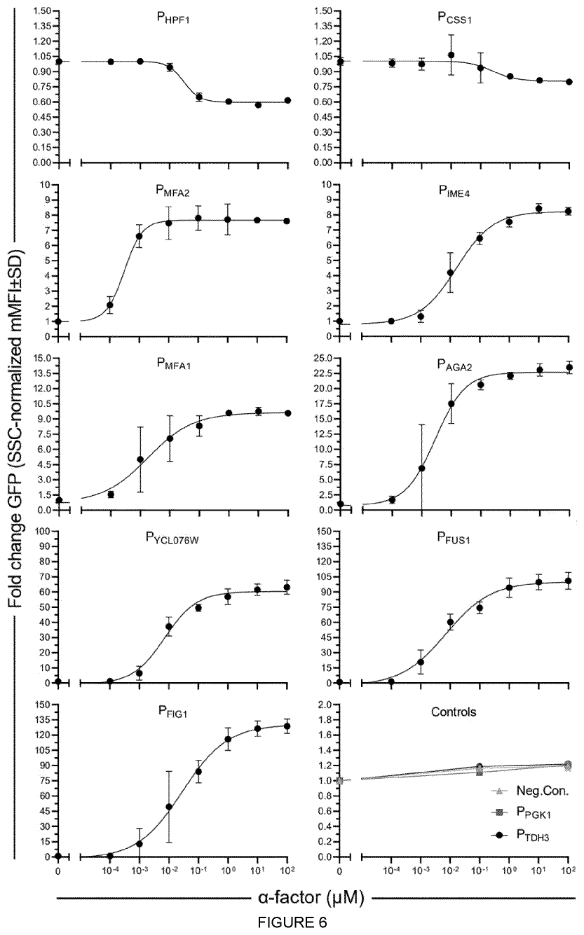

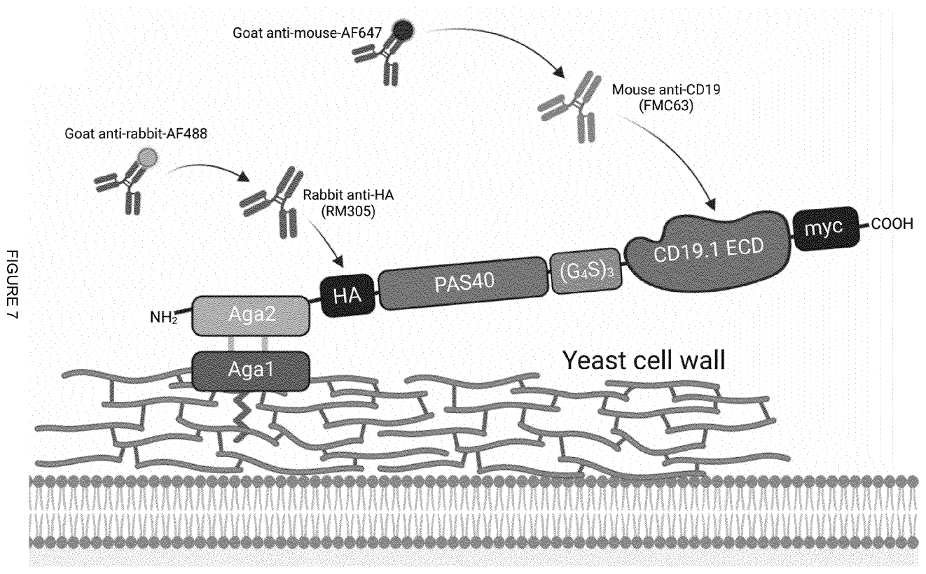

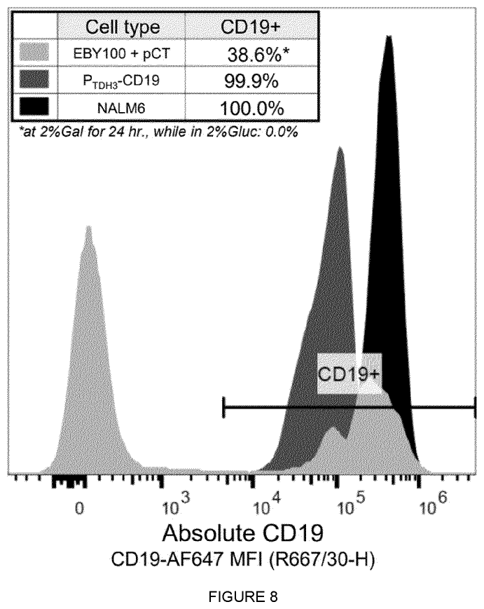

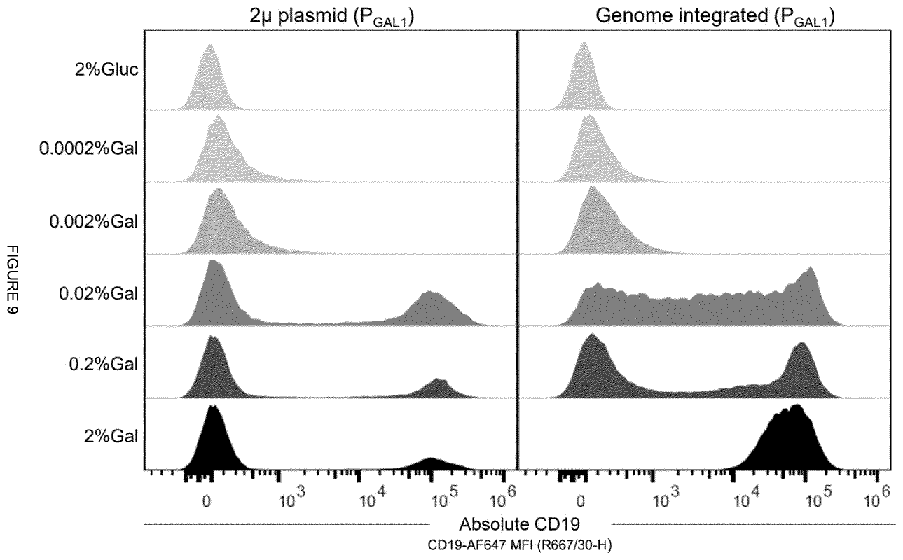

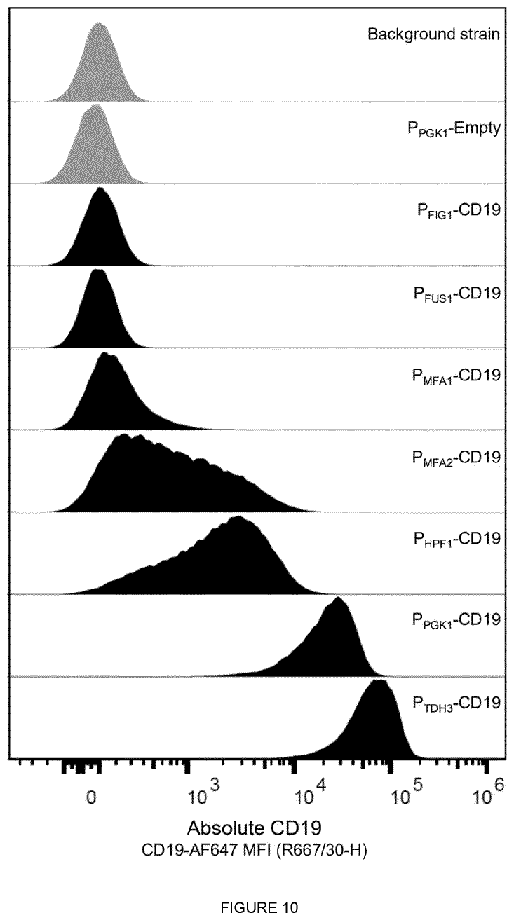

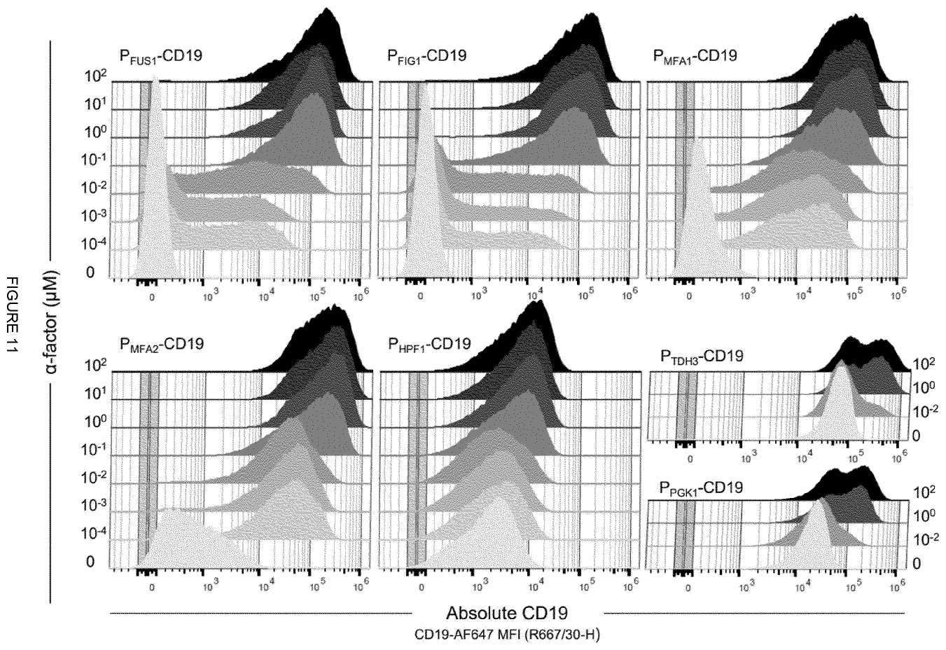

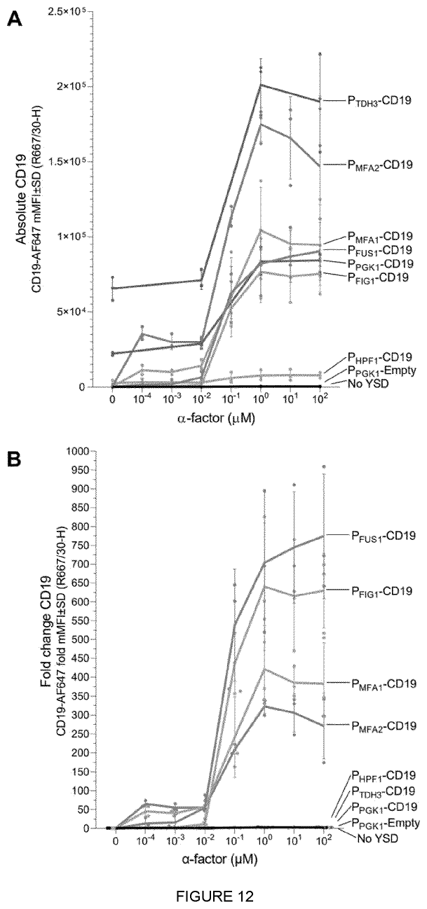

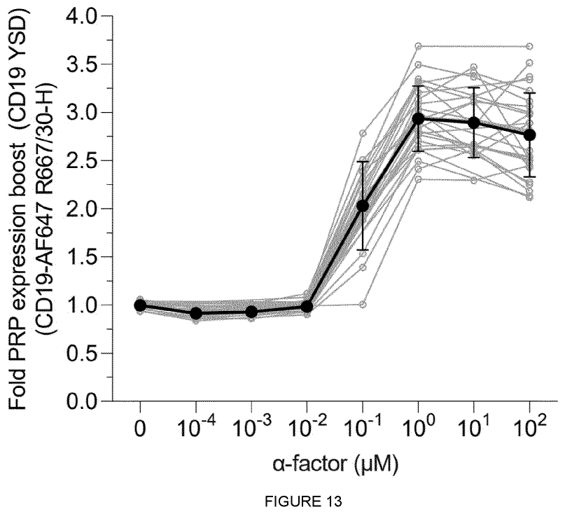

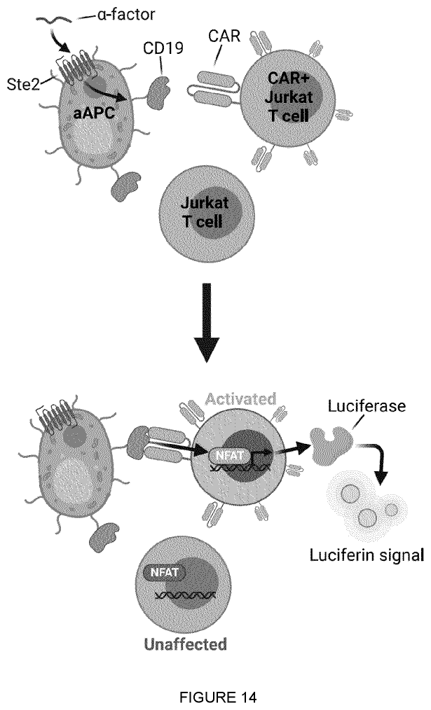

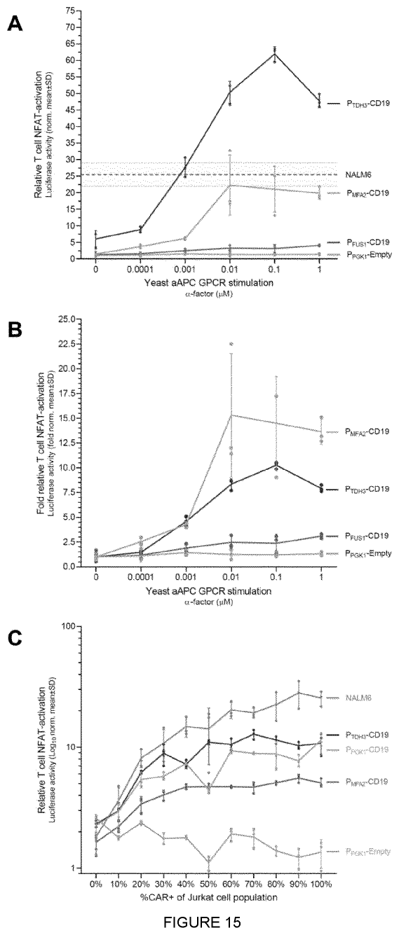

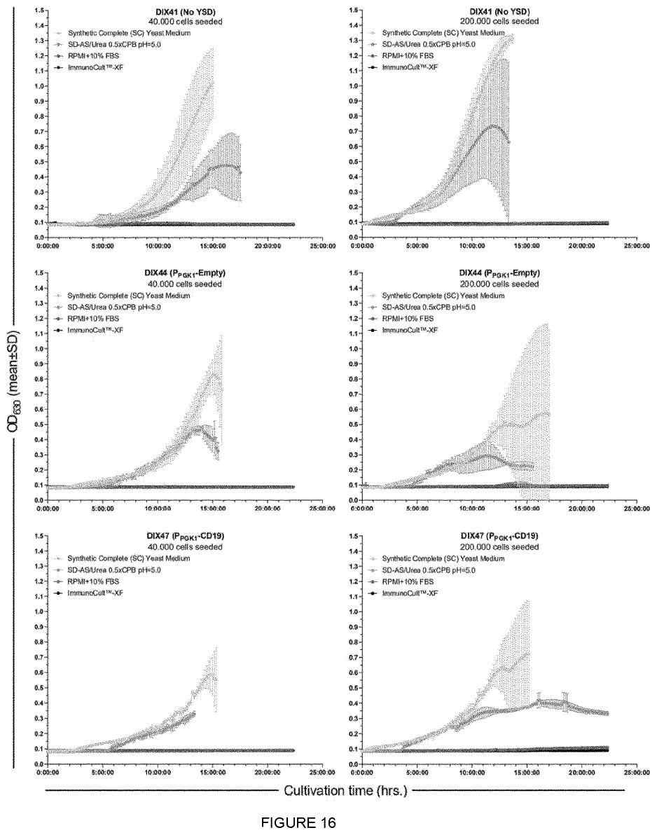

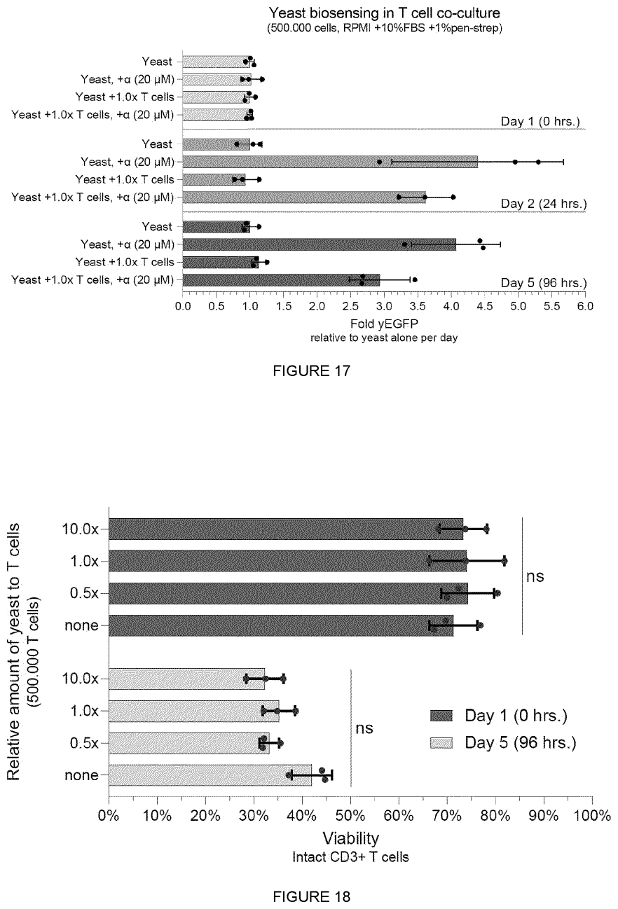

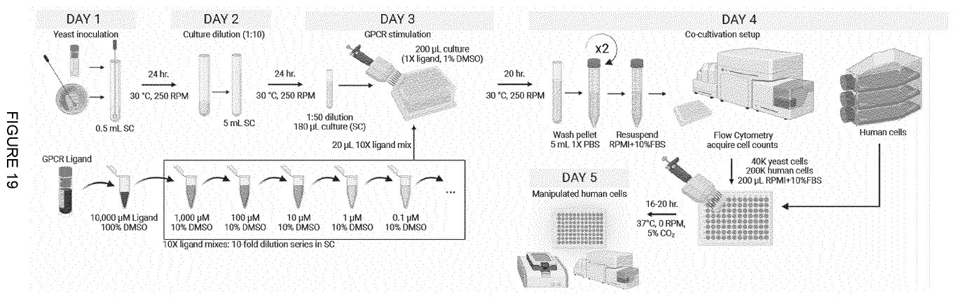

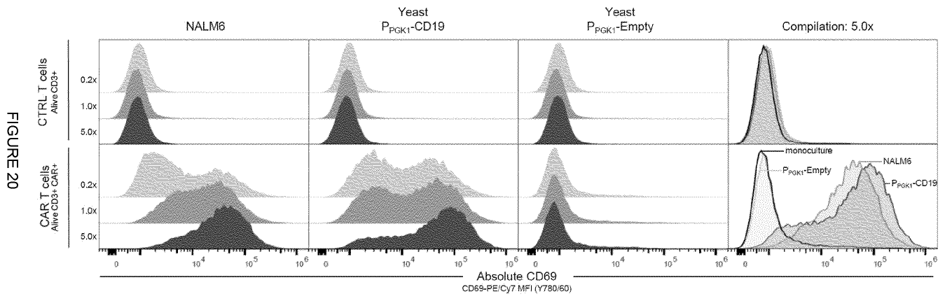

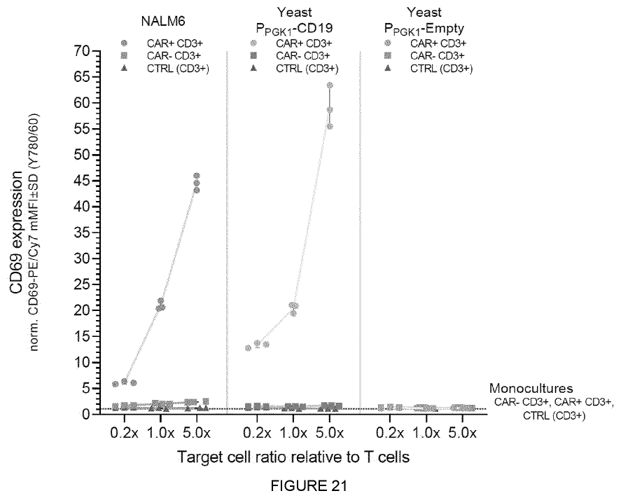

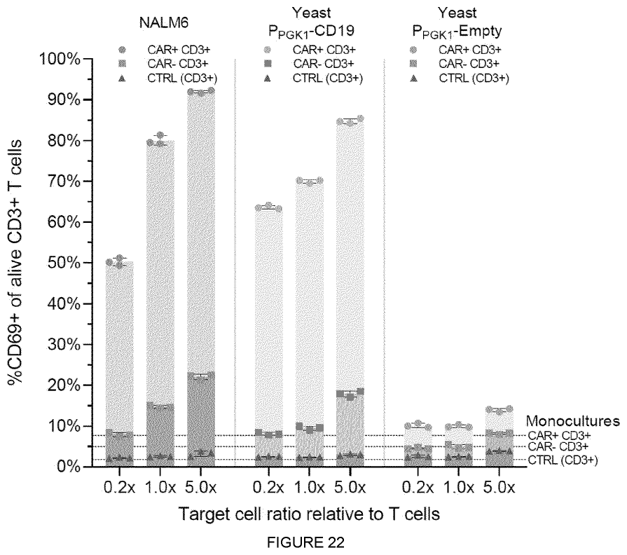

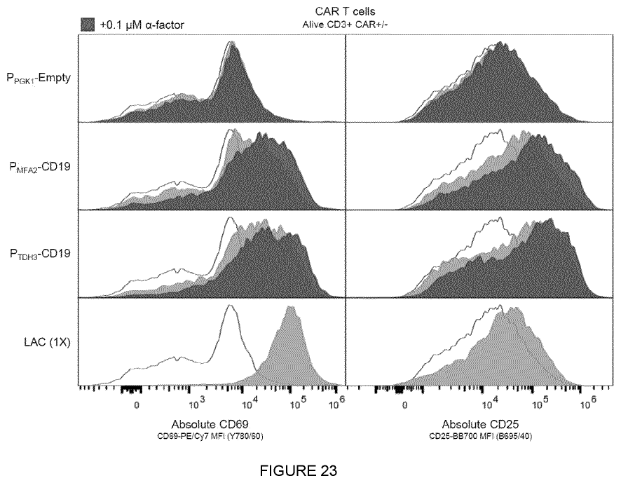

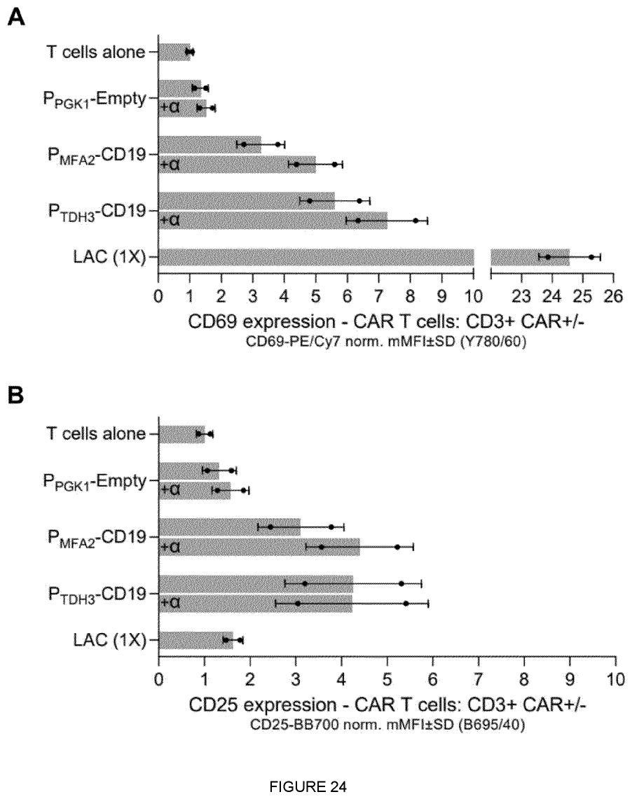

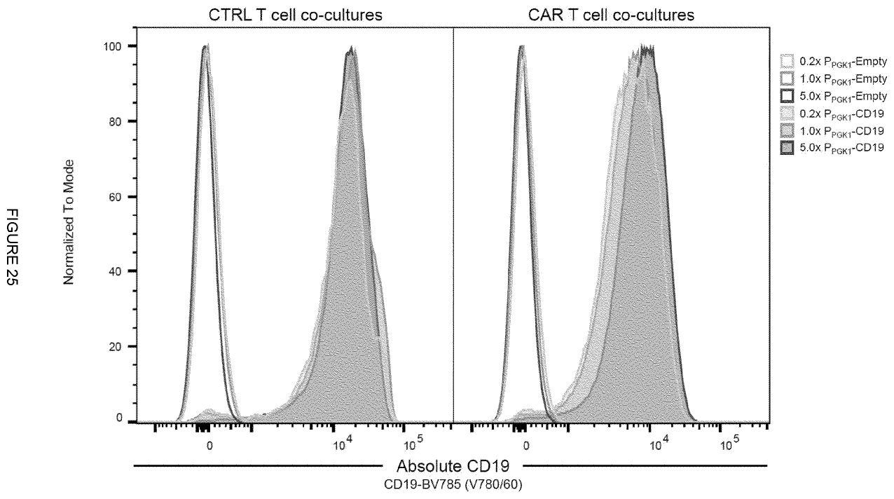

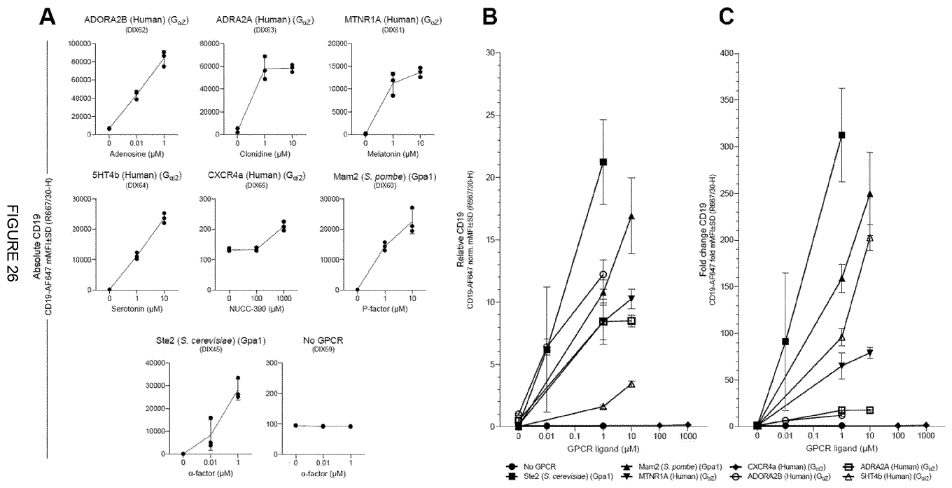

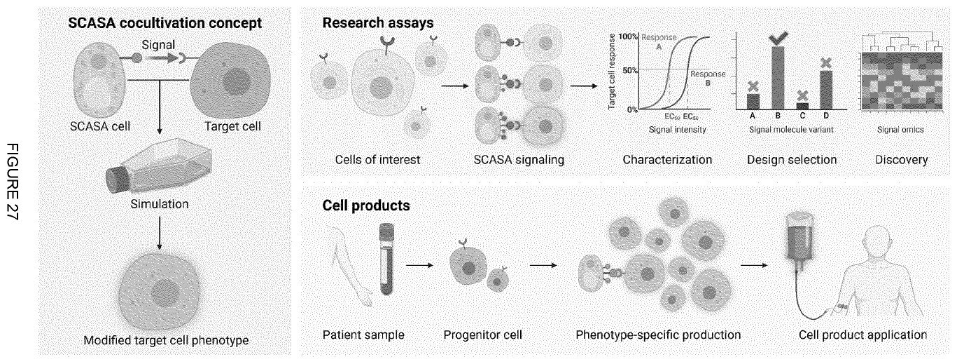

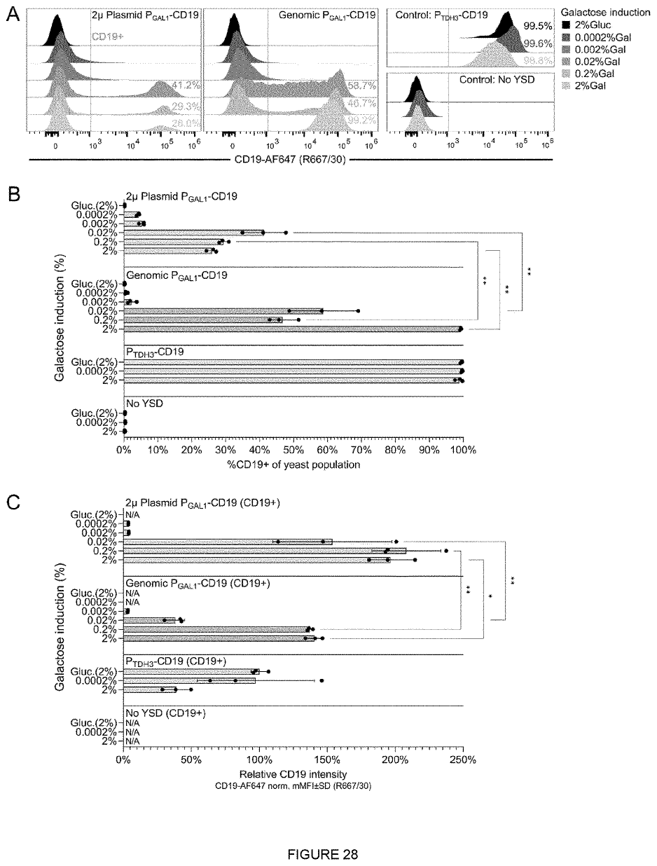

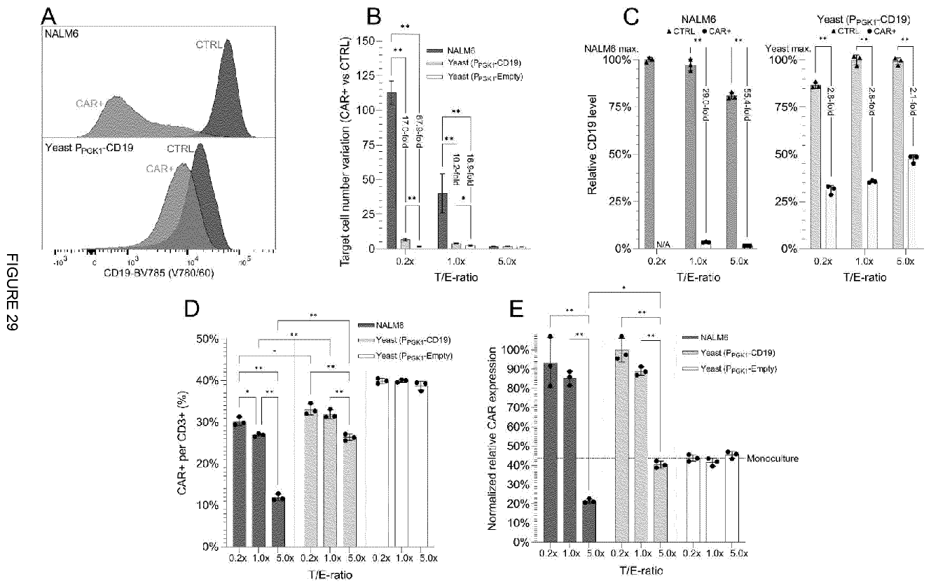

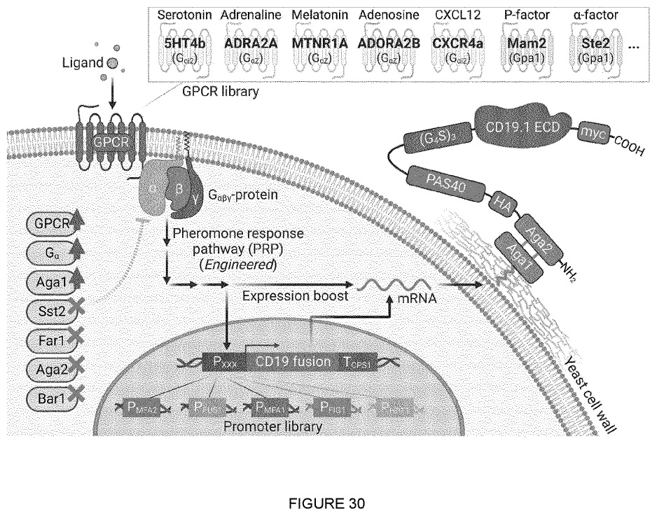

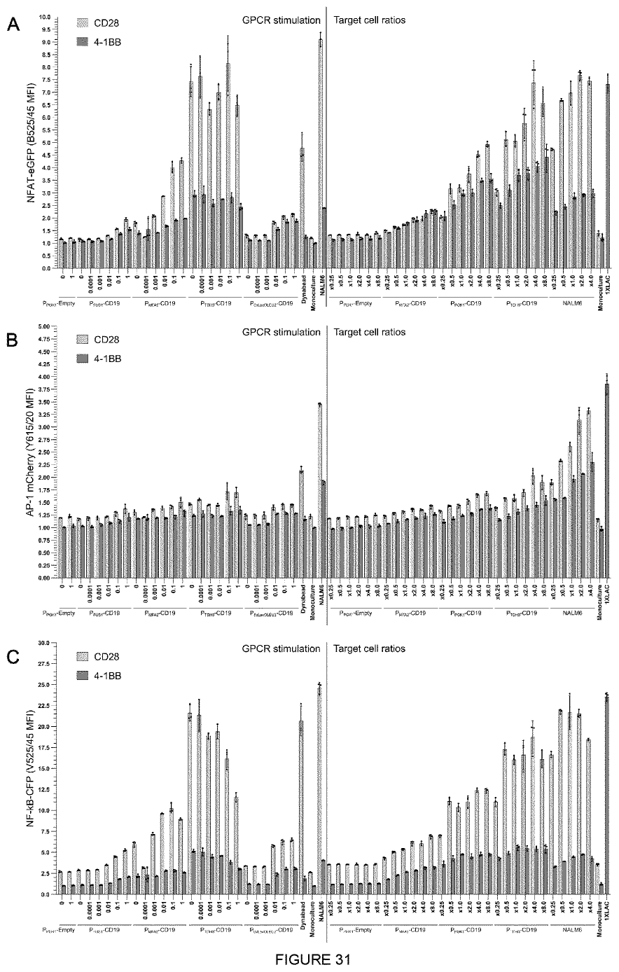

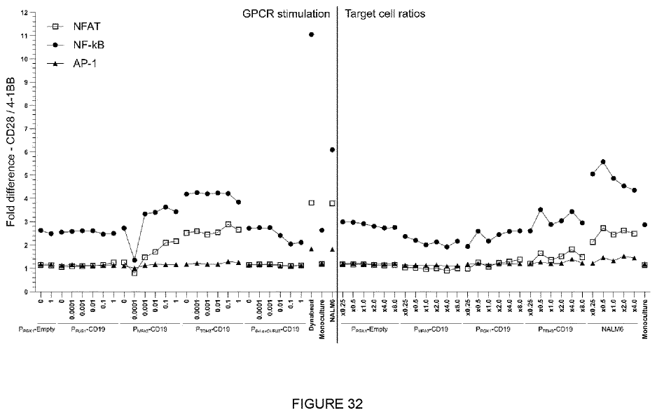

Accordingly, upon changes in the activity of the receptor and/or transcription factor, the signal molecule will be expressed to a higher or lower degree, such as e.g., is exemplified in Example 1 and 2, which shows that the expression of yEGFP (Example 1) and CD19 (Example 2) could be modulated in response to activation of a G protein-coupled receptor (Ste2) with a ligand (α-factor) which activates the PRP, which in turn initiates the transcription of the yEGFP (Example 1) or CD19 (Example 2), and thereby drives the expression of the signal molecule, in these cases yEGFP (see FIG. 2) or CD19 (see FIG. 7 and FIG. 30). It was also shown in Example 1 (see FIGS. 3-6) and Example 2 (see FIGS. 10-12) that the expression of the signal molecule could be further modified by changing the promoter sequence, in response to stimulation with a ligand. Example 1 and Example 2 serves as a proof of concept, and it is to be understood that the examples shown herein are not to be understood as limiting for alternative embodiments.

The signal from the genetically modified yeast cell may be relayed from the yeast cell to the mammalian cell via secretion of the signal molecule from the yeast cell to the extracellular medium, where it may be recognized by a receptor and/or transcription factor and in turn may initiate a cellular response resulting in modulation of the phenotype of the mammalian cell. Accordingly, the signal molecule(s) may be ions, such as e.g., Calcium (Ca2+), Sodium (Na+), Potassium (K+), Chloride (Cl−), Hydrogen ions (H+ or protons), Magnesium (Mg2+), Iron (Fe2+ and Fe3+), Zinc (Zn2+), Copper (Cu2+) or Manganese (Mn2+). The signal molecule(s) may alternatively be small molecules such as e.g., amino acids, vitamins, lipids, nucleotides, sugars, small molecule hormones, small molecule drugs, and combinations and synthetic variants thereof. The signal molecule(s) may alternatively be co-factors such as e.g., Nicotinamide adenine dinucleotide (NAD+/NADH), Nicotinamide adenine dinucleotide phosphate (NADP+/NADPH), Adenosine triphosphate (ATP), Flavin adenine dinucleotide (FAD/FADH2), Coenzyme A (CoA), S-adenosylmethionine (SAM), Tetrahydrofolate (THF), prostaglandins (PG), and Pyridoxal phosphate (PLP). The signal molecule(s) may alternatively be peptides such as e.g., synthetic signalling peptides, adrenocorticotropic hormone (ACTH), growth hormone-releasing hormone (GHRH), gonadotropin-releasing hormone (GnRH), thyrotropin-releasing hormone (TRH), oxytocin, vasopressin (antidiuretic hormone, ADH), angiotensin II, bradykinin, calcitonin gene-related peptide (CGRP) and substance P.

In embodiments, the genetically modified yeast cell expresses one or more proteins required to produce said signal molecule. In embodiments, the proteins required to produce said signal molecule are heterologous with respect to the genetically engineered yeast cell.

The signal molecule(s) may alternatively be proteins, such as e.g., Cytokines, Chemokines, Interleukins, Growth factors, Hormones, Neurotransmitters, Extracellular matrix (ECM) proteins, Wnt proteins, Hedgehog proteins, Transforming growth factor-beta (TGF-β), Interleukins, Fibroblast growth factors (FGFs), Bone morphogenetic proteins (BMPs), Insulin-like growth factors (IGFs), Vascular endothelial growth factors (VEGFs) and Platelet-derived growth factors (PDGFs), or antigens, such as e.g., CD19, CD79b, BCMA, and HER2

The signal molecule(s) may alternatively consist of or comprise antibodies and antibody fragments, such as e.g., IgG, IgA, IgM, IgE, IgD, Fab, Fc, F(ab′)2, scFv, Fv, Diabodies, Nanobodies, Chimeric antibodies, Humanized antibodies and Polyclonal antibodies.

Additionally, the signal molecules may also be a fusion peptide or protein, comprising a combination of any of the signal molecules as described above.

The SCACA system as presented herein offers a genetically modified yeast cell which can relay a signal from said yeast cell to a mammalian cell, via the expression and presentation and/or secretion of a signal molecule. In some embodiments, the signal molecule is presented on the surface of the yeast cell, such that the presented signal molecule may be recognized by or interact with a molecule present in or on the surface of the receiver cell.

The method for modulating, manipulating and/or influencing the phenotype of human immune cells as presented herein utilizes a genetically modified yeast cell which relay a signal from said yeast cell to a mammalian cell, via the expression and presentation and/or secretion of a signal molecule. In some embodiments, the signal molecule is presented on the surface of the yeast cell, such that the presented signal molecule may be recognized by or interact with a molecule present in or on the surface of the receiver cell.

Methods to generate a genetically modified yeast cell capable of presenting a signal molecule on the surface is generally known in the art (Tanaka et al., Appl Microbiol Biotechnol. 2012 August; 95(3):577-91).

In embodiments, the genetically modified yeast cell expresses a signal molecule. In embodiments, the genetically modified yeast cell expresses one or more signal molecule(s), such as e.g., at least 1 type of signal molecule such as e.g., at least 2, 3, 4, 5, 6, 7, 8, 9, 10, 20, 50 or such as e.g., at least 100 different signal molecules.

In embodiments, the signal molecule is a native signal molecule. In other embodiments, the signal molecule is a heterologous signal molecule with respect to the genetically modified yeast cell. In embodiments, the signal molecule is encoded by a nucleic acid sequence. Said nucleic acid sequence may be synthetic, recombinant, or heterologous. Preferably, the nucleic acid sequence encoding the signal molecule is expressed via a synthetic or recombinant nucleic acid construct.

Preferably, the expression of the signal molecule is regulated by a promoter and/or terminator.

In embodiments, the expression of the signal molecule is under control of a promoter and/or terminator selected from Table 2 and/or Table 3.

Preferably, the expression of the signal molecule is under control of a promoter selected from the group consisting of P_{MFA2}, P_{MFA1}, P_{AGA2}, P_{YCL076W}, P_{IME4}, P_{HPF1}, P_{CSS1}, P_{FUS1}, P_{FIG.1}, P_{TDH3}, P_{PGK1}, P_{SNZ1}, P_{CPS1}, P_{CCW12}, P_{6xLexO-LEU2}, P_{GAL1}, P_{TEF1} and P_{RAD27} comprising or consisting of nucleic acid sequence according to any one of SEQ ID NOs: 1-18 or functional variants thereof with a nucleic acid sequence that is at least 80% identical to any one of SEQ ID NOs: 1-18.

In embodiments the expression of said signal molecule(s) is/are regulated by the activity of said transcription factor. In additional embodiments, said signal molecule(s) is/are presented on the surface and/or secreted.

In embodiments, the process of secretion involves one or more of the following steps, including synthesis, packaging of the signal molecule, transport to the cell membrane, and release into the extracellular space.

In additional embodiments, the signal molecule may be synthesized in the cytoplasm or in specialized organelles, such as e.g., the endoplasmic reticulum or Golgi apparatus.

Once secreted from the genetically modified yeast cell, the signal molecule may diffuse and bind to receptors on the surface of the receiver cell, preferably a human immune cell, initiating a signalling cascade that leads to one or more cellular responses.

In other embodiments, the signal molecule is presented on the surface of the genetically modified yeast cell. Presentation of the signal molecule may involve fusion of the signal molecule to a presentation module, such as e.g., the yeast Aga2 presentation system (Mei et al., Microbiol Res. 2017 March; 196:118-128). Other means of surface presentation by presentation modules may involve fusion of the signal molecule to other molecules presented on the extracellular surface of yeast cells, such as e.g., Sag1, Aga1, Aga2, Cwp1, Cwp2, Tip1, Flo1, Sed1, Fig2, Pir and/or Tir1, and modified variants thereof. Suitable alternative presentation modules and methods to implement such surface presentation modules are well known in the art. Examples of such are presented in e.g., Kondo et al., Applied Microbiology and Biotechnology volume 64, pages 28-40 (2004), Cherf et al. Methods Mol Biol. 2015; 1319: 155-175, or Teymennet-Ramirez et al. Front Bioeng Biotechnol. 2022 Jan. 10; 9:794742, which are incorporated herein by reference.

In embodiments, the presentation of the signal molecule comprises fusion of the signal molecule to the yeast Aga2 protein. In embodiments, the signal molecule is a fusion protein comprising a presentation module, one or more antibody tags, one or more linker regions and one or more signal molecules. In embodiment, the signal molecule fusion protein comprises the Aga2, a HA tag, a myc tag, a PAS40 linker, a repetitive G4S linker (GGGGS) and a signal molecule such as e.g., CD19. In embodiments, the linker region is selected from PAS40 linkers and GS linkers (see FIG. 7 and FIG. 30). Alternative suitable linkers are well known in the art, and the mentioned linkers serve as mere examples of suitable linkers. In embodiments, the signal molecule fusion proteins comprise an antibody tag, such as, e.g., GFP, FLAG, HA, Myc, V5, T7, His, RFP, GST, MBP, mCherry and Luciferase. In preferred embodiments, the antibody is an HA tag. Suitable alternative antibody tags and methods to implement such antibody tags are well known in the art.

In embodiments, the signal molecule(s) is/are selected from the group consisting of cancer antigens, MHC-I protein, MHC-II proteins, B-cell receptors, adhesive proteins, cytokines, hormones, neurotransmitters, fluorescent proteins, antibodies, Fabs, scFVs, nanobodies, and antibody tags.

In embodiments, the signal molecule(s) binds to one or more target(s) on the receiver cell, such as e.g., a human immune cell. In cancer there are several examples in the prior art of receptors of signal molecules being targeted on e.g., cancer cells. Some non-limiting examples of signal molecules targeting or being targeted in the receiver cells are e.g., CD3, CD4, CD8, CD19, CD21, CD28, CD14, CD56, CD40, CD45, CD80/CD86 and CD95/Fas.

In mammals, such as e.g., humans, CD19 is a cell surface protein that is expressed on B cells, from early pre-B cells to mature B cells but not on plasma cells. It is a member of the immunoglobulin superfamily and acts as a co-receptor along with the B cell receptor (BCR) to activate B cells. Upon binding to its ligand, CD21, CD19 activates intracellular signalling pathways that enhance the activation of the BCR and promote B cell proliferation and survival. CD19 signalling is also involved in the regulation of B cell tolerance and the production of high-affinity antibodies.

CD19 has been widely used as a B cell marker in flow cytometry and immunohistochemistry studies, and its expression is also a target for immunotherapy of B cell malignancies such as e.g., B cell lymphoma and leukaemia.

Accordingly, in preferred embodiments, the signal molecule is CD19. In embodiments, the genetically modified cell presents CD19 on the surface of the genetically modified yeast cell.

In further embodiments the signal molecule(s) is/are selected from the group consisting of avidin, streptavidin, IFN-α, IFN-β, IFN-γ, TGF-β, VEGFA, IDO, IL-1 family (e.g., IL-1β), IL-2, IL-4, IL-5, IL-6, IL-10, IL-12, IL-1β, IL-15, IL-17 IL-21, IL-22, IL-23, IL-35, ICOS-L, PD-L1, PD-L2, Anti-CTLA-4, CD79b, BAFF-R, TD2, BCMA, SAMF7, NKG2D, NKG2DL, CD19, CD20, CD22, CD23, CD30, CD33, CD38, CD73, CD80/CD86, CD123, CD133, CD138, CD171, CD229, CD319, GPRC5D, LeY, FRβ, FLT3, EBV, FRα, MSLN, EGFR, CEA, MUC1, GD2, EpCAM, CAIX, L1-CAM, EGFRvIII, IL13Rα2, PSMA, PSCA, PAP, TARP, FAP, c-MET, EphA2, GPC3, VEGFR-2, ROR1, MUC16, ROBO1, K light chain, A light chain, APRIL, 5 mesothelin, CTAG1B, CA125, KK-LC-1, MAGE-A1, MAGE-A4, TAG-72, Claudin 18.2, FOLR1, CCT301-38, and CCT301-59, yEGFP, anti-CD3, anti-CD28, IL-2, HER2 and PD-1.

In preferred embodiments, the signal molecule(s) is/are selected from the group consisting of:
a) CD19 with an amino acid sequence according to SEQ ID NO: 53, or a functional homologue thereof with an amino acid sequence which is at least 80% identical, such as e.g., at least 85%, at least 90%, at least 95%, at least 98%, or such as e.g., at least 99% identical to SEQ ID NO: 53,
b) yEGFP, with an amino acid sequence according to SEQ ID NO: 38, or a functional homologue thereof with an amino acid sequence which is at least 80% identical such as e.g., at least 85%, at least 90%, at least 95%, at least 98%, or such as e.g., at least 99% identical to SEQ ID NO: 38,
c) anti-CD3, with an amino acid sequence according to SEQ ID NO: 59, or a functional homologue thereof with an amino acid sequence which is at least 80% identical such as e.g., at least 85%, at least 90%, at least 95%, at least 98%, or such as e.g., at least 99% identical to SEQ ID NO: 59,
d) anti-CD28, with an amino acid sequence according to SEQ ID NO: 60, or a functional homologue thereof with an amino acid sequence which is at least 80% identical such as e.g., at least 85%, at least 90%, at least 95%, at least 98%, or such as e.g., at least 99% identical to SEQ ID NO: 60,
e) IL-2, with an amino acid sequence according to SEQ ID NO: 54, or a functional homologue thereof with an amino acid sequence which is at least 80% identical such as e.g., at least 85%, at least 90%, at least 95%, at least 98%, or such as e.g., at least 99% identical to SEQ ID NO: 54,
f) HER2 with an amino acid sequence according to SEQ ID NO: 55, or a functional homologue thereof with an amino acid sequence which is at least 80% such as e.g., at least 85%, at least 90%, at least 95%, at least 98%, or such as e.g., at least 99% identical to SEQ ID NO: 55, and
g) PD-L1 with an amino acid sequence according to SEQ ID NO: 57, or a functional homologue thereof with an amino acid sequence which is at least 80% such as e.g., at least 85%, at least 90%, at least 95%, at least 98%, or such as e.g., at least 99% identical to SEQ ID NO: 57.

In embodiments, the genetically modified yeast cell
a) recombinantly expresses an endogenous receptor,
b) recombinantly expresses an endogenous G_{α} protein and
c) expresses a fusion protein comprising a signal molecule.

In further embodiments, the genetically modified yeast cell
a) recombinantly expresses the Ste2 receptor or expresses the MTNR1A, ADORA2B, ADRA2A, 5HT4B, MAM2 or CXCR4A receptor or functional variants thereof,
b) recombinantly expresses the G_{α} protein Gpa1 or expresses a functional variant thereof, such as e.g., chimeric human Gpa1/Gaz and Gpa1/G_{α2}, and
c) expresses a fusion protein comprising a signal molecule, such as e.g., CD19, and a surface presentation module, such as e.g., Aga2 from Saccharomyces cerevisiae.

In embodiments, the genetically modified yeast cell expresses a combination of signal molecules, such as, e.g., the combination of a small molecule and a peptide, a combination of peptide and a protein, or combinations of small molecules, peptides and/or proteins.

In embodiments, the genetically modified yeast cell expresses a combination of signal molecules, wherein the expressed signal molecules are,
anti-CD3 and anti-CD28, or
IL-2 and anti-CD3, or
IL-2 and anti-CD28, or
anti-CD3 and anti-CD28 and IL-2, or
any combination thereof.

In embodiments, the genetically modified yeast cell expresses a combination of signal molecules, wherein the expressed signal molecules are CD19 and PD-1.

In embodiments, the genetically modified yeast cell expresses a combination of signal molecules, wherein the expressed signal molecules are HER2 and PD-1.

In embodiments, the genetically modified yeast cell expresses a combination of signal molecules, wherein the expressed signal molecules are anti-CD3, anti-CD28, IL-2, CD19, HER2, anti-CD3 and anti-CD28.

In embodiments, the genetically modified yeast cell expresses a combination of signal molecules, wherein the expressed signal molecules are
IL-2 and anti-CD3, or
IL-2 and anti-CD28, or
anti-CD3, anti-CD28 and IL-2, or
CD19 and PD-L1, or
HER2 and PD-L1, or
any combination thereof.

In embodiments, the genetically modified yeast cell expresses a combination of signal molecules and additionally expresses and iron oxide binding protein, such as e.g., SsoFe2,

### Controlling the Expression

In the present context the term “controlling the expression” relates to gene expression where the transcription of a gene into mRNA and its subsequent translation into protein is controlled. Gene expression is primarily controlled at the level of transcription, largely because of binding of proteins to specific sites on DNA, such as e.g., regulatory elements.

As described above, engineering strategy can be applied in multiple ways:
1) the copy number,
2) controlling the expression of any copy of these genes at the transcriptional, post-transcriptional, and/or the translational level,
3) controlling the stability of mRNA,
4) the deletion of regulators that repress the expression of key genes respective pathway,
5) the heterologous expression of regulators that activate and/or enhance the expression of key genes relevant to obtain a dynamic expression of the signal molecule

### Modified Expression

A variety of molecular mechanisms ensures that genes are expressed at the appropriate level and under conditions of relevance to the applied production process. For instance, the regulation of transcription can be summarized into the following routes of influence; genetic (direct interaction of a control factor with the gene of interest), modulation and/or interaction of a control factor within the transcriptional machinery and epigenetic (non-sequence changes in DNA structure that influence transcription).

It is known that a reduction in gene expression below a critical threshold for any gene will result in a mutant phenotype, since such a defect essentially mimics either a partial or complete loss of function of the target gene, whereas increased expression of a native gene can be both beneficial or disruptive to a cell or organism.

In the present disclosure, “heterologous” relates to elements of heterologous origin to the genetically engineered cell. In the present disclosure, “recombinant” relates to the recombination of elements, which may or may not be of heterologous origin with respect to the genetically engineered cell. Accordingly, recombinant expression may entail exchanging an endogenous promoter with another endogenous or heterologous promoter, which e.g., results in an altered expression of the gene of interest.

As such “modified expression profile” relates to an expression level of a gene of interest, wherein the expression is modified in the sense that the expression of the gene either higher or lower, compared to the native expression of the gene. A modified expression may be obtained in a plethora of ways known to the skilled person, and methods for establishing the expression level of a gene or genes of interest is well known in the art, and may entail measuring e.g., a gene of interest's mRNA levels (transcription product) or actual protein levels of the translated protein (translation product).

Furthermore, the expression levels of genes of interest may also be modified indirectly by transcriptional activators that bind to key gene regulatory sequences to promote transcription or enhancers that constitute sequence elements positively affecting transcription, thus not necessarily modifying the nucleic acid sequence encoding the gene of interest but modifying the transcription machinery leading to a modified expression profile of the gene of interest.

Similarly, a more direct overexpression of a gene may be achieved by simply increasing the gene's copy number in the genome. As described above, overexpression of a gene of interest may also be obtained by replacing the gene's native promoter with a promoter leading to higher expression of the gene. Such promoter replacement may e.g., be done using an endogenous promoter from a different genomic locus than that of the target gene, a heterologous promoter not originating in the genetically engineered cell, or it may be a synthetic promoter of synthetic origin. Such exchange or replacement of promoters may increase or decrease the expression of the gene or may be used to control the expression of said gene such that the gene is responsive to specific ques, such as e.g., signalling molecules.

Moreover, a modified expression profile of a gene may also be achieved indirectly through the partial or full inactivation of transcriptional repressors that normally bind key regulatory sequences around the coding sequence of the gene of interest and thereby inhibit its transcription.

Thus, in one or more exemplary embodiments, the modified expression of a protein(s) can be provided by increasing the copy number of the genes coding said protein(s), and/or by choosing an appropriate regulatory tag or adding an extra genomic copy, and/or conferring a non-functional (or absent) gene product that normally binds to and repress the expression

In the present disclosure, it is to be understood that “modified expression” includes but is not limited to regulated expression of native genes e.g., at their native genomic locus via. promoter exchange, where the native promoter controlling expression of the gene of interest is exchanged with a promoter which is not native to the gene of interest.

Increasing the copy number Copy number variation is a type of structural variation: specifically, it is a type of duplication or multiplication of a considerable number of base pairs.

In one or more exemplary embodiments, expression of the receptor, transcription factor and/or signal molecule is/are controlled by increasing the copy number of the desired nucleic acid sequence encoding receptor, transcription factor and/or signal molecule.

Thus, in one or more exemplary embodiments, the present disclosure relates to a method, wherein the heterologous, recombinant or modified expression of the genes(s) is provided by increasing the copy number of the genes coding for said receptor, transcription factor and/or signal molecule and/or by choosing an appropriate promoter and/or terminator.

### Regulatory Element

The genetically engineered yeast cell may comprise recombinant genes and/or nucleic acids of homologous or heterologous origin. The expression of said recombinant genes and/or nucleic acids can be regulated by one or more nucleic acid sequences comprising a regulatory element.

The term “regulatory element” is to be understood as a regulatory nucleic acid sequence that modulates the expression of a nucleic acid sequence comprising e.g., a coding sequence.

In general, the transcriptional and translational regulatory sequences include, but are not limited to, promoter sequences, ribosomal binding sites, transcriptional start and stop sequences, translational start and stop sequences, and enhancer or activator sequences.

In one or more exemplary embodiments, the expression of the recombinant nucleic acid(s) encoding the receptor, transcription factor and/or signal molecule is/are regulated by one or more nucleic acid sequences comprising a regulatory element.

### Promoters

In Examples 1-3, Example 5, and Example 8 it is shown that different promoters may be employed to regulate the expression of the signal molecule. The examples show that the system can be tailored such that the promoter and/or terminator regulating the expression of the signal molecule responds in a dynamic fashion in response to the input signal, which in Examples 1-3, Example 5, and Example 8 is obtained from the stimulation of the Ste2 receptor, and is mitigated by the Ste12 transcription factor, which stimulates expression from the specific promoters. In Example 1 and Example 8, this is also shown from stimulation of the Ste2 receptor and mitigated by the LexA-Ste12PRD synthetic transcription factor, which stimulates the expression from the P_{6xLexO-LEU2} promoter. In Example 2, this is also shown from stimulation of the Gal3 receptor, and mitigated by the Gal4 transcription factor, which stimulates expression from the P_{GAL1} promoter. In addition, the promoters used to promote the expression of the receptor and/or transcription factor may also influence the efficiency of the system and the dynamic range. In the examples the inventors have shown that expression of the Ste2 receptor under control of the P_{CCW12} promoter is well attuned to the expression of the Ga protein Gpa1 under control of the P_{PGK1} promoter. It is evident for the skilled person that many such pairs of promoters are available.

Promoters are in general recognised as specific sequences of nucleotides in DNA that signal the beginning of a gene or a transcription unit. They are recognized and bound by RNA polymerase, the enzyme responsible for synthesizing RNA from a DNA template during transcription, and other transcription factors, which initiate the process of transcription. Promoters are generally located upstream of the gene or transcription unit they regulate and contain distinct regions, including a core promoter that contains the transcription start site and binding sites for RNA polymerase and other transcription factors, along with other regulatory regions that can enhance or repress transcription. As is also shown in Examples 1-8 promoter plays a crucial role in determining the level and timing of gene expression, such as e.g., the expression of the signal molecule.

In addition, the system may also be tuned by selection of specific promoters that are regulated by the pheromone response pathway. Further, the method for modulating, manipulating, and/or influencing the phenotype of human immune cells in vitro as disclosed herein, may also be tuned by selection of specific promoters that are regulated by the pheromone response pathway. As is shown in the examples, examples of such promoters are P_{MFA2}, P_{FUS1}, P_{AGA2}, P_{FIG.1}, P_{MFA1}, P_{YCL076W}, P_{HPF1}, P_{IME4} and P_{CSS1}. In embodiments, the promoter promoting the expression of the signal molecule is selected from the group of promoters consisting of P_{MFA2}, P_{MFA1}, P_{AGA2}, P_{YCL076W}, P_{IME4}, P_{HPF1}, P_{CSS1}, P_{FUS1}, P_{FIG.1}, P_{TDH3}, P_{PGK1}, P_{SNZ1}, P_{CPS1}, P_{CCW12}, P_{6xLexO-LEU2}, P_{GAL1}, and P_{TEF1}.

Such a promoter may also be a non-native promoter. A non-native promoter refers to a DNA sequence that regulates the transcription of a gene or protein of interest but is not the endogenous or native promoter normally associated with that gene or protein. This promoter can be from the same species, but not the specific promoter that normally regulates expression of the gene of interest. Alternatively, the non-native promoter can be a heterologous promoter derived from another species or source altogether such as e.g., a synthetic promoter. Non-native promoters are often used in genetic engineering and synthetic biology to control the expression of genes or proteins in a desired manner and can be engineered to respond to specific environmental cues, such as e.g., ligand induced activation of the yeast mating pathway, or to produce protein at specific times. In relation to that there are no limits to the origin of the promoters used herein i.e., they may be endogenous, heterologous, recombinant, or synthetic.

In embodiments, the expression of the receptor is under control of a first promoter. In additional embodiments, the expression of the transcription factor is under control of said first, or a second promoter. In further embodiments, the expression of the signal molecule is under control of said first, said second, or a third promoter. In embodiments, said first, said second, or a third promoter are the same promoters. In other embodiments, two of the said first, said second, or a third promoter are identical. In further embodiments the said first, said second, or a third promoter are the same.

In embodiments, the first, second, and/or third promoter sequence is selected from the group of promoters consisting of P_{MFA2}, P_{MFA1}, P_{AGA2}, P_{YCL076W}, P_{IME4}, P_{HPF1}, P_{CSS1}, P_{FUS1}, P_{FIG.1}, P_{TDH3}, P_{PGK1}, P_{SNZ1}, P_{CPS1}, P_{CCW12}, P_{6xLexO-LEU2}, P_{GAL1}, P_{TEF1} and P_{RAD27} comprising or consisting of nucleic acid sequence according to any one of SEQ ID NOs: 1-18 or functional variants thereof with a nucleic acid sequence that is at least 80% identical, such as e.g., at least 85%, at least 90%, at least 95%, at least 98%, or such as e.g., at least 99% identical to any one of SEQ ID NOs: 1-18.

In embodiments, the promoter(s) is/are selected from Table 2.

TABLE 2
Promoters.

| Promoter name | Origin | SEQ ID NO |
|---|---|---|
| P_{MFA2} | Saccharomyces cerevisiae | 1 |
| P_{MFA1} | Saccharomyces cerevisiae | 2 |
| P_{AGA2} | Saccharomyces cerevisiae | 3 |
| P_{YCL076W} | Saccharomyces cerevisiae | 4 |
| P_{IME4} | Saccharomyces cerevisiae | 5 |
| P_{HPF1} | Saccharomyces cerevisiae | 6 |
| P_{CSS1} | Saccharomyces cerevisiae | 7 |
| P_{FUS1} | Saccharomyces cerevisiae | 8 |
| P_{FIG1} | Saccharomyces cerevisiae | 9 |
| P_{TDH3} | Saccharomyces cerevisiae | 10 |
| P_{PGK1} | Saccharomyces cerevisiae | 11 |
| P_{SNZ1} | Saccharomyces cerevisiae | 12 |
| P_{CPS1} | Saccharomyces cerevisiae | 13 |
| P_{CCW12} | Saccharomyces cerevisiae | 14 |
| P_{6xLexO-LEU2} | Synthetic construct | 15 |
| P_{GAL1} | Saccharomyces cerevisiae | 16 |
| P_{TEF1} | Saccharomyces cerevisiae | 17 |
| P_{RAD27} | Saccharomyces cerevisiae | 18 |

In embodiments the promoter(s) is/are selected from Table 2, or functional variants thereof with a nucleic acid sequence that is at least 80% identical to any one of SEQ ID NOs: 1-18.

### Terminators

In general, terminators are to be understood as specific sequences of nucleotides in DNA that signal the end of a gene or a transcription unit. They act as stop signals for RNA polymerase, the enzyme responsible for synthesizing RNA from a DNA template during transcription. Generally, terminators contain a stretch of DNA that forms a stable hairpin loop, followed by a run of uridine residues that cause RNA polymerase to pause and release the RNA transcript. The presence of terminators helps ensure accurate transcription termination and prevent the production of truncated or aberrant RNA molecules.

Accordingly, in embodiments, the nucleic acid sequence encoding the receptor, transcription factor and/or signal molecule comprises a terminator.

In embodiments, the terminators(s) is/are selected from Table 3.

TABLE 3
Terminators.

| Terminator name | Origin | SEQ ID NO |
|---|---|---|
| T_{CPS1} | Saccharomyces cerevisiae | 19 |
| T_{CYC1} | Saccharomyces cerevisiae | 20 |
| T_{ADH1} | Saccharomyces cerevisiae | 21 |
| T_{ENO1} | Saccharomyces cerevisiae | 22 |
| T_{MF(ALPHA)1} | Saccharomyces cerevisiae | 23 |

In embodiments the terminators(s) is/are selected from Table 3, or functional variants thereof with a nucleic acid sequence that is at least 80% identical, such as e.g., at least 85%, at least 90%, at least 95%, at least 98%, or such as e.g., at least 99% identical to any one of SEQ ID NOs: 19-23.

### Nucleic Acid Construct

An aspect of the present invention also relates to an expression cassette comprising one or more nucleic acid sequence(s), referred to here as coding sequence(s) (CDS(s)), encoding a receptor, a transcription factor, a signal molecule, and/or a protein regulating or affecting the function of the SCASA system, wherein said expression cassette further comprises one or more recombinant nucleic acid sequences comprising one or more promoter sequences and/or one or more terminator sequences.

Another aspect of the present invention also relates to an expression cassette comprising one or more nucleic acid sequence(s), referred to here as coding sequence(s) (CDS(s)), encoding a receptor, a transcription factor, a signal molecule, and/or a protein regulating or affecting the function of the method as disclosed herein, wherein said expression cassette further comprises one or more recombinant nucleic acid sequences comprising one or more promoter sequences and/or one or more terminator sequences.

In embodiments, the CDS(s) is/are selected from Table 4.

TABLE 4
Nucleic acid and amino acid sequence(s) of CDS(s)

| | | Amino acid | Nucleic acid |
|---|---|---|---|
| CDS Name | Origin | SEQ ID NO | SEQ ID NO |
| α-factor pheromone receptor (Ste2/ | Saccharomyces cerevisiae | 24 | 81 |
| STE2) | | | |
| P-factor pheromone receptor | Schizosaccharomyces pombe | 25 | 82 |
| (Mam2/MAM2) | | | |
| Melatonin receptor 1A (MTNR1A/ | Human | 26 | 83 |
| MT1) | | | |
| Adenosine A_{2B} receptor (ADORA2B/ | Human | 27 | 84 |
| A2bR) | | | |
| Alpha-2A adrenergic receptor | Human | 28 | 85 |
| (ADRA2A/α_{2A} adrenoceptor) | | | |
| Serotonin/5-hydroxytryptamine | Human | 29 | 86 |
| receptor isoform b (HTR4 variant b/ | | | |
| 5HT4B) | | | |
| C-X-C chemokine receptor type 4 | Human | 30 | 87 |
| isoform a (CD184/fusin/CXCR4A) | | | |
| Gpa1 (GPA1) | Saccharomyces cerevisiae | 31 | 88 |
| Chimeric human Gpa1/Gαz | Synthetic construct, modified from | 32 | 89 |
| (GPA1/GαZ) | Saccharomyces cerevisiae | | |
| Chimeric human Gpa1/Gαi2 | Synthetic construct, modified from | 33 | 90 |
| (GPA1/Gαi2) | Saccharomyces cerevisiae | | |
| Aga1 (AGA1) | Saccharomyces cerevisiae | 34 | 91 |
| Ste12 (STE12) | Saccharomyces cerevisiae | 35 | 92 |
| Ste12 PRD (Ste12PRD) | Saccharomyces cerevisiae | 36 | 93 |
| Gal4 | Saccharomyces cerevisiae | 193 | 192 |
| LexA-PRD Ste12 synthetic | Synthetic construct, components of | 37 | 94 |
| transcription factor (LexA- | Saccharomyces cerevisiae and | | |
| Ste12PRD) | Escherichia coli | | |
| yEGFP | Synthetic construct | 38 | 95 |
| mKate2 | Synthetic construct | 39 | 96 |
| Aga2 | Saccharomyces cerevisiae | 40 | 97 |
| Leader | Synthetic construct | 41 | 98 |
| Pre-pro-α | Saccharomyces cerevisiae | 42 | 99 |
| Pre-OST1-pro-α | Synthetic construct, modified from | 43 | 100 |
| | Saccharomyces cerevisiae | | |
| Pre-linker | Synthetic construct | 44 | 101 |
| HA tag (HA) | Human influenza | 45 | 102 |
| PAS40 linker (PAS40) | Synthetic construct | 46 | 103 |
| (G₄S)₃ linker 1 ((G4S)3) | Synthetic construct | 47 | 104 |
| (G₄S)₃ linker 2 ((G4S)3) | Synthetic construct | 48 | 105 |
| (G₄S)₃ linker 3 ((G4S)3) | Synthetic construct | 49 | 106 |
| c-myc tag (myc) | Human | 50 | 107 |
| V5 tag (V5) | Simian Virus 5 | 51 | 108 |
| FLAG tag (FLAG) | Synthetic construct | 52 | 109 |
| CD19.1 ECD (CD19) | Synthetic construct, modified from | 53 | 110 |
| | human | | |
| IL-2 | Human | 54 | 111 |
| Her2 (HER2) | Human | 55 | 112 |
| Her2 ECD (HER2 ECD) | Human | 56 | 113 |
| PD-L1 | Human | 57 | 114 |
| PD-L1 ECD | Human | 58 | 115 |
| anti-CD3 scFv (aCD3) | Synthetic construct | 59 | 116 |
| anti-CD28 scFv (aCD28) | Synthetic construct | 60 | 117 |
| SsoFe2 | Synthetic construct, modified from | 61 | 118 |
| | Sulfolobus solfataricus | | |
| Aga2-HA-(G4S)3-myc | Synthetic construct | 62 | 119 |
| Aga2-HA-PAS40-(G4S)3-myc | Synthetic construct | 63 | 120 |
| Aga2-HA-(G4S)3-CD19-myc | Synthetic construct | 64 | 121 |
| Aga2-HA-PAS40-(G4S)3-CD19-myc | Synthetic construct | 65 | 122 |
| Aga2-HA-(G4S)3-yEGFP-myc | Synthetic construct | 66 | 123 |
| Aga2-HA-PAS40-(G4S)3-yEGFP- | Synthetic construct | 67 | 124 |
| myc | | | |
| Aga2-FLAG-(G4S)3-SsoFe2 | Synthetic construct | 68 | 125 |
| Leader-Aga2-FLAG-(G4S)3- | Synthetic construct | 69 | 126 |
| SsoFe2-V5 | | | |
| Aga2-HA-(G4S)3-aCD3 | Synthetic construct | 70 | 127 |
| aCD3-(G4S)3-HA-Aga2 | Synthetic construct | 71 | 128 |
| Aga2-HA-(G4S)3-aCD28 | Synthetic construct | 72 | 129 |
| aCD28-(G4S)3-HA-Aga2 | Synthetic construct | 73 | 130 |
| Aga2-HA-(G4S)3-IL-2 | Synthetic construct | 74 | 131 |
| Aga2-HA-(G4S)3-(PD-L1 ECD) | Synthetic construct | 75 | 132 |
| Aga2-HA-PAS40-(G4S)3-(PD-L1 | Synthetic construct | 76 | 133 |
| ECD) | | | |
| Aga2-HA-(G4S)3-(HER2 ECD) | Synthetic construct | 77 | 134 |
| Aga2-HA-PAS40-(G4S)3-(HER2 | Synthetic construct | 78 | 135 |
| ECD) | | | |
| Pre-pro-α-IL-2 | Synthetic construct | 79 | 136 |
| Pre-OST1-pro-α-IL-2 | Synthetic construct | 80 | 137 |

In embodiments the expression cassettes are selected from Table 5.

TABLE 5
examples of expression cassettes

| Construct name | Promoter | CDS Name | Terminator | Origin | SEQ ID NO |
|---|---|---|---|---|---|
| P_{PGK1}-Aga2-HA-(G4S)3- | P_{PGK1} | Aga2-HA-(G4S)3- | T_{CPS1} | Recombinant | 138 |
| myc-T_{CPS1} | | myc | | synthetic | |
| | | | | construct | |
| P_{PGK1}-Aga2-HA-PAS40- | P_{PGK1} | Aga2-HA-PAS40- | T_{CPS1} | Recombinant | 139 |
| (G4S)3-myc-T_{CPS1} | | (G4S)3-myc | | synthetic | |
| | | | | construct | |
| P_{MFA2}-Aga2-HA-PAS40- | P_{MFA2} | Aga2-HA-PAS40- | T_{CPS1} | Recombinant | 140 |
| (G4S)3-CD19-T_{CPS1} | | (G4S)3-CD19 | | synthetic | |
| | | | | construct | |
| P_{FUS1}-Aga2-HA-PAS40- | P_{FUS1} | Aga2-HA-PAS40- | T_{CPS1} | Recombinant | 141 |
| (G4S)3-CD19-T_{CPS1} | | (G4S)3-CD19 | | synthetic | |
| | | | | construct | |
| P_{FIG1}-Aga2-HA-PAS40- | P_{FIG1} | Aga2-HA-PAS40- | T_{CPS1} | Recombinant | 142 |
| (G4S)3-CD19-T_{CPS1} | | (G4S)3-CD19 | | synthetic | |
| | | | | construct | |
| P_{MFA1}-Aga2-HA-PAS40- | P_{MFA1} | Aga2-HA-PAS40- | T_{CPS1} | Recombinant | 143 |
| (G4S)3-CD19-T_{CPS1} | | (G4S)3-CD19 | | synthetic | |
| | | | | construct | |
| P_{HPF1}-Aga2-HA-PAS40- | P_{HPF1} | Aga2-HA-PAS40- | T_{CPS1} | Recombinant | 144 |
| (G4S)3-CD19-T_{CPS1} | | (G4S)3-CD19 | | synthetic | |
| | | | | construct | |
| P_{TDH3}-Aga2-HA-PAS40- | P_{TDH3} | Aga2-HA-PAS40- | T_{CPS1} | Recombinant | 145 |
| (G4S)3-CD19-T_{CPS1} | | (G4S)3-CD19 | | synthetic | |
| | | | | construct | |
| P_{PGK1}-Aga2-HA-PAS40- | P_{PGK1} | Aga2-HA-PAS40- | T_{CPS1} | Recombinant | 146 |
| (G4S)3-CD19-T_{CPS1} | | (G4S)3-CD19 | | synthetic | |
| | | | | construct | |
| P_{GAL1}-Aga2-HA-PAS40- | P_{GAL1} | Aga2-HA-PAS40- | T_{CPS1} | Recombinant | 147 |
| (G4S)3-CD19-T_{CPS1} | | (G4S)3-CD19 | | synthetic | |
| | | | | construct | |
| P_{6xLexOLEU2}-Aga2-HA- | P_{6xLexOLEU2} | Aga2-HA-PAS40- | T_{CPS1} | Recombinant | 148 |
| PAS40-(G4S)3-CD19- | | (G4S)3-CD19- | | synthetic | |
| myc-T_{CPS1} | | myc | | construct | |
| P_{FIG1}-Aga2-HA-PAS40- | P_{FIG1} | Aga2-HA-PAS40- | T_{CPS1} | Recombinant | 149 |
| (G4S)3-yEGFP-myc-T_{CPS1} | | (G4S)3-yEGFP- | | synthetic | |
| | | myc | | construct | |
| P_{GAL1}-Aga2-HA-PAS40- | P_{GAL1} | Aga2-HA-PAS40- | T_{MF(ALPHA)1} | Recombinant | 150 |
| (G4S)3-CD19-myc- | | (G4S)3-CD19- | | synthetic | |
| T_{MF(ALPHA)1} | | myc | | construct | |
| P_{GAL1}-Aga2-HA-PAS40- | P_{GAL1} | Aga2-HA-PAS40- | T_{MF(ALPHA)1} | Recombinant | 151 |
| (G4S)3-yEGFP-myc- | | (G4S)3-yEGFP- | | synthetic | |
| T_{MF(ALPHA)1} | | myc | | construct | |
| P_{TDH3}-Aga2-HA-(G4S)3- | P_{TDH3} | Aga2-HA-(G4S)3- | T_{CPS1} | Recombinant | 152 |
| aCD3-T_{CPS1} | | aCD3 | | synthetic | |
| | | | | construct | |
| P_{TDH3}-aCD3-(G4S)3-HA- | P_{TDH3} | aCD3-(G4S)3- | T_{CPS1} | Recombinant | 153 |
| Aga2-T_{CPS1} | | HA-Aga2 | | synthetic | |
| | | | | construct | |
| P_{TDH3}-Aga2-HA-(G4S)3- | P_{TDH3} | Aga2-HA-(G4S)3- | T_{CPS1} | Recombinant | 154 |
| aCD28-T_{CPS1} | | aCD28 | | synthetic | |
| | | | | construct | |
| P_{TDH3}-aCD28-(G4S)3-HA- | P_{TDH3} | aCD28-(G4S)3- | T_{CPS1} | Recombinant | 155 |
| Aga2-T_{CPS1} | | HA-Aga2 | | synthetic | |
| | | | | construct | |
| P_{TDH3}-Aga2-HA-(G4S)3- | P_{TDH3} | Aga2-HA-(G4S)3- | T_{CPS1} | Recombinant | 156 |
| IL-2-T_{CPS1} | | IL-2 | | synthetic | |
| | | | | construct | |
| P_{TDH3}-Aga2-FLAG- | P_{TDH3} | Aga2-FLAG- | T_{CYC1} | Recombinant | 157 |
| (G4S)3-SsoFe2-T_{CYC1} | | (G4S)3-SsoFe2 | | synthetic | |
| | | | | construct | |
| P_{FUS1}-Aga2-FLAG- | P_{FUS1} | Aga2-FLAG- | T_{CYC1} | Recombinant | 158 |
| (G4S)3-SsoFe2-T_{CYC1} | | (G4S)3-SsoFe2 | | synthetic | |
| | | | | construct | |
| P_{TDH3}-Aga2-HA-(G4S)3- | P_{TDH3} | Aga2-HA-(G4S)3- | T_{CPS1} | Recombinant | 159 |
| (PD-L1 ECD)-T_{CPS1} | | (PD-L1 ECD) | | synthetic | |
| | | | | construct | |
| P_{TDH3}-Aga2-HA-PAS40- | P_{TDH3} | Aga2-HA-PAS40- | T_{CPS1} | Recombinant | 160 |
| (G4S)3-(PD-L1 ECD)- | | (G4S)3-(PD-L1 | | synthetic | |
| T_{CPS1} | | ECD) | | construct | |
| P_{TDH3}-Aga2-HA-(G4S)3- | P_{TDH3} | Aga2-HA-(G4S)3- | T_{CPS1} | Recombinant | 161 |
| (HER2 ECD)-T_{CPS1} | | (HER2 ECD) | | synthetic | |
| | | | | construct | |
| P_{TDH3}-Aga2-HA-PAS40- | P_{TDH3} | Aga2-HA-PAS40- | T_{CPS1} | Recombinant | 162 |
| (G4S)3-(HER2 ECD)- | | (G4S)3-(HER2 | | synthetic | |
| T_{CPS1} | | ECD) | | construct | |
| P_{MFA2}-yEGFP-T_{CPS1} | P_{MFA2} | yEGFP | T_{CPS1} | Recombinant | 163 |
| | | | | synthetic | |
| | | | | construct | |
| P_{FUS1}-yEGFP-T_{CPS1} | P_{FUS1} | yEGFP | T_{CPS1} | Recombinant | 164 |
| | | | | synthetic | |
| | | | | construct | |
| P_{AGA2}-yEGFP-T_{CPS1} | P_{AGA2} | yEGFP | T_{CPS1} | Recombinant | 165 |
| | | | | synthetic | |
| | | | | construct | |
| P_{FIG1}-yEGFP-T_{CPS1} | P_{FIG1} | yEGFP | T_{CPS1} | Recombinant | 166 |
| | | | | synthetic | |
| | | | | construct | |
| P_{MFA1}-yEGFP-T_{CPS1} | P_{MFA1} | yEGFP | T_{CPS1} | Recombinant | 167 |
| | | | | synthetic | |
| | | | | construct | |
| P_{YCL076W}-yEGFP-T_{CPS1} | P_{YCL076W} | yEGFP | T_{CPS1} | Recombinant | 168 |
| | | | | synthetic | |
| | | | | construct | |
| P_{HPF1}-yEGFP-T_{CPS1} | P_{HPF1} | yEGFP | T_{CPS1} | Recombinant | 169 |
| | | | | synthetic | |
| | | | | construct | |
| P_{IME4}-yEGFP-T_{CPS1} | P_{IME4} | yEGFP | T_{CPS1} | Recombinant | 170 |
| | | | | synthetic | |
| | | | | construct | |
| P_{CPS1}-yEGFP-T_{CPS1} | P_{CPS1} | yEGFP | T_{CPS1} | Recombinant | 171 |
| | | | | synthetic | |
| | | | | construct | |
| P_{CSS1}-yEGFP-T_{CPS1} | P_{CSS1} | yEGFP | T_{CPS1} | Recombinant | 172 |
| | | | | synthetic | |
| | | | | construct | |
| P_{GAL1}-yEGFP-T_{CPS1} | P_{GAL1} | yEGFP | T_{CPS1} | Recombinant | 173 |
| | | | | synthetic | |
| | | | | construct | |
| P_{PGK1}-yEGFP-T_{CPS1} | P_{PGK1} | yEGFP | T_{CPS1} | Recombinant | 174 |
| | | | | synthetic | |
| | | | | construct | |
| P_{TDH3}-yEGFP-T_{CPS1} | P_{TDH3} | yEGFP | T_{CPS1} | Recombinant | 175 |
| | | | | synthetic | |
| | | | | construct | |
| P_{SNZ1}-yEGFP-T_{CPS1} | P_{SNZ1} | yEGFP | T_{CPS1} | Recombinant | 176 |
| | | | | synthetic | |
| | | | | construct | |
| P_{6xLexOLEU2}-yEGFP-T_{CPS1} | P_{6xLexOLEU2} | yEGFP | T_{CPS1} | Recombinant | 177 |
| | | | | synthetic | |
| | | | | construct | |
| P_{TDH3}-AGA1-T_{ADH1} | P_{TDH3} | AGA1 | T_{ADH1} | Recombinant | 178 |
| | | | | synthetic | |
| | | | | construct | |
| P_{MFA2}-AGA1-T_{ADH1} | P_{MFA2} | AGA1 | T_{ADH1} | Recombinant | 179 |
| | | | | synthetic | |
| | | | | construct | |
| P_{PGK1}-AGA1-T_{ADH1} | P_{PGK1} | AGA1 | T_{ADH1} | Recombinant | 180 |
| | | | | synthetic | |
| | | | | construct | |
| P_{RAD27}-LexA-Ste12PRD- | P_{RAD27} | LexA-Ste12PRD | T_{ENO1} | Recombinant | 181 |
| T_{ENO1} | | | | synthetic | |
| | | | | construct | |
| P_{PGK1}-GPA1-T_{CYC1} | P_{PGK1} | GPA1 | T_{CYC1} | Recombinant | 182 |
| | | | | synthetic | |
| | | | | construct | |
| P_{PGK1}-GPA1/GαZ-T_{CYC1} | P_{PGK1} | GPA1/GαZ | T_{CYC1} | Recombinant | 183 |
| | | | | synthetic | |
| | | | | construct | |
| P_{PGK1}-GPA1/Gαi2-T_{CYC1} | P_{PGK1} | GPA1/Gαi2 | T_{CYC1} | Recombinant | 184 |
| | | | | synthetic | |
| | | | | construct | |
| P_{CCW12}-STE2-T_{CYC1} | P_{CCW12} | STE2 | T_{CYC1} | Recombinant | 185 |
| | | | | synthetic | |
| | | | | construct | |
| P_{CCW12}-MAM2-T_{CYC1} | P_{CCW12} | MAM2 | T_{CYC1} | Recombinant | 186 |
| | | | | synthetic | |
| | | | | construct | |
| P_{CCW12}-MTNR1A-T_{CYC1} | P_{CCW12} | MTNR1A | T_{CYC1} | Recombinant | 187 |
| | | | | synthetic | |
| | | | | construct | |
| P_{CCW12}-ADORA2B-T_{CYC1} | P_{CCW12} | ADORA2B | T_{CYC1} | Recombinant | 188 |
| | | | | synthetic | |
| | | | | construct | |
| P_{CCW12}-ADRA2A-T_{CYC1} | P_{CCW12} | ADRA2A | T_{CYC1} | Recombinant | 189 |
| | | | | synthetic | |
| | | | | construct | |
| P_{CCW12}-5HT4B-T_{CYC1} | P_{CCW12} | 5HT4B | T_{CYC1} | Recombinant | 190 |
| | | | | synthetic | |
| | | | | construct | |
| P_{CCW12}-CXCR4A-T_{CYC1} | P_{CCW12} | CXCR4A | T_{CYC1} | Recombinant | 191 |
| | | | | synthetic | |
| | | | | construct | |

In embodiments, the genetically engineered yeast cell comprises,
a receptor expression construct,
a transcription factor construct, and/or
a signal molecule construct.

In embodiments, the genetically engineered yeast cell comprises,
a receptor expression construct, and/or
a signal molecule construct.

In embodiments, the genetically engineered yeast cell comprises,
a transcription factor construct, and/or
a signal molecule construct.

In further embodiments, two or more of the
a receptor expression construct,
a transcription factor construct, and/or
a signal molecule construct,
are encompassed in a single expression cassette.

The nucleic acid construct can be a recombinant, heterologous or endogenous nucleic acid sequence. By the term “recombinant nucleic acid sequence”, “recombinant gene/nucleic acid/DNA encoding”, “heterologous nucleic acid sequence”, “heterologous gene/nucleic acid/DNA encoding” or “coding nucleic acid sequence” it is generally understood that such nucleic acid relates to an artificial nucleic acid sequence (i.e. produced in vitro using standard laboratory methods for making nucleic acid sequences) that comprises a set of consecutive, non-overlapping triplets (codons) which is transcribed into mRNA and translated into a protein when under the control of the appropriate control sequences, i.e. a promoter sequence.

The boundaries of the coding sequence are generally determined by a ribosome binding site 5 located just upstream of the open reading frame at the 5′end of the mRNA, a transcriptional start codon (AUG, GUG or UUG), and a translational stop codon (UAA, UGA or UAG). A coding sequence can include, but is not limited to, genomic DNA, cDNA, synthetic, and recombinant nucleic acid sequences.

The term “nucleic acid” includes RNA, DNA and cDNA molecules. It is understood that, as a result of the degeneracy of the genetic code, a multitude of nucleic acid sequences encoding a given protein may be produced.

In embodiments, the expressed receptor, transcription factor, signal molecule, and/or proteins regulating or affecting the function of the SCASA system is/are encoded by a recombinant nucleic acid sequence.

In embodiments, the expressed receptor, transcription factor, signal molecule, and/or proteins regulating or affecting the function of the method defined herein is/are encoded by a recombinant nucleic acid sequence.

### Episomal Element

The term “episomal element” refers to an extrachromosomal nucleic acid sequence that can replicate autonomously or integrate into the genome of the genetically engineered yeast cell.

Thus, an episomal nucleic acid sequence may be a plasmid that can integrate into the chromosome of the genetically engineered cell, i.e., not all plasmids are episomal elements.

In embodiments, the expression cassette or expression construct is encompassed in an episomal element.

In embodiments, episomal nucleic acid sequences may be a plasmid that is not integrated into the chromosome. In the present context, the episomal element refers to plasmid DNA sequences that carry an expression cassette of interest, with the cassette consisting of a promoter sequence, the coding sequence of the gene of interest and a terminator sequence.

In one or more embodiments, the episomal nucleic acid sequences may be a plasmid with only a part of it being integrated into the chromosome. In the present context, the expression cassette resembles the one described above but it further comprises two DNA segments that are homologous to the DNA regions up- and downstream of the locus that the gene of interest is to be integrated.

### Sequence Identity

The term “sequence identity” as used herein describes the relatedness between two amino acid sequences or between two nucleotide sequences, i.e., a candidate sequence (e.g., a sequence of the invention) and a reference sequence (such as e.g., a prior art sequence) based on their pairwise alignment. For purposes disclosed herein, the sequence identity between two amino acid sequences is determined using the Needleman-Wunsch algorithm (Needleman and Wunsch, 1970, J. Mo/. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later (available at https://www.ebi.ac.uk/Tools/psa/emboss needle/). The parameters used are gap open penalty of 10, gap extension penalty of 0.5, and the EBLOSUM62 (EMBOSS version of 30 BLOSUM62) substitution matrix. The output of Needle labelled “longest identity” (obtained using the −nobrief option) is used as the percent identity and is calculated as follows: (Identical Residues×100)/(total alignment length). When assessing the “total alignment length”, any N and/or C-terminal overhangs/gaps, which does not align between the queried sequences is also included when counting the “total alignment length”.

For purposes disclosed herein, the sequence identity between two nucleotide sequences is determined using the Needleman-Wunsch algorithm (Needleman and Wunsch, 1 970, supra) as implemented in the Needle program of the EMBOSS package (EMBOSS: The European Molecular Biology Open Software Suite, Rice et al., 2000, Trends Genet. 16: 276-277), 10 preferably version 5.0.0 or later. The parameters used are gap open penalty of 10, gap extension penalty of 0.5, and the DNAFULL (EMBOSS version of NCBI NUC4.4) substitution matrix. The output of Needle labelled “longest identity” (obtained using the −nobrief option) is used as the percent identity and is calculated as follows: (Identical nucleotide residues×100)/(total alignment length). When assessing the “total alignment length”, any 5′-end and/or 3′-end overhangs/gaps, which does not align between the queried sequences, is also included when counting the “total alignment length”.

### Functional Homologue

A functional homologue or functional variant of a protein/nucleic acid sequence as described herein is a protein/nucleic acid sequence with alterations in the genetic code, which retain its original functionality. A functional homologue may be obtained by mutagenesis or may be natural occurring variants from the same or other species. The functional homologue should have a remaining functionality of at least 50%, such as e.g., at least 60%, 70%, 80%, 90% or 100% compared to the functionality of the protein/nucleic acid sequence.

A functional homologue of any one of the disclosed amino acid or nucleic acid sequences can also have a higher functionality. A functional homologue of any one of the amino acid sequences, or a recombinant nucleic acid as disclosed herein, are considered as genes, proteins, or other biological molecule that performs a similar or equivalent function to any one of the amino acid sequences, or a recombinant nucleic acid as disclosed herein, in a different species or biological context, but remains functional upon expression in the genetically modified yeast cell of the present disclosure.

### A Receiver Cell

As such the Synthetic Cellular Advanced Signal Adapter (SCASA) system and method described herein does not restrict the receiver cell (SCASA yeast cell) to any specific cell types and may be used on a wide range of receiver cells, such as e.g., bacterial, human, animal or plant cells. In preferred embodiments, the cell is a human cell. In preferred embodiments, the receiver cell is a human immune cell.

As such the function of the SCASA system or method may be to enable the generation of receiver cells with specific phenotypes, wherein the SCASA system may be used to induce or promote such phenotypes, e.g., by modulating, manipulating, and/or influencing the phenotype of specific receiver cells.

As such the method for in vitro modulating, manipulating, and/or influencing the phenotype of human immune cells described herein does not restrict the receiver cell to any specific cell types and may be used on a wide range of receiver cells, such as e.g., bacterial, human, animal or plant cells. In preferred embodiments, the cell is a human cell. In further preferred embodiments, the receiver cell is a human immune cell.

Accordingly, the purpose of the for in vitro modulating, manipulating, and/or influencing the phenotype of human immune cells may be to enable the generation of human immune cells with specific phenotypes, wherein the method may be used to induce or promote such phenotypes, e.g., by modulating, manipulating, and/or influencing the phenotype of specific human immune cells.

An induced or promoted phenotype relates to the alteration of cells and refers to changes in the specific characteristics of a receiver cell, including changes in e.g., its morphology, behaviour, and/or function, as a result stimulation with a genetically modified yeast cell as described herein. Such alterations can be temporary or permanent. Altering the phenotype of cells can be used to either study the roles of specific genes or pathways in cellular function, or to create cells with desired characteristics for use in biotechnology or medical applications.

Examples of phenotype alteration of cells include inducing differentiation of stem cells into specific cell types, expanding T cells, differentiating T cells, promoting the activation of CAR T cells, promoting specific responses in T cells or CAR T cells, or modifying receiver cells to produce specific proteins or other molecules, such as e.g., specific CARs, cytokines, interleukins, chemokines, enzymes, receptors, hormones, or antigens.

In embodiments the receiver cells are selected from immune cells, preferably human immune cells. Preferably, the human immune cells are cells which are directly isolated from a human and not modified further, or cells which are derived from cells isolated from a human, which may be genetically modified.

There are several types of immune cells in the human body, each with a unique function in defending against pathogens and maintaining immune homeostasis. Preferably, the human immune cells are selected from T cells, B cells, natural killer cells, dendritic cells, Macrophages, Neutrophils, Eosinophils, Basophils, innate lymphoid cells, and Mast cells.

T cells are a type of lymphocyte that is responsible for recognizing and attacking infected or cancerous cells. There are several types of T cells, including helper T cells, cytotoxic T cells, and regulatory T cells. B cells is a type of lymphocyte that produces antibodies in response to specific pathogens. B cells can also present antigens to T cells, helping to initiate an immune response. NK cells are a type of lymphocyte that recognizes and destroys infected or cancerous cells without prior exposure to them. NK cells also produce cytokines that help to regulate immune responses. Dendritic cells are antigen-presenting cells that capture and present antigens to T cells, initiating an adaptive immune response. Dendritic cells also play a role in immune tolerance and preventing autoimmunity. Macrophages are phagocytic cells that engulf and destroy pathogens and cellular debris. Macrophages also produce cytokines that help to recruit other immune cells to sites of infection or inflammation. Neutrophils are also phagocytic cells that are typically the first to respond to an infection. Neutrophils can release antimicrobial agents and cytokines but can also contribute to tissue damage in chronic inflammation. Eosinophils are a type of white blood cell that is involved in allergic responses and defence against parasitic infections. Basophils are a type of white blood cell that releases histamine and other mediators in response to allergens and parasites. Innate lymphoid cells are immune cells in the lymphoid lineage that lack antigen-specific receptors and play a role in maintaining tissue homeostasis, defending against pathogens, and regulating immune responses through the production of specific cytokines. Mast cells are a type of immune cell involved in immune responses against some bacteria and parasites and in allergic reactions, releasing inflammatory substances such as e.g., histamine.

In addition to the above immune cells, there are a plethora of subtypes and variations of these cells, and the immune system is a complex and dynamic system that involves many other cell types and signal molecules.

Especially the T cells have gained impasse on in the field of cancer therapeutics helped along with their modification to express chimeric antigen receptors (CARs), which can be used to focus the targeting of the T cells towards specific cancers, such as e.g., cancer expressing the CD19 antigen, such as e.g., B cell lymphomas, acute lymphoblastic leukaemia (ALL), and chronic lymphocytic leukaemia (CLL). T cells expressing CARs are commonly referred to as CAR T cells. In embodiments, the human immune cell is selected from the group consisting of T lymphocytes (T cells), engineered T cells (CAR T cells), Jurkat cells, B lymphocytes (B cells), Natural killer (NK) cells, Dendritic cells, Macrophages, Neutrophils, Eosinophils, Basophils, innate lymphoid cells and Mast cells.

In general, CAR T cells are engineered to express CARs on their surface, which are designed to recognize specific antigens e.g., CD19, present on the surface of cancer cells. Generally, a CAR is composed of an extracellular antigen-binding domain, a transmembrane domain, and intracellular signalling domains that activate the T cell when the CAR binds to its target antigen. Once activated, CAR T cells can rapidly proliferate and attack cancer cells that express the target antigen.

The functionality of the SCASA system to obtain a controllable and orthogonal dynamic activation of CAR-T cells is shown in Example 3, Example 5, Example 7, and Example 8, which shows that the CD19-presenting SCASA yeast aAPCs may be used to communicate with the human cells and activate CAR-T cells. In specific it was shown in example 3 that CAR Jurkat cells could be activated by SCASA yeast aAPCs presenting CD19, in a manner that was; I) CD19-specific, II) CAR-specific, III) CD19 antigen-density sensitive in regards to the intensity of the Jurkat activation, IV) different for each SCASA yeast aAPC design, V) controllable through the SCASA yeast aAPC GPCR, VI) capable of surpassing activation levels of NALM6 cancer cells, and VII) unaffected by the presence of yeast itself (see FIG. 15). In addition, it was found in example 3 that the non-CD19 expressing yeast did not activate T cells, clearly showing the specificity of the system.

In embodiments, the human immune cell is selected from the group consisting of CAR T cells, naïve T cells and NK cells.

It is to be understood that the SCASA system presented herein is not limited to human immune cells but may also be used in the modulation or manipulation of other cell types, such as e.g., pluripotent cells, such as e.g., embryonic stem cells (ESCs) or induced pluripotent stem cells (iPSCs).

The present invention is also suitable for induction of morphological and/or phenotypic changes in other mammalian cells, such as e.g., human pluripotent cells.

Generally human pluripotent cells are a type of stem cell that can differentiate into any cell type in the human body. Such cells are suitable for modification in a co-culture with a genetically engineered cell as described herein.

Pluripotent cells are generally divided into two main types: embryonic stem cells, which are derived from the inner cell mass of the early embryo, and induced pluripotent stem cells, which are generated from adult somatic cells through a process of reprogramming. In embodiments the a Synthetic Cellular Advanced Signal Adapter (SCASA) system for use in modulating, manipulating, and/or influencing the phenotype of human pluripotent cells, comprising in vitro co-culturing of a genetically engineered yeast cell and a human pluripotent cell, wherein said human receiver cell, which is a human pluripotent cell, is modulated, manipulated and/or influenced such that it obtains an altered phenotype in response to said presentation and/or secretion of said signal molecule from said yeast cell.

Accordingly, in embodiments, the receiver cell is an embryonic stem cell (ESC). In other embodiments, the receiver cell is an induced pluripotent stem cell (iPSC).

T Cell Stimulation, Differentiation and/or Expansion

In general, there are several ways to stimulate, differentiate, and/or expand a population of T cells. T cells can be stimulated, differentiated, and/or expanded in vitro by exposing them to cytokines, such as e.g., interleukin-2 (IL-2), which promotes T cell proliferation and survival. T cells can also be stimulated, differentiated and/or expanded using antibodies or antibody-like molecules targeting e.g., the CD3 and CD28 molecules on the T cell surface, which activates the T cells and promotes their proliferation. T cells can also be stimulated in a clonal- or (TCR) T-cell-receptor-specific manner, such as e.g. by using specific MHC-I or MHC-II molecules which can be loaded or co-expressed with specific antigenic peptides. An approach which may include either secretion of cytokines and/or presentation of specific artificial antigens and/or MHCs from a cell or a non-living structure, which is commonly referred to aAPCs. aAPCs are engineered cells or structures that express or display specific co-stimulatory molecules and antigen-presenting proteins, allowing them to activate, differentiate, and/or expand T cells in vitro. In the present disclosure, the yeast cell as described herein may also be considered an aAPC, which expresses on the surface of said cell and/or secrets from said cell one or more functional signal molecule(s). Furthermore, T cells can be genetically modified to express specific genes or receptors that promote their expansion and survival in vitro or provide them with new abilities. For example, T cells can be engineered to express CARs that recognize specific antigens, allowing for targeted expansion of CAR T cells.

In embodiments, the genetically engineered yeast cell of the present invention can be designed to express specific signal molecule(s) to target T cell and/or CAR T cell expansion and/or differentiation.

### Activation of CAR T Cells

In general, treatment using CAR T cells include a number of steps between collection of the T cells from a patient to reintroduction of the T cells into a patient. The process of CAR T cell therapy may in some embodiments be summarized as follows:
1. Collection of T cells: T cells are collected from the patient through a process called leukapheresis, in which blood is removed and passed through a machine that separates out the white blood cells, including T cells.
2. Genetic modification: The T cells are genetically modified in the laboratory to express chimeric antigen receptors (CARs) on their surface. The CAR is designed to recognize specific antigens present on the surface of cancer cells.
3. Expansion and activation: The modified T cells are grown and expanded in the laboratory using elements that promote cell growth and survival, such elements may e.g., be a functional signal molecule, such as e.g., cancer antigens, MHC-I, MHC-II, B-cell receptors, adhesive proteins, cytokines, hormones, neurotransmitters, fluorescent proteins, and antibody tags which presentation and/or secretion triggers their proliferation and enhances the killing ability of the T cells.
4. Infusion: The activated CAR T cells are infused back into the patient, typically via an intravenous drip. The CAR T cells then circulate in the patient's bloodstream and migrate to the site of the cancer.
5. Recognition and killing of cancer cells: Once the CAR T cells encounter cancer cells that express the target antigen, the CAR binds to the antigen and activates the T cell. The activated T cell then releases cytotoxic molecules that kill the cancer cell and can also trigger the recruitment of other immune cells to attack the cancer.

Example 4 describes the co-cultivation conditions and T cell activation protocol, wherein the compatibility and conditions of co-cultivations with human immune cells, specifically T cells, and yeast cells for SCASA system applications are investigated. Importantly, it was shown in example 4 that SCASA yeast aAPC do not grow in T cell cultivation medium (See FIG. 16), thus the SCASA yeast aAPC of the disclosure did not outcompete the T cells. This is highly favourable since competition between the two cells would inhibit the efficiency of the SCASA system.

In addition, it was found that the SCASA yeast aAPC maintains their ability to sense and respond in T cell cultivation medium using SCASA designs, and that human immune cells are inert to the presence of SCASA yeast aAPC cells when no intentional signal molecules are present (see FIGS. 17-19).

The clinical co-cultivation of the SCASA yeast aAPC using donor-derived CAR T cells is shown in Example 5 and Example 7. The results presented in example 5 and example 7 shows that the SCASA yeast aAPCs are robust CAR T cell activators, suitable for applications in testing clinical immunotherapeutic cell products, in a clinical setting (see FIGS. 23 and 24). In embodiments, the SCASA system is for use in testing clinical immunotherapeutic cell products.

Accordingly, in embodiments the SCASA system and method of the present disclosure is for use in assaying the signals influencing activation, responses, and/or phenotypes of T cells and/or CAR T cells, such as e.g., cancer antigens, MHC-I, MHC-II, B-cell receptors, adhesive proteins, cytokines, hormones, neurotransmitters, fluorescent proteins, and antibody tags which presentation and/or secretion triggers immune cell proliferation, differentiation and/or enhances their killing ability.

Furthermore, in embodiments the methods of the present disclosure is for in vitro use in assaying the signals influencing activation, responses, and/or phenotypes of T cells and/or CAR T cells, such as e.g., cancer antigens, MHC-I, MHC-II, B-cell receptors, adhesive proteins, cytokines, hormones, neurotransmitters, fluorescent proteins, and antibody tags which presentation and/or secretion triggers immune cell proliferation, differentiation and/or enhances their killing ability.

The results presented in examples 5 and example 7 show that CAR T cells recognize SCASA yeast aAPCs as cancer cells, however, the CAR T cells were found to be unable to kill the SCASA yeast aAPCs. Additionally, as the number of SCASA yeast aAPCs were shown to be more robustly maintained in CAR T cell co-cultures than cancer cell lines. Consequently, this shows that SCASA yeast aAPCs are more robust for antigen-density studies than cell lines that are sensitive to cytotoxic responses by T cells. In addition, Example 5 also shows that CAR T cells may also be activated by yeast aAPCs after cryopreservation, and that higher CD19 antigen-densities were associated with increased expression of CD69 and CD25.

Accordingly, in embodiments the Synthetic Cellular Advanced Signal Adapter (SCASA) system and methods disclosed herein provides a method for expanding, differentiating and/or activating T cells and/or CAR T cells. Expanding, differentiating, and/or activating T cells and/or CAR T cells may e.g., include Culturing T cells or CAR T cells, Co-stimulatory signalling of the T cells or CAR T cells e.g., by activating the T-cell receptor (TCR), by activating CARs, or by stimulating other T cell receptors with signal molecule(s), such as e.g., co-stimulatory receptors.

Furthermore, in embodiments the methods disclosed herein are methods for expanding, differentiating and/or activating T cells and/or CAR T cells. Expanding, differentiating, and/or activating T cells and/or CAR T cells may e.g., include Culturing T cells or CAR T cells, Co-stimulatory signalling of the T cells or CAR T cells e.g., by activating the T-cell receptor (TCR), by activating CARs, or by stimulating other T cell receptors with signal molecule(s), such as e.g., co-stimulatory receptors.

Accordingly, the signal molecule described herein may have a co-stimulatory effect on the T cell or CAR T cell and/or activate the TCR on the T cell or CAR T cell.

Accordingly, the present disclosure also relates to a method for T cell, such as e.g., naïve T cell and engineered CAR T cell, expansion and/or differentiation comprising use of a Synthetic Cellular Advanced Signal Adapter (SCASA) system according to the present disclosure.

Such expansion and/or differentiation may e.g., be done by isolating T cells from a blood or tissue sample and stimulating them via co-culturing with the genetically modified yeast cell as disclosed herein presenting and/or secreting specific molecules that activate T cells growth, proliferation, and/or differentiation. In such circumstances, signal molecules may include cytokines, such as e.g., interleukin-2 (IL-2), or specific antigens. The co-cultivation of the genetically modified yeast cell and the T cells may be conducted in a suitable environment, such as e.g., a bioreactor, wherein e.g., a medium provides the necessary nutrients and conditions for their growth. Following co-cultivation, the expanded and/or differentiated T cells may then be harvested. The expanded and/or differentiated T cells may be used for various applications, such as e.g., immunotherapy for cancer, or infectious diseases. Accordingly, the SCASA system described herein allows for tailored functionality of the cultivated T cells, depending on the level of presentation and/or secretion of the signal molecule(s) and the type of signal molecule used for the specific expansion and/or differentiation.

In embodiments the methods and/or use of the SCASA system and methods presented herein comprises co-culturing of the genetically modified yeast cell with the receiver cell, such as e.g., a human immune cell. In embodiments, the receiver cell and the genetically modified yeast cell are co-cultured for at least 1 day, such as e.g., at least 2 days, 3 days, 4 days, 5 days, 8 days, 12 days, 2 weeks, 4 weeks or 8 weeks, or for about 1 day to about 8 weeks.

In addition, the SCASA system and methods presented herein may also be used to prepare a human immune cell with a favourable phenotype. In example, the SCASA system may be used to promote the presentation of CARs on T cells, which makes them more suitable for use in treatment of cancer. In the same manner the SCASA system may be used to promote the expansion of specific human cells, such as e.g., human immune cells, wherein the human immune cells may be used in the treatment of diseases like cancer. Accordingly, in embodiments, the present disclosure also relates to a human immune cell and/or human immune cell with a phenotype, produced by the system as disclosed herein, for use as a medicament.

Furthermore, the methods presented herein may also be used to prepare a human immune cell with a favourable phenotype in vitro. In example, the methods may be used in vitro to promote the presentation of CARs on T cells, which makes them more suitable for use in treatment of cancer. In the same manner methods may be used in vitro to promote the expansion of specific human cells, such as e.g., human immune cells, wherein the human immune cells may be used in the treatment of diseases like cancer. Accordingly, in embodiments, the present disclosure also relates to a human immune cell and/or human immune cell with a phenotype, produced in vitro by the method as disclosed herein, for use as a medicament.

Additionally, examples of possible applications of the SCASA system/method could be within in vitro assays or in the generation of cell products (see FIG. 27). In general, the concept of SCASA is to co-cultivate SCASA yeast cells with target cells of interest to obtain a target cell with a different phenotype. For example, the SCASA yeast cells and the associated methods may be used characterize different phenotypic responses of target cell population by e.g. varying signal intensities, select optimal cell designs based on different target cell responses to different signal molecule variants, or to discover new types of signal modalities for specific target cells or types of signals. Within cell product generation, the methods using the SCASA yeast cells could for example be used to change the phenotype of cells isolated from a patient sample, such as to produce therapeutically relevant phenotypes or provide proliferative stimulation to increase the amount of cell product.

Example 8 demonstrates practical applications of the SCASA system and methods disclosed herein in assaying and characterizing CAR T cell designs. The example demonstrates that SCASA yeast cells can effectively activate CAR T cells based on different designs, in this specific example CD19 CAR T cells employing either CD28- or 4-1BB co-stimulation and provide means to characterize their performance under different conditions, such as different antigen densities or the total antigen load. Additionally, example 8 shows that CAR T cell response intensities can be controlled by modulating antigen density and target cell ratios of SCASA yeast cells. One exemplary finding of results indicate that CD28-based CARs generally elicit stronger signalling responses compared to 4-1BB-based CARs, with differences in the downstream NF-κB, NFAT, and AP-1 activities dependent on antigen levels. Hence, example 8 demonstrates the utility of SCASA yeast cells in assays, such as evaluating CAR T cell dynamics and co-stimulatory domain effects.

### EXAMPLES

### Example 1—Design of a Processing Module Based on the Pheromone-Response Pathway

### Aim

Selection and testing of suitable promoters and signalling pathway designs for driving the expression of effector signal molecules from yeast cells for communication with receiver cells, and illustrating the modularity and possible diversification of the processing module.

### Materials and Methods

### Selection of Suitable Promoters

In order to generate a diverse portfolio of processing module parts for signal tuning, a group of 11 promoters was selected based on transcriptome analysis of genes activated by the native yeast mating pheromone response pathway (PRP) using heterologous or native GPCRs (Jensen et al. Nat Commun. 2022 Oct. 19; 13(1):6201.). Selection was based on features of their native mRNA: 1) their transcriptional diversity across GPCR stimulation levels, and II) absolute transcript levels. In addition, a LexO-containing promoter (P_{6xLexOLEU2}) was included for testing an orthogonal GPCR signalling pathway (Shaw et al. Cell. 2019 Apr. 18,177(3):782-796.e27.).

### Construct Generation

Genomic DNA (gDNA) was purified using the Yeast DNA Extraction Kit (Thermo Scientific). Custom DNA synthesis was done using gBlocks™ Gene Fragment synthesis service (Integrated DNA Technologies). For amplification of DNA fragments for cloning or genome integration, and inverse PCR plasmid construction, Phusion High-Fidelity (HF) PCR Master Mix with HF Buffer (Thermo Scientific) was used, and, specifically, for amplification of Uracil-Specific Excision Reagent (USER) cloning DNA fragments, Phusion U Hot Start PCR Master Mix (Thermo Scientific) was used with primers with USER-compatible uracil-containing tails. PCRs were conducted according to the manufacturer's protocols using a S1000 Thermal Cycler (Bio-Rad). All primers were synthesized using a Custom DNA Oligos service (Integrated DNA Technologies). Gel electrophoresis was done in 1% w/v agarose gels of 1× Tris-acetate-EDTA buffer at 90v for min., using TriTrack DNA 6× Loading Dye (Thermo Scientific), GeneRuler 1 kb DNA Ladder (Thermo Scientific), and RedSafe™ Nucleic Acid Staining Solution (iNtRON Biotechnology). Gel imaging was conducted on a Gel Doc XR+ System (Bio-Rad). PCR amplicons were purified via gel purification or column purification using the NucleoSpin Gel and PCR Clean-up kit (Macherey-Nagel). Amplicon sequences were verified by Sanger sequencing using the Overnight Mix2Seq Kit service (Eurofins Genomics). Plasmid vectors containing genome-integration expression cassettes were based on the EasyClone-MarkerFree system compatible with CRISPR/Cas9 engineering (Jessop-Fabre et al. Biotechnol J. 2016 August; 11(8):1110-7.) and were constructed with USER-cloning and assembly techniques (Nour-Eldin et al. Nucleic Acids Res. 2006; 34(18):e122.; Bitinaite et al. Nucleic Acids Res. 2007 March; 35(6): 1992-2002). Linearized USER-ready backbones were generated by purification of inverse PCR-amplified EasyClone-MarkerFree integrative vectors using primers with USER-compatible tails. Employed promoters and terminators were amplified from yeast gDNA. Some entire genes and CDSs were amplified from yeast gDNA or ordered as gBlocks™, and in some cases modified by in-frame fusion using USER-cloning, and all were modified to contain the AAAACA Kozak sequence. Each cassette assembled by USER-cloning contained standardized interchangeable parts: 1) a promoter, II) a protein coding sequence (CDS), III) a terminator, IV) a linearized backbone, which for genome integration contained homology arms directed at EasyClone-MarkerFree genome integration sites flanked by NotI-sites. Plasmids for integration of GPCR expression cassettes were assembled as previously described (Jensen et al. Nat Commun. 2022 Oct. 19; 13(1):6201). For CRISPR/Cas9 engineering, gRNA was expressed from cassettes using the snoRNA SNR52 promoter and a SUP4 terminator (DiCarlo et al. Nucleic Acids Research, Volume 41, Issue 7, 1 Apr. 2013, Pages 4336-4343; Jakočīunas et al. Metabolic Engineering, Volume 28, March 2015, Pages 213-222). Custom yeast gRNA-expression plasmids were generated by inverse PCR of template gRNA-expression plasmids using a 5′-phosphorylated primer and a primer with a tail for replacing the 20 bp gRNA target sequence. Purified amplicons were then blunt-end ligated using T4 DNA Ligase (Thermo Scientific) and template plasmid was then removed by DpnI-digestion (New England BioLabs). Plasmid propagation and assembly were conducted in E. coli strain DH5a, which was cultivated in Terrific Broth or Luria-Bertani with ampicillin (100 mg/L) at 37° C. in liquid media at 300 r.p.m. or on agar plates for 16-20 hrs. All E. coli transformants for plasmid assembly were genotyped by colony PCR to screen for successful assembly, and correct assembly was verified by Sanger sequencing of purified plasmids using the Overnight Mix2Seq Kit service (Eurofins Genomics). For colony PCR of E. coli, OneTaq® Quick-Load® 2× Master Mix with Standard Buffer (New England BioLabs) was used, and PCRs and gel electrophoresis were conducted as described above. Plasmids were purified from E. coli cultures using the NucleoSpin Plasmid kit (Macherey-Nagel). All plasmids contained ampicillin resistance (ampR) for propagation in E. coli, and retention in yeast relied on auxotrophic markers (HIS3, URA3, LEU2, TRP1) or nourseothricin antibiotic resistance (natMX). Primers, DNA fragments, amplicons, plasmids, and gDNA were always dissolved in Milli-Q H₂O and stored at −20° C.

### Yeast Cultivation

All yeast strains were engineered from Saccharomyces cerevisiae strain BY4741. Strains were grown in YPD media with 2% (w/v) glucose except for during experiments or for plasmid retention, in which strains were grown in SC with 2% (w/v) glucose using appropriate amino acid dropout mixes and/or with the addition of antibiotic nourseothricin (100 mg/L) (Jena Bioscience). Yeast strains were generally grown at 30° C. at 250 r.p.m. or on 1.5% (w/v) agar plates, unless otherwise stated for specific experiments. All strains were stored as cryostocks in media with 25% (v/v) glycerol at −80° C.

### Yeast Transformation and Engineering

All transformations of yeast were done using the LiAc/ssDNA/PEG method (Gietz & Schiestl, Nature Protocols volume 2, pages 35-37 (2007)). Plasmid-based expression utilized either 2μ- or CEN/ARS-type plasmids. All genome engineering relied on CRISPR/Cas9-based methods, by having cells constitutively expressing Cas9 from pEDJ391 (Jensen et al. Nucleic Acids Res. 2021 Sep. 7; 49(15):e88.). Genome integrations relied on transformation of I) FastDigest™ NotI-linearized (Thermo Fisher) cassette-containing USER-assembled plasmids with homology arms and II) gRNA helper vectors, both targeting the characterized integration sites described for the EasyClone MarkerFree system (Jessop-Fabre et al. Biotechnol J. 2016 August; 11(8):1110-7). Gene knockouts relied on transformation of I) a gRNA expression vector targeting the site of interest and II) a repair template with homology flanking the cut site. Similarly, scarless gene alterations were done by using a repair template containing the novel sequence of interest. Genomic engineering was confirmed by genotyping via PCR, using OneTaq® Quick-Load® 2× Master Mix with Standard Buffer (New England BioLabs) and Sanger sequencing of cassettes, as described above.

### Model Reporter Protein

Each promoter was defined as the 1,000 bp sequence upstream of the CDS, except for P_{6xLexOLEU2} (Shaw et al. Cell. 2019 Apr. 18; 177(3):782-796.e27), and USER-assembled with yEGFP and T_{CPS1}, to form reporter cassettes. These cassettes were then genome-integrated in strain DIX14, as described above.

### Strain Genotypes

The precursor DIX14 strain was obtained by modification of the background strain Saccharomyces cerevisiae BY4741. The precursor DIX14 strain was genetically optimized for GPCR biosensing by recombinant expression of native α-factor pheromone-sensing mating GPCR Ste2 (P_{CCw12}-STE2-T_{CYC1}), and of native G protein Ga-subunit (P_{PGK1}-GPA1-T_{CYC1}), which is the Ga-subunit of the heterotrimeric G_{αβγ}-protein that couples the GPCR to pheromone response pathway (PRP). Additionally, several native genes were knocked out: the native PRP-coupled GPCRs (ste2Δ0 and ste3Δ0), the native Ga-subunit (gpa1Δ0), a negative feedback regulator for PRP-activation (sst2Δ0), an α-factor protease (bar1Δ0), and a cyclin-dependent kinase inhibitor that induce G1 cell cycle arrest (far1Δ0). Additionally, Aga2 was deleted (aga2Δ0) (see Table 6).

Several PRP-regulated promoters, also commonly referred to as pheromone-responsive, were characterized as processing units: P_{MFA2}, P_{FUS1}, P_{AGA2}, P_{FIG.1}, P_{MFA1}, P_{YCL076W}, P_{HPF1}, P_{IME4}, and P_{CSS1}, as well as two constitutive promoters: P_{TDH3} and P_{PGK1} that initially served as positive controls. A negative control without a yEGFP expression cassette was included. Additionally, an orthogonal GPCR-signalling response was assessed, by knocking out the main PRP-response transcription factor Ste12 (ste12Δ0), and insertion of a cassette containing a synthetic transcription factor (sTF) composed of the pheromone-responsive domain (PRD) of Ste12 and LexA, the latter which binds LexA operator sites (LexO) in DNA (P_{RAD27}-LexA-Ste12PRD-T_{ENO1}) (Shaw et al. Cell. 2019 Apr. 18; 177(3):782-796.e27.). For this sTF, a synthetic promoter containing LexO was characterized as a processing unit: P6xLexOLEU2 (Shaw et al. Cell. 2019 Apr. 18; 177(3):782-796.e27) (see Table 6).

TABLE 6
Genotype of employed strains

| Strain | Genotype |
|---|---|
| Background strain: | MATa, his3Δ1, leu2Δ0, met15Δ0, ura3Δ0 |
| Saccharomyces | |
| cerevisiae BY4741 | |
| DIX14 | Saccharomyces cerevisiae BY4741, P_{CCW12}-STE2-T_{CYC1}, P_{PGK1}- |
| | GPA1-T_{CYC1}, ste2Δ0, ste3Δ0, gpa1Δ0, sst2Δ0, bar1Δ0, far1Δ0, |
| | aga2Δ0^{1,2,3,4,5,6} |
| DIX17 | DIX14, P_{MFA2}-yEGFP-T_{CPS1}^{7,8,9} |
| DIX24 | DIX14, P_{FUS1}-yEGFP-T_{CPS1}^{7,8,10} |
| DIX19 | DIX14, P_{AGA2}-yEGFP-T_{CPS1}^{7,8,11} |
| DIX25 | DIX14, P_{FIG1}-yEGFP-T_{CPS1}^{7,8,12} |
| DIX18 | DIX14, P_{MFA1}-yEGFP-T_{CPS1}^{7,8,13} |
| DIX20 | DIX14, P_{YCL076W}-yEGFP-T_{CPS1}^{7,8,14} |
| DIX22 | DIX14, P_{HPF1}-yEGFP-T_{CPS1}^{7,8,15} |
| DIX21 | DIX14, P_{IME4}-yEGFP-T_{CPS1}^{7,8,16} |
| DIX23 | DIX14, P_{CSS1}-yEGFP-T_{CPS1}^{7,8,17} |
| DIX27 | DIX14, P_{PGK1}-yEGFP-T_{CPS1}^{7,8,18} |
| DIX28 | DIX14, P_{TDH3}-yEGFP-T_{CPS1}^{7,8,19} |
| DIX34 | DIX14, ste12Δ0, P_{RAD27}-LexA-Ste12PRD-T_{ENO1}^{20,21,22}, P_{6xLexOLEU2}- |
| | yEGFP-T_{CPS1}^{7,8,23} |

¹P_{CCW12} promoter sequence, encoded by the nucleotide sequence of SEQ ID NO: 14.
²STE2 amino acid sequence as shown in SEQ ID NO: 24, encoded by the nucleotide sequence of SEQ ID NO: 81.
³T_{CYC1} terminator sequence, encoded by the nucleotide sequence of SEQ ID NO: 20.
⁴P_{PGK1} promoter sequence, encoded by the nucleotide sequence of SEQ ID NO: 11.
⁵GPA1 amino acid sequence as shown in SEQ ID NO: 31, encoded by the nucleotide sequence of SEQ ID NO: 88.
⁶T_{CYC1} terminator sequence, encoded by the nucleotide sequence of SEQ ID NO: 20.
⁷yEGFP amino acid sequence as shown in SEQ ID NO: 38, encoded by the nucleotide sequence of SEQ ID NO: 95.
⁸T_{CPS1} terminator sequence, encoded by the nucleotide sequence of SEQ ID NO: 19.
⁹P_{MFA2} promoter sequence, encoded by the nucleotide sequence of SEQ ID NO: 1.
¹⁰P_{FUS1} promoter sequence, encoded by the nucleotide sequence of SEQ ID NO: 8.
¹¹P_{AGA2} promoter sequence, encoded by the nucleotide sequence of SEQ ID NO: 3.
¹²P_{FIG1} promoter sequence, encoded by the nucleotide sequence of SEQ ID NO: 9.
¹³P_{MFA1} promoter sequence, encoded by the nucleotide sequence of SEQ ID NO: 2.
¹⁴P_{YCL076W} promoter sequence, encoded by the nucleotide sequence of SEQ ID NO: 4.
¹⁵P_{HPF1} promoter sequence, encoded by the nucleotide sequence of SEQ ID NO: 6.
¹⁶P_{IME4} promoter sequence, encoded by the nucleotide sequence of SEQ ID NO: 5.
¹⁷P_{CSS1} promoter sequence, encoded by the nucleotide sequence of SEQ ID NO: 7.
¹⁸P_{PGK1} promoter sequence, encoded by the nucleotide sequence of SEQ ID NO: 11.
¹⁹P_{TDH3} promoter sequence, encoded by the nucleotide sequence of SEQ ID NO: 10.
²⁰P_{RAD27} promoter sequence, encoded by the nucleotide sequence of SEQ ID NO: 18.
²¹LexA-Ste12PRD amino acid sequence as shown in SEQ ID NO: 37, encoded by the nucleotide sequence of SEQ ID NO: 94.
²²T_{ENO1} terminator sequence, encoded by the nucleotide sequence of SEQ ID NO: 22.
²³P_{6xLexOLEU2} promoter sequence, encoded by the nucleotide sequence of SEQ ID NO: 15.

### Dose Response Assay and Flow Cytometry

To investigate the dose-response features of the selected promoters P_{MFA2}, P_{FUS1}, P_{AGA2}, P_{FIG.1}, P_{MFA1}, P_{YCL076W}, P_{HPF1}, P_{IME4}, P_{CSS1}, P_{TDH3}, P_{PGK1} and P_{6XLexOLEU2} upon GPCR stimulation the strains were pre-grown in SC (24 hrs.), whereafter, for each replicate, cells were transferred to 200 μL volumes comprising a 10-fold dilution series of α-factor (0-100 μM) (GenScript) dissolved in SC+1% DMSO, and stimulated for 5 hrs. (30° C., 250 r.p.m.). Flow cytometric measurement was done to quantify yEGFP levels (B525/45-A) and PRP-regulated morphological changes, denoted shmooing (SSC-A) using a NovoCyte Quanteon flow cytometer (Agilent). Data represents means of median fluorescence intensity (mMFI) for three biological replicates and standard deviations hereof. Two-way ANOVA with Tukey's multiple comparisons test was conducted for all conditions.

### Results

The processing module translates the GPCR activation, e.g., Ste2 stimulation with α-factor, into expressed output. The GPCR signals through the heterotrimeric Gαβγ-protein that couples the receptor to the PRP, which in turn results in regulated expression of the cassettes, here characterized by a yEGFP reporter, but to be applied for driving expression of signal molecules for cellular communication (see FIG. 2). After 5 hrs. of stimulation all strains displayed significant ligand concentration-dependent changes in yEGFP expression (see FIG. 3A) and morphological changes (shmooing) (see FIG. 3B), as of GPCR stimulation. Shmooing, measured as cellular complexity (SSC-A), was most intense at ≥0.01 μM, and generally peaking at 1 μM, across all strains, and the effect was mainly attributed to the concentration of α-factor (SV=76.5%), indicating that the pathway was indeed activated similarly across all designs (see FIG. 3B).

The significant increase in absolute yEGFP levels, however, displayed very diverse intensities dependent on the chosen promoter. In fact, across all strains, the yEGFP expression changes could be attributed mainly to the choice of promoter (SV=81.6%), rather than α-factor concentration, emphasizing the importance of choice of promoter for a given output (see FIG. 3A).

P_{MFA2} was the strongest promoter and displayed the largest span in absolute intensity, exemplified by comparison to P_{TDH3}, which is commonly regarded as the strongest constitutive yeast promoter; at 0 μM P_{MFA2} had 0.3-fold strength of P_{TDH3}, whilst at 100 μM it was 6.7-fold stronger than conventional P_{TDH3} expression. In regard to absolute intensity, P_{MFA2} was followed by P_{FUS1}, P_{AGA2}, P_{FIG.1}, P_{MFA1}, and P_{YCL076W} that displayed substantial intensity, and finally P_{HPF1}, P_{IME4}, and P_{CSS1} that showed low intensities (see FIG. 3A).

In summary, diverse processing modules could be generated from responsive endogenous yeast promoters to control system output, which provided different response profiles in relation to GPCR stimulation.

General protein expression can be post-transcriptionally amplified by GPCR stimulation Remarkably, it was discovered that PRP activation causes a general amplification of expression of any gene by any promoter in a yeast cell: a PRP-induced expression boost post-transcriptionally amplifies the expression of genes and is not limited to pheromone-responsive promoters. It was estimated from the yEGFP data that this PRP-induced expression boost can generally amplify expression by 4.01±0.86-fold after 5 hrs of GPCR stimulation (see FIG. 4). It could be observed that P_{PGK1} and P_{TDH3} increased yEGFP expression levels in response to GPCR stimulation (see FIG. 3), despite being commonly regarded as constitutive and predicted to be so from the previous transcriptomics analysis (Jensen et al. Nat Commun. 2022 Oct. 19; 13(1):6201). Under further scrutiny of the dataset, it became evident that this expression amplification was time- and GPCR ligand concentration-dependent, in this case α-factor, and had intensity patterns analogous to the level of shmooing (see FIG. 3-4).

To confirm that this effect is dependent on the main transcription factor of the PRP, Ste12, a STE12 knock-out (ste12Δ0) that eliminates PRP activation despite the presence of a GPCR was performed and GPCR stimulation was assessed by employing an orthogonal signal pathway relying on a LexA-based synthetic transcription factor and LexO-containing promoter (P_{6xLexOLEU2}) (Shaw et al. Cell. 2019 Apr. 18; 177(3):782-796.e27). Indeed, ste12Δ0 prevented downstream PRP-related phenotypes, such as e.g., shmooing and the PRP-induced expression boost, in the strain with orthogonal GPCR signalling (see FIG. 5).

In fact, the PF_{US1} STE12+ strain had a 4.2-fold higher maximal absolute output than the orthogonal P_{6xLexOLEU2} ste12Δ0 strain (see FIG. 5), even though PF_{US1} was merely 1.4-fold stronger than P_{6xLexOLEU2} at maximal induction (see FIG. 5), emphasizing the significance of Ste12-dependent PRP-mediated expression boost contribution to output.

Accordingly, it is considered that two sequential separate effects contribute to the control of gene expression levels when utilizing PRP-coupled GPCR sensory input, namely I) a transcriptional activity profile, which is increased or decreased with intensity specific for a chosen PRP-regulated promoter, 1l) a general PRP-induced expression boost caused by phenotypic changes to the yeast cell attributed to Ste12-dependent PRP activation, likely occurring at a post-transcriptional level caused by gene products of Ste12-regulated genes.

### Different Promoters Provide Different Dose-Response Kinetics

To inspect the response kinetics of PRP-regulated promoters, a separation of the two effects can be approximated by normalizing the absolute output, in this case yEGFP expression levels, to the cell complexities (SSC-A). Upon normalization, very different expression profiles could be obtained from the processing module promoters, with different fold changes of induction ranging from 128.81±7.17-fold (P_{FIG.1}) to 0.62±0.01-fold (P_{HPF1}) and hence also enabling both up- and downregulation (see FIG. 3A and 6). It should be noted that fold changes describe the ability for the promoter to change, relative to its ordinary expression level, making it a characteristic important to consider if one wants a processing unit that can create extensive variation (e.g., P_{FIG.1}). Hence, promoters with low fold change might still display immense changes in absolute expression output, which are masked by a high background level of expression (e.g., P_{MFA2}). Hence, if an intense output is desired for an application, it might be worth considering promoters of high absolute output for the processing units and neglect fold changes, or alternatively find a middle ground (e.g., P_{FUS1}). SSC-normalization changes the dynamics of the curves and lowers the fold change, by isolating promoter contribution from the PRP-induced expression boost. For example, unnormalized P_{FIG.1} peaked at 1 μM with a maximum fold change of 516.8±83.0, then decreasing to 482.6±98.5 at 100 μM, whilst SSC-normalized P_{FIG.1} peaked at the highest concentration (100 μM) with a maximum fold change of 128.8±7.2 (see FIG. 6). Curiously, even though their absolute response is upregulated, both P_{HPF1} and P_{CSS1} are in fact downregulated upon PRP stimulation, as predicted from the transcriptome, and display inverse agonist behaviour (see FIG. 6). This discovery provides the first reporting of a functional inverse agonist GPCR-based yeast biosensor, which may be applicable for any heterologous GPCR.

In summary, it became evident that system output was not solely determined by transcriptional activity, but that the sense-response system itself changes towards a state of amplified expression with increased stimulation. It is envisioned that this PRP-dependent effect can be employed to increase the expression levels of any protein in yeast upon stimulation, of interest to applications in for example biotechnology, metabolic engineering, and health. Importantly, the PRP-induced expression boost enables the conversion of outputs from any constitutive promoters to being GPCR-regulated, enabling the likely use of any yeast promoter for the SCASA system.

### Example 2—Design of an Effector Module

### Aim

The aim of the present example was to design and test the effector module of the SCASA system by functionally linking it to the sensory module and different processing modules, exemplified by the signalling molecule CD19 for communication with anti-CD19 CAR T cells.

### Materials and Methods

Constructs were generated by the methods described in Example 1. Yeast strains were engineered by the methods described in Example 1. Yeast strains were cultivated by the methods described in Example 1.

### Effector Module Construct Description

For yeast surface display (YSD), a highly mutated CD19 extracellular domain (CD19.1 ECD) was employed for correct folding and expression in yeast (Klesmith et al. 2019. Retargeting CD19 Chimeric Antigen Receptor T Cells via Engineered CD19-Fusion Proteins. Molecular Pharmaceutics, 16(8), 3544-3558., Klesmith et al. 2019. Fine Epitope Mapping of the CD19 Extracellular Domain Promotes Design. Biochemistry, 58(48), 4869-4881). The display construct for the effector module was generated by fusion of a hemagglutinin (HA) tag, a PAS40-linker, a (G₄S)₃-linker, and the CD19.1 ECD, hereafter also denoted CD19, to the C-terminal of Aga2, also containing the native N-terminal Aga2 signal peptide (see FIG. 7 and FIG. 30).

### Strain Genotypes and Cell Lines

The precursor DIX41 strain was obtained by modification of DIX14 strain, described in Example 1. DIX41 is an optimized GPCR-based biosensor (as described for DIX14), with optimization of YSD capabilities by recombinant expression of Aga1 (P_{TDH3}-AGA1-T_{ADH1}) and deletion of native AGA2 (aga2Δ0) to avoid potential competitive binding to Aga1. Several CD19-expression cassettes were generated using different processing module promoters; P_{MFA2}, P_{FUS1}, P_{FIG.1}, P_{MFA1}, P_{HPF1}, P_{TDH3}, P_{PGK1} and P_{GAL1}, and genome-integrated in DIX41 (see FIG. 30). In addition, plasmid-based expression of the P_{GAL1}-CD19 cassette from a multicopy 2μ plasmid with URA3-selection (pMAD58), based on pEDJ400 (Jensen et al. Nucleic Acids Res. 2021 Sep. 7; 49(15):e88), was assessed by transforming it into the DIX41. Two negative control strains were used; background strain DIX41 without any display (‘No YSD’) and DIX44 with P_{PGK1}-controlled display of the Aga2-fusion protein, but without the CD19 sequence (‘P_{PGK1}-Empty’) (see Table 7). Conventional YSD using S. cerevisiae strain EBY100 and pCT-plasmid-based expression was also assessed (Chao et al. 2006, Isolating and engineering human antibodies using yeast surface display. Nature Protocols, 1(2), 755-768). In addition to yeast strains, the human CD19+ NALM6 B cell precursor leukemia cell line (DSMZ, no.: ACC 128) was included as a benchmark positive control.

TABLE 7
Genotype of employed yeast strains

| Strain | Genotype |
|---|---|
| DIX14 | See Table 6. |
| Background | DIX14, P_{TDH3}-AGA1-T_{ADH1} |
| strain; DIX41 | |
| (‘No YSD’) | |
| DIX44 (‘P_{PGK1}- | DIX41, P_{PGK1}-Aga2-HA-PAS40-(G₄S)₃-myc- |
| Empty’) | T_{CPS1}^{1,2,3,4,12,14,15,16} |
| DIX48 (‘P_{MFA2}- | DIX41, P_{MFA2}-Aga2-HA-PAS40-(G₄S)₃-CD19-myc- |
| CD19’) | T_{CPS1}^{1,2,3,4,5,6,14,15,17} |
| DIX45 (‘P_{FUS1}- | DIX41, P_{FUS1}-Aga2-HA-PAS40-(G₄S)₃-CD19-myc- |
| CD19’) | T_{CPS1}^{1,2,3,4,5,7,14,15,17} |
| DIX56 (‘P_{FIG1}- | DIX41, P_{FIG1}-Aga2-HA-PAS40-(G₄S)₃-CD19-myc- |
| CD19’) | T_{CPS1}^{1,2,3,4,5,8,14,15,17} |
| DIX46 (‘P_{MFA1}- | DIX41, P_{MFA1}-Aga2-HA-PAS40-(G₄S)₃-CD19-myc- |
| CD19’) | T_{CPS1}^{1,2,3,4,5,9,14,15,17} |
| DIX55 (‘P_{HPF1}- | DIX41, P_{HPF1}-Aga2-HA-PAS40-(G₄S)₃-CD19-myc- |
| CD19’) | T_{CPS1}^{1,2,3,4,5,10,14,15,17} |
| DIX49 (‘P_{TDH3}- | DIX41, P_{TDH3}-Aga2-HA-PAS40-(G₄S)₃-CD19-myc- |
| CD19’) | T_{CPS1}^{1,2,3,4,5,11,14,15,17} |
| DIX47 (‘P_{PGK1}- | DIX41, P_{PGK1}-Aga2-HA-PAS40-(G₄S)₃-CD19-myc- |
| CD19’) | T_{CPS1}^{1,2,3,4,5,12,14,15,17} |
| DIX74 (‘P_{GAL1}- | DIX41, P_{GAL1}-Aga2-HA-PAS40-(G₄S)₃-CD19-myc- |
| CD19’) | T_{CPS1}^{1,2,3,4,5,13,14,15,17} |

¹Aga2 amino acid sequence as shown in SEQ ID NO: 40, encoded by the nucleotide sequence of SEQ ID NO: 97.
²HA tag as shown in SEQ ID NO: 45, encoded by the nucleotide sequence of SEQ ID NO: 102.
³PAS40 linker as shown in SEQ ID NO: 46, encoded by the nucleotide sequence of SEQ ID NO: 103.
⁴(G4S)3 linker as shown in SEQ ID NO: 47, encoded by the nucleotide sequence of SEQ ID NO: 104.
⁵CD19 amino acid sequence as shown in SEQ ID NO: 53, encoded by the nucleotide sequence of SEQ ID NO: 110.
⁶P_{MFA2} promoter sequence, encoded by the nucleotide sequence of SEQ ID NO: 1.
⁷P_{FUS1} promoter sequence, encoded by the nucleotide sequence of SEQ ID NO: 8.
⁸P_{FIG1} promoter sequence, encoded by the nucleotide sequence of SEQ ID NO: 9.
⁹P_{MFA1} promoter sequence, encoded by the nucleotide sequence of SEQ ID NO: 2.
¹⁰P_{HPF1} promoter sequence, encoded by the nucleotide sequence of SEQ ID NO: 6.
¹¹P_{TDH3} promoter sequence, encoded by the nucleotide sequence of SEQ ID NO: 10.
¹²P_{PGK1} promoter sequence, encoded by the nucleotide sequence of SEQ ID NO: 11.
¹³P_{GAL1} promoter sequence, encoded by the nucleotide sequence of SEQ ID NO: 16.
¹⁴Myc tag as shown in SEQ ID NO: 50, encoded by the nucleotide sequence of SEQ ID NO: 107.
¹⁵T_{CPS1} terminator sequence, encoded by the nucleotide sequence of SEQ ID NO: 19.
¹⁶Full Aga2p-HA-PAS40-(G4S)3-myc CDS sequence as shown in SEQ ID NO: 63, encoded by the nucleotide sequence of SEQ ID NO: 120.
¹⁷Full Aga2p-HA-PAS40-(G4S)3-CD19-myc CDS sequence as shown in SEQ ID NO: 65, encoded by the nucleotide sequence of SEQ ID NO: 122.

### Assays, Staining, and Flow Cytometry

To investigate the different variants of CD19 modules, yeast cells were grown and, in some cases, stimulated to present CD19 differentially, whereafter they were stained with antibodies and assessed by flow cytometry. Flow cytometric measurement was done using a NovoCyte Quanteon flow cytometer (Agilent) to quantify CD19 by staining with mouse anti-CD19 (FMC63) (Absolute Antibody; Ab00613) and goat anti-mouse-AF647 (polyclonal) (Thermo Fisher; A-21236), detected in R667/30-H, as well as rabbit anti-HA (RM305) (Thermo Fisher; MA5-27915) and goat anti-rabbit-AF488 (polyclonal) (Thermo Fisher; A-11008), detected in B525/45-H (see FIG. 7), and to measure PRP-regulated morphological changes (SSC-H). To confirm clinically relevant folding of CD19 display, as well as quantification of display, the FMC63-clone anti-CD19 antibody that uses the same binding domain as FDA-approved CAR T cell designs was used. Data represents means of median fluorescence intensity (mMFI) for three biological replicates and standard deviations hereof. Two-way ANOVA with multiple comparisons tests was conducted for all conditions. Histograms are representative replicates. No SSC-normalization was used in the evaluation of the effector module, unlike in Example 1, as it is the absolute response of all effects combined that constitute the total output that CAR T cells interpret and react to.

For conventional YSD using strain EBY100 and pCT-plasmid-based expression (Chao et al. 2006, Isolating and engineering human antibodies using yeast surface display. Nature Protocols, 1(2), 755-768), strains were pre-grown in SC −trp, then transferred to 5 mL SC −trp +2% Gal for induction of YSD or +2% Gluc as a negative control (24 hrs., 30° C., 250 r.p.m.). NALM6 was cultured in RPMI 1640 medium (ATCC modification) (Thermo Fisher) with 10% fetal bovine serum and 1% pen-strep (RPMI+10% FBS) at 37° C. with 5% CO₂. For the investigation of P_{GAL1}-titratability of DIX41 with pMAD58 and DIX74, strains were pre-grown in SC −ura and SC, respectively, then transferred to SC −ura and SC, respectively, with varying concentrations of galactose (0.0002%-2% Gal), using raffinose as compensating carbon source, and YSD was induced for 24 hrs. (30° C., 250 r.p.m.). Strains were similarly grown in 2% Gluc as negative controls. For the investigation of the strain library with constitutive and dynamically regulated expression of CD19 (Background strain/No YSD, P_{PGK1}-Empty, P_{MFA2}-CD19, P_{FUS1}-CD19, P_{FIG.1}-CD19, P_{MFA1}-CD19, P_{HPF1}-CD19, P_{TDH3}-CD19, P_{PGK1}-CD19) the strains were pre-grown in SC, whereafter, for each replicate, 50.000 cells were transferred to 300 μL volumes comprising a 10-fold dilution series of α-factor (0-100 μM) (GenScript) dissolved in SC+1% DMSO, and stimulated for 20 hrs. (30° C., 250 r.p.m.).

### Results

To exemplify the ability of the system to differentially present on the surface a clinically relevant signal molecule, an immuno-oncology relevant approach was evaluated by creating artificial antigen-presenting cells (aAPCs) from yeast cells, specifically for ex vivo activation of human CAR+ T cells targeted against the antigen CD19. The yeast-based aAPCs can simulate different cancer cell phenotypes by varying densities of surface antigen and hence address questions of CD19 antigen density regarding the intensity of CAR+ T cell responses for specific CAR designs, which has recently been shown to be a question of clinical interest (Majzner et al. Cancer Discov. 2020 May; 10(5):702-723). Using the system presented herein, the yeast aAPCs (SCASA yeast aAPC) display signal molecule CD19 on the cell surface (effector module), proportionally to the signal transmitted (processing module) from the sensed amount of stimuli intentionally added to the cells (sensory module), herein exemplified using a GPCR sensor with different processing modules (see FIG. 1 and FIG. 30) and using a galactose-inducible system.

### Conventional YSD Systems are not Optimal for High-Precision Cellular Communication Systems

CD19 was successfully displayed on aAPCs by using genome-integrated designs, at which ~99.9% of aAPCs displayed CD19 (P_{TDH3}-CD19), with no non-effective population, comparable to the phenotype of the human CD19+ cancer cell line NALM6 (100.0%) (see FIG. 8). In comparison, for conventional EBY100 pCT-plasmid-based YSD using galactose-induced expression from P_{GAL1}, only 38.6% of cells displayed CD19 after 2% Gal induction for 24 hrs, meaning that a majority of the yeast cells did not display CD19 (see FIG. 8).

For applications in cellular communication, a controlled homogenous uniform response is desired for delivering a signal of specified intensity to receiver cells as interactions occur at the cell-to-cell level, thus approximating a better imitation of cancer cells (see FIG. 8). However, conventional methods for YSD often result in heterogeneous population behaviour, seen as multimodal display distributions, where cells behave drastically differently within a single culture, as also demonstrated here (see FIG. 8-9 and FIG. 28A), which expectantly is disruptive to a signal of fine-tuned intensity. This behavioural variation of YSD may be attributed to, firstly, the common use of plasmid-based expression, which often leave a residual non-effective population, and, secondly, the use of galactose-inducible promoters (e.g., P_{GAL1}), which are titratable at a population-wide level, however, not at the single cell level, at which the response is binary. To further understand the limitations of galactose induction and plasmid-based expression, the expression of the P_{GAL1}-CD19 cassette from a genome-integrated site and from a multicopy 2μ plasmid with URA3-selection (pMAD58) was assessed by introduction into the DIX41 background strain after induction in varying concentrations of galactose (0.0002%-2% Gal) for 24 hrs.

For both the genome-integrated and plasmid-based expression, a bimodal distribution of activated and un-activated cells was seen for ≥0.02% Gal at similar intensities, confirming that P_{GAL1} was not titratable at the single-cell level (see FIG. 9 and FIG. 28A). For plasmid-based expression, the maximum amount of displaying cells was 41.2% of the population (0.02% Gal), while for integrated expression, a responding population of 99.2% could be obtained, however, limited to only occurring at maximal galactose induction (2% Gal), as of the binary response kinetics of P_{GAL1} (see FIG. 9 and FIG. 28A-B). The relative intensity of CD19 varied for the CD19+ populations, dependent on whether the cassette was genome-integrated or expressed from a 2μ plasmid (see FIG. 28C). No CD19 was detected for the negative control cultured in glucose (2% Gluc) (see FIG. 9 and FIG. 2).

In summary, conventional YSD methods give large population-level variation and are not tuneable to the same extent as the GPCR-linked YSD methods presented herein.

### Programmable GPCR-Based Systems for YSD Allows for Tighter Control and Dynamic Regulation

The ability to tune the CD19 antigen density on SCASA yeast aAPCs was investigated. This was possible by three non-mutually exclusive individual effects by I) different constitutive or background expression levels from chosen promoters, II) through upregulation of promoters via GPCR stimulation, and III) through PRP-mediated expression boost via GPCR stimulation (see FIG. 30).

The different default expression levels of promoters in the processing module enabled the generation of a small library of strains with different constitutive CD19 densities (see FIG. 10). Interestingly, at unstimulated conditions, PFIG1 and PFUS1 provided extremely low levels of CD19 at the limit of detection, while PMFA1, PMFA2, PHFP1, PPGK1, and PTDH3 provided CD19 at distinct higher intensities. Next, tuneable CD19 antigen density via GPCR stimulation was explored, by exposing the strains to a 10-fold dilution series of α-factor (0-100 μM).

Each strain's CD19 density could be further diversified in a continuous manner by dynamic control through stimulation of the GPCR (Ste2) by titration of the ligand stimuli to provide a multitude of different CD19+ profiles (see FIG. 11). All strain designs showed significant increases of CD19 from their background levels upon stimulation of their GPCR, importantly, at different intensities for the individual concentrations of α-factor dependent on the specific processing module of the strain (see FIGS. 11 and 12). Importantly, the response control for CD19 density was non-binary, i.e., a continuous response, dependent on the α-factor concentration, across the different levels of stimulation (see FIG. 11), unlike conventional YSD systems.

The highest acquired CD19 antigen density was obtained for P_{TDH3} at 1 μM α-factor, while the largest span in absolute CD19 antigen density relative to the unstimulated condition was seen for P_{MFA2} at 1 μM α-factor (see FIG. 12A). P_{MFA1}, P_{FUS1}, P_{PGK1}, and P_{FIG.1} provided similar intermediate output profiles at >0.1 μM α-factor, whilst P_{HPF1} only provided modest levels CD19 (see FIG. 12A). Interestingly, fold changes in CD19 display levels revealed that only P_{FUS1}, P_{FIG.1}, P_{MFA1}, and P_{MFA2} could provide drastic changes in CD19 display relative to the unstimulated condition (see FIG. 12B). For example, P_{FIG.1} provided a 629.6±98.6-fold upregulation (see FIG. 12B), from undetectable levels of CD19 to levels comparable to P_{TDH3} (see FIG. 12A), meaning that this single strain was capable of spanning the entire CD19 intensity range provided by the unstimulated strain library (see FIG. 10). PF_{US1} provided the highest relative change of 774±165-fold (see FIG. 12B). The maximal general PRP-induced expression boost for all strain designs was 2.9±0.3-fold (1 μM α-factor) (see FIG. 13). Interestingly, the output fold changes of non-upregulated promoters P_{HPF1}, P_{TDH3}, and P_{PGK1} all lied in the range of maximal 2.9-3.8-fold (see FIG. 12B), indicating that the sole contribution to their increase in absolute CD19 levels was the PRP-induced expression boost (see FIG. 13). Remarkably, the importance of the seemingly modest PRP-induced expression boost should not be neglected, as the change of absolute output from the 3.1±0.3-fold upregulation of P_{TDH3} was much greater (150.2%) than the 774±165-fold upregulation of PF_{US1} (see FIG. 12). Upon inspection of morphological changes, it became evident that the largest increases in antigen density coincided with the morphological shmoo-response, indicating the importance of the PRP-mediated expression boost for obtaining high-intensity antigen presentation and large diversity in the library (see FIG. 13). Hence, PRP-induced boost is crucial for obtaining extreme changes in signal output, however, depending on the expression level of the specific promoter chosen, supporting the findings of processing module behaviour using the yEGFP reporter (see Example 1).

In summary, dynamic CD19 antigen density is controllable on individual SCASA yeast aAPCs and different CD19 profiles could be generated via different processing modules responding to GPCR stimulation, allowing for different types of dynamic control.

### Example 3—Synthetic Activation of Human Immune Cells Via the SCASA System

### Aim

The aim of the present example was to validate the CD19-presenting SCASA yeast aAPCs functionality in communication with human cells for immuno-oncological applications and to confirm that response intensities were dependent on controlled antigen-densities.

### Materials and Methods

### CAR Jurkat NFAT-Luc Cell Generation

To investigate the activation of CAR T cells, a Jurkat cell line equipped with a nuclear factor of activated T cell (NFAT) transcription factor luciferase reporter system (Jurkat NFAT-Luc) (Nordic BioSite, BPS-60621) was further modified by insertion of an anti-CD19 CAR (FMC63-CD8α-4-1 BB-CD3ζ) (Ormhoj et al. 2019, Chimeric antigen receptor T cells targeting CD79b show efficacy in lymphoma with or without cotargeting CD19. Clinical Cancer Research, 25(23), 7046-7057), hence providing a bioluminescent luciferin signal of an intensity proportional to the level of T cell activation (see FIG. 14). NFAT proteins are natively activated by T-cell receptor engagement in immunological responses and are key regulators of driving the activation of T cells, as well as their further differentiation. Consequently, the designed activation of NFAT by CARs is an essential part of the functionality of CAR+ T cells in killing cancer cells.

Lentiviral particles containing the CD19 CAR element were produced by lipofectamine-based co-transfection of HEK293 cells with 3^{rd} generation packaging plasmids pMD2.G (Addgene, #12259), pMDLg/pRRE (Addgene, #12251), pRSV-Rev (Addgene, #12253), and the CAR transfer vector (pDTU), a modified version of pLenti-puro (Addgene, #39481), optimized to include a cPPT-CTS sequence, the EF-1α promoter, and a Woodchuck Hepatitis Virus (WHV) Post-transcriptional Regulatory Element (WPRE). 24 hrs after transfection, lentiviral particles were harvested and concentrated using Lenti-X concentrator (Takara Bio) and stored at −80° C. Jurkat NFAT-Luc cells were transduced with lentivirus at an MOI of 5. After transduction, CD19 CAR+ Jurkat NFAT-Luc cells were single-cell sorted and expanded to adequate numbers before cryopreservation in liquid nitrogen.

### Target Cells: Yeast Strains and NALM6

SCASA yeast aAPC designs were chosen from the library of strains presented in Example 2 (see Table 7), with different constitutive CD19 densities (see FIG. 10) and with possibility for dynamic regulation of CD19 densities (see FIG. 11-12) for human cell co-cultivation, namely the strains P_{FUS1}-CD19, P_{MFA2}-CD19, P_{PGK1}-CD19, and P_{TDH3}-CD19, along with a negative control; P_{PGK1}-Empty, lacking only the CD19 CDS. In addition, the human CD19+ NALM6 B cell leukaemia cell line (NALM6), described in Example 2, was included as a benchmark positive control.

### Co-Cultivation and Assay Conditions

All yeast cells were pre-grown in SC at 30° C. (250 RPM) and washed in 1×PBS before initiation of the co-cultures. For GPCR stimulation of SCASA yeast aAPCs, the yeast cells were additionally pre-cultured in SC+1% DMSO with 0.0001 μM, 0.001 μM, 0.01 μM, 0.1 μM, 1 μM, or no α-factor (GenScript) for 20 hrs at 30° C. (250 RPM). NALM6 and Jurkat cells were cultured in RPMI+10% FBS with 1% pen-strep at 37° C. with 5% CO₂. For each replicate, 200.000 Jurkat NFAT-Luc cells were co-cultured with 40.000 target cells (SCASA yeast aAPCs or NALM6) for 18 hrs. in 200 μL RPMI+10% FBS with 1% pen-strep at 37° C. with 5% CO₂. After co-cultivation, cells were lysed using Pierce™ Luciferase Cell Lysis Buffer (Thermo Fisher) and the luciferase activity was examined using the Luciferase Assay System (Promega). Three replicates were made for each tested condition. Relative activation of Jurkat NFAT-Luc cells was calculated by subtraction of background luminescence and normalization to a 100% CAR+ Jurkat cell mono-culture. ANOVAs with multiple comparisons tests were conducted for all conditions, both for comparison within SCASA yeast aAPCs and comparison to NALM6.

### Results

The specificity and intensity of activation of anti-CD19 CAR+ Jurkat NFAT-Luc cells was investigated using the SCASA yeast aAPCs presenting CD19 (see FIG. 14-15). It was validated that SCASA yeast aAPC designs could be dynamically controlled to simulate different CD19 antigen densities and consequently cause different CAR T cell activation profiles, through SCASA yeast aAPC GPCR stimulation at 0-1 μM α-factor in a 100% CAR+ Jurkat NFAT-Luc co-culture. To examine if T cell activation was specific to the presence of CD19 on yeast and the expression of CARs in T cells, cultures were made where the amount of Jurkat NFAT-Luc cells that contained CARs was graded in 10-percentiles from 0-100% CAR+ in a constant population of 200.000 Jurkat NFAT-Luc cells. Each of these culture compositions was then co-cultivated with 40.000 of the different aAPCs or NALM6 cells, which further enabled to distinguish if the activation intensity was dependent on the antigen-density of CD19.

### CAR T Cell Activation is Specific to CD19-Presentation of Yeast Cells and is Dependent on the CD19 Antigen-Density

CAR Jurkat cells could be activated by SCASA yeast aAPCs presenting CD19, in a manner that was; I) CD19-specific, II) CAR-specific, III) CD19 antigen-density sensitive in regards to the intensity of the Jurkat activation, IV) different for each SCASA yeast aAPC design, V) controllable through the SCASA yeast aAPC GPCR, VI) capable of surpassing activation levels of NALM6 cancer cells, and VII) unaffected by the presence of yeast itself (see FIG. 15).

For the investigation of activation specificity and intensity, the relative specific activation of Jurkat NFAT-Luc cells was calculated by normalization to the 100% CAR+ Jurkat NFAT-Luc cell mono-culture. To demonstrate the tunability of antigen-density, the CD19 presentation of SCASA yeast aAPCs by controlled stimulation of their GPCRs was diversified, in this case using Ste2 with α-factor ligand. All SCASA yeast aAPC designs with CD19 could be controlled to significantly induce differential specific activation of CAR+ Jurkat cells; P_{FUS1}-CD19 (p=0.0045), P_{MFA2}-CD19 (P=0.0002), and P_{TDH3}-CD19 (p<0.0001)(see FIG. 15A). The different SCASA yeast aAPC designs could induce different maximal specific activation intensities throughout their controlled CD19 display, which all occurred at different concentrations; most intense was P_{TDH3}-CD19 at 61.9±2.2-fold (0.1 μM, p<0.0001), followed by P_{MFA2}-CD19 at 22.3±9.1-fold (0.01 μM, p=0.0007), and the least intense was P_{FUS1}-CD19 at 4.1±0.3-fold (1 μM, p=0.0026)(see FIG. 15A). These patterns of CAR+ Jurkat cells activation corresponded to the previously quantified CD19 display profiles of the SCASA yeast aAPCs during GPCR stimulation (see FIGS. 11 and 12). Surprisingly, the SCASA yeast aAPCs could seemingly surpass the specific activation intensity of CAR+ Jurkat cells when using the NALM6 cancer cell line—by comparison, SCASA yeast aAPC with P_{TDH3}-CD19 could achieve a 2.2±0.6-fold higher maximal specific activation than NALM6. The lowest concentration of α-factor for significant detected difference in fold specific activation, relative to 0 μM for each SCASA yeast aAPC, differed dependent on the processing module; P_{TDH3}-CD19 at 0.001 μM giving 27.7±3.0-fold (p<0.0001), P_{MFA2}-CD19 at 0.01 μM giving 22.3±9.1-fold (p=0.0007), and P_{FUS1}-CD19 at 0.01 μM giving 3.2±0.8-fold (p=0.0285). Hence, processing modules with high background levels, e.g., P_{TDH3}-CD19, provide higher sensitivity, as they seemingly reach the threshold of CAR activation at lower levels of GPCR stimulation, likely due to their higher constitutive levels of CD19 (see FIG. 10). High dynamic range of the control of SCASA yeast aAPC-induced CAR+ Jurkat cell activation relies partly on the ability to produce high maximal activation, as seen for PTDH3-CD19 that could increase the activation 10.3±0.4-fold at 0.1 μM relative to the uninduced condition (see FIG. 15B). However, a low constitutive level of CD19 combined with the ability to drastically increase CD19 levels, through the use of PRP-regulated promoters, provided a higher dynamic range, as seen for PMFA2-CD19 that reached 15.3±6.2-, 14.5±4.7-, and 13.6±1.3-fold increased specific activation of CAR+ Jurkat cells at 0.01, 0.1, and 1 μM, respectively, relative to the unstimulated condition (see FIG. 15B). This corroborates the conclusions from the characterizations of processing modules (see FIGS. 2-6) and GPCR-inducible YSD of CD19 (see FIGS. 7-8,10-13). However, the dynamic range in controlling CAR activation is seemingly limited to a necessity to reach a CD19 display level that lies above a threshold for efficient CAR activation, which may explain why PFUS1-CD19 did not display high fold changes in specific activation relative to the unstimulated state (3.1±0.2-fold at 1 μM) (see FIG. 15A), despite containing the processing module promoter (PFUS1) with the highest fold change (see FIG. 12).

By comparison to an unstimulated control mono-culture of 100% CAR+ Jurkat cells, only SCASA yeast aAPCs containing the CD19 CDS in the display design could significantly increase the absolute activation profile of 100% CAR+ Jurkat cells; P_{MFA2}-CD19 (p=0.0135), P_{PGK1}-CD19 (p<0.0001), and P_{TDH3}-CD19 (p<0.0001), while the negative control SCASA yeast aAPC (P_{PGK1}-Empty) showed no significant change in absolute activation (see FIG. 15C). Hence, activation of CAR+ Jurkat cells was dependent on the presence of CD19 on SCASA yeast aAPCs.

Activation by SCASA yeast aAPCs was CD19-specific and depended on the presence of a CAR (see FIG. 15C). In general, no significantly activated Jurkat cells was detected in the range of 0-10% CAR+ for any co-cultivation, however any increase at >10% CAR+ provided significantly higher responses (p<0.0001), and at around >60% CAR+ the detection of increased activation intensity tended to saturate (see FIG. 15C). Significant activation was detected at ≥20% CAR+ for P_{PGK1}-CD19 (p<0.0001) and P_{TDH3}-CD19 (p<0.0001), while ≥30% CAR+ was required for P_{MFA2}-CD19 (p=0.0059) consistent with P_{MFA2}-CD19 having the lowest level of CD19, when GPCRs remain unstimulated.

Each SCASA yeast aAPC design of different constitutive CD19 levels provided significantly different activation profiles in the 0-100% CAR+ range (p<0.0001) (see FIG. 15C), with each activation intensity level being consistent with the level of CD19 displayed on the specific SCASA yeast aAPC (see FIG. 10-12). This was also evident from the maximal fold specific activation acquired for each SCASA yeast aAPC design without any GPCR stimulation; P_{TDH3}-CD19 at 12.8±1.2-fold (70% CAR+, p<0.0001), P_{PGK1}-CD19 at 11.5±1.4-fold (100% CAR+, p<0.0001), and P_{MFA2}-CD19 at 5.5±0.4-fold (90% CAR+, p<0.0001) (see FIG. 15C). Hence, different SCASA yeast aAPC designs provided different CAR+ T cell activation profiles, and SCASA yeast aAPC-displayed CD19 activated CAR+ T cells in an antigen-density-dependent manner.

By comparison to SCASA yeast aAPCs, NALM6 caused a significant increase in the absolute activation of 100% CAR+ Jurkat cells (p<0.0001), caused significant specific activation at >20% CAR+(p=0.0054), and had a peaking activation intensity at 28.1±7.2-fold (90% CAR+, p<0.0001) (see FIG. 15C). Hence, NALM6 provided a maximal specific activation that was 2.2±0.6-fold higher than any SCASA yeast aAPC, but importantly, without SCASA yeast aAPC GPCR stimulation that otherwise could provide more intense activation. Unlike NALM6, the SCASA yeast aAPCs have controllable CD19 antigen densities and are not limited to a fixed amount of CD19 (see FIG. 11-13,15A).

Lastly, significant activation of T cells was dependent on CD19, and the negative control aAPC (P_{PGK1}-Empty) did not elicit any significant Jurkat NFAT-Luc activation under any conditions, for both % CAR+ and GPCR stimulation (see FIG. 15). Hence, it is considered that yeast itself does not activate T cells through NFAT-associated pathways. In summary, the present examples show a fully controllable orthogonal system for dynamic activation of CAR T cells.

### Example 4—Co-Cultivation Conditions and T Cell Activation Protocol

### Aim

The aim of the present example was to investigate the compatibility and conditions of co-cultivations with human immune cells, specifically T cells, and yeast cells for SCASA system applications.

### Materials and Methods

For yeast growth assays, three different yeast strains were assessed; the background strain for YSD strains (DIX41), a strain with P_{PGK1}-controlled display of the Aga2-fusion protein, but without the CD19 sequence (‘P_{PGK1}-Empty’) (DIX44), and a strain displaying the CD19 construct (‘P_{PGK1}-CD19’) (DIX47) (strains presented in Example 2; see Table 7). Yeast strains were seeded at 40.000 and 200.000 cells in 200 μL media and grown at 37° C. for 24 hrs in yeast medias; synthetic complete (SC) and pH=5-buffered synthetic defined (SD) medium (Prins & Billerbeck 2021, A buffered media system for yeast batch culture growth. Bmc Microbiology, 21(1), 127), as well as in human immune cell medias; RPMI+10% FBS with 1% pen-strep and ImmunoCult™-XF T Cell Expansion Medium (Stemcell Technologies). Yeast cells were grown in a BioTek Epoch 2 (Agilent) microplate reader to monitor growth at OD₆₃₀.

For assessing yeast biosensing and T cell viability during long-term co-cultivation, T cells separated from a donor-derived isolate of peripheral blood mononuclear cells (PBMCs) was co-cultured with a yeast strain equipped with the GPCR Ste2 and the PRP-activation reporter cassette PFUS1-yEGFP (strains presented in Example 1; see Table 6). PBMCs were isolated from healthy donor buffy coats collected at the central blood bank at Rigshospitalet (Copenhagen, Denmark), by density centrifugation using LymphoPrep Solution (Axis Shield Poc As) and cryopreserved at −150° C. in FBS (Gibco)+10% DMSO. T cells were isolated from PBMCs by negative selection, using the EasySep™ Human T Cell Isolation Kit (Stemcell Technologies). For co-cultivation, a constant cell number of 500.000 T cells was supplied with ratios of 0.5× (250.000), 1.0× (500.000), or 10× (5.000.000) yeast cells and cultivated in 500 μL RPMI+10% FBS at 37° C. with 5% CO2 for 5 days (96 hrs).

In addition, the 1.0× co-cultures were assessed with 20 μM α-factor. The co-cultures were sampled at day 1 (0 hrs), day 2 (24 hrs), and day 5 (96 hrs) by flow cytometry, after labelling with anti-CD3-BV421 (BD Biosciences, 562426) (V445/45) and a Fixable Near-IR Dead Cell Stain Kit (Thermo Fisher) (R780/60) for assessing T cell viability. All experiments were run in three replicates. ANOVAs with multiple comparisons tests were conducted.

### Results

Firstly, it was investigated whether yeast cells were able to grow in media applied for immune cells to assess if yeast cells could outcompete the growth of immune cells during co-cultivations. The yeast strains could not enter detectable growth phases in immune cell media RPMI+10% FBS or ImmunoCult™-XF for any yeast strain design at any initial seeding cell number (see FIG. 16). Hence, it could be concluded that yeast cells could not grow in mammalian media at 37° C., indicating that yeast was not capable of overgrowing or outcompeting immune cells in a co-cultivation. In addition, this implies that the initial amount of yeast cells seeded remains constant. All yeast strain designs showed growth in conventional yeast medias at 37° C., and yeast cells with YSD designs grew to a lower density than the background strain, with P_{PGK1}-CD19 growing to the lowest density (see FIG. 16).

Next, it was investigated whether yeast cells with GPCRs were able to sense ligands in mammalian media and during co-cultivation with T cells at 37° C. Yeast cells with GPCR Ste2 could sense α-factor in RPMI+10% FBS during both mono-culture and in co-cultures with 1.0× T cells, evident from significantly increased yEGFP signals that lasted until day 5 (96 hrs) (see FIG. 17). Hence yeast cells remain alive and are capable of sensing and responding to ligands using GPCRs via the PRP during T cell co-cultivation.

Lastly, it was investigated whether the presence of yeast in T cell co-cultures affected the viability of T cells. The viability of T cells was not affected by the presence of yeast at any amount of yeast cells, as no significant difference in viability was detected for the 0.5×, 1.0×, and 10.0× ratios of yeast cells to T cells compared to the T cell monoculture after 5 days (see FIG. 18). Hence, yeast cells could be present at cell numbers 10-fold higher (10×10⁶ yeast cells/mL) than the amount of T cells (1×10⁶ T cells/mL) without affecting the T cell viability.

With these novel understandings of yeast behaviour in immune cell media and the lack of effects on T cells by yeast, a 5-day protocol was developed for establishing yeast and immune cell co-cultures with the possibility for pre-stimulating yeast cells to provide communication signals of a predefined intensity (see FIG. 19).

### Example 5—SCASA Yeast aAPCs as Robust Clinically Relevant CAR T Cell Activators

### Aim

The aim of the present example was to illustrate the clinical relevance and compatibility of applying SCASA yeast aAPCs in characterizing CAR T cell products by evaluating response profiles of CAR T cells after SCASA yeast aAPC co-cultivation.

### Materials and Methods

T cells were isolated from PBMCs from buffy coats collected at the central blood bank at Rigshospitalet (Copenhagen, Denmark), analogously to as described in Example 4, using SepMate-50 (IVD) tubes (Stemcell Technologies, 85460), Lymphoprep (Stemcell Technologies, 07811), and Dulbecco's Phosphate Buffered Saline with 2% Fetal Bovine Serum (Stemcell Technologies, 07905), according to manufacturer's protocol. CAR T cells were generated by applying a novel CRISPR-MAD7 method for CAR transgene insertion via electroporation into the isolated T cells from a healthy donor (Mohr et al. The CRISPR-Cas12a Platform for Accurate Genome Editing, Gene Disruption, and Efficient Transgene Integration in Human Immune Cells. ACS Synth. Biol. 12, 375-389 (2023)). The activation of CAR T cells was assayed after 20 hrs co-cultivation with a SCASA yeast aAPC (P_{PGK1}-CD19), the negative control SCASA yeast aAPC (P_{PGK1}-Empty) (strains presented in Example 2; see Table 7), and the benchmark cancer cell line NALM6 (described in Example 2) under three different co-cultivation ratios; 0.2×, 1.0×, and 5.0× target cells to 100,000 alive CAR T cells. The employed CAR was an anti-CD19 CAR with fully-human scFv, containing CD8a hinge and transmembrane domains, a CD28 costimulatory domain, and a CD3ζ activation domain (Hu19-CD828Z) (Clinical Trial: NCT02659943; Brudno et al. Safety and feasibility of anti-CD19 CAR T cells with fully human binding domains in patients with B-cell lymphoma. Nat. Med. 26, 270-280 (2020)). The CAR was expressed using an EF-1α promoter and a bovine growth hormone polyadenylation (bgh-PolyA) signal and was inserted into the AAVS1 safe-harbour site using CRISPR-MAD7. Along with the CAR T cells, a control (CTRL) T cell culture was established from the same Pan T cell isolate, which were exposed to the same engineering treatment as the CAR T cells but without insertion of the CAR construct. The co-cultures were examined at different gatings (alive, CD3+, CAR+, CAR−) by flow cytometry after staining with anti-CD69-PE/Cy7 (BioLegend, 310912) (Y780/60), anti-CD3-perCP (BioLegend, 300326) (B690/50), anti-c-myc-Dylight488 (Abcam, ab117499) (B525/40), anti-CD19-BV785 (BioLegend, 302240) (V780/60), and the Zombie Violet Fixable Viability Kit (BioLegend, 423114) (V445/45), with the activation of CAR T cells quantified through monitoring the expression of the early activation marker CD69.

After the first response evaluation, the CAR T cells were cryopreserved in liquid nitrogen for 5 months, and then thawed and reassessed with different yeast aAPCs. Here CD69 and CD25 expression was monitored after co-cultivation of 75,000 alive CAR T cells with SCASA yeast aAPC designs P_{MFA2}-CD19 and P_{TDH3}-CD19, along with the negative control (P_{PGK1}-Empty), and a Leukocyte Activation Cocktail (LAC), with GolgiPlug (BD Biosciences, 550583) as positive control. The SCASA yeast aAPCs were here added at a target cell ratio of 0.3× (25,000 yeast cells) with and without GPCR-stimulation for increased CD19 antigen-density, by pre-cultivation of the SCASA yeast aAPCs in 0 μM and 0.1 μM α-factor in SC media for 20 hrs, respectively. The response of the entire CAR T cell culture was assessed by flow cytometry after staining with anti-CD69-PE/Cy7 (Y780/60), anti-CD25-BB700 (BD Biosciences, 566448) (B695/40), anti-CD3-FITC (BD Biosciences, 349201) (B530/30), and the Zombie Violet Fixable Viability Kit (V445/45). All experiments were run in three replicates. ANOVAs with multiple comparisons tests were used for statistical testing.

### Results

From the co-cultivation experiments, activation of donor-derived CAR T cells was observed by upregulation of CD69 for both SCASA yeast aAPCs (P_{PGK1}-CD19) and NALM6, across all target cell ratios (see FIG. 20). SCASA yeast aAPCs activated CAR T cells in a manner that resembled that of cancer cell line NALM6, hence verifying a successful simulation of cancer cells by the SCASA yeast aAPCs. Furthermore, the functionality of antigen recognition of the CAR T cell product resulting from the novel CRISPR-MAD7 method for CAR transgene insertion could be verified equally well with SCASA yeast aAPCs and NALM6 cancer cell lines (see FIG. 20-22). To further characterize CAR T cells activated by SCASA yeast aAPCs compared to NALM6, the relative increase in CD69 expression in comparison to CTRL T cell monocultures was investigated as a proxy for the intensity of CAR T cell activation (see FIG. 21), as well as the relative amounts of activated CAR T cells at population levels (% CD69+) (see FIG. 22).

One important aspect of emerging cellular immunotherapies is the ability to characterize and iteratively improve the characterization and activation of CAR T cells in a patient-centric manner. For this purpose, we wanted to assess whether SCASA yeast aAPCs could be used for testing CAR antigen-dependent activation of donor-derived CAR T cells that had been cryopreserved in liquid nitrogen tanks. For this purpose, the same CRISPR-MAD7-engineered CAR T cell batch was thawed and co-cultivated with SCASA yeast aAPCs and re-assessed after 5 months of cryopreservation. Here we monitored CD69 and CD25 expression after co-cultivation of 75,000 alive CAR T cells with SCASA yeast aAPC designs P_{MFA2}-CD19 and P_{TDH3}-CD19, along with the negative control (P_{PGK1}-Empty), and a lymphocyte activation cocktail (LAC) as positive control. We assessed the SCASA yeast aAPCs at a target cell ratio of 0.3× (25,000 yeast cells) with and without GPCR-stimulation for increased CD19 antigen-density, by pre-cultivation of the SCASA yeast aAPCs in 0 μM and 0.1 μM α-factor for 20 hrs, respectively. The response of the entire CAR T cell culture was assessed by staining for alive CD3+ T cells.

In summary, these results indicate that CAR T cells could still be activated with SCASA yeast aAPCs after cryopreservation, and that higher CD19 antigen-densities were associated with increased expression of CD69 and CD25.

In addition to the antigen-specific effects, the experimental set-up allowed assessment of CAR T cell activation at different cell-to-cell ratios of immune cells to SCASA yeast aAPCs. Here it was found that increasing the ratio of SCASA yeast aAPC significantly increased both the population size of activated CAR T cells (p<0.0001) and the intensity of their activation (p<0.0001) (see FIG. 21-22). For SCASA yeast aAPCs, the populations of activated CAR T cells could be increased from 63.6±0.5% (0.2×) to 70.0±0.4% (1.0×, p<0.0001), and further to 84.7±0.6% (5.0×, p<0.0001) (see FIG. 22). The activation intensity of CAR T cells also increased with an increase in SCASA yeast aAPC ratio, from 13.3±0.49-fold in the 0.2×SCASA yeast aAPC co-cultures, to 20.4±0.9-fold (1.0×, p<0.0001), and further to 59.2±4.0-fold (5.0×, p<0.0001) (see FIG. 21). In comparison, the most intense activation observed by NALM6 was 44.6±1.4-fold (5.0×, p<0.0001) (see FIG. 21). Importantly, the CD19-levels of SCASA yeast aAPCs were maintained across all samples independent of the target cell ratio (see FIG. 25), and consequently the changes in activation could be attributed only to changes in the target cell ratios. Hence, CD19 SCASA yeast aAPCs activated CAR T cells in a cell-to-cell ratio dependent manner, highlighting that per CAR T cell a higher number of SCASA yeast aAPCs with a fixed amount CD19 provided i) activation of more CAR T cells, and i) higher intensity of activation (CD69 expression). The same significant increase in CAR T cell activation was seen for increasing ratios of NALM6 (p<0.0001), albeit with the most intense relative activation observed from the co-culture experiment was with SCASA yeast aAPC P_{PGK1}-CD19 at a 5.0× cell-to-cell ratio (see FIG. 21-22).

While the overall activation pattern of CAR T cells based on cell-to-cell ratio was similar between SCASA yeast aAPCs and cancer cell line NALM6, differences in the population sizes of activated CAR T cells and their intensity of activation were observed. These differences were most likely caused by a combination of two key differences between the target cells, namely that i) NALM6 had a higher CD19 antigen-density than SCASA yeast aAPC design P_{PGK1}-CD19, and ii) NALM6 cells were efficiently killed by CAR T cells, while yeast cells remained present throughout the co-cultivations. Hence, not only are yeast cells an orthogonal “canvas” for antigen displays in co-cultures between yeast and immune cells, but also resistant to the killing mechanisms of CAR T cells.

In summary, as CAR T cell activation is antigen-density dependent (see FIG. 15A) and cell-to-cell ratio dependent in relation to target cells (see FIG. 21-23), the killing of cancer cells by CAR T cells may distort the exact contribution of antigen-density to activation from target cells that disappear and change the cell-to-cell ratio during co-cultivation. The results indicate that CAR T cells recognize SCASA yeast aAPCs as cancer cells, but that CAR T cells cannot kill SCASA yeast aAPCs. The resistance of SCASA yeast aAPCs to T cell cytotoxic responses could eliminate the loss of activation caused by decreasing numbers of target cells as a result of killing, as seen for NALM6, and hence attribute the activation mainly to antigen-density. In addition, yeast does not display growth in T cell media (see FIG. 16). Therefore, SCASA yeast aAPCs numbers are more robustly maintained in CAR T cell co-cultures than cancer cell lines, where they can deliver activation signals continuously to CAR T cells dependent on their antigen-densities. Consequently, this could indicate that SCASA yeast aAPCs are more robust for antigen-density studies than cell lines that are sensitive to cytotoxic responses by T cells (see Example 7 for an extended analysis on this matter).

Next, it was assessed whether SCASA yeast aAPCs could be used for testing CAR antigen-dependent activation of donor-derived CAR T cells that had been cryopreserved in liquid nitrogen. Here, SCASA yeast aAPCs P_{MFA2}-CD19 and P_{TDH3}-CD19 could induce increased expression of CD69 and CD25 in the CAR T cell product after 20 hrs of cultivation (see FIG. 23-24). This was despite a seemingly high background activation level seen in both CD69 and CD25, possibly caused by the cryopreservation and thawing. The negative control P_{PGK1}-Empty did not increase expression of CD69 or CD25, and the activation state resembled that of CAR T cell monocultures (see FIG. 23). The intensity of activation was seemingly dependent on the antigen-density of the SCASA yeast aAPCs. P_{TDH3}-CD19 produced a stronger response in CAR T cells than P_{MFA2}-CD19 for CD69 (see FIG. 24A), corresponding to a higher CD19 antigen-density of P_{TDH3}-CD19 (see FIG. 8, 10-12). This was not evident from the CD25 expression at which the increase was more similar for P_{MFA2}-CD19 and P_{TDH3}-CD19, but in both cases higher than the negative control P_{PGK1}-Empty (see FIG. 24B). Furthermore, the CAR T cells seemed to react more intensely to SCASA yeast aAPCs that had been stimulated with 0.1 μM α-factor to increase CD19 levels (see FIG. 23-24), providing a stronger CD69 response for both P_{MFA2}-CD19 and P_{TDH3}-CD19, while the CD25 response only seemed to increase for stimulated P_{MFA2}-CD19 (see FIG. 24).

In summary, these results indicate that CAR T cells could still be activated with SCASA yeast aAPCs after cryopreservation, and that higher CD19 antigen-densities were associated with increased expression of CD69 and CD25.

### Example 6—Diversifying GPCR-Based Sensory Modules for the SCASA System

### Aim

The aim of the present example was to illustrate the modularity and diversity of the sensory module, by implementing various different GPCRs sensing different ligands for controlling the presentation of CD19.

### Materials and Methods

Constructs were generated by the methods described in Example 1. Yeast strains were cultivated by the methods described in Example 1. Yeast strains were engineered by the methods described in Example 1.

### Strain Genotype

The precursor and control DIX45 strain was described in Example 2 (see Table 7). From DIX45, two different precursor strains were created by changing the P_{PGK1}-GPA1-T_{CYC1} cassette to express alternative G_{α}-subunits of chimeric human Gpa1/GαZ and Gpa1/Gαi2 allowing for coupling other GPCRs to the PRP. From these strains, a small library was constructed of diverse GPCRs; MTNR1A, ADORA2B, ADRA2A, 5HT4B, MAM2 and CXCR4A, which covered sensing of small molecules (melatonin, adenosine, adrenaline, serotonin), peptides (P-factor), and complex proteins (CXCL12/SDF-1), respectively (see FIG. 30). These GPCRs were expressed like STE2 using the P_{CCW12} promoter and the T_{CYC1} terminator (Example 1 and 2) (see Table 8). In addition, a negative control was made by knocking out the P_{CCW12}-STE2-T_{CYC1}, cassette of DIX45 (see Table 8). In all cases did the strains retain the P_{FUS1}-Aga2-HA-PAS40-(G₄S)₃—CD19-myc-T_{CPS1} cassette, hence only diversifying the G_{α}-subunit and the GPCR.

TABLE 8
Genotype of employed yeast strains

| Strain | Genotype |
|---|---|
| DIX45 | See Table 7. |
| DIX57 | DIX45, P_{PGK1}-GPA1/GαZ-T_{CYC1}^{1,2,3} |
| DIX58 | DIX45, P_{PGK1}-GPA1/Gαi2-T_{CYC1}^{1,2,4} |
| DIX59 | DIX45, (P_{CCW12}-STE2-T_{CYC1})Δ0 |
| DIX60 | DIX45, P_{CCW12}-MAM2-T_{CYC1}^{2,5,6} |
| DIX61 | DIX57, P_{CCW12}-MTNR1A-T_{CYC1}^{2,5,7} |
| DIX62 | DIX57, P_{CCW12}-ADORA2B-T_{CYC1}^{2,5,8} |
| DIX63 | DIX57, P_{CCW12}-ADRA2A-T_{CYC1}^{2,5,9} |
| DIX64 | DIX58, P_{CCW12}-5HT4B-T_{CYC1}^{2,5,10} |
| DIX65 | DIX58, P_{CCW12}-CXCR4A-T_{CYC1}^{2,5,11} |

¹P_{PGK1} promoter sequence, encoded by the nucleotide sequence of SEQ ID NO: 11.
²T_{CYC1} terminator sequence, encoded by the nucleotide sequence of SEQ ID NO: 20.
³Chimeric human Gpa1/Gαz amino acid sequence as shown in SEQ ID NO: 32, encoded by the nucleotide sequence of SEQ ID NO: 89.
⁴Chimeric human Gpa1/Gαi2 amino acid sequence as shown in SEQ ID NO: 33, encoded by the nucleotide sequence of SEQ ID NO: 90.
⁵P_{CCW12} promoter sequence, encoded by the nucleotide sequence of SEQ ID NO: 14.
⁶Mam2 amino acid sequence as shown in SEQ ID NO: 25, encoded by the nucleotide sequence of SEQ ID NO: 82.
⁷MTNR1A amino acid sequence as shown in SEQ ID NO: 26, encoded by the nucleotide sequence of SEQ ID NO: 83.
⁸ADORA2B amino acid sequence as shown in SEQ ID NO: 27, encoded by the nucleotide sequence of SEQ ID NO: 84.
⁹ADRA2A amino acid sequence as shown in SEQ ID NO: 28, encoded by the nucleotide sequence of SEQ ID NO: 85.
¹⁰5HT4B amino acid sequence as shown in SEQ ID NO: 29, encoded by the nucleotide sequence of SEQ ID NO: 86.
¹¹CXCR4A amino acid sequence as shown in SEQ ID NO: 30, encoded by the nucleotide sequence of SEQ ID NO: 87.

### Assay and Flow Cytometry

To investigate the possibility to control the SCASA system using different types of ligands, the dose-response features of the selected GPCRs coupled to their respective G_{α}-subunits was assessed in relation to regulating CD19 expression from the P_{FUS1}-Aga2-HA-PAS40-(G₄S)₃—CD19-myc-T_{CPS1} cassette. DIX45 was included as a positive control, and DIX59 as a negative control. The strains were pre-grown in SC, whereafter, for each replicate, 50.000 cells were transferred to 300 μL volumes comprising a dilution series of respective ligands dissolved in pH-buffered synthetic defined (SD) medium (Prins & Billerbeck (2021), A buffered media system for yeast batch culture growth. Bmc Microbiology, 21(1), 127.), and stimulated for 20 hrs. (30° C., 250 r.p.m.). DIX45 and DIX59 was stimulated at pH=5 with 0 μM, 0.01 μM, and 1 μM α-factor (GenScript), DIX60 at pH=5 with 0 μM, 1 μM, and 10 μM P-factor (GenScript), DIX61 at pH=5 with 0 μM, 1 μM, and 10 μM melatonin (Sigma-Aldrich, M5250), DIX62 at pH=7 with 0 μM, 0.01 μM, and 1 μM adenosine (Sigma-Aldrich, 01890), DIX63 at pH=7 with 0 μM, 1 μM, and 10 μM clonidine hydrochloride (adrenaline agonist) (Sigma-Aldrich, C7897), DIX64 at pH=5 with 0 μM, 1 μM, and 10 μM serotonin hydrochloride (Sigma-Aldrich, H9523), and DIX65 at pH=7 by 0 μM, 100 μM, and 1000 μM NUCC-390 hydrochloride (CXCL12 agonist) (Cayman Chemical Company, 30957). After stimulation, flow cytometric measurement was done using a NovoCyte Quanteon flow cytometer (Agilent) to quantify CD19 by staining with anti-HA.11-AF647 (16B12) (BioLegend, 682404) (R667/30-H). Data represents means of median fluorescence intensity (mMFI) for three biological replicates and standard deviations hereof. ANOVA with multiple comparisons tests was conducted for all conditions.

### Results

One important component of engineered living systems is the ability to create sense-response systems that can accommodate different input signals (Cubillos-Ruiz et al. Nat Rev Drug Discov. 2021 December; 20(12):941-960). In order to make a system that can employ different ligands as controlling inputs, it was assessed whether the sensory module could comprise different heterologously expressed GPCRs. Naturally, GPCRs display exceptional diversity to sense various types of ligands, ranging from light rays and small molecules to peptides and larger complex proteins (Alexander et al. Br J Pharmacol. 2017 December; 174 Suppl 1(Suppl Suppl 1):S17-S129). GPCRs often sense in a titratable manner, allowing for concentration-dependent transduction of the signal input to provide an output of regulated intensity, hence constituting suitable sensory modules for the system. Concentration-dependent control of CD19 density was possible for all tested GPCRs using the respective cognate ligands, with all strains showing significant increases in CD19 (see FIG. 26A). The GPCRs provided significantly different CD19 expression profiles at different operational ranges from the same P_{FUS1}-Aga2-HA-PAS40-(G₄S)₃-CD19-myc-T_{CPS1} cassette, emphasizing that the specific sensory module also functions as a mechanism to diversify and program the intensities of signal molecules (see FIG. 26). In the tested ranges, Ste2 could provide the relatively highest level of CD19, followed by Mam2, ADORA2B, MTNR1A, ADRA2A, 5HT4b, and finally CXCR4a (see FIG. 26B) and different receptors also provided different background levels of CD19 expression (see FIG. 26). Hence, the fold change CD19 expression varied for each receptor, with Ste2 having the highest (312.5±50.0-fold), followed by Mam2 (249.4±44.9-fold), 5HT4b (202.6±13.8-fold), MTNR1A (79.2±5.9-fold), ADRA2A (17.6±0.9-fold), ADORA2B (12.2±1.2-fold), and lastly CXCR4a (1.6±0.1-fold) (see FIG. 26C). In summary, these results show that the sensory module is customizable by expression of heterologous GPCRs enabling a myriad of specific signal inputs for the system. For example, it allows the use of ligands orthogonal to mammalian cells to shield these from any undesirable phenotypes (e.g., fungal mating peptides), or clinically relevant molecules to sense applied drugs (e.g., serotonin) for combinatorial changes to the phenotype, or even signals emitted from the mammalian cells themselves (e.g., chemokines) to enable feedback regulation of the SCASA system.

### Example 7—SCASA Yeast aAPCs as Robust Clinically Relevant CAR T Cell Activators

### Aim

The aim of the present example was to illustrate the clinical relevance and compatibility of applying SCASA yeast aAPCs in characterizing CAR T cell products by evaluating response profiles of CAR T cells after SCASA yeast aAPC co-cultivation.

### Materials and Methods

T cells were isolated from PBMCs from buffy coats collected at the central blood bank at Rigshospitalet (Copenhagen, Denmark), analogously to as described in Example 4, using SepMate-50 (IVD) tubes (Stemcell Technologies, 85460), Lymphoprep (Stemcell Technologies, 07811), and Dulbecco's Phosphate Buffered Saline with 2% Fetal Bovine Serum (Stemcell Technologies, 07905), according to manufacturer's protocol. CAR T cells were generated by applying a novel CRISPR-MAD7 method for CAR transgene insertion via electroporation into the isolated T cells from a healthy donor (Mohr et al. The CRISPR-Cas12a Platform for Accurate Genome Editing, Gene Disruption, and Efficient Transgene Integration in Human Immune Cells. ACS Synth. Biol. 12, 375-389 (2023)). The activation of CAR T cells was assayed after 20 hrs co-cultivation with a SCASA yeast aAPC (P_{PGK1}-CD19), the negative control SCASA yeast aAPC (P_{PGK1}-Empty) (strains presented in Example 2; see Table 7), and the benchmark cancer cell line NALM6 (described in Example 2) under three different co-cultivation ratios; 0.2×, 1.0×, and 5.0× target cells to 100,000 alive CAR T cells. The employed CAR was an anti-CD19 CAR with fully-human scFv, containing CD8a hinge and transmembrane domains, a CD28 costimulatory domain, and a CD3ζ activation domain (Hu19-CD828Z) (Clinical Trial: NCT02659943; Brudno et al. Safety and feasibility of anti-CD19 CAR T cells with fully human binding domains in patients with B-cell lymphoma. Nat. Med. 26, 270-280 (2020)). The CAR was expressed using an EF-1α promoter and a bovine growth hormone polyadenylation (bgh-PolyA) signal and was inserted into the AAVS1 safe-harbour site using CRISPR-MAD7. Along with the CAR T cells, a control (CTRL) T cell culture was established from the same Pan T cell isolate, which were exposed to the same engineering treatment as the CAR T cells but without insertion of the CAR construct. The co-cultures were examined at different gatings (alive, CD3+, CAR+, CAR−) by flow cytometry after staining with anti-CD69-PE/Cy7 (BioLegend, 310912) (Y780/60), anti-CD3-perCP (BioLegend, 300326) (B690/50), anti-c-myc-Dylight488 (Abcam, ab117499) (B525/40), anti-CD19-BV785 (BioLegend, 302240) (V780/60), and the Zombie Violet Fixable Viability Kit (BioLegend, 423114) (V445/45), with the activation of CAR T cells quantified through monitoring the expression of the early activation marker CD69.

After the first response evaluation, the CAR T cells were cryopreserved in liquid nitrogen for 5 months, and then thawed and reassessed with different yeast aAPCs. Here CD69 and CD25 expression was monitored after co-cultivation of 75,000 alive CAR T cells with SCASA yeast aAPC designs P_{MFA2}-CD19 and P_{TDH3}-CD19, along with the negative control (P_{PGK1}-Empty), and a Leukocyte Activation Cocktail (LAC), with GolgiPlug (BD Biosciences, 550583) as positive control. The SCASA yeast aAPCs were here added at a target cell ratio of 0.3× (25,000 yeast cells) with and without GPCR-stimulation for increased CD19 antigen-density, by pre-cultivation of the SCASA yeast aAPCs in 0 μM and 0.1 μM α-factor in SC media for 20 hrs, respectively. The response of the entire CAR T cell culture was assessed by flow cytometry after staining with anti-CD69-PE/Cy7 (Y780/60), anti-CD25-BB700 (BD Biosciences, 566448) (B695/40), anti-CD3-FITC (BD Biosciences, 349201) (B530/30), and the Zombie Violet Fixable Viability Kit (V445/45). All experiments were run in three replicates. ANOVAs with multiple comparisons tests were used for statistical testing.

### Results

One important aspect of advancing cellular immunotherapies is the ability to robustly characterize responses of CAR T cell products resulting from novel designs or manufacturing methods. For this purpose, we proceeded to demonstrate the relevance of applying SCASA yeast cells by characterizing a CAR T cell product from a healthy donor made using a novel CRISPR-MAD7 method for CAR transgene insertion and based on the alternative Hu19-CD8α-CD28-CD3ζ CAR that contains fully-human antigen-binding domains.

The SCASA yeast P_{PGK1}-CD19 design activated the CAR T cells in a manner that was analogous to the cancer cell line NALM6, across all examined target-to-effector cell ratios, hence verifying successful yeast-based simulation of CD19+ cancer cells towards the clinical Hu19-CD8α-CD28-CD3ζ CAR T cell product (see FIG. 20). Specifically, CAR+CD3+ T cells showed up-regulation of activation marker CD69 in co-cultures for all target cell ratios with P_{PGK1}-CD19 yeast (p<0.0001) and NALM6 (p:0.0001) (see FIG. 20-22). Meanwhile, no CAR− CD3+ T cells were activated for any target cell or condition, as also seen for the non-engineered control (CTRL) T cells (see FIG. 20-22). Hence, the CAR T cell product was functional towards CD19+ targets, as T cells activated solely upon CAR expression resulting from successful insertion via the CRISPR-MAD7 method. Most importantly, this was equally validated by the use of SCASA yeast cells or NALM6 cancer cells (see FIG. 20-22).

Furthermore, we re-tested the CAR antigen-dependent activation of the donor-derived CAR T cells after cryopreservation in liquid nitrogen tanks. The same CRISPR-MAD7-engineered CAR T cell batch was thawed and co-cultivated with SCASA yeast cells and re-assessed after 5 months of cryopreservation. Here we monitored CD69 and CD25 expression after cocultivation of 75,000 alive CAR T cells with SCASA yeast cell designs P_{MFA2}-CD19 and P_{TDH3}-CD19, along with the negative control (P_{PGK1}-Empty), and a lymphocyte activation cocktail (LAC) as positive control. We assessed the SCASA yeast cells at a target cell ratio of 0.3× (25,000 yeast cells) with and without GPCR-stimulation for increased CD19 antigen density, by pre-cultivation of the SCASA yeast cells in 0 μM and 0.1 μM α-factor for 20 hrs, respectively. The response of the entire CAR T cell culture was assessed by staining for alive CD3+ T cells. For the SCASA yeast cell designs P_{MFA2}-CD19 and P_{TDH3}-CD19, up-regulation of CD69 and CD25 was detected after 5 months of cryopreservation in liquid nitrogen (see FIG. 23-24). P_{TDH3}-CD19 produced a stronger response in CAR T cells than P_{MFA2}-CD19 for CD69 (see FIG. 24), corresponding to a higher CD19 antigen-density of P_{TDH3}-CD19 (see FIG. 10-12), however this was not evident from the CD25 expression at which the increase was similar (see FIG. 24). Furthermore, the CAR T cells indicated to react more intensely to SCASA yeast cells that had been stimulated with 0.1 μM α-factor to increase CD19 levels (see FIG. 23-24), providing a stronger CD69 response for both P_{MFA2}-CD19 and P_{TDH3}-CD19, while the CD25 response only seemed to increase for stimulated P_{MFA2}-CD19. In summary, these results indicate that CAR T cells can still be activated with SCASA yeast cells after cryopreservation, and that higher CD19 antigen-densities were associated with increased expression of CD69 and CD25.

Having confirmed that CAR T cell products could be verified with SCASA yeast, we next sought to investigate the efficiency and robustness of the activation. Here, we found that increasing the ratio of SCASA yeast cells per CAR T cell intensified CAR activation, with significantly increased CD69 expression levels per CAR T cell, rising from 13.3±0.5-fold to 59.2±4.0-fold the level of CTRL T cells (p<0.0001) (see FIG. 21), as well as enlargement of the population of activated CAR T cells (% CD69+) from 63.6±0.5% to 84.7±0.6% (p<0.0001)(see FIG. 22). A similar behavior was seen for the response to NALM6 at increasing ratios, however with significant differences to SCASA yeast, as NALM6 provided lower or equal intensity of CAR activation, spanning from 6.1±0.3-fold to 44.6±1.4-fold CD69 expression (p<0.0001) (see FIG. 21), and had a larger spread in the number of activated CAR T cells, spanning from 50.3±0.9% to 91.9±0.3% (p<0.0001)(see FIG. 22). Overall, functional antigen recognition and activation of the CAR T cell product could be verified both using SCASA yeast and NALM6, with a shared conclusion that CAR T cells are activated in a cell-to-cell ratio-dependent manner in relation to the amount of activated CAR T cells and their response intensity.

Interestingly, P_{PGK1}-CD19 SCASA yeast cells could activate CAR T cells more intensely than NALM6 (see FIG. 21), yet had a CD19 density of merely 23.35±1.65% compared to that of NALM6, which led us to investigate the target cells' performance and robustness throughout co-cultivation. Here, we discovered that NALM6 CD19 levels were highly unstable throughout co-cultivation, evidenced by the occurrence of a CAR T cell-resistant CD19^{low} relapse-like phenotype of the NALM6 cancer cells during co-cultivation, as also observed by others (see FIG. 29A). The appearance of the drastic CD19^{low} NALM6 phenotype was fully dependent on CAR expression (SV=98.2%, p<0.0001), and hence developed only in CAR T cell co-cultivations, while being undetectable in NALM6 monocultures and in co-cultivations with CTRL T cells (see FIG. 29A). Specifically, NALM6 CD19 levels dropped 98.54±0.05% within 20 hrs of co-cultivation to a level that was 55.4±2.2-fold lower than in the CTRL co-cultivation (p<0.0001) (see FIG. 29C), resulting in a large alive CD19^{low} population (19.8±0.9% of all cells detected) for the 5.0× co-cultivation with CAR T cells. When equal amounts of NALM6 and CAR T cells were co-cultivated the effect was less drastic (29.0±1.9-fold lower CD19 levels) (see FIG. 29C), resulting in killing of most NALM6 cells (0.5±0.1% of all cells detected). When CAR T cells were in excess, all NALM6 cells were killed. Comparatively, SCASA yeast cells showed up to 26.5-fold more robust CD19 density presentation than NALM6 throughout co-cultivations (see FIG. 29C), with only 2.1±0.1-fold to 2.8±0.1-fold differences in CD19 levels between CTRL and CAR T cell co-cultivations (see FIG. 29C). Additionally, CD19+ SCASA yeast cell numbers were up to 17.0-fold more robustly maintained during CAR T cell co-cultivations than observed for NALM6 cells (p<0.0001) (see FIG. 29B), and there was no indication that SCASA yeast cells were killed by the CAR T cell cytotoxic response, contrary to NALM6. Hence, the difference in CAR T cell activation patterns could be explained by a higher robustness in both antigen density and cell numbers of SCASA yeast cells compared to NALM6, allowing yeast to be a more consistent target cell population to continuously provide activation signals to the CAR T cells.

Upon further inspection of the CAR T cells, it became apparent that CAR expression patterns and the amount of CAR+ cells per CD3+ T cells were alternating throughout co-cultivation in a CD19-dependent manner (see FIG. 29D-E). CAR expression was elevated at low and equal target-to-effector cell ratios (p<0.0001), while target cell outnumbering of CAR T cells was associated with lower CAR expression intensities (p<0.0001) (see FIG. 29E). This antigen-dependent up- and down-modulation of CAR surface levels has previously been shown to be dependent on CAR stimulation levels, and reported to be caused by at least CAR expression regulation and CAR internalization. The % CAR+ of CD3+ T cells dropped with an increase in target cell numbers (p<0.0001), indicating a loss of CAR+ T cells (see FIG. 29D). Importantly, these effects were seen for both SCASA yeast cells and NALM6, but were in both cases more pronounced for NALM6 (see FIG. 29D-E).

Orthogonality was confirmed by the CD19^{neg} yeast control (P_{PGK1}-Empty), which did not activate CAR T cells or CTRL T cells under any circumstances, showing no increase in CD69 or CD25 (see FIG. 20-24), did not affect CAR expression intensity (see FIG. 29E), or change the number % CAR+ cells per CD3+ cells (see FIG. 29D), collectively showing that the activation of CAR T cells by SCASA yeast cells was specifically caused by the interaction between surface-displayed CD19 and successfully expressed CARs, and that yeast could not activate CD3+ T cells unspecifically, corroborating the CAR Jurkat NFAT-Luc observations (see FIG. 15).

In summary, the method disclosed herein using SCASA yeast cells provided an efficient method for activating and confirming functionality of a novel donor-derived CAR T cell product. SCASA yeast cells perform on par with cancer cells in terms of CAR T cell response dynamics, yet with higher robustness and consistency in regard to antigen density and target cell numbers throughout co-cultivation compared to cancer cells.

### Example 8—Assaying Different CAR T Cell Designs Using SCASA Yeast Cells

### Aim

The aim of the present example was to illustrate that methods disclosed herein using the SCASA yeast cells can be used to assay and characterize different cell products, such as CAR T cell designs. It is a demonstration of practical applicability.

### Materials and Methods

### CAR TPR Jurkat Cell Lines

Reporter cell lines for CAR T cell activation were generated to enable the characterization of responses resulting from different CAR designs. In this example, two different co-stimulatory domains was employed in the CAR designs; CD28 and 4-1BB. To allow easy, direct, and high-throughput screening of CAR T cell activation, a Triple Parameter Reporter Jurkat 76 T cell lines (TPR Jurkat cells) was used as the base cell line (Jutz et al. 2016, Assessment of costimulation and co-inhibition in a triple parameter T cell reporter line: Simultaneous measurement of NF-κB, NFAT and AP-1. Journal of immunological methods, 430, 10-20. https://doi.org/10.1016/j.jim.2016.01.007). The TPR Jurkat cells contains three reporter genes; NF-κB-CFP, NFAT-eGFP, and AP-1-mCherry. The two CARs, FMC63-CD8α-4-1BB-CD3ζ and FMC63-CD28-CD28-CD3ζ, were inserted as described in Example 3.

### Target Cells: Yeast Strains and NALM6

The applied SCASA yeast cell designs have been previously described in Example 2 (see Table 7); DIX44 (P_{pGK1}-Empty), DIX45 (P_{FUS1}-CD19), DIX48 (P_{MFA2}-CD19), DIX47 (P_{PGK1}-CD19), DIX49 (P_{TDH3}-CD19). Another strain was generated for displaying CD19 with regulation by orthogonal GPCR signalling, using the same background strain as for DIX34, as described in Example 1. This strain had Ste12 knocked out (ste12A0), a synthetic transcription factor inserted (P_{RAD27}-LexA-Ste12PRD-T_{ENO1}). In addition, Aga1 was overexpressed (P_{TDH3}-AGA1-T_{ADH1}) and native AGA2 deleted (aga2A0), as described in Example 2, and a CD19 cassette was inserted with a responsive synthetic promoter: P_{6xLexOLEU2}-Aga2-HA-PAS40-(G4S)3-CD19-myc-T_{CPS1}. This new strain was named DIX67 (See Table 9). The human CD19+ NALM6 B cell leukemia cell line (NALM6) was included as a control, also previously described in Example 2.

TABLE 9
Genotype of employed yeast strains

| Strain | Genotype |
|---|---|
| DIX41 | See table 7. |
| DIX67 | DIX41, ste12Δ0, P_{RAD27}-LexA-Ste12PRD-T_{ENO1} ^{1, 2, 3}, |
| | P_{6xLexOLEU2}-Aga2-HA-PAS40-(G₄S)₃-CD19-myc-T_{CPS1} ^{4, 5, 6} |

¹ P_{RAD27} promoter sequence, encoded by the nucleotide sequence of SEQ ID NO: 18.
² LexA-Ste12PRD amino acid sequence as shown in SEQ ID NO: 37, encoded by the nucleotide sequence of SEQ ID NO: 94.
³ T_{ENO1} terminator sequence, encoded by the nucleotide sequence of SEQ ID NO: 22.
⁴ P_{6xLexOLEU2} promoter sequence, encoded by the nucleotide sequence of SEQ ID NO: 15.
⁵ Full Aga2p-HA-PAS40-(G4S)3-CD19-myc CDS sequence as shown in SEQ ID NO: 65, encoded by the nucleotide sequence of SEQ ID NO: 122.
⁶ T_{CPS1} terminator sequence, encoded by the nucleotide sequence of SEQ ID NO: 19.

### Co-Cultivation and Assay

CD28- and 4-1 BB-type CAR TPR Jurkat cells were co-cultivated with SCASA yeast cells at different cellular ratios, to control the antigen load, and different GPCR stimulation levels, to control the antigen densities. Jurkat cells and NALM6 were cultured in RPMI+10% FBS with 1% pen-strep at 37° C. with 5% CO₂. All co-cultivations were initiated with 50.000 alive CAR T cells in 200 μL RPMI+10% FBS with 1% pen-strep and lasted for 24 hrs. at 37° C. with 5% CO₂. All yeast cells were pre-grown in SC at 30° C. (250 RPM) and washed twice in 1×PBS before initiation of the co-cultures. Yeast cells were equalized to exact cell numbers before initiation of co-cultures. DIX44 (P_{PGK1}-Empty), DIX48 (P_{MFA2}-CD19), DIX47 (P_{PGK1}-CD19), DIX49 (P_{TDH3}-CD19) were co-cultivated at different target cell ratios relative to the fixed amount of CAR T cells; ×0.25, ×0.5, ×1.0, ×2.0, ×4.0, and ×8.0. For GPCR stimulation of SCASA yeast cells, the yeast cells were additionally pre-cultured in SC with 0.0001 μM, 0.001 μM, 0.01 μM, 0.1 μM, 1 μM, or no α-factor (GenScript) for 20 hrs at 30° C. (250 RPM). GPCR stimulation was implemented for DIX44 (P_{PGK1}-Empty), DIX45 (P_{FUS1}-CD19), DIX48 (P_{MFA2}-CD19), DIX49 (P_{TDH3}-CD19), and DIX67 (P_{6xLexOLEU2}-CD19). All stimulated SCASA yeast cells were co-cultivated with CAR TPR Jurkats at equal cell-cell ratios (×1.0). CAR TPR Jurkat monocultures, NALM6 co-cultivations, stimulations with Dynabeads™ Human T-Activator CD3/CD28 for T Cell Expansion and Activation (Gibco) at a ×1.0 ratio, and 1×LAC were implemented as controls. After co-cultivation, the CAR TPR Jurkat response was evaluated by quantification of the expression of reporter genes: NFAT-eGFP (B525/45), NF-κB-CFP (V525/45), and AP-1-mCherry (Y615/20) on a NovoCyte Quanteon flow cytometer (Agilent). Data represents means of median fluorescence intensity (mMFI) for three biological replicates of gated CAR TPR Jurkat cells and standard deviations hereof.

### Results

The experiment yielded insight to a variety of parameters for the dynamics of SCASA yeast-based CAR activation in relation to target cell ratios and antigen densities, as well as CAR response differences in relation to CD28- and 4-1 BB-based co-stimulatory domains. We here summarize some of the main results.

### SCASA Yeast Dynamics

Yeast itself (P_{PGK1}-Empty) did not activate NFAT (see FIG. 31A), AP-1 (see FIG. 31B), or NF-κB (see FIG. 31C) signalling at any GPCR stimulation level or any target cell ratio for either the CD28- or 4-1BB-based CARs, as evident by comparison to the CAR T cell monocultures. However, CD19+ yeast could activate both the CD28- and 4-1BB-costimulatory CARs to provide responses at similar intensities to NALM6 (see FIG. 31). This was especially pronounced for the NFAT and NF-κB signalling, but for AP-1 signalling the yeast-based activation was not as pronounced as NALM6-based activation (see FIG. 31). The exact reason why AP-1 activation tended to be lower for yeast than NALM6 is yet to be determined. In that regard, it should be noted that CAR T cell activation is not a simple single-input system, but rather a multi-input complex response with downstream signalling affected and regulated by other factors, such as the engagement of other response-modifying receptors. For example, the Dynabead- and LAC-based activation of NFAT, NF-κB, and AP-1 occur through CAR-independent mechanisms. Here, they served as positive controls for the CAR TPR Jurkat responses (see FIG. 31). In summary, CD19+ yeast could activate NFAT, NF-κB, and AP-1 signalling, which was directly caused by active YSD of CD19.

The intensities of NFAT, NF-κB, and AP-1 signalling was controllable with yeast through two different parameters: 1) single-cell antigen-density control through the choice of promoter (i.e. processing module design) and the GPCR stimulation level (i.e. sensory module), and II) total antigen-load control through modulation of the target cell ratios (see FIG. 31). Interestingly, the span of 0-1 μM α-factor stimulation for antigen-density control enabled the acquisition of the same response intensities as having 0.25×-8.0× target cells, however, without changing the cell numbers. All CD19+ SCASA yeast strains—PFUS1-CD19, PMFA2-CD19, PPGK1-CD19, PTDH3-CD19, and P6xLexOLEU2-CD19—caused CAR activation using such types of control. As previously characterized by yEGFP responses, P_{FUS1} and P6xLexOLEU2 provide similar yeast outputs. However, P6xLexOLEU2-based expression is not subject to the PRP-induced expression boost and the yeast does not change morphology due to the STE12 knock-out (ste12Δ0). These differences did not appear to affect the activation of NFAT, NF-κB, and AP-1 signalling (see FIG. 31).

### Co-Stimulatory Domains Affect CAR Activation Responses

The experiment revealed large differences in the activation dynamics of CAR T cells, which were dependent on the type of co-stimulatory domain. Generally, the CD28-based CAR enabled more intense absolute activities for NFAT (see FIG. 31A), AP-1 (see FIG. 31B), and NF-κB (see FIG. 31C), compared to 4-1BB. The mere expression of the CD28-based CAR caused higher background activation of the downstream signalling pathways compared to 4-1BB. This background activity was mostly pronounced for NF-κB (see FIG. 31C), less for AP-1 (see FIG. 31B), and the least for NFAT (see FIG. 31A).

To understand which conditions affected differences in signalling between the two CAR designs, we examined the fold difference in responses between CD28 and 4-1 BB for each investigated condition (see FIG. 32). It became apparent that CD28 co-stimulation generally causes higher NF-κB activity than 4-1 BB co-stimulation, even at baseline, and that this activity is further enhanced dependent on increases in antigen densities and total antigen load. Hence, NF-κB activity is dependent on the choice of co-stimulation, both at states with and without stimulation of the CAR (see FIG. 32). NFAT activity was indifferent between CD28- and 4-1 BB-co-stimulation at baseline. However, upon increase in antigen-density through GPCR stimulation of the SCASA yeast, it became evident that NFAT was more preferentially activated by CD28-costimulation. Interestingly, this effect was not as pronounced for increases in total antigen load, as controlled by target cell ratios. Overall, this indicated that NFAT activity was dependent on the choice between CD28 and 4-1BB co-stimulation, but only upon CAR engagement with the antigen and activation (see FIG. 32). Interestingly, AP-1 activity did not differ greatly between CD28 and 4-1 BB co-stimulation, and the effect was mostly constant across changes in antigen densities and total antigen load. This indicated that AP-1 activity is mostly independent of the choice between these co-stimulatory domains (see FIG. 32).

In summary, the experiment indicated that the choice between CD28- and 4-1 BB-based co-stimulatory domains in CARs affect NFAT, NF-κB, and AP-1 signalling in three different ways, and that CD28-based co-stimulation provides more sensitive and more intense CAR T cell responses than 4-1BB. Importantly, these conclusions could be derived solely using the methods of the present disclosure utilizing the SCASA yeast cells.

### Items

1. A Synthetic Cellular Advanced Signal Adapter (SCASA) system for use in modulating, manipulating, and/or influencing the phenotype of human immune cells, comprising in vitro co-culturing of:
   a) a genetically modified yeast cell which,
      i. expresses at least one receptor whose activity regulates the activity of a signalling pathway, and/or
      ii. expresses a transcription factor, whose activity is regulated by said signalling pathway,
      iii. expresses one or more signal molecule(s), which expression is/are regulated by the activity of said transcription factor, and
      wherein said signal molecule(s) is/are presented on the surface and/or secreted from said cell in response to said activity of said transcription factor, and
   b) a human immune cell, wherein said cell is modulated, manipulated and/or obtains an altered phenotype in response to said presentation and/or secretion of said signal molecule from said yeast cell.
2. The SCASA system for use according to item 1, wherein the human immune cell is selected from the group of immune cells consisting of T lymphocytes (T cells), engineered T cells, CAR T cells, Jurkat cells, B lymphocytes (B cells), Natural killer (NK) cells, Dendritic cells, Macrophages, Neutrophils, Eosinophils, Basophils, innate lymphoid cells, Mast cells.
3. The SCASA system for use according to item 1 or 2, wherein the human immune cell is selected from the group consisting of T cells, CAR T cells, and NK cells.
4. The SCASA system for use according to any of the preceding Items, wherein the genus of the yeast cell is selected from the group consisting of Saccharomyces, Schizosaccharomyces, Eurospora, Gibberella, Fusarium, Podospora, Cryphonectria, and Magnaporthe.
5. The SCASA system for use according to any of the preceding Items, wherein the yeast cell is a cell of the Saccharomyces genus.
6. The SCASA system for use according to any of the preceding Items, wherein the receptor responds to stimulation from one or more signalling stimuli selected from the group consisting of light, pH, heat, electrochemical changes, small molecules, peptides, proteins, and/or combinations thereof.
7. The SCASA system for use according to any of the preceding items, wherein the receptor is a G protein-coupled receptor (GPCR).
8. The SCASA system for use according to any of the preceding items wherein
   a) the transcription factor is Gal4 with an amino acid sequence according to SEQ ID NO: 193, or a functional homologue thereof with an amino acid sequence which is at least 80% identical to SEQ ID NO: 193, and/or
   b) Ste12 with an amino acid sequence according to SEQ ID NO: 35 or a functional homologue thereof with an amino acid sequence which is at least 80% identical to SEQ ID NO: 35, or
   c) the transcription factor is a synthetic transcription factor which comprises synthetic transcription factors containing the pheromone-responsive domain (PRD) of Ste12 with an amino acid sequence according to SEQ ID NO: 36, or a functional homologue thereof with an amino acid sequence which is at least 80% identical to SEQ ID NO: 36.
9. The SCASA system for use according to any of the preceding items, wherein the signal molecule(s) is/are selected from the group consisting of small molecules, peptides, proteins, and antibodies.
10. The SCASA system for use according to any of the preceding items, wherein the signal molecule(s) is/are selected from the group consisting of:
   a) CD19 with an amino acid sequence according to SEQ ID NO: 53, or a functional homologue thereof with an amino acid sequence which is at least 80% identical to SEQ ID NO: 53,
   b) yEGFP, with an amino acid sequence according to SEQ ID NO: 38, or a functional homologue thereof with an amino acid sequence which is at least 80% identical to SEQ ID NO: 38,
   c) anti-CD3, with an amino acid sequence according to SEQ ID NO: 59, or a functional homologue thereof with an amino acid sequence which is at least 80% identical to SEQ ID NO: 59,
   d) anti-CD28, with an amino acid sequence according to SEQ ID NO: 60, or a functional homologue thereof with an amino acid sequence which is at least 80% identical to SEQ ID NO: 60,
   e) IL-2, with an amino acid sequence according to SEQ ID NO: 54, or a functional homologue thereof with an amino acid sequence which is at least 80% identical to SEQ ID NO: 54,
   f) HER2 with an amino acid sequence according to SEQ ID NO: 55, or a functional homologue thereof with an amino acid sequence which is at least 80% identical to SEQ ID NO: 55, and
   g) PD-L1 with an amino acid sequence according to SEQ ID NO: 57, or a functional homologue thereof with an amino acid sequence which is at least 80% identical to SEQ ID NO: 57.
11. The SCASA system for use according to any of the preceding items, wherein
   a) the expression of said receptor is under control of a first promoter,
   b) the expression of said transcription factor is under control of said first, or a second promoter,
   c) the expression of said signal molecule(s) is/are under control of said first, said second, or a third promoter.
12. The SCASA system for use according to any of item 11 wherein, the first, second and/or third promoter is selected from the group of promoters consisting of P_{MFA2}, P_{MFA1}, P_{AGA2}, P_{YCL076W}, P_{IME4}, P_{HPF1}, P_{CSS1}, P_{FUS1}, P_{FIG.1}, P_{TDH3}, P_{PGK1}, P_{SNZ1}, P_{CPS1}, P_{CCW12}, P_{6xLexO-LEU2}, P_{GAL1}, P_{TEF1} and P_{RAD27} comprising or consisting of nucleic acid sequence according to any one of SEQ ID NOs: 1-18 or functional variants thereof with a nucleic acid sequence that is at least 80% identical to any one of SEQ ID NOs: 1-18.
13. The SCASA system for use according to any of the preceding items, for use in assaying the signals influencing the phenotype of human immune cells.
14. A human immune cell and/or human immune cell with a phenotype, produced by the system according to any of items 1-12, for use as a medicament.
15. A method for T cell expansion and/or differentiation, such as e.g., naïve T cell expansion and/or differentiation, engineered CAR T cell expansion and/or differentiation, and/or differentiation of immune cells, said method comprises the use of a Synthetic Cellular Advanced Signal Adapter (SCASA) system according to any of Items 1-12.

1. A method for modulating, manipulating, and/or influencing the phenotype of human immune cells, comprising in vitro co-culturing of:
a) a genetically modified yeast cell which is specifically engineered to facilitate direct intercellular signalling to a human immune cell, wherein said yeast cell comprises:
i) a modified signalling pathway comprising a receptor which facilitates and optionally regulates the intensity and/or type of direct intercellular signalling between said yeast cell and said human immune cell, and
ii) expression of one or more signal molecule(s), which expression is/are regulated by said receptor and which signal molecule(s) facilitates the direct intercellular signalling between said yeast cell and said human immune cells, and
b) a human immune cell, which is modulated, manipulated and/or obtains an altered phenotype in response to said direct intercellular signalling with said yeast cell,
wherein said method does not comprise antigen processing of the genetically modified yeast cell or the signal molecule(s) by human antigen-presenting cells.

2-23. (canceled)

24. The method according to claim 1, wherein said signal molecule is displayed on the surface of said yeast cell.

25. The method according to claim 1, wherein the human immune cell is selected from the group of immune cells consisting of T lymphocytes (T cells), engineered T cells, CAR T cells, Jurkat cells, B lymphocytes (B cells), Natural killer (NK) cells, Dendritic cells, Macrophages, Neutrophils, Eosinophils, Basophils, innate lymphoid cells, and Mast cells.

26. The method according to claim 1, wherein the human immune cell is selected from the group consisting of T cells, CAR T cells, and NK cells.

27. The method according to claim 1, wherein the genus of the yeast cell is selected from the group consisting of Saccharomyces, Eurospora, Gibberella, Fusarium, Podospora, Cryphonectria, and Magnaporthe.

28. The method according to claim 1, wherein the yeast cell is a cell of the Saccharomyces genus.

29. The method according to claim 1, wherein the receptor is a G-protein coupled receptor (GPCR).

30. The method according to claim 1, wherein the signal molecule(s) is/are selected from the group consisting of small molecules, peptides, proteins, and antibodies.

31. The method according to claim 1, wherein the signal molecule(s) is/are selected from the group consisting of:
a) CD19 with an amino acid sequence according to SEQ ID NO: 53, or a functional homologue thereof with an amino acid sequence which is at least 80% identical to SEQ ID NO: 53,
b) yEGFP, with an amino acid sequence according to SEQ ID NO: 38, or a functional homologue thereof with an amino acid sequence which is at least 80% identical to SEQ ID NO: 38,
c) anti-CD3, with an amino acid sequence according to SEQ ID NO: 59, or a functional homologue thereof with an amino acid sequence which is at least 80% identical to SEQ ID NO: 59,
d) anti-CD28, with an amino acid sequence according to SEQ ID NO: 60, or a functional homologue thereof with an amino acid sequence which is at least 80% identical to SEQ ID NO: 60,
e) IL-2, with an amino acid sequence according to SEQ ID NO: 54, or a functional homologue thereof with an amino acid sequence which is at least 80% identical to SEQ ID NO: 54,
f) HER2 with an amino acid sequence according to SEQ ID NO: 55, or a functional homologue thereof with an amino acid sequence which is at least 80% identical to SEQ ID NO: 55, and
g) PD-L1 with an amino acid sequence according to SEQ ID NO: 57, or a functional homologue thereof with an amino acid sequence which is at least 80% identical to SEQ ID NO: 57.

32. The method according to claim 1, wherein
a) the expression of said receptor is under control of a first promoter,
b) the expression of said receptor is under control of said first, or a second promoter, and
c) the expression of said signal molecule(s) is/are under control of said first, said second, or a third promoter.

33. The method according to claim 32 wherein, the first, second and/or third promoter is selected from the group of promoters consisting of P_{MFA2}, P_{MFA1}, P_{AGA2}, P_{YCL076W}, P_{IME4}, P_{HPF1}, P_{CSS1}, P_{FUS1}, P_{FIG.1}, P_{TDH3}, P_{PGK1}, P_{SNZ1}, P_{CPS1}, P_{CCW12}, P_{6xLexO-LEU2}, P_{GAL1}, P_{TEF1} and P_{RAD27} and comprising a nucleic acid sequence according to any one of SEQ ID NOs: 1-18 or functional variants thereof with a nucleic acid sequence that is at least 80% identical to any one of SEQ ID NOs: 1-18.

34. The method according to claim 1, wherein said method comprises culturing multiple different modified yeast cells and/or multiple different human immune cells.

35. An in vitro method for T cell expansion and/or differentiation comprising performing the method according to claim 1 and expanding or differentiating T cells.

36. A genetically modified yeast cell as defined in the method of claim 1.

37. A method of cell therapy comprising administering the human immune cell produced by the method of claim 1 to a subject in need thereof.

38. The method of claim 37, wherein the subject has a cancer.
